(19) United States
(12) Patent Application Publication
(10) Pub. No.: US 2026/0034215 A1
(43) Pub. Date: Feb. 5, 2026

(54) CELL THERAPIES FOR MULTIPLE SCLEROSIS

(71) Applicant: Abata Therapeutics, Inc., Watertown, MA (US)

(72) Inventors: Stephen SOFEN, Watertown, MA (US); Andrea VAN ELSAS, Boston, MA (US); Richard RANSOHOFF, Boston, MA (US); Devapregasan MOODLEY, Watertown, MA (US); Jefte DRIJVERS, Watertown, MA (US); Eugene ANTIPOV, Watertown, MA (US); Michael BIRNBAUM, Watertown, MA (US); Lawrence SCHWEITZER, Watertown, MA (US); Enoch KISUBIKA, Watertown, MA (US); Christina STRANGE, Watertown, MA (US); Fang XIA, Lexington, MA (US); Harish ADONI, Waltham, MA (US); Yanbo ZHANG, Watertown, MA (US)

(21) Appl. No.: 19/050,528

(22) Filed: Feb. 11, 2025

Related U.S. Application Data
(63) Continuation of application No. PCT/US2023/072049, filed on Aug. 10, 2023.
(60) Provisional application No. 63/397,715, filed on Aug. 12, 2022.
(60) Provisional application No. 63/412,142, filed on Sep. 30, 2022.
(60) Provisional application No. 63/415,362, filed on Oct. 12, 2022.
(60) Provisional application No. 63/417,465, filed on Oct. 19, 2022.
(60) Provisional application No. 63/432,304, filed on Dec. 13, 2022.

(51) Int. Cl. A61K 40/11; A61K 35/17; A61K 40/32; A61K 40/42; A61P 25/28; C07K 14/725; C12N 5/0783

(52) U.S. Cl. CPC A61K 40/11; A61K 35/17; A61K 40/32; A61K 40/4213; A61P 25/28; C07K 14/7051; C12N 5/0637; A61K 2239/31

(57) ABSTRACT

The present disclosure is directed to isolated cell populations comprising stable regulatory T cells and methods of producing the same.

### CROSS-REFERENCE TO RELATED APPLICATIONS

The present application is a continuation of International Application No. PCT/US2023/072049, filed Aug. 10, 2023, which claims priority to U.S. Provisional Application 63/397,715, filed Aug. 12, 2022; U.S. Provisional Application 63/412,142, filed Sep. 30, 2022; U.S. Provisional Application 63/415,362, filed Oct. 12, 2022; U.S. Provisional Application 63/417,465, filed Oct. 19, 2022; U.S. Provisional Application 63/432,304, filed Dec. 13, 2022; the contents each of which are incorporated herein by reference in their entireties.

### REFERENCE TO THE ELECTRONIC SEQUENCE LISTING

The Sequence Listing XML associated with this application is provided in XML file format and is hereby incorporated by reference into the specification. The name of the XML file containing the Sequence Listing XML is ABTH_003_04US_SeqList_ST26.xml. The XML file is 129,941 bytes, and created on Feb. 6, 2025, and is being submitted electronically via USPTO Patent Center.

### BACKGROUND

Myelin, a critical component of a functioning central nervous system (CNS), wraps and insulates nerve fibers to protect them and enable proper transmission of electrical signals. In the CNS of Multiple Sclerosis patients, myelin is damaged by the body's own immune cells, thereby exposing nerve fibers. This myelin damage disrupts neural circuits in the brain, hampers communication between the brain and the rest of the body, and makes nerves vulnerable to cell death and neurodegeneration, with long-lasting, often irreversible impact. Progressive Multiple Sclerosis is characterized by increasing clinical disability independent of acute relapsing pathology. There are no approved therapies for non-relapsing progressive MS. The human leukocyte antigen (HLA) DRB1*1501 haplotype is strongly associated with MS and there are estimated to be at least 45,000 patients in the U.S. today with the DRB1*1501 haplotype and affected with non-relapsing progressive MS. Regulatory T cells have potential for the treatment of this disease and other autoimmune diseases because they can selectively target diseased cell types and tissues, generating a local immune response via an antigen-specific mechanism.

### SUMMARY

In some aspects disclosed herein is an isolated population of cells comprising stable CD4+ T regulatory cells (T regs) derived from a subject having multiple sclerosis, wherein at least 80% of the cells are stable CD4+ T regs comprising a hypomethylated T cell specific demethylated region (TSDR) at the FOXP3 locus. In some embodiments, the cells comprise an exogenous human T cell receptor (TCR) that binds specifically to myelin basic protein (MBP) peptide complexed with a major histocompatibility complex (MHC). In some embodiments, the MBP peptide is an MBP 83-99 peptide comprising the amino acid sequence of SEQ ID NO: 61. In some embodiments, the MHC comprises HLA-DRB1*15:01. In some embodiments, the MHC further comprises HLA-DRA*01:01. In some embodiments, the exogenous TCR comprises (a) a TCRα chain variable region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and (b) a TCRβ chain variable region comprising a CDR1 comprising the amino acid sequence of SEQ ID NO: 7, a CDR2 comprising the amino acid sequence of SEQ ID NO: 8, and a CDR3 comprising the amino acid sequence of SEQ ID NO: 9. In some embodiments, the TCRα chain variable region comprises an amino acid sequence that is at least 90%, at least 95%, or at least 99% identity to SEQ ID NO: 4. In some embodiments, the TCRα chain variable region comprises or consists of SEQ ID NO: 4. In some embodiments, the TCRα chain comprises an amino acid sequence that is at least 90%, at least 95%, or at least 99% identity to SEQ ID NO: 5. In some embodiments, the TCRα chain comprises or consists of SEQ ID NO: 5. In some embodiments, the TCRα chain comprises an amino acid sequence that is at least 90%, at least 95%, or at least 99% identical to SEQ ID NO: 73. In some embodiments, the TCRα chain comprises or consists of SEQ ID NO: 73 In some embodiments, the TCRα chain comprises an amino acid sequence that is at least 90%, at least 95%, or at least 99% identity to SEQ ID NO: 91. In some embodiments, the TCRα chain comprises or consists of SEQ ID NO: 91. In some embodiments, the TCRβ chain variable region comprises an amino acid sequence that is at least 90%, at least 95%, or at least 99% identity to SEQ ID NO: 10. In some embodiments, the TCRβ chain variable region comprises or consists of SEQ ID NO: 10. In some embodiments, the TCRβ chain comprises an amino acid sequence that is at least 90%, at least 95%, or at least 99% identical to SEQ ID NO: 11. In some embodiments, the TCRβ chain comprises or consists of SEQ ID NO: 11. In some embodiments, the TCRβ chain comprises an amino acid sequence that is at least 90%, at least 95%, or at least 99% identical to SEQ ID NO: 75. In some embodiments, the TCRβ chain comprises or consists of SEQ ID NO: 75. In some embodiments, the TCRβ chain comprises an amino acid sequence that is at least 90%, at least 95%, or at least 99% identical to SEQ ID NO: 93. In some embodiments, the TCRβ chain comprises or consists of SEQ ID NO: 93. In some embodiments, the exogenous TCR is encoded as a single polypeptide comprising an amino acid sequence having at least 90%, 95%, or 100% identity to the amino acid sequence of SEQ ID NO: 17. In some embodiments, the single polypeptide comprises or consists of SEQ ID NO: 17. In some embodiments, the exogenous TCR is encoded as a single polypeptide comprising an amino acid sequence having at least 90%, 95%, or 100% identity to the amino acid sequence of SEQ ID NO: 79. In some embodiments, the single polypeptide comprises or consists of SEQ ID NO: 79. In some embodiments, the exogenous TCR is encoded as a single polypeptide comprising an amino acid sequence having at least 90%, 95%, or 100% identity to the amino acid sequence of SEQ ID NO: 97. In some embodiments, the single polypeptide comprises or consists of SEQ ID NO: 97. In some embodiments, the single polypeptide comprises the amino acid sequence of SEQ ID NO: 79 and is encoded by a nucleotide sequence having at least 90%, 95%, or 100% identity to the nucleotide sequence of SEQ ID NO: 87. In some embodiments, the exogenous TCR comprises: (a) a TCRα chain variable region comprising a CDR1 comprising the amino acid sequence of SEQ ID NO: 18, a CDR2 comprising the amino acid sequence of SEQ ID NO: 19, and a CDR3 comprising the amino acid sequence of SEQ ID NO: 20; and (B) a TCRβ chain variable region comprising a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26. In some embodiments, the TCRα chain variable region comprises an amino acid sequence that is at least 90%, at least 95%, or at least 99% identity to SEQ ID NO: 21. In some embodiments, the TCRα chain variable region comprises or consists of SEQ ID NO: 21. In some embodiments, the TCRα chain comprises an amino acid sequence that is at least 90%, at least 95%, or at least 99% identity to SEQ ID NO: 22. In some embodiments, the TCRα chain comprises or consists of SEQ ID NO: 22. In some embodiments, the TCRα chain comprises an amino acid sequence that is at least 90%, at least 95%, or at least 99% identical to SEQ ID NO: 80. In some embodiments, the TCRα chain variable region comprises or consists of SEQ ID NO: 80. In some embodiments, the TCRβ chain variable region comprises an amino acid sequence that is at least 90%, at least 95%, or at least 99% identity to SEQ ID NO: 27. In some embodiments, the TCRβ chain variable region comprises or consists of SEQ ID NO: 27. In some embodiments, the TCRβ chain comprises an amino acid sequence that is at least 90%, at least 95%, or at least 99% identical to SEQ ID NO: 28. In some embodiments, the TCRβ chain comprises or consists of SEQ ID NO: 28. In some embodiments, the TCRβ chain comprises an amino acid sequence that is at least 90%, at least 95%, or at least 99% identical to SEQ ID NO: 82. In some embodiments, the TCRβ chain comprises or consists of SEQ ID NO: 82. In some embodiments, the exogenous TCR is encoded as a single polypeptide comprising an amino acid sequence having at least 90%, 95%, or 100% identity to the amino acid sequence of SEQ ID NO: 31. In some embodiments, the single polypeptide comprises or consists of SEQ ID NO: 31. In some embodiments, the exogenous TCR is encoded as a single polypeptide comprising an amino acid sequence having at least 90%, 95%, or 100% identity to the amino acid sequence of SEQ ID NO: 86. In some embodiments, the single polypeptide comprises or consists of SEQ ID NO: 86. In some embodiments, the single polypeptide comprises the amino acid sequence of SEQ ID NO: 86 and is encoded by a nucleotide sequence having at least 90%, 95%, or 100% identity to the nucleotide sequence of SEQ ID NO: 88. In some embodiments, the stable CD4+ T regs do not express a FOXP3 protein from an engineered FOXP3 locus. In some embodiments, the TSDR is the CNS2 region of FOXP3. In some embodiments, the MHC is MHC Class I or MHC Class II. In some embodiments, at least 85%, at least 90%, at least 95%, at least 96%, at least 97%, at least 98%, or at least 99% of the cells are stable CD4+ T regs comprising a hypomethylated TSDR at an endogenous FOXP3 locus. In some embodiments, at least 80%, at least 85%, at least 90%, or at least 95% of the cells are CD4⁺CD25+CD127^{-/lo}. In some embodiments, at least 80%, at least 85%, at least 90%, or at least 95% of the cells are CD4⁺CD25+CD127^{-/lo}FOXP3+. In some embodiments, the isolated population comprises at least 4×10⁷ stable CD4+ T regs. In some embodiments, the isolated population comprises 4×10⁷ to 1×10¹⁰ stable CD4+ T regs. In some embodiments, less than 25%, less than 20%, less than 15%, less than 10%, less than 5%, less than 2%, less than 1%, less than 0.5%, less than 0.1%, or less than 0.01% of the cells in the isolated population are conventional CD4+ T cells. In some embodiments, less than 60%, less than 50%, less than 40%, less than 30%, less than 20%, less than 10%, less than 5%, or less than 1% of the conventional T cells comprise the exogenous human TCR. In some embodiments, the ratio of stable CD4+ T regs to conventional T cells in the isolated population is at least 50:1, at least 60:1, at least 70:1, at least 80:1, at least 90:1, at least 100:1, at least 500:1, at least 1000:1, or at least 10000:1. In some embodiments, less than 2%, less than 1%, less than 0.5%, less than 0.1%, or less than 0.01% of the cells of the isolated population are CD8+ T cells. In some embodiments, the isolated population does not comprise a detectable percentage of CD8+ T cells by fluorescence activated cell sorting (FACS). In some embodiments, at least 10% of the cells express the exogenous human TCR. In some embodiments, at least 15%, at least 20%, at least 30%, at least 40%, at least 50%, at least 60%, at least 70%, at least 80%, or at least 90% of the cells express the exogenous human TCR. In some embodiments, the percentage of stable CD4+ T regs comprising a hypomethylated TSDR at the FOXP3 locus decreases by 10% or less, 9% or less, 8% or less, 7% or less, 6% or less, 5% or less, 4% or less, 3% or less, 2% or less, or 1% or less following a cryopreservation freeze-thaw cycle. In some embodiments, the stable CD4+ Tregs exhibit one or more functions selected from: (a) regulatory cytokine secretion activity; (b) expression of activation markers associated with regulatory T cells; and/or (c) suppression activity. In some embodiments, the exogenous TCR is encoded as a single polypeptide, optionally wherein the polypeptide comprises an N-terminal beta domain and a C-terminal alpha domain. In some embodiments, the single polypeptide comprises a TCR alpha chain and a TCR beta chain, and wherein the polypeptide comprises a self-cleaving peptide sequence positioned between the TCR alpha chain and the TCR beta chain. In some embodiments, the self-cleaving peptide sequence is a 2A peptide sequence, optionally wherein the 2A peptide sequence is a P2A, E2A, F2A, or T2A peptide sequence. In some embodiments, the exogenous TCR comprises a TCR alpha chain comprising a constant region comprising an amino acid sequence having at least 90%, 95%, or 100% identity to the amino acid sequence of SEQ ID NO: 58. In some embodiments, the exogenous TCR comprises a TCR beta chain comprising a constant region comprising an amino acid sequence having at least 90%, 95%, or 100% identity to the amino acid sequence of SEQ ID NO: 60. In some embodiments, the exogenous human TCR comprises one or more amino acid substitutions to cysteine residues in the TCR alpha chain constant region and the TCR beta chain constant region, and wherein the cysteine residues are capable of forming one or more disulfide bonds. In some embodiments, the TCR alpha chain constant region comprises a T48C amino acid substitution relative to a TCR alpha chain constant region comprising the amino acid sequence of SEQ ID NO: 58, and wherein the TCR beta chain constant region comprises a S57C amino acid substitution relative to a TCR beta chain constant region comprising the amino acid sequence of SEQ ID NO: 59 or 60. In some embodiments, the stable CD4+ T regs have a human leukocyte antigen (HLA)-DR15 haplotype. In some embodiments, the stable CD4+ T regs comprise an HLA-DRB1*15:01 allele. In some embodiments, the stable CD4+ T regs further comprise an HLA-DRB5*01:01 allele.

In some aspects disclosed herein is a method of producing a population of cells comprising stable CD4⁺ T regulatory cells (T regs) comprising: (a) removing CD8⁺ cells, CD19⁺ cells, and optionally CD14⁺ cells from a biological sample obtained from a subject having multiple sclerosis to produce a depleted biological sample; (b) enriching the depleted biological sample for CD25⁺ cells to produce an enriched population; (c) isolating CD4⁺CD25⁺CD127^{-/lo} cells from the enriched population; (d) expanding the enriched population to produce an expanded enriched population of cells; and (e) quantifying the methylation status of a T cell specific demethylated region (TSDR) at the FOXP3 locus in the population of cells, wherein at least 80% of the cells are stable CD4⁺ T regs comprising a hypomethylated TSDR at the FOXP3 locus, thereby producing a population of cells comprising stable CD4⁺ T regs. In some embodiments, at least 85%, at least 90%, or at least 95% of the cells are stable CD4⁺ T regs comprising a hypomethylated TSDR at the FOXP3 locus. In some embodiments, the isolated population is selected for therapeutic use if the percentage of cells comprising a hypomethylated TSDR at the FOXP3 locus is 80% or greater. In some embodiments, step (c) is performed at least twice. In some embodiments, the method further comprises activating the population of cells of step (c). In some embodiments, the activating comprises culture of the population of cells with an anti-CD3 antibody and an anti-CD28 antibody. In some embodiments, a second activating step is performed between 4 and 8 days after the first activating step. In some embodiments, the cells are expanded in a culture media comprising IL-2 and TNFα. In some embodiments, the activating and expanding comprises culturing the population of cells for at least 5, 6, 7, 8, 9, 10, 11, or 12 days. In some embodiments, the activating and expanding comprises culturing the population of cells for no more than 15, 14, 13, or 12 days.

In some aspects disclosed herein is a method of producing a population of cells comprising engineered stable CD4⁺ T regs comprising: (a) removing CD8⁺ cells, CD19⁺ cells, and optionally CD14⁺ cells from a biological sample obtained from a subject having multiple sclerosis to produce a depleted biological sample; (b) enriching the depleted biological sample for CD25⁺ cells to produce an enriched population; (c) isolating CD4⁺CD25⁺CD127^{-/lo} cells from the enriched population; (d) delivering a vector comprising a nucleic acid encoding an exogenous human T cell receptor (TCR) to the isolated population of (c) to produce a population of engineered cells; (e) expanding the population of engineered cells to produce an expanded population of engineered cells; and (f) quantifying the methylation status of a T cell specific demethylated region (TSDR) at the FOXP3 locus in the expanded population of engineered cells, wherein at least 80% of the cells are stable CD4⁺ T regs comprising a hypomethylated TSDR at the FOXP3 locus. In some embodiments, at least 85%, at least 90%, or at least 95% of the cells are stable CD4⁺ T regs comprising a hypomethylated TSDR at the FOXP3 locus. In some embodiments, step (c) is performed at least twice. In some embodiments, the isolated population is selected for therapeutic use if the percentage of cells comprising a hypomethylated TSDR at the FOXP3 locus is 80%, 85%, 90%, 95%, or greater. In some embodiments of the method, further comprises activating the population of engineered cells step (c). In some embodiments, the activating comprises culture of the population of cells with an anti-CD3 antibody and an anti-CD28 antibody. In some embodiments, the activation step is performed at least twice. In some embodiments, a second activating step is performed between 4 and 8 days after the first activating step. In some embodiments, the cells are expanded in a culture media comprising IL-2 and TNFα. In some embodiments, the expanded population comprises at least at least 1×10⁷ engineered stable CD4+ T regs. In some embodiments, the methylation status of the TSDR in step (f) is assessed at least 24 hours after a cryopreservation freeze-thaw cycle. In some embodiments, the percentage of CD4⁺CD25^{+/high}CD127^{-/lo} regulatory T cells is assessed at least 24 hours after a cryopreservation freeze-thaw cycle. In some embodiments, less than 5%, less than 4%, or less than 3% of the cells of the depleted biological sample comprise CD8⁺ cells, CD19⁺ cells, and/or CD14⁺ cells. In some embodiments, wherein 2% or less of the population of cells are CD8⁺ cells. In some embodiments, 20% or less of the population of cells are conventional T cells. In some embodiments, the population of cells in step (c) comprises CD25^{high}CD45RA; cells and CD25⁺CD45RA⁺ cells and does not comprise CD25⁺CD45RA cells. In some embodiments, the isolating of (c) comprises: (i) identifying a first subpopulation of CD4⁺ cells from the enriched population of step (b); (ii) identifying a second subpopulation of CD25^{+/high}CD127^{-/lo} cells from the first subpopulation; and (iii) selecting CD25^{high}CD45RA⁻ and CD25^{+/high}CD45RA⁺ cells from the second subpopulation for isolation and excluding CD25⁺CD45RA⁻, thereby isolating the population of CD4⁺ T regs. In some embodiments, the TSDR is the CNS2 region of FOXP3. In some embodiments, the subject has a human leukocyte antigen (HLA)-DR15 haplotype. In some embodiments, the regulatory T cells comprise an HLA-DRB1*15:01 allele. In some embodiments, the subject has progressive Multiple Sclerosis or relapsing remitting multiple sclerosis. In some embodiments, the subject has primary progressive Multiple Sclerosis (PPMS) or non-relapsing progressive Multiple Sclerosis. In some embodiments, the human subject is a male subject. In some embodiments, the human subject is a female subject. In some embodiments, the delivering the vector comprising a nucleic acid encoding the exogenous human TCR comprises transducing the cell population with the vector. In some embodiments, the exogenous TCR binds to the MBP83-99 peptide complexed with MHC. In some embodiments, the exogenous TCR is the exogenous TCR defined in any one of the above aspects or embodiments. In some embodiments, the exogenous human TCR is encoded as a single polypeptide comprising a TCRα chain and a TCRβ chain. In some embodiments, the polypeptide comprises an N-terminal TCRβ chain and a C-terminal TCRα chain. In some embodiments, the polypeptide comprises a self-cleaving peptide sequence positioned between the TCRα chain and the TCRβ chain. In some embodiments, the self-cleaving peptide sequence is a 2A peptide sequence. In some embodiments, the 2A peptide sequence is a P2A, E2A, F2A, or T2A peptide sequence. In some embodiments, the vector is a viral vector. In some embodiments, the viral vector is a lentiviral vector. In some embodiments, the lentiviral vector is a VSVg pseudotyped, self-inactivating, 3^{rd} generation lentiviral vector. In some embodiments, the nucleic acid comprises a promoter operably linked to a coding sequence encoding the exogenous human TCR, optionally wherein the promoter is an EF-1 alpha promoter or an MND promoter. In some embodiments, the nucleic acid further comprises an enhancer element, optionally an optimized post-transcriptional regulatory element (oPRE) or a woodchuck hepatitis virus post-transcriptional regulatory element (WPRE), further optionally WPRE-mut6. In some embodiments, the coding sequence is codon-optimized.

In some aspects disclosed herein is an engineered polynucleotide encoding a polypeptide comprising a TCRα chain and a TCRβ chain, wherein (a) the TCRα chain comprises a variable region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and (b) the TCRβ chain comprises variable region comprising a CDR1 comprising the amino acid sequence of SEQ ID NO: 7, a CDR2 comprising the amino acid sequence of SEQ ID NO: 8, and a CDR3 comprising the amino acid sequence of SEQ ID NO: 9. In some embodiments, the TCRα chain variable region comprises an amino acid sequence that is at least 95% identical to SEQ ID NO: 4 and the TCRβ chain variable region comprises an amino acid sequence that is at least 95% identical to SEQ ID NO: 10. In some embodiments, the TCRα chain comprises an amino acid sequence that is at least 95% identical to SEQ ID NO: 73 and the TCRβ chain comprises an amino acid sequence that is at least 95% identical to SEQ ID NO: 75. In some embodiments, the polypeptide comprises an amino acid sequence that is at least 95% identical to SEQ ID NO: 79. In some embodiments, the engineered polynucleotide comprises a nucleotide sequence that is at least 95% identical to SEQ ID NO: 87.

In some aspects disclosed herein is a engineered polynucleotide encoding a polypeptide comprising a TCRα chain and a TCRβ chain, wherein (a) the TCRα chain comprises a variable region comprising a CDR1 comprising the amino acid sequence of SEQ ID NO: 18, a CDR2 comprising the amino acid sequence of SEQ ID NO: 19, and a CDR3 comprising the amino acid sequence of SEQ ID NO: 20; and (b) the TCRβ chain comprises variable region comprising a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26. In some embodiments, the TCRα chain variable region comprises an amino acid sequence that is at least 95% identical to SEQ ID NO: 21 and the TCRβ chain variable region comprises an amino acid sequence that is at least 95% identical to SEQ ID NO: 27. In some embodiments, the TCRα chain comprises an amino acid sequence that is at least 95% identical to SEQ ID NO: 80 and the TCRβ chain comprises an amino acid sequence that is at least 95% identical to SEQ ID NO: 82. In some embodiments, the polypeptide comprises an amino acid sequence that is at least 95% identical to SEQ ID NO: 86. In some embodiments, the engineered polynucleotide comprises a nucleotide sequence that is at least 95% identical to SEQ ID NO: 88.

In some aspects disclosed herein is an expression cassette comprising the polynucleotide of any one the preceding aspects or embodiments, operably linked to a promoter, optionally wherein the promoter is an EF-1 alpha promoter or an MND promoter. In some embodiments, the expression cassette comprises an enhancer element, optionally an optimized post-transcriptional regulatory element (oPRE) or a woodchuck hepatitis virus post-transcriptional regulatory element (WPRE), further optionally WPRE-mut6.

In some aspects disclosed herein is a vector comprises the expression cassette of any of the above aspects or embodiments. In some embodiments, the vector is a viral vector. In some embodiments, the viral vector is a lentiviral vector. In some embodiments, the lentiviral vector is a VSVg pseudotyped, self-inactivating, 3^{rd} generation lentiviral vector.

In some aspects disclosed herein is an engineered polypeptide comprising a human TCRα chain and a human TCRβ chain, wherein (a) the TCRα chain is at least 95% identical to SEQ ID NO: 73 and the TCRβ chain is at least 95% identical to SEQ ID NO: 75; or (b) the TCRα chain is at least 95% identical to SEQ ID NO: 80 and the TCRβ chain is at least 95% identical to SEQ ID NO: 82. In some embodiments of the engineered polypeptide wherein (a) the TCRα chain comprises or consists of SEQ ID NO: 73 and the TCRβ chain comprises or consists of SEQ ID NO: 75; or (b) the TCRα chain comprises or consists of SEQ ID NO: 80 and the TCRβ chain comprises or consists of SEQ ID NO: 82. In some embodiments, the engineered polypeptide comprises from N-terminus to C-terminus: the TCRβ chain, a self-cleaving peptide sequence, and the TCRα chain. In some embodiments, the self-cleaving peptide is a P2A sequence. In some embodiments, the engineered comprises an amino acid sequence that is at least 95% identical to SEQ ID NO: 79 or 86. In some embodiments, the engineered comprises an amino acid sequence comprising or consisting of SEQ ID NO: 79 or 86.

In some aspects disclosed herein is a pharmaceutical composition comprising the isolated population of any one of the preceding aspects or embodiments and a pharmaceutically acceptable excipient.

In some aspects disclosed herein is a pharmaceutical composition comprising the isolated population of any one of the preceding aspects or embodiments and a cryopreservative.

In some aspects disclosed herein is a method of treating multiple sclerosis in a subject in need thereof, comprising administering to a subject the pharmaceutical composition of any of the above aspects or embodiments or the isolated population of any one of the above aspects or embodiments. In some embodiments, the pharmaceutical composition is administered in an effective amount to alleviate one or more symptoms of multiple sclerosis. In some embodiments, the administering comprises intravenous administration. In some embodiments, the administering comprises one or more infusions. In some embodiments, the cells of the isolated population are autologous relative to the subject. In some embodiments, the subject has progressive Multiple Sclerosis or relapsing remitting multiple sclerosis. In some embodiments, the subject has primary progressive Multiple Sclerosis (PPMS) or non-relapsing progressive Multiple Sclerosis.

### BRIEF DESCRIPTION OF THE DRAWINGS

FIGS. 1A-1B provide an exemplary method of producing a cell population comprising stable regulatory T cells. FIG. 1A shows a schematic of the overall process of isolating regulatory T cells having a CD4⁺CD45RA⁺CD25^{+/high}CD127^{lo/-} phenotype. FIG. 1B shows data depicting a fluorescence-activated cell sorting (FACS) strategy to enrich a cell population for cells having a CD45RA⁺CD4⁺CD25⁺CD127^{lo/-} phenotype from the CD25-enriched sample to produce an isolated cell population comprising stable regulatory T cells. As is shown in FIG. 1A, this sorting strategy is employed in two consecutive steps to obtain high levels of purity.

FIG. 2 shows graphs depicting the recovery of regulatory T cells following depletion and CD25 enrichment steps in an exemplary method of producing an isolated cell population comprising stable regulatory T cells.

FIG. 3A-FIG. 3B shows data depicting relative amount of various cell phenotypes following depletion and CD25 enrichment steps in an exemplary method of producing an isolated cell population comprising stable regulatory T cells.

FIG. 4 shows graphs depicting the growth of cells following transduction with an exogenous TCR. Cells that were not transduced (“Untransduced”) are included as a control.

FIG. 5 shows graphs depicting the phenotype and TCR expression of regulatory T cells in a cell population produced by an exemplary method of the disclosure.

FIG. 6 shows data demonstrating that regulatory T cells in a cell population produced by an exemplary method of the disclosure maintain a hypomethylated T cell-specific demethylation region (TSDR).

FIG. 7 shows a graph depicting the activation of lentivirus-transduced regulatory T cells of the disclosure following a cryopreservation freeze-thaw cycle.

FIG. 8 shows graphs of the gating strategy for the purification of the CD4⁺CD45RA⁺CD127^{-/low}CD25⁺ cell population purification.

FIG. 9 shows the percent TSDR hypomethylation of different runs purified by gating strategy depicted in FIG. 8.

FIG. 10A-FIG. 10D show expression of CTLA-4 (FIG. 10A), CD69 (FIG. 10B), TGFβ-1 (FIG. 10C), and IL-10 (FIG. 10D) after incubation of the cell populations with and without anti-CD3 and anti-CD28 beads following a cryopreservation freeze-thaw cycle.

FIG. 11 shows the change in the percent TSDR hypomethylation over time for the expansion of three different MS donors.

FIG. 12A-FIG. 12C show the population doubling level (FIG. 12A and FIG. 12B) and the percent TSDR hypomethylation for CD45RA⁺ and CD45RA cells over eight days of expansion. The spike-in shows that CD45RA⁺ cells can expand within the CD45RA population.

FIG. 13A-FIG. 13C show the gating strategy for CD25+CD127−/CD45; and CD25^{+/high}CD127^{-lo} CD45⁻ selection.

FIG. 14A-FIG. 14E shows the histogram approach shown in FIG. 13A-FIG. 13C.

FIG. 15 shows the percent TSDR hypomethylation over the course of expansion for the same donor based on different sorting strategies.

FIG. 16 shows the expansion of cells after restimulating on days 6, 8, or 10.

FIG. 17 shows the effect of the fold expansion with TNF-α, a second stimulation, or both.

FIG. 18A-FIG. 18B show the viability and purity of the cells expanding by both a TNF-α and second stimulation.

FIG. 19A-FIG. 19E show an alternate histogram approach for the gating strategy shown in FIG. 13A-FIG. 13C.

FIG. 20 shows the percent TSDR hypomethylation over the course of expansion for the same donor based on different sorting strategies.

FIG. 21 shows the percent TSDR hypomethylation for three different donors based the gating strategy depicted in FIG. 13A-FIG. 13C and FIG. 19A-FIG. 19E.

FIG. 22A-FIG. 22D show the percent TSDR hypomethylation and percent FOXP3⁺ cells according to the proportion of Tregs in the initial population based on two trials.

FIG. 23 shows the proportion of FOXP3⁺ and FOXP3; that expressed IL-2 and IFN-γ post-activation with PMA and ionomycin.

FIG. 24 shows the expression of CD3, cMYC, and GFP in transduced cells.

FIG. 25A-FIG. 25C show the percent of cells that were positive for CD3 (FIG. 25A), GFP (FIG. 25B), and cMYC (FIG. 25C) after transduction with different TCR constructs.

FIG. 26 show graphs depicting the ability of lentivirus-transduced Jurkat cells to express an MPB-specific TCR at the cell surface.

FIG. 27A-FIG. 27C show graphs depicting the activation of lentivirus-transduced Jurkat cells.

FIG. 28A-FIG. 28C show graphs depicting of the activation of lentivirus-transduced conventional T cells.

FIG. 29A-FIG. 29C show graphs depicting the activation of lentivirus-transduced regulatory T cells of the disclosure.

FIG. 30A-FIG. 30D show graphs depicting the ability of lentivirus-transduced regulatory T cells of the disclosure to suppress conventional T cells.

FIG. 31A-FIG. 31C show the suppression of polyclonal conventional T cells expressing TCR-A by regulatory T cells expressing TCR-A.

FIG. 32A-FIG. 32B show graphs depicting the activation of Jurkat transduced with engineered TCRs.

FIG. 33A-FIG. 33C show graphs depicting of the activation of conventional T cells transduced with engineered TCRs.

FIG. 34A-FIG. 34C show expression data for cells expressing engineered TCRs. FIG. 18A shows flow cytometry data for Vβ2 expression and CD69 expression. FIGS. 18B-18C show quantification of Vβ2 expression in cells expressing engineered TCRs.

FIG. 35A-FIG. 35C show graphs depicting activation (FIGS. 35A-FIG. 35B) and Vβ2 expression (FIG. 35C) for Tregs expressing an engineered MBP-specific TCR. FIGS. 35A and 35B show graphs depicting activation of Tregs expressing an engineered MBP-specific TCR when contacted with BLS2b cells (B cells expressing only HLA-DRA*01:01 and HLA-DRB1*15:01) and MBP 83-99 peptide. The Tregs in FIG. 35A express TCR-E engineered v1, TCR-E engineered v2, or TCR-A. The Tregs in FIG. 35B express TCR-F engineered v1, TCR-F engineered v2, or TCR-B. Expression of an activation marker (CD69) increased with increased dose of MBP 83-99 peptide. FIG. 35C shows the proportion of Tregs expressing an MBP-specific TCR that express Vβ2 under non-stimulating conditions.

FIG. 36A-FIG. 36B show graphs depicting the activation of suppressive capacity in regulatory T cells transduced with engineered TCRs (as measured by 4-1BB expression) when contacted with BLS2b cells (B cells expressing only HLA-DRA*01:01 and HLA-DRB1*15:01) and MBP 83-99 peptide.

FIG. 37A-FIG. 37C show the ability of T cells transduced with TCR-E v2 to suppress conventional T cells.

FIG. 38 shows a graph depicting the stability of the regulatory T cells of the disclosure in the presence of pro-inflammatory cytokines. Cells were expanded in the presence of 1000 units/mL of IL-2 with a mixture of pro-inflammatory cytokines (IL-1β, IL-6, IL-12, IL-17A, IFN-β, IFN-γ, TGF-β1, and TNF-α) at a concentration of 1-10,000 μg/ml each. The percentage of regulatory T cells having CD4⁺/FOXP3⁺ expression remained constant for at least 23 days in the presence of pro-inflammatory cytokines (with a concentration of up to 1,000 μg/mL of each cytokine in the mixture).

FIG. 39A-FIG. 39B show the percent FOXP3⁺ cells and percent TSDR hypomethylation after incubation with the cytokine mixtures used in FIG. 38 (Mix-8) or with IL-1β, IL-6, IL-12, IL-17A, IFN-γ, and TNF-α (Mix-6.).

FIG. 40 provides a schematic of a human/mouse hybrid MBP-targeting T cell receptor (TCR) of the disclosure.

FIG. 41 provides an EAE study protocol.

FIG. 42 provides flow cytometry data demonstrating that human/mouse hybrid TCRs are highly expressed in murine regulatory T cells (Tregs) following transduction.

FIG. 43A provides data demonstrating that murine FOXP3-GFP⁺ donor Tregs transduced with a human/mouse hybrid TCR are enriched in the central nervous system (CNS) of recipient mice experiencing active EAE.

FIG. 43B provides data suggesting that murine FOXP3-GFP⁺ donor Tregs transduced with a human/mouse hybrid TCR are capable of affecting EAE disease severity.

### DETAILED DESCRIPTION

### Overview

The present disclosure provides methods and compositions related to the treatment of multiple sclerosis through the production and utilization of stable regulatory T cells. The present disclosure also provides methods and compositions related to the treatment of multiple sclerosis through the production and utilization of engineered stable regulatory T cells comprising myelin basic protein (MBP)-specific T cell receptor (TCR). Such engineered regulatory T cells are capable of specifically targeting discrete cell types and tissues associated with degraded myelin, for example, in order to prevent immune-mediated destruction, restore homeostasis, and promote repair in affected tissues. Additionally, the engineered regulatory T cells of the disclosure suppress local inflammation at the target and do not cause systemic immune suppression. Furthermore, the engineered regulatory T cells described herein are stable (e.g., committed to a Treg phenotype) thymically-derived regulatory T cells that are capable of persisting in vivo for extended periods and may provide therapeutic benefit for months or years following a single dose. These stable regulatory T cells are also, in some embodiments, resistant to pro-inflammatory triggers (e.g., pro-inflammatory cytokines). In some embodiments, the stable regulatory T cells of the disclosure are autologous cells, meaning they are obtained directly from a patient's own cells, engineered to express an exogenous antigen-specific TCR, and subsequently administered back into the patient. The use of autologous cells minimizes the risk of a rejection (e.g., graft-versus-host disease) by the patient.

Isolation of a highly stable and pure population of engineered regulatory T cells produced in the thymus and obtaining a sufficiently large and pure population of said engineered regulatory T cells to treat a patient has proved challenging, both because the population of stable thymic regulatory T cells is so small relative to the total population of lymphocytes, and because there are no cell surface markers that are solely associated with regulatory T cells. Although isolated polyclonal regulatory T cells have proved effective in clinical therapies, when engineering regulatory T cells with an exogenous TCR, a higher level of purity and stability are required because conventional T cells and non-stable regulatory T cells (i.e., peripheral Tregs) engineered with the exogenous TCR, in sufficient numbers, could have a deleterious effect by causing, rather than suppressing, an immune response at the site of autoimmune disease.

These stable engineered regulatory T cells and methods of production provide advantages over several current methods of producing regulatory T cells for cellular therapies. Alternative approaches have been employed to generate populations of engineered regulatory T cells, or regulatory-like T cells, but these cells have disadvantages relative to the populations of cells described herein.

One possible approach is to increase the proportion of regulatory-like T cells by driving the expression of FOXP3 via engineering of conventional T cells or induced stem cells, either via engineering the FOXP3 promoter or introducing an ectopic FOXP3 gene into the cells. FOXP3 expression confers a regulatory-like phenotype. However, FOXP3 is only one of several genes that have upregulated expression in stable regulatory T cells, and it has been shown that these regulatory-like T cells differ from natural Treg. Indeed, when CD4+ T cells were edited to exhibit a Treg-like phenotype by introducing an exogenous promoter upstream of FOXP3, the engineered cells were shown to have a reduced suppressive capacity relative to natural Treg (Buckner, Science Translational Medicine, 2022), suggesting that these cells will not exhibit the same level of efficacy as true stable regulatory T cells when administered in a clinical setting.

Conversion of conventional CD4+ T cells has also been attempted, for example, by stimulation with TGF-β and IL-2, with retinoic acid, short-chain fatty acids, and TGF-β, or with rapamycin. However, these induced regulator T cells lack epigenomic changes associated with stable regulatory T cells, especially regulatory T cell-specific demethylation at the Conserved Non-coding Sequence 2 (CNS2) of the FOXP3 gene, and hence are functionally unstable and would not retain a stable phenotype when administered in a clinical setting. Moreover, proteomic analysis has shown that these induced Treg like cells have very little overlap in protein expression profile with stable, thymic Treg, and instead, share signaling and metabolic proteins with conventional T cells (Mensink et al., Sci Rep. 2022).

Contemplated herein is a population of cells and a strategy for isolating said population of cells that comprises highly pure and stable thymic regulatory T cells engineered with an exogenous TCR at sufficient purity and in sufficient numbers to be administered to a subject in a therapeutic amount. In some embodiments, the optimal population of said regulatory T cells are CD25^{+/high}CD4⁺CD127^{-/lo}CD45RA⁺ following isolation, prior to introduction of the exogenous TCR, as this phenotype has been associated with thymic naïve regulatory T cells. In some embodiments, the optimal population of said regulatory T cells further comprises CD25^{high}CD4⁺CD127^{-/lo}CD45RA⁻ following isolation, prior to introduction of the exogenous TCR, as this phenotype has been associated with thymic antigen-experienced regulatory T cells. These cells further exhibit a stable TSDR phenotype following introduction of the exogenous TCR, indicating that there is no outgrowth of non-regulatory T cell sub-populations or loss of regulatory T cell stability (e.g., no change in cell fate-cells remain terminally differentiated).

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between and including the upper and lower ends of the range are specifically contemplated and described herein.

### Regulatory T Cells

In some embodiments, the present disclosure provides isolated populations of cells comprising stable CD4⁺ T regulatory cells derived from a subject having multiple sclerosis, as well as compositions thereof. These isolated populations may be used, for example, to treat multiple sclerosis in a subject in need thereof.

The terms “Regulatory T cell”, “T regulatory cell” and “Treg” are used interchangeably herein and refer to T cells that suppress the effector functions of other cell populations of the immune system (e.g., conventional CD4⁺ T cells, effector CD8+ T cells, antigen presenting cells, and/or granulocytes). Regulatory T cells are defined by expression of the cell surface markers CD4 and CD25, as well as expression of the transcription factor FOXP3. Further, regulatory T cells do not express, or express at low levels, the cell surface marker CD127. Therefore, regulatory T cells are characterized by the following protein expression profile: CD4⁺CD25^{high/+}CD127^{low/-}FOXP3⁺.

Throughout the present disclosure, the expression of an indicated protein by a cell or population of cells may include reference to various expression indicators such as “+” (e.g., CD4⁺), “−” (e.g., CD127⁻), “high” (e.g., CD25^{High}), “int” or “intermediate” (e.g., CD25^{int}), “low” (e.g., CD127^{low}), “High/+” (e.g., CD25^{High/+}), or “low/−” (e.g., CD127^{low/-}). Herein, the various expression indicators refer to the presence or absence of the indicated protein (e.g., “+” or “−”, respectively) or the relative level of protein expression as measured by a convention protein expression assay (e.g., flow cytometry, fluorescence active cell sorting (FACS), or Western blot). Unless otherwise indicated, protein expression as described throughout the present application is determined by FACS.

(a) A positive (+) indicator refers to a detectable level of expression of the indicated protein by FACS. A population of cells that is positive (*) for a particular protein may be further divided into populations of “low” and/or “high” subpopulations.

(b) A subpopulation of “low” cells expresses the indicated protein but at a lower level than the other cells in the population (e.g., at least 50%, at least 60%, at least 70%, at least 80%, or at least 90% lower than the expression level of the other cells in the population) or at a lower level than expression in a control cell population (e.g., relative to a CD4⁺ conventional T cell or a CD8⁺ effector T cell).

(c) A subpopulation of “high” cells expresses the indicated protein at a higher level than the other cells in the population (e.g., at least 50%, at least 60%, at least 70%, at least 80%, or at least 90% higher than the expression level of the other cells in the population) or at a higher level than expression in a control cell population (e.g., relative to a CD4⁺ conventional T cell or a CD8⁺ effector T cell).

(d) A negative (.) indicator refers to an absence of expression of the indicated protein, or an expression level of the indicated protein that is below the limit of detection for the particular detection assay (e.g., below the limit of detection for a particular fluorescent antibody and/or flow cytometer).

(e) An indicator of “−/Low” refers to a cell population comprising cells that are (−) for the indicated protein and cells that are express “low” levels of the indicated protein.

(f) An indicator of “+/High” refers to a cell population comprising cells that are (+) for the indicated protein, including cells that express a high level of the indicated protein.

In some embodiments, a regulatory T cell (e.g., a stable regulatory T cell) is CD25⁺. In some embodiments, a regulatory T cell (e.g., a stable regulatory T cell) is CD25^{+/high}. In some embodiments, a regulatory T cell (e.g., a stable regulatory T cell) is CD4⁺. In some embodiments, a regulatory T cell (e.g., a stable regulatory T cell) is CD45RA⁺. In some embodiments, a regulatory T cell (e.g., a stable regulatory T cell) is FOXP3⁺.

In some embodiments, a regulatory T cell (e.g., a stable regulatory T cell) is a CD25^{+/high}CD4⁺CD127^{-/low} cell. That is, the regulatory T cell expresses, or expresses a high level of CD25, expresses CD4, and does not express, or expresses a low level of, CD127. In some embodiments, a regulatory T cell does not express CD127. In some embodiments, a regulatory T cell (e.g., a stable regulatory T cell) is a CD25^{+/high}CD4⁺CD127^{-/low}CD45RA⁺ cell. Thus, the regulatory T cells expresses, or expresses a high level of CD25, expresses CD4 and CD45RA, and does not express, or expresses a low level of, CD127. In some embodiments, a regulatory T cell expresses FOXP3. In some embodiments, a regulatory T cell is a CD25^{+/high}CD4⁺CD127^{-/low}FOXP3⁺ cell. Thus, the regulatory T cells expresses, or expresses a high level of CD25, expresses CD4 and FOXP3, and does not express, or expresses a low level of, CD127. In some embodiments, a regulatory T cell is a CD25^{+/high}CD4⁺CD45RA+CD127^{-/low}FOXP3⁺ cell. Thus, the regulatory T cells expresses, or expresses a high level of CD25, expresses CD4, CD45RA and FOXP3, and do not express, or expresses a low level of, CD127.

In thymic regulatory T cell development, the genome organizer SATB1 (special AT-rich sequence-binding protein) binds to specific genomic sites from the CD4⁺CD8⁺ thymocyte stage to open up the chromatin and activate super-enhancers associated with many regulatory T cell signature genes such as FOXP3, IL2RA, (CD25), CTLA4, IKZF2 (HELIOS), and IFZF4 (EOS). SATB1 and MLL4 (myeloid/lymphoid or mixed-lineage leukemia 4), an enzyme involved in enhancer priming, commonly occupy the newly identified conserved enhancer region, designated conserved noncoding sequence 0 (CNSO), at the FOXP3 locus, with subsequent activation of the enhancers at CNS3 and CNS2, and then the promoter. This results in stable hypomethylation and expression of FOXP3 and other regulator T cell-associated genes, thereby resulting in a stable regulatory T cell phenotype (Piotrowska, et al.; Int J Mol Sci. 2021).

After hematopoietic development, populations of T cells are able to transition between one phenotype to another under various conditions (See e.g., Kitagawa et al, Nat Immunol. 2017 February; 18 (2): 173-183). As used herein, a “stable regulatory T cell” is a regulatory T cell that comprises a hypomethylated T cell-specific demethylation region (TSDR) at the FOXP3 locus. In some embodiments, a stable regulatory T cell is further defined by expression of CD4, CD25, and/or FOXP3. Tregs that originate in the thymus (e.g., thymic T regs) are stable and a regulatory T cell that is no longer able to transition between T cell phenotypes by virtue of a change in the methylation status of one or more loci in the FOXP3 locus is also considered stable. In contrast, peripheral Tregs, which are not stable and can be induced in response to pro-inflammatory states, do not exhibit stable hypomethylation of the TSDR region of the FOXP3 locus. Hypomethylation of a TSDR, which is an evolutionary conserved CpG-rich regulatory element of the FOXP3 gene, is associated with expression of FOXP3. A TSDR of an endogenous FOXP3 locus is hypomethylated when the methyl group from one or more methylated cytosines in the TSDR have been removed to replace the methylated cytosine(s) with cytosine. Therefore, the stability of a T regulatory cell is determined by the presence of a hypomethylated T cell-specific demethylation region (TSDR) at the FOXP3 locus.

In some embodiments, measurement of the methylation status of the TSDR of a FOXP3 locus is as described in Kressler et. al. “Targeted De-Methylation of the FOXP3-TSDR Is Sufficient to Induce Physiological FOXP3 Expression but Not a Functional Regulatory T Phenotype” Frontiers in Immunology, 7 Jan. 2021.; or Schreiber, et. al. “The Regulatory T-Specific Demethylated Region Stabilizes Foxp3 Expression Independently of NF-κB Signaling” PLOS One, Feb. 5, 2014. In some embodiments, the TSDR of the FOXP3 locus is selected from conserved noncoding sequence 0 (CNSO), CNS3, and CNS2. In some embodiments, the TSDR of the FOXP3 locus is CNS2.

In some embodiments, a stable regulatory T cell maintains a hypomethylated regulatory T cell-specific demethylation region (TSDR) at an endogenous FOXP3 locus in the presence of pro-inflammatory conditions (e.g., in presence of one or more pro-inflammatory cytokines). In some embodiments, a stable regulatory T cell comprises a hypomethylated regulatory T cell-specific demethylation region (TSDR) at an endogenous FOXP3 locus in the presence of pro-inflammatory conditions for at least 3, 4, 5, 6, 7, 8, 9, 10, 11, 12, 13, 14, 15, 20, 21, 22, 23, 24, or 25 days.

In some embodiments, a stable regulatory T cell further exhibits one or more of the following functions: (i) regulatory cytokine secretion activity (e.g., secretion of IL-10, TGFβ, and IL-35); (ii) expression or activation markers associated with regulatory T cells (e.g., expression of CD69, 4-1BB, CD25, CD71, and/or CTLA-4); and/or (iii) suppression activity (e.g., the ability of the stable regulatory T cell to suppress the activation and/or proliferation of other effector cells of the immune system).

Thus, in some embodiments, isolated cell populations provided herein comprise stable T regulatory cells comprising a hypomethylated TSDR of the FOXP3 locus, expression of CD4, CD25, and FOXP3, and/or exhibit cytokine secretion, activation, and/or suppression activity.

In some embodiments, the stable regulatory T cells of the isolated cell populations provided herein maintain a hypomethylated TSDR at the FOXP3 locus over time after isolation from a biological sample. For example, in some embodiments, the stable regulatory T cells of the isolated cell populations maintain a hypomethylated TSDR at the FOXP3 locus for at least 1, 2, 3, 4, 5, 6, 7, 8, 9, 10, 11, 12, 13, 14, 15, 16, 17, 18, 19, or 20 days after isolation from a biological sample. In some embodiments, the stable regulatory T cells maintain a hypomethylated TSDR at the FOXP3 locus for more than 5 days, more than 10 days, more than 15 days, or more than 20 days after isolation from a biological sample. In some embodiments, the stable regulatory T cells maintain a hypomethylated TSDR at the FOXP3 locus for 1-20 days, 1-10 days, 1-5 days, 5-30 days, 5-20 days, 10-40 days, or 25-50 days after isolation from a biological sample.

In some embodiments, the stable regulatory T cells of the isolated cell populations provided herein maintain a hypomethylated TSDR at the FOXP3 locus over time after transduction with a nucleic acid encoding an exogenous TCR. For example, in some embodiments, the stable regulatory T cells of the isolated cell populations maintain a hypomethylated TSDR at the FOXP3 locus for at least 1, 2, 3, 4, 5, 6, 7, 8, 9, 10, 11, 12, 13, 14, 15, 16, 17, 18, 19, or 20 days after transduction with a nucleic acid encoding an exogenous TCR. In some embodiments, the stable regulatory T cells maintain a hypomethylated TSDR at the FOXP3 locus for more than 5 days, more than 10 days, more than 15 days, or more than 20 days after transduction with a nucleic acid encoding an exogenous TCR. In some embodiments, the stable regulatory T cells maintain a hypomethylated TSDR at the locus for 1-20 days, 1-10 days, 1-5 days, 5-30 days, 5-20 days, 10-40 days, or 25-50 days after transduction with a nucleic acid encoding an exogenous TCR.

In some embodiments, the stable regulatory T cells of the isolated cell populations provided herein maintain a hypomethylated TSDR at the FOXP3 locus over time after cryopreservation. For example, in some embodiments, the isolated cell populations described herein are cryopreserved and later thawed for use and/or analysis, referred to herein as a cryopreservation freeze-thaw cycle. In such embodiments, the stable regulatory T cells of the isolated cell populations maintain a hypomethylated TSDR at the FOXP3 locus for at least 1, 2, 3, 4, 5, 6, 7, 8, 9, 10, 11, 12, 13, 14, 15, 16, 17, 18, 19, or 20 days after a cryopreservation freeze-thaw cycle. In some embodiments, the stable regulatory T cells maintain a hypomethylated TSDR at the FOXP3 locus for more than 5 days, more than 10 days, more than 15 days, or more than 20 days after a cryopreservation freeze-thaw cycle. In some embodiments, the stable regulatory T cells maintain a hypomethylated TSDR at the locus for 1-20 days, 1-10 days, 1-5 days, 5-30 days, 5-20 days, 10-40 days, or 25-50 days after a cryopreservation freeze-thaw cycle.

In some embodiments, the stable regulatory T cells of the isolated cell populations provided herein maintain a hypomethylated TSDR at the FOXP3 locus over time after administration to a subject. For example, in some embodiments, the stable regulatory T cells of the isolated cell populations maintain a hypomethylated TSDR at the FOXP3 locus for at least 1, 2, 3, 4, 5, 6, 7, 8, 9, 10, 11, 12, 13, 14, 15, 16, 17, 18, 19, or 20 days after administration to a subject. In some embodiments, the stable regulatory T cells maintain a hypomethylated TSDR at the FOXP3 locus for more than 5 days, more than 10 days, more than 15 days, or more than 20 days after administration to a subject. In some embodiments, the stable regulatory T cells maintain a hypomethylated TSDR at the FOXP3 locus for 1-20 days, 1-10 days, 1-5 days, 5-30 days, 5-20 days, 10-40 days, or 25-50 days after administration to a subject.

In some embodiments, the isolated populations described herein are autologous cell populations. The term autologous in this context refers to cells that have been obtained from the same subject to which they are subsequently administered. For example, a population of cells may be obtained from a subject, subjected to the methods described herein, and then administered to the same subject (from which the population of cells was originally obtained) to treat multiple sclerosis. In such embodiments, the population of cells administered to the subject comprise autologous regulatory T cells.

In some embodiments, the isolated populations described herein are allogenic cell populations. The term allogenic in this context refers to cells that have been obtained from one subject and then administered to another subject. For example, a population of cells may be obtained from a subject, subjected to the methods described herein, and then administered to another subject in order to treat multiple sclerosis.

In some embodiments, the isolated populations of cells described herein (e.g., isolated populations comprising stable CD4+ T regulatory cells) are isolated from a biological sample obtained from a subject diagnosed with, or suspected of having, multiple sclerosis.

### Exogenous TCRs and Engineered Regulatory T Cells

In some embodiments, the present disclosure provides an isolated population of cells comprising regulatory T cells (e.g., stable regulatory T cells) comprising an exogenous human T cell receptor (TCR) that binds to a target peptide. This binding occurs when the target peptide is complexed with a major histocompatibility complex (MHC) (e.g., MHC Class I or MHC Class II). Such populations are referred to herein as “engineered regulatory T cells” or “stable engineered regulatory T cells”.

A T cell receptor (TCR) is a transmembrane heterodimer that includes an alpha chain and beta chain linked by a disulfide bond. Within these chains are complementary determining regions (CDRs) that determine the target peptide to which the TCR will bind. TCRs activate T cells in which they reside leading to a plethora of immune responses. Antigen presenting cells digest certain proteins (antigens) and display their fragments (peptides) on major histocompatibility complexes (MHC). This peptide-MHC (pMHC) complex binds to the TCR while other co-stimulatory molecules are activated leading to T cell activation, proliferation, differentiation, apoptosis, or cytokine release.

An exogenous TCR may be any TCR that is introduced to a regulatory T cell, wherein the TCR is not endogenous to (i.e., naturally occurring in) that regulatory T cell. For example, in some embodiments, an exogenous TCR is encoded by a nucleic acid that is not endogenous to a regulatory T cell (i.e., not naturally occurring in the genome of the regulatory T cell). In some embodiments, the nucleic acid is an engineered nucleic acid, for example, a recombinant or synthetic nucleic acid.

The TCR binds specifically to a target peptide complexed with an MHC. A TCR is considered to bind “specifically” to a target peptide complexed with an MHC if the TCR has a higher binding affinity for the target peptide complexed with an MHC relative to a non-target peptide complexed with an MHC. A TCR may bind to a target peptide complexed with an MHC with a binding affinity of at least 10⁻⁴ M, 10⁻⁵ M, 10⁻⁶ M, 10⁻⁷ M, 10⁻⁸ M, 10⁻⁹ M, or 10⁻¹⁰ M (e.g., 10⁻⁴ M to 10⁻¹⁰ M). In some embodiments, a TCR is considered to bind “specifically” to a target peptide complexed with an MHC if a T cell expressing the TCR becomes activated (e.g., as assessed by increased CD69 expression) when contacted with the target peptide complexed with an MHC, or becomes more highly activated relative to a non-target peptide complexed with an MHC. In some embodiments, the peptide is presented by a cell expressing the MHC.

The exogenous TCR may be a human TCR. In some embodiments, the TCR is a TCR from a monkey, mouse, rat, or any other animal.

A target peptide may be a peptide associated with multiple sclerosis. In some embodiments, a target peptide associated with Multiple Sclerosis may be a peptide belonging to myelin basic protein (MBP), such as MBP (83-99). In some embodiments, a target peptide is a peptide that is overexpressed in a population of cells associated with multiple sclerosis relative to a control (e.g., relative to a population of cells that are not associated with multiple sclerosis).

In some embodiments, a target peptide is subject to an increased autoimmune reaction in a subject having multiple sclerosis relative to a control. In some embodiments, a target peptide is a peptide that is present at a site of autoimmune disease within a subject relative to unaffected sites within the same subject. In some embodiments, a target peptide is specifically presented by an MHC allele that is associated with the presence of the autoimmune disease in the subject.

In some embodiments, a target peptide is a peptide that is overexpressed in cells of a subject having multiple sclerosis relative to a control (e.g., relative to a healthy subject). A target peptide is considered to be overexpressed in cells (e.g., associated with multiple sclerosis) if expression of the target peptide in the cells is at least 10%, at least 15%, at least 20%, at least 25%, at least 30%, at least 40%, at least 50%, at least 60%, at least 70%, at least 80%, or at least 90% higher in the cells relative to control cells (e.g., a population of cells that are not associated with the autoimmune disease). In some embodiments, a target peptide is overexpressed in cells of a subject having multiple sclerosis if expression of the target peptide in cells of the subject having multiple sclerosis is at least 10%, at least 15%, at least 20%, at least 25%, at least 30%, at least 40%, at least 50%, at least 60%, at least 70%, at least 80%, or at least 90% higher in the subject having multiple sclerosis relative to a healthy subject (e.g., a subject that does not have the autoimmune disease).

In some embodiments, a target peptide is a peptide that is highly expressed in cells at the site of disease in a subject having multiple sclerosis relative to a control (e.g., target peptide expression in an unaffected/non-disease site in the subject). A target peptide is considered to be highly expressed in cells (e.g., associated with multiple sclerosis) at the site of disease if expression of the target peptide in the cells the site of disease is at least 10%, at least 15%, at least 20%, at least 25%, at least 30%, at least 40%, at least 50%, at least 60%, at least 70%, at least 80%, or at least 90% higher in the cells relative to control cells (e.g., cells not at the site of disease).

In some embodiments, a target peptide is a peptide that is complexed with an MHC having an HLA haplotype associated with multiple sclerosis. In some embodiments, the HLA haplotype is HLA-DRB1*15.01. In some embodiments, the HLA haplotype is HLA-DRA*01.01.

An exogenous TCR, in some embodiments, is (or is encoded as) a single polypeptide (e.g., comprising a beta chain and an alpha chain). In some embodiments, a TCR comprises an N-terminal beta chain and a C-terminal alpha chain. In other embodiments, a TCR comprises an N-terminal alpha chain and a C-terminal beta chain.

A TCR may comprise a linker domain positioned between an alpha chain and a beta chain. In some embodiments, a linker domain comprises a self-cleaving peptide sequence (e.g., a self-cleaving peptide sequence positioned between an alpha chain and a beta chain). A self-cleaving peptide sequence is a peptide sequence that induces a polypeptide to separate into two peptides using a non-classical mechanism. In some embodiments, a self-cleaving peptide sequence can induce ribosomal skipping during translation of a polypeptide. In some embodiments, a self-cleaving peptide sequence is 10-30, 10-25, 15-30, 15-25, or 18-22 amino acids in length. In some embodiments, a self-cleaving peptide sequence may be a 2A peptide sequence. A 2A peptide sequence may comprise, for example, a DXEXNPGP (SEQ ID NO: 102) amino acid motif, wherein X can be any amino acid. In some embodiments, a 2A peptide sequence is a P2A (derived from porcine teschovirus-1 2A), E2A (derived from equine rhinitis A virus), F2A (derived from foot-and-mouth disease virus), or T2A (derived from Thosea asigna virus 2A) peptide sequence. A T2A peptide sequence may comprise, for example, the amino acid sequence of EGRGSLLTCGDVEENPGP (SEQ ID NO: 103). A P2A peptide sequence may comprise, for example, the amino acid sequence of ATNFSLLKQAGDVEENPGP (SEQ ID NO: 104). An E2A peptide sequence may comprise, for example, the amino acid sequence of QCTNYALLKLAGDVESNPGP (SEQ ID NO: 105). A F2A peptide sequence may comprise, for example, the amino acid sequence of VKQTLNFDLLKLAGDVESNPGP (SEQ ID NO: 106).

In some embodiments, an exogenous TCR comprises two or more polypeptides. For example, in some embodiments, an exogenous TCR comprises a first polypeptide comprising an alpha chain and a second polypeptide comprising a beta chain.

In some embodiments, an exogenous TCR comprises one or more cysteine residues present in the alpha chain of the TCR that are capable of forming one or more disulfide bonds with one or more cysteine residues in the beta chain of the TCR. In some embodiments, an exogenous TCR comprises one or more cysteine residues present in the alpha chain constant region of the TCR that are capable of forming one or more disulfide bonds with one or more cysteine residues in the beta chain constant region of the TCR.

In some embodiments, the TCR alpha chain constant region comprises an amino acid substitution at position 48 to introduce a cysteine (e.g., T48C) relative to a TCR alpha chain constant region comprising the amino acid sequence of SEQ ID NO: 58. In some embodiments, the TCR beta chain constant region comprises an amino acid substitution at position 57 to introduce a cysteine (e.g., S57C) amino acid substitution relative to a TCR beta chain constant region comprising the amino acid sequence of SEQ ID NO: 60. In some embodiments, the TCR alpha chain constant region comprises an amino acid substitution at position 48 to introduce a cysteine (e.g., T48C) relative to a TCR alpha chain constant region comprising the amino acid sequence of SEQ ID NO: 58, and the TCR beta chain constant region comprises an amino acid substitution at position 57 to introduce a cysteine (e.g., S57C) amino acid substitution relative to a TCR beta chain constant region comprising the amino acid sequence of SEQ ID NO: 60, wherein the cysteine residue at position 48 of the alpha chain is capable of forming a disulfide bond with the cysteine residue at position 57 of the beta chain.

In some embodiments, the TCR alpha chain constant region comprises an amino acid substitution at position 45 to introduce a cysteine (e.g., T45C) relative to a TCR alpha chain constant region comprising the amino acid sequence of SEQ ID NO: 58. In some embodiments, the TCR beta chain constant region comprises an amino acid substitution at position 77 to introduce a cysteine (e.g., S77C) amino acid substitution relative to a TCR beta chain constant region comprising the amino acid sequence of SEQ ID NO: 60. In some embodiments, the TCR alpha chain constant region comprises an amino acid substitution at position 45 to introduce a cysteine (e.g., T45C) relative to a TCR alpha chain constant region comprising the amino acid sequence of SEQ ID NO: 58, and the TCR beta chain constant region comprises an amino acid substitution at position 77 to introduce a cysteine (e.g., S77C) amino acid substitution relative to a TCR beta chain constant region comprising the amino acid sequence of SEQ ID NO: 60, wherein the cysteine residue at position 45 of the alpha chain is capable of forming a disulfide bond with the cysteine residue at position 77 of the beta chain.

In some embodiments, the TCR alpha chain constant region comprises an amino acid substitution at position 10 to introduce a cysteine (e.g., Y10C) relative to a TCR alpha chain constant region comprising the amino acid sequence of SEQ ID NO: 58. In some embodiments, the TCR beta chain constant region comprises an amino acid substitution at position 17 to introduce a cysteine (e.g., S17C) amino acid substitution relative to a TCR beta chain constant region comprising the amino acid sequence of SEQ ID NO: 60. In some embodiments, the TCR alpha chain constant region comprises an amino acid substitution at position 10 to introduce a cysteine (e.g., Y10C) relative to a TCR alpha chain constant region comprising the amino acid sequence of SEQ ID NO: 58, and the TCR beta chain constant region comprises an amino acid substitution at position 17 to introduce a cysteine (e.g., S17C) amino acid substitution relative to a TCR beta chain constant region comprising the amino acid sequence of SEQ ID NO: 60, wherein the cysteine residue at position 10 of the alpha chain is capable of forming a disulfide bond with the cysteine residue at position 17 of the beta chain.

In some embodiments, the TCR alpha chain constant region comprises an amino acid substitution at position 45 to introduce a cysteine (e.g., T45C) relative to a TCR alpha chain constant region comprising the amino acid sequence of SEQ ID NO: 58. In some embodiments, the TCR beta chain constant region comprises an amino acid substitution at position 59 to introduce a cysteine (e.g., D59C) amino acid substitution relative to a TCR beta chain constant region comprising the amino acid sequence of SEQ ID NO: 60. In some embodiments, the TCR alpha chain constant region comprises an amino acid substitution at position 45 to introduce a cysteine (e.g., T45C) relative to a TCR alpha chain constant region comprising the amino acid sequence of SEQ ID NO: 58, and the TCR beta chain constant region comprises an amino acid substitution at position 59 to introduce a cysteine (e.g., D59C) amino acid substitution relative to a TCR beta chain constant region comprising the amino acid sequence of SEQ ID NO: 60, wherein the cysteine residue at position 45 of the alpha chain is capable of forming a disulfide bond with the cysteine residue at position 59 of the beta chain.

In some embodiments, the TCR alpha chain constant region comprises an amino acid substitution at position 15 to introduce a cysteine (e.g., S15C) relative to a TCR alpha chain constant region comprising the amino acid sequence of SEQ ID NO: 58. In some embodiments, the TCR beta chain constant region comprises an amino acid substitution at position 15 to introduce a cysteine (e.g., E15C) amino acid substitution relative to a TCR beta chain constant region comprising the amino acid sequence of SEQ ID NO: 60. In some embodiments, the TCR alpha chain constant region comprises an amino acid substitution at position 15 to introduce a cysteine (e.g., S15C) relative to a TCR alpha chain constant region comprising the amino acid sequence of SEQ ID NO: 58, and the TCR beta chain constant region comprises an amino acid substitution at position 15 to introduce a cysteine (e.g., E15C) amino acid substitution relative to a TCR beta chain constant region comprising the amino acid sequence of SEQ ID NO: 60, wherein the cysteine residue at position 15 of the alpha chain is capable of forming a disulfide bond with the cysteine residue at position 15 of the beta chain.

| | |
|---|---|
| TRAC | NIQNPDPAVYQLRDSKSSDKSVCLFTDFDSQTNVSQSKDSDVYITD |
| (alpha | KTVLDMRSMDFKSNSAVAWSNKSDFACANAFNNSIIPEDTFFPSPE |
| constant | SSCDVKLVEKSFETDTNLNFQNLSVIGFRILLLKVAGFNLLMTLRL |
| domain) | WSS (SEQ ID NO: 58) |
| TRBC1 | EDLNKVFPPEVAVFEPSEAEISHTQKATLVCLATGFFPDHVELSWW |
| (beta | VNGKEVHSGVSTDPQPLKEQPALNDSRYCLSSRLRVSATFWQNPRN |
| constant | HFRCQVQFYGLSENDEWTQDRAKPVTQIVSAEAWGRADCGFTSVSY |
| domain) | QQGVLSATILYEILLGKATLYAVLVSALVLMAMVKRKDF (SEQ |
| | ID NO: 59) |
| TRBC2 | EDLKNVFPPEVAVFEPSEAEISHTQKATLVCLATGFYPDHVELSWW |
| (beta | VNGKEVHSGVSTDPQPLKEQPALNDSRYCLSSRLRVSATFWQNPRN |
| constant | HFRCQVQFYGLSENDEWTQDRAKPVTQIVSAEAWGRADCGFTSESY |
| domain) | QQGVLSATILYEILLGKATLYAVLVSALVLMAMVKRKDSRG (SEQ |
| | ID NO: 60) |

In some embodiments, the TCR comprises one or more amino acid sequences as described in Table A (e.g., one or more amino acid sequences belonging to any one of TCR-A, TCR-B, TCR-C, TCR-D, TCR-E, or TCR-F). A TCR may comprise the amino acid sequence of any alpha chain CDR1, CDR2, or CDR3 as provided in Table 1. In some embodiments, the alpha chain CDR1 of the TCR is any one of SEQ ID NOs: 1, 18, 32, or 46. In some embodiments, the alpha chain CDR2 of the TCR is any one of SEQ ID NOs: 2, 19, 33, or 47. In some embodiments, the alpha chain CDR3 of the TCR is any one of SEQ ID NOs: 3, 20, 34, or 48. A TCR may comprise the amino acid sequence of any beta chain CDR1, CDR2, or CDR3 as provided in Table A. In some embodiments, the beta chain CDR1 of the TCR is any one of SEQ ID NOs: 7, 24, 38, or 52. In some embodiments, the beta chain CDR2 of the TCR is any one of SEQ ID NOs: 8, 25, 39, or 53. In some embodiments, the beta chain CDR3 of the TCR is any one of SEQ ID NOs: 9, 26, 40, or 54.

In some embodiments, the alpha chain variable region of the TCR comprises an amino acid sequence having at least 90%, 95%, or 100% identity to the amino acid sequence of SEQ ID NO: 4, 21, 35, or 49. In some embodiments, the beta chain variable region of the TCR comprises an amino acid sequence having at least 90%, 95%, or 100% identity to the amino acid sequence of SEQ ID NO: 10, 14, 27, 41, or 55. In some embodiments, the alpha chain of the TCR comprises an amino acid sequence having at least 90%, 95%, or 100% identity to the amino acid sequence of SEQ ID NO: 5, 22, 36, 50, or 73. In some embodiments, the beta chain of the TCR comprises an amino acid sequence having at least 90%, 95%, or 100% identity to the amino acid sequence of SEQ ID NO: 11, 15, 28, 42, 56, or 75.

In some embodiments, the exogenous human TCR comprises (a) a TCRα chain variable region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and (b) a TCRβ chain variable region comprising a CDR1 comprising the amino acid sequence of SEQ ID NO: 7, a CDR2 comprising the amino acid sequence of SEQ ID NO: 8, and a CDR3 comprising the amino acid sequence of SEQ ID NO: 9. In some embodiments, the exogenous human TCR comprises (a) a TCRα chain variable region comprising a CDR1 consisting of SEQ ID NO: 1, a CDR2 consisting of SEQ ID NO: 2, and a CDR3 consisting of SEQ ID NO: 3; and (b) a TCRβ chain variable region comprising a CDR1 consisting of SEQ ID NO: 7, a CDR2 consisting of SEQ ID NO: 8, and a CDR3 comprising the amino acid sequence of SEQ ID NO: 9.

In some embodiments, the TCRα chain variable region comprises an amino acid sequence that is at least 90%, at least 95%, or at least 99% identity to SEQ ID NO: 4. In some embodiments, the TCRα chain variable region comprises SEQ ID NO: 4. In some embodiments, the TCRα chain variable region consists of SEQ ID NO: 4. In some embodiments, the TCRβ chain variable region comprises an amino acid sequence that is at least 90%, at least 95%, or at least 99% identity to SEQ ID NO: 10. In some embodiments, the TCRβ chain variable region comprises SEQ ID NO: 10. In some embodiments, the TCRβ chain variable region consists of SEQ ID NO: 10.

In some embodiments, the exogenous human TCR comprises (a) a TCRα chain variable region comprising an amino acid sequence that is at least 90%, at least 95%, or at least 99% identity to SEQ ID NO: 4, and b) a TCRβ chain variable region comprising an amino acid sequence that is at least 90%, at least 95%, or at least 99% identity to SEQ ID NO: 10. In some embodiments, the exogenous human TCR comprises (a) a TCRα chain variable region comprising SEQ ID NO: 4, and b) a TCRβ chain variable region comprising SEQ ID NO: 10. In some embodiments, the exogenous human TCR comprises (a) a TCRα chain variable region consisting of SEQ ID NO: 4, and b) a TCRβ chain variable region consisting of SEQ ID NO: 10.

In some embodiments, the exogenous human TCR comprises (a) a TCRα chain comprising an amino acid sequence that is at least 90%, at least 95%, or at least 99% identity to SEQ ID NO: 5, and b) a TCRβ chain comprising an amino acid sequence that is at least 90%, at least 95%, or at least 99% identity to SEQ ID NO: 11. In some embodiments, the exogenous human TCR comprises (a) a TCRα chain comprising SEQ ID NO: 5, and b) a TCRβ chain comprising SEQ ID NO: 11. In some embodiments, the exogenous human TCR comprises (a) a TCRα chain consisting of SEQ ID NO: 5, and b) a TCRβ chain consisting of SEQ ID NO: 11.

In some embodiments, the exogenous human TCR is expressed as a single polypeptide comprising at least 90%, at least 95%, or at least 99% identity to SEQ ID NO: 17. In some embodiments, the exogenous human TCR is expressed as a single polypeptide comprising SEQ ID NO: 17. In some embodiments, the exogenous human TCR is expressed as a single polypeptide consisting of SEQ ID NO: 17.

In some embodiments, the exogenous human TCR comprises (a) a TCRα chain comprising an amino acid sequence that is at least 90%, at least 95%, or at least 99% identity to SEQ ID NO: 73, and b) a TCRβ chain comprising an amino acid sequence that is at least 90%, at least 95%, or at least 99% identity to SEQ ID NO: 75. In some embodiments, the exogenous human TCR comprises (a) a TCRα chain comprising SEQ ID NO: 73, and b) a TCRβ chain comprising SEQ ID NO: 75. In some embodiments, the exogenous human TCR comprises (a) a TCRα chain consisting of SEQ ID NO: 73, and b) a TCRβ chain consisting of SEQ ID NO: 75.

In some embodiments, the exogenous human TCR is expressed as a single polypeptide comprising at least 90%, at least 95%, or at least 99% identity to SEQ ID NO: 79. In some embodiments, the exogenous human TCR is expressed as a single polypeptide comprising SEQ ID NO: 79. In some embodiments, the exogenous human TCR is expressed as a single polypeptide consisting of SEQ ID NO: 79.

In some embodiments, the exogenous human TCR comprises (a) a TCRα chain variable region comprising a CDR1 comprising the amino acid sequence of SEQ ID NO: 18, a CDR2 comprising the amino acid sequence of SEQ ID NO: 19, and a CDR3 comprising the amino acid sequence of SEQ ID NO: 20; and (b) a TCRβ chain variable region comprising a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26. In some embodiments, the exogenous human TCR comprises (a) a TCRα chain variable region comprising a CDR1 consisting of SEQ ID NO: 18, a CDR2 consisting of SEQ ID NO: 19, and a CDR3 consisting of SEQ ID NO: 20; and (b) a TCRβ chain variable region comprising a CDR1 consisting of SEQ ID NO: 24, a CDR2 consisting of SEQ ID NO: 25, and a CDR3 comprising the amino acid sequence of SEQ ID NO: 26.

In some embodiments, the TCRα chain variable region comprises an amino acid sequence that is at least 90%, at least 95%, or at least 99% identity to SEQ ID NO: 21. In some embodiments, the TCRα chain variable region comprises SEQ ID NO: 21. In some embodiments, the TCRα chain variable region consists of SEQ ID NO: 21. In some embodiments, the TCRβ chain variable region comprises an amino acid sequence that is at least 90%, at least 95%, or at least 99% identity to SEQ ID NO: 27. In some embodiments, the TCRβ chain variable region comprises SEQ ID NO: 27. In some embodiments, the TCRβ chain variable region consists of SEQ ID NO: 27.

In some embodiments, the exogenous human TCR comprises (a) a TCRα chain variable region comprising an amino acid sequence that is at least 90%, at least 95%, or at least 99% identity to SEQ ID NO: 21, and b) a TCRβ chain variable region comprising an amino acid sequence that is at least 90%, at least 95%, or at least 99% identity to SEQ ID NO: 27. In some embodiments, the exogenous human TCR comprises (a) a TCRα chain variable region comprising SEQ ID NO: 21, and b) a TCRβ chain variable region comprising SEQ ID NO: 27. In some embodiments, the exogenous human TCR comprises (a) a TCRα chain variable region consisting of SEQ ID NO: 21, and b) a TCRβ chain variable region consisting of SEQ ID NO: 27.

In some embodiments, the exogenous human TCR comprises (a) a TCRα chain comprising an amino acid sequence that is at least 90%, at least 95%, or at least 99% identity to SEQ ID NO: 22, and b) a TCRβ chain comprising an amino acid sequence that is at least 90%, at least 95%, or at least 99% identity to SEQ ID NO: 28. In some embodiments, the exogenous human TCR comprises (a) a TCRα chain comprising SEQ ID NO: 22, and b) a TCRβ chain comprising SEQ ID NO: 28. In some embodiments, the exogenous human TCR comprises (a) a TCRα chain consisting of SEQ ID NO: 22, and b) a TCRβ chain consisting of SEQ ID NO: 28.

In some embodiments, the exogenous human TCR is expressed as a single polypeptide comprising at least 90%, at least 95%, or at least 99% identity to SEQ ID NO: 31. In some embodiments, the exogenous human TCR is expressed as a single polypeptide comprising SEQ ID NO: 31. In some embodiments, the exogenous human TCR is expressed as a single polypeptide consisting of SEQ ID NO: 31.

In some embodiments, the exogenous human TCR comprises (a) a TCRα chain comprising an amino acid sequence that is at least 90%, at least 95%, or at least 99% identity to SEQ ID NO: 80, and b) a TCRβ chain comprising an amino acid sequence that is at least 90%, at least 95%, or at least 99% identity to SEQ ID NO: 82. In some embodiments, the exogenous human TCR comprises (a) a TCRα chain comprising SEQ ID NO: 80, and b) a TCRβ chain comprising SEQ ID NO: 82. In some embodiments, the exogenous human TCR comprises (a) a TCRα chain consisting of SEQ ID NO: 80, and b) a TCRβ chain consisting of SEQ ID NO: 82.

In some embodiments, the exogenous human TCR is expressed as a single polypeptide comprising at least 90%, at least 95%, or at least 99% identity to SEQ ID NO: 86. In some embodiments, the exogenous human TCR is expressed as a single polypeptide comprising SEQ ID NO: 86. In some embodiments, the exogenous human TCR is expressed as a single polypeptide consisting of SEQ ID NO: 86.

In some embodiments, the exogenous human TCR comprises (a) a TCRα chain variable region comprising a CDR1 comprising the amino acid sequence of SEQ ID NO: 32, a CDR2 comprising the amino acid sequence of SEQ ID NO: 33, and a CDR3 comprising the amino acid sequence of SEQ ID NO: 34; and (b) a TCRβ chain variable region comprising a CDR1 comprising the amino acid sequence of SEQ ID NO: 38, a CDR2 comprising the amino acid sequence of SEQ ID NO: 39, and a CDR3 comprising the amino acid sequence of SEQ ID NO: 40. In some embodiments, the exogenous human TCR comprises (a) a TCRα chain variable region comprising a CDR1 consisting of SEQ ID NO: 32, a CDR2 consisting of SEQ ID NO: 33, and a CDR3 consisting of SEQ ID NO: 34; and (b) a TCRβ chain variable region comprising a CDR1 consisting of SEQ ID NO: 38, a CDR2 consisting of SEQ ID NO: 39, and a CDR3 comprising the amino acid sequence of SEQ ID NO: 40.

In some embodiments, the TCRα chain variable region comprises an amino acid sequence that is at least 90%, at least 95%, or at least 99% identity to SEQ ID NO: 35. In some embodiments, the TCRα chain variable region comprises SEQ ID NO: 35. In some embodiments, the TCRα chain variable region consists of SEQ ID NO: 35. In some embodiments, the TCRβ chain variable region comprises an amino acid sequence that is at least 90%, at least 95%, or at least 99% identity to SEQ ID NO: 41. In some embodiments, the TCRβ chain variable region comprises SEQ ID NO: 41. In some embodiments, the TCRβ chain variable region consists of SEQ ID NO: 41.

In some embodiments, the exogenous human TCR comprises (a) a TCRα chain variable region comprising an amino acid sequence that is at least 90%, at least 95%, or at least 99% identity to SEQ ID NO: 35, and b) a TCRβ chain variable region comprising an amino acid sequence that is at least 90%, at least 95%, or at least 99% identity to SEQ ID NO: 41. In some embodiments, the exogenous human TCR comprises (a) a TCRα chain variable region comprising SEQ ID NO: 35, and b) a TCRβ chain variable region comprising SEQ ID NO: 41. In some embodiments, the exogenous human TCR comprises (a) a TCRα chain variable region consisting of SEQ ID NO: 35, and b) a TCRβ chain variable region consisting of SEQ ID NO: 41.

In some embodiments, the exogenous human TCR comprises (a) a TCRα chain comprising an amino acid sequence that is at least 90%, at least 95%, or at least 99% identity to SEQ ID NO: 36, and b) a TCRβ chain comprising an amino acid sequence that is at least 90%, at least 95%, or at least 99% identity to SEQ ID NO: 42. In some embodiments, the exogenous human TCR comprises (a) a TCRα chain comprising SEQ ID NO: 36, and b) a TCRβ chain comprising SEQ ID NO: 42. In some embodiments, the exogenous human TCR comprises (a) a TCRα chain consisting of SEQ ID NO: 36, and b) a TCRβ chain consisting of SEQ ID NO: 42.

In some embodiments, the exogenous human TCR is expressed as a single polypeptide comprising at least 90%, at least 95%, or at least 99% identity to SEQ ID NO: 45. In some embodiments, the exogenous human TCR is expressed as a single polypeptide comprising SEQ ID NO: 45. In some embodiments, the exogenous human TCR is expressed as a single polypeptide consisting of SEQ ID NO: 45.

In some embodiments, the exogenous human TCR comprises (a) a TCRα chain variable region comprising a CDR1 comprising the amino acid sequence of SEQ ID NO: 46, a CDR2 comprising the amino acid sequence of SEQ ID NO: 47, and a CDR3 comprising the amino acid sequence of SEQ ID NO: 48; and (b) a TCRβ chain variable region comprising a CDR1 comprising the amino acid sequence of SEQ ID NO: 52, a CDR2 comprising the amino acid sequence of SEQ ID NO: 53, and a CDR3 comprising the amino acid sequence of SEQ ID NO: 54. In some embodiments, the exogenous human TCR comprises (a) a TCRα chain variable region comprising a CDR1 consisting of SEQ ID NO: 46, a CDR2 consisting of SEQ ID NO: 47, and a CDR3 consisting of SEQ ID NO: 48; and (b) a TCRβ chain variable region comprising a CDR1 consisting of SEQ ID NO: 52, a CDR2 consisting of SEQ ID NO: 53, and a CDR3 comprising the amino acid sequence of SEQ ID NO: 54.

In some embodiments, the TCRα chain variable region comprises an amino acid sequence that is at least 90%, at least 95%, or at least 99% identity to SEQ ID NO: 49. In some embodiments, the TCRα chain variable region comprises SEQ ID NO: 49. In some embodiments, the TCRα chain variable region consists of SEQ ID NO: 49. In some embodiments, the TCRβ chain variable region comprises an amino acid sequence that is at least 90%, at least 95%, or at least 99% identity to SEQ ID NO: 55. In some embodiments, the TCRβ chain variable region comprises SEQ ID NO: 55. In some embodiments, the TCRβ chain variable region consists of SEQ ID NO: 55.

In some embodiments, the exogenous human TCR comprises (a) a TCRα chain variable region comprising an amino acid sequence that is at least 90%, at least 95%, or at least 99% identity to SEQ ID NO: 49, and b) a TCRβ chain variable region comprising an amino acid sequence that is at least 90%, at least 95%, or at least 99% identity to SEQ ID NO: 55. In some embodiments, the exogenous human TCR comprises (a) a TCRα chain variable region comprising SEQ ID NO: 49, and b) a TCRβ chain variable region comprising SEQ ID NO: 55. In some embodiments, the exogenous human TCR comprises (a) a TCRα chain variable region consisting of SEQ ID NO: 49, and b) a TCRβ chain variable region consisting of SEQ ID NO: 55.

In some embodiments, the exogenous human TCR comprises (a) a TCRα chain comprising an amino acid sequence that is at least 90%, at least 95%, or at least 99% identity to SEQ ID NO: 50, and b) a TCRβ chain comprising an amino acid sequence that is at least 90%, at least 95%, or at least 99% identity to SEQ ID NO: 56. In some embodiments, the exogenous human TCR comprises (a) a TCRα chain comprising SEQ ID NO: 50, and b) a TCRβ chain comprising SEQ ID NO: 56. In some embodiments, the exogenous human TCR comprises (a) a TCRα chain consisting of SEQ ID NO: 50, and b) a TCRβ chain consisting of SEQ ID NO: 56.

TABLE A
TCR Sequences

| | TCR | | SEQ |
|---|---|---|---|
| Name | component | Sequence | ID NO: |
| TCR-A | TCRa | KTSINNL | 1 |
| | CDR1 | | |
| | TCRa | LIRSNEREK | 2 |
| | CDR2 | | |
| | TCRa | CATDTTSGTYKYIF | 3 |
| | CDR3 | | |
| | TCRa | SQQGEEDPQALSIQEGENATMNCSYKTSINNLQWYRQNSGRGLV | 4 |
| | variable | HLILIRSNEREKHSGRLRVTLDTSKKSSSLLITASRAADTASYF | |
| | chain | CATDTTSGTYKYIFGTGTRLKVLA | |
| | TCRa full | SQQGEEDPQALSIQEGENATMNCSYKTSINNLQWYRQNSGRGLV | 5 |
| | ORF | HLILIRSNEREKHSGRLRVTLDTSKKSSSLLITASRAADTASYF | |
| | | CATDTTSGTYKYIFGTGTRLKVLANIQNPDPAVYQLRDSKSSDK | |
| | | SVCLFTDFDSQTNVSQSKDSDVYITDKTVLDMRSMDFKSNSAVA | |
| | | WSNKSDFACANAFNNSIIPEDTFFPSPESSCDVKLVEKSFETDT | |
| | | NLNFQNLSVIGFRILLLKVAGFNLLMTLRLWSS | |
| | TCRa full | METLLGVSLVILWLQLARVNSQQGEEDPQALSIQEGENATMNCS | 6 |
| | ORF | YKTSINNLQWYRQNSGRGLVHLILIRSNEREKHSGRLRVTLDTS | |
| | (with leader) | KKSSSLLITASRAADTASYFCATDTTSGTYKYIFGTGTRLKVLA | |
| | | NIQNPDPAVYQLRDSKSSDKSVCLFTDFDSQTNVSQSKDSDVYI | |
| | | TDKTVLDMRSMDFKSNSAVAWSNKSDFACANAFNNSIIPEDTFF | |
| | | PSPESSCDVKLVEKSFETDTNLNFQNLSVIGFRILLLKVAGFNL | |
| | | LMTLRLWSS | |
| | TCRb | LDFQATTM | 7 |
| | CDR1 | | |
| | TCRb | TSNEGSKAT | 8 |
| | CDR2 | | |
| | TCRb | CSARDLTSGANNEQFF | 9 |
| | CDR3 | | |
| | TCRb | GAVVSQHPSWVICKSGTSVKIECRSLDFQATTMFWYRQFPKQSL | 10 |
| | variable | MLMATSNEGSKATYEQGVEKDKFLINHASLTLSTLTVTSAHPED | |
| | chain | SSFYICSARDLTSGANNEQFFGPGTRLTVL | |
| | TCRb full | GAVVSQHPSWVICKSGTSVKIECRSLDFQATTMFWYRQFPKQSL | 11 |
| | ORF | MLMATSNEGSKATYEQGVEKDKFLINHASLTLSTLTVTSAHPED | |
| | | SSFYICSARDLTSGANNEQFFGPGTRLTVLEDLKNVFPPEVAVF | |
| | | EPSEAEISHTQKATLVCLATGFYPDHVELSWWVNGKEVHSGVST | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRG | |
| | TCRb full | GAVVSQHPSWVICKSGTSVKIECRSLDFQATTMFWYRQFPKQSL | 12 |
| | ORF with | MLMATSNEGSKATYEQGVEKDKFLINHASLTLSTLTVTSAHPED | |
| | P2A | SSFYICSARDLTSGANNEQFFGPGTRLTVLEDLKNVFPPEVAVF | |
| | overhang | EPSEAEISHTQKATLVCLATGFYPDHVELSWWVNGKEVHSGVST | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRGGSGATNFSLLK | |
| | | QAGDVEENPG | |
| | TCRb full | MLLLLLLLGPGSGLGAVVSQHPSWVICKSGTSVKIECRSLDFQA | 13 |
| | ORF | TTMFWYRQFPKQSLMLMATSNEGSKATYEQGVEKDKFLINHASL | |
| | (with leader) | TLSTLTVTSAHPEDSSFYICSARDLTSGANNEQFFGPGTRLTVL | |
| | | EDLKNVFPPEVAVFEPSEAEISHTQKATLVCLATGFYPDHVELS | |
| | | WWVNGKEVHSGVSTDPQPLKEQPALNDSRYCLSSRLRVSATFWQ | |
| | | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRG | |
| | TCRb full | MLLLLLLLGPGSGLGAVVSQHPSWVICKSGTSVKIECRSLDFQA | 67 |
| | ORF as | TTMFWYRQFPKQSLMLMATSNEGSKATYEQGVEKDKFLINHASL | |
| | expressed | TLSTLTVTSAHPEDSSFYICSARDLTSGANNEQFFGPGTRLTVL | |
| | (with leader | EDLKNVFPPEVAVFEPSEAEISHTQKATLVCLATGFYPDHVELS | |
| | and P2A | WWVNGKEVHSGVSTDPQPLKEQPALNDSRYCLSSRLRVSATFWQ | |
| | overhang) | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRGGSGATNFSLLKQAGDVEENPG | |
| | TCRb | GAVVSQHPSWVISKSGTSVKIECRSLDFQATTMFWYRQFPKQSL | 14 |
| | variable | MLMATSNEGSKATYEQGVEKDKFLINHASLTLSTLTVTSAHPED | |
| | chain | SSFYICSARDLTSGANNEQFFGPGTRLTVL | |
| | (serine) | | |
| | TCRb full | GAVVSQHPSWVISKSGTSVKIECRSLDFQATTMFWYRQFPKQSL | 15 |
| | ORF | MLMATSNEGSKATYEQGVEKDKFLINHASLTLSTLTVTSAHPED | |
| | (serine) | SSFYICSARDLTSGANNEQFFGPGTRLTVLEDLKNVFPPEVAVF | |
| | | EPSEAEISHTQKATLVCLATGFYPDHVELSWWVNGKEVHSGVST | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRG | |
| | TCRb full | GAVVSQHPSWVISKSGTSVKIECRSLDFQATTMFWYRQFPKQSL | 16 |
| | ORF (serine | MLMATSNEGSKATYEQGVEKDKFLINHASLTLSTLTVTSAHPED | |
| | with P2A | SSFYICSARDLTSGANNEQFFGPGTRLTVLEDLKNVFPPEVAVF | |
| | overhang) | EPSEAEISHTQKATLVCLATGFYPDHVELSWWVNGKEVHSGVST | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRGGSGATNFSLLK | |
| | | QAGDVEENPG | |
| | TCRb full | MLLLLLLLGPGSGLGAVVSQHPSWVISKSGTSVKIECRSLDFQA | 68 |
| | ORF | TTMFWYRQFPKQSLMLMATSNEGSKATYEQGVEKDKFLINHASL | |
| | (serine with | TLSTLTVTSAHPEDSSFYICSARDLTSGANNEQFFGPGTRLTVL | |
| | leader) | EDLKNVFPPEVAVFEPSEAEISHTQKATLVCLATGFYPDHVELS | |
| | | WWVNGKEVHSGVSTDPQPLKEQPALNDSRYCLSSRLRVSATFWQ | |
| | | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRG | |
| | TCRb full | MLLLLLLLGPGSGLGAVVSQHPSWVISKSGTSVKIECRSLDFQA | 69 |
| | ORF as | TTMFWYRQFPKQSLMLMATSNEGSKATYEQGVEKDKFLINHASL | |
| | expressed | TLSTLTVTSAHPEDSSFYICSARDLTSGANNEQFFGPGTRLTVL | |
| | (with leader | EDLKNVFPPEVAVFEPSEAEISHTQKATLVCLATGFYPDHVELS | |
| | and P2A | WWVNGKEVHSGVSTDPQPLKEQPALNDSRYCLSSRLRVSATFWQ | |
| | overhang) | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRGGSGATNFSLLKQAGDVEENPG | |
| | Beta-P2A- | MLLLLLLLGPGSGLGAVVSQHPSWVICKSGTSVKIECRSLDFQA | 17 |
| | alpha | TTMFWYRQFPKQSLMLMATSNEGSKATYEQGVEKDKFLINHASL | |
| | | TLSTLTVTSAHPEDSSFYICSARDLTSGANNEQFFGPGTRLTVL | |
| | | EDLKNVFPPEVAVFEPSEAEISHTQKATLVCLATGFYPDHVELS | |
| | | WWVNGKEVHSGVSTDPQPLKEQPALNDSRYCLSSRLRVSATFWQ | |
| | | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRGGSGATNFSLLKQAGDVEENPGPMETLLGVSLVILWLQLARV | |
| | | NSQQGEEDPQALSIQEGENATMNCSYKTSINNLQWYRQNSGRGL | |
| | | VHLILIRSNEREKHSGRLRVTLDTSKKSSSLLITASRAADTASY | |
| | | FCATDTTSGTYKYIFGTGTRLKVLANIQNPDPAVYQLRDSKSSD | |
| | | KSVCLFTDFDSQTNVSQSKDSDVYITDKTVLDMRSMDFKSNSAV | |
| | | AWSNKSDFACANAFNNSIIPEDTFFPSPESSCDVKLVEKSFETD | |
| | | TNLNFQNLSVIGFRILLLKVAGFNLLMTLRLWSS | |
| TCR-B | TCRa | KTSINNL | 18 |
| | CDR1 | | |
| | TCRa | LIRSNEREK | 19 |
| | CDR2 | | |
| | TCRa | CATDATSGTYKYIF | 20 |
| | CDR3 | | |
| | TCRa | SQQGEEDPQALSIQEGENATMNCSYKTSINNLQWYRQNSGRGLV | 21 |
| | variable | HLILIRSNEREKHSGRLRVTLDTSKKSSSLLITASRAADTASYF | |
| | chain | CATDATSGTYKYIFGTGTRLKVLA | |
| | TCRa full | SQQGEEDPQALSIQEGENATMNCSYKTSINNLQWYRQNSGRGLV | 22 |
| | ORF | HLILIRSNEREKHSGRLRVTLDTSKKSSSLLITASRAADTASYF | |
| | | CATDATSGTYKYIFGTGTRLKVLANIQNPDPAVYQLRDSKSSDK | |
| | | SVCLFTDFDSQTNVSQSKDSDVYITDKTVLDMRSMDFKSNSAVA | |
| | | WSNKSDFACANAFNNSIIPEDTFFPSPESSCDVKLVEKSFETDT | |
| | | NLNFQNLSVIGFRILLLKVAGFNLLMTLRLWSS | |
| | TCRa full | METLLGVSLVILWLQLARVNSQQGEEDPQALSIQEGENATMNCS | 23 |
| | ORF | YKTSINNLQWYRQNSGRGLVHLILIRSNEREKHSGRLRVTLDTS | |
| | (with leader) | KKSSSLLITASRAADTASYFCATDATSGTYKYIFGTGTRLKVLA | |
| | | NIQNPDPAVYQLRDSKSSDKSVCLFTDFDSQTNVSQSKDSDVYI | |
| | | TDKTVLDMRSMDFKSNSAVAWSNKSDFACANAFNNSIIPEDTFF | |
| | | PSPESSCDVKLVEKSFETDTNLNFQNLSVIGFRILLLKVAGENL | |
| | | LMTLRLWSS | |
| | TCRb | LDFQATTM | 24 |
| | CDR1 | | |
| | TCRb | TSNEGSKAT | 25 |
| | CDR2 | | |
| | TCRb | CSARDLTSGSLNEQFF | 26 |
| | CDR3 | | |
| | TCRb | GAVVSQHPSWVICKSGTSVKIECRSLDFQATTMFWYRQFPKQSL | 27 |
| | variable | MLMATSNEGSKATYEQGVEKDKFLINHASLTLSTLTVTSAHPED | |
| | chain | SSFYICSARDLTSGSLNEQFFGPGTRLTVL | |
| | TCRb full | GAVVSQHPSWVICKSGTSVKIECRSLDFQATTMFWYRQFPKQSL | 28 |
| | ORF | MLMATSNEGSKATYEQGVEKDKFLINHASLTLSTLTVTSAHPED | |
| | | SSFYICSARDLTSGSLNEQFFGPGTRLTVLEDLKNVFPPEVAVF | |
| | | EPSEAEISHTQKATLVCLATGFYPDHVELSWWVNGKEVHSGVST | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRG | |
| | TCRb full | GAVVSQHPSWVICKSGTSVKIECRSLDFQATTMFWYRQFPKQSL | 29 |
| | ORF with | MLMATSNEGSKATYEQGVEKDKFLINHASLTLSTLTVTSAHPED | |
| | P2A | SSFYICSARDLTSGSLNEQFFGPGTRLTVLEDLKNVFPPEVAVF | |
| | overhang | EPSEAEISHTQKATLVCLATGFYPDHVELSWWVNGKEVHSGVST | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRGGSGATNFSLLK | |
| | | QAGDVEENPG | |
| | TCRb full | MLLLLLLLGPGSGLGAVVSQHPSWVICKSGTSVKIECRSLDFQA | 30 |
| | ORF | TTMFWYRQFPKQSLMLMATSNEGSKATYEQGVEKDKFLINHASL | |
| | (with leader) | TLSTLTVTSAHPEDSSFYICSARDLTSGSLNEQFFGPGTRLTVL | |
| | | EDLKNVFPPEVAVFEPSEAEISHTQKATLVCLATGFYPDHVELS | |
| | | WWVNGKEVHSGVSTDPQPLKEQPALNDSRYCLSSRLRVSATFWQ | |
| | | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRG | |
| | TCRb full | MLLLLLLLGPGSGLGAVVSQHPSWVICKSGTSVKIECRSLDFQA | 70 |
| | ORF as | TTMFWYRQFPKQSLMLMATSNEGSKATYEQGVEKDKFLINHASL | |
| | expressed | TLSTLTVTSAHPEDSSFYICSARDLTSGSLNEQFFGPGTRLTVL | |
| | (with leader | EDLKNVFPPEVAVFEPSEAEISHTQKATLVCLATGFYPDHVELS | |
| | and P2A | WWVNGKEVHSGVSTDPQPLKEQPALNDSRYCLSSRLRVSATFWQ | |
| | overhang) | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRGGSGATNFSLLKQAGDVEENPG | |
| | Beta-P2A- | MLLLLLLLGPGSGLGAVVSQHPSWVICKSGTSVKIECRSLDFQA | 31 |
| | alpha | TTMFWYRQFPKQSLMLMATSNEGSKATYEQGVEKDKFLINHASL | |
| | | TLSTLTVTSAHPEDSSFYICSARDLTSGSLNEQFFGPGTRLTVL | |
| | | EDLKNVFPPEVAVFEPSEAEISHTQKATLVCLATGFYPDHVELS | |
| | | WWVNGKEVHSGVSTDPQPLKEQPALNDSRYCLSSRLRVSATFWQ | |
| | | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRGGSGATNFSLLKQAGDVEENPGPMETLLGVSLVILWLQLARV | |
| | | NSQQGEEDPQALSIQEGENATMNCSYKTSINNLQWYRQNSGRGL | |
| | | VHLILIRSNEREKHSGRLRVTLDTSKKSSSLLITASRAADTASY | |
| | | FCATDATSGTYKYIFGTGTRLKVLANIQNPDPAVYQLRDSKSSD | |
| | | KSVCLFTDFDSQTNVSQSKDSDVYITDKTVLDMRSMDFKSNSAV | |
| | | AWSNKSDFACANAFNNSIIPEDTFFPSPESSCDVKLVEKSFETD | |
| | | TNLNFQNLSVIGFRILLLKVAGFNLLMTLRLWSS | |
| TCR-C | TCRa | TSINN | 32 |
| | CDR1 | | |
| | TCRa | IRSNERE | 33 |
| | CDR2 | | |
| | TCRa | CATDSGGSYIPTF | 34 |
| | CDR3 | | |
| | TCRa | SQQGEEDPQALSIQEGENATMNCSYKTSINNLQWYRQNSGRGLV | 35 |
| | variable | HLILIRSNEREKHSGRLRVTLDTSKKSSSLLITASRAADTASYF | |
| | chain | CATDSGGSYIPTFGRGTSLIVHP | |
| | TCRa full | SQQGEEDPQALSIQEGENATMNCSYKTSINNLQWYRQNSGRGLV | 36 |
| | ORF | HLILIRSNEREKHSGRLRVTLDTSKKSSSLLITASRAADTASYF | |
| | | CATDSGGSYIPTFGRGTSLIVHPYIQNPDPAVYQLRDSKSSDKS | |
| | | VCLFTDFDSQTNVSQSKDSDVYITDKTVLDMRSMDFKSNSAVAW | |
| | | SNKSDFACANAFNNSIIPEDTFFPSPESSCDVKLVEKSFETDTN | |
| | | LNFQNLSVIGFRILLLKVAGFNLLMTLRLWSS | |
| | TCRa full | METLLGVSLVILWLQLARVNSQQGEEDPQALSIQEGENATMNCS | 37 |
| | ORF | YKTSINNLQWYRQNSGRGLVHLILIRSNEREKHSGRLRVTLDTS | |
| | (with leader) | KKSSSLLITASRAADTASYFCATDSGGSYIPTFGRGTSLIVHPY | |
| | | IQNPDPAVYQLRDSKSSDKSVCLFTDFDSQTNVSQSKDSDVYIT | |
| | | DKTVLDMRSMDFKSNSAVAWSNKSDFACANAFNNSIIPEDTFFP | |
| | | SPESSCDVKLVEKSFETDTNLNFQNLSVIGFRILLLKVAGENLL | |
| | | MTLRLWSS | |
| | TCRb | SQVTM | 38 |
| | CDR1 | | |
| | TCRb | ANQGSEA | 39 |
| | CDR2 | | |
| | TCRb | CSAWPSGQGTYGYTF | 40 |
| | CDR3 | | |
| | TCRb | SAVISQKPSRDICQRGTSLTIQCQVDSQVTMMFWYRQQPGQSLT | 41 |
| | variable | LIATANQGSEATYESGFVIDKFPISRPNLTFSTLTVSNMSPEDS | |
| | chain | SIYLCSAWPSGQGTYGYTFGSGTRLTVV | |
| | TCRb full | SAVISQKPSRDICQRGTSLTIQCQVDSQVTMMFWYRQQPGQSLT | 42 |
| | ORF | LIATANQGSEATYESGFVIDKFPISRPNLTFSTLTVSNMSPEDS | |
| | (processed) | SIYLCSAWPSGQGTYGYTFGSGTRLTVVEDLNKVFPPEVAVFEP | |
| | | SEAEISHTQKATLVCLATGFFPDHVELSWWVNGKEVHSGVSTDP | |
| | | QPLKEQPALNDSRYCLSSRLRVSATFWQNPRNHFRCQVQFYGLS | |
| | | ENDEWTQDRAKPVTQIVSAEAWGRADCGFTSVSYQQGVLSATIL | |
| | | YEILLGKATLYAVLVSALVLMAMVKRKDF | |
| | TCRb full | SAVISQKPSRDICQRGTSLTIQCQVDSQVTMMFWYRQQPGQSLT | 43 |
| | ORF | LIATANQGSEATYESGFVIDKFPISRPNLTFSTLTVSNMSPEDS | |
| | | SIYLCSAWPSGQGTYGYTFGSGTRLTVVEDLNKVFPPEVAVFEP | |
| | | SEAEISHTQKATLVCLATGFFPDHVELSWWVNGKEVHSGVSTDP | |
| | | QPLKEQPALNDSRYCLSSRLRVSATFWQNPRNHFRCQVQFYGLS | |
| | | ENDEWTQDRAKPVTQIVSAEAWGRADCGFTSVSYQQGVLSATIL | |
| | | YEILLGKATLYAVLVSALVLMAMVKRKDFGSGATNFSLLKQAGD | |
| | | VEENPG | |
| | TCRb full | MLSLLLLLLGLGSVFSAVISQKPSRDICQRGTSLTIQCQVDSQV | 44 |
| | ORF | TMMFWYRQQPGQSLTLIATANQGSEATYESGFVIDKFPISRPNL | |
| | (with leader) | TFSTLTVSNMSPEDSSIYLCSAWPSGQGTYGYTFGSGTRLTVVE | |
| | | DLNKVFPPEVAVFEPSEAEISHTQKATLVCLATGFFPDHVELSW | |
| | | WVNGKEVHSGVSTDPQPLKEQPALNDSRYCLSSRLRVSATFWQN | |
| | | PRNHFRCQVQFYGLSENDEWTQDRAKPVTQIVSAEAWGRADCGF | |
| | | TSVSYQQGVLSATILYEILLGKATLYAVLVSALVLMAMVKRKDF | |
| | Beta-P2A- | MLSLLLLLLGLGSVFSAVISQKPSRDICQRGTSLTIQCQVDSQV | 45 |
| | alpha | TMMFWYRQQPGQSLTLIATANQGSEATYESGFVIDKFPISRPNL | |
| | | TFSTLTVSNMSPEDSSIYLCSAWPSGQGTYGYTFGSGTRLTVVE | |
| | | DLNKVFPPEVAVFEPSEAEISHTQKATLVCLATGFFPDHVELSW | |
| | | WVNGKEVHSGVSTDPQPLKEQPALNDSRYCLSSRLRVSATFWQN | |
| | | PRNHFRCQVQFYGLSENDEWTQDRAKPVTQIVSAEAWGRADCGF | |
| | | TSVSYQQGVLSATILYEILLGKATLYAVLVSALVLMAMVKRKDF | |
| | | GSGATNFSLLKQAGDVEENPGPMETLLGVSLVILWLQLARVNSQ | |
| | | QGEEDPQALSIQEGENATMNCSYKTSINNLQWYRQNSGRGLVHL | |
| | | ILIRSNEREKHSGRLRVTLDTSKKSSSLLITASRAADTASYFCA | |
| | | TDSGGSYIPTFGRGTSLIVHPYIQNPDPAVYQLRDSKSSDKSVC | |
| | | LFTDFDSQTNVSQSKDSDVYITDKTVLDMRSMDFKSNSAVAWSN | |
| | | KSDFACANAFNNSIIPEDTFFPSPESSCDVKLVEKSFETDTNLN | |
| | | FQNLSVIGFRILLLKVAGFNLLMTLRLWSS | |
| TCR-D | TCRa | KTSINNL | 46 |
| | CDR1 | | |
| | TCRa | LIRSNEREK | 47 |
| | CDR2 | | |
| | TCRa | CATDGNGNQFYF | 48 |
| | CDR3 | | |
| | TCRa | SQQGEEDPQALSIQEGENATMNCSYKTSINNLQWYRQNSGRGLV | 49 |
| | variable | HLILIRSNEREKHSGRLRVTLDTSKKSSSLLITASRAADTASYF | |
| | chain | CATDGNGNQFYFGTGTSLTVIP | |
| | TCRa full | SQQGEEDPQALSIQEGENATMNCSYKTSINNLQWYRQNSGRGLV | 50 |
| | ORF | HLILIRSNEREKHSGRLRVTLDTSKKSSSLLITASRAADTASYF | |
| | | CATDGNGNQFYFGTGTSLTVIPNIQNPDPAVYQLRDSKSSDKSV | |
| | | CLFTDFDSQTNVSQSKDSDVYITDKTVLDMRSMDFKSNSAVAWS | |
| | | NKSDFACANAFNNSIIPEDTFFPSPESSCDVKLVEKSFETDTNL | |
| | | NFQNLSVIGFRILLLKVAGFNLLMTLRLWSS | |
| | TCRa full | METLLGVSLVILWLQLARVNSQQGEEDPQALSIQEGENATMNCS | 51 |
| | ORF | YKTSINNLQWYRQNSGRGLVHLILIRSNEREKHSGRLRVTLDTS | |
| | (with leader) | KKSSSLLITASRAADTASYFCATDGNGNQFYFGTGTSLTVIPNI | |
| | | QNPDPAVYQLRDSKSSDKSVCLFTDFDSQTNVSQSKDSDVYITD | |
| | | KTVLDMRSMDFKSNSAVAWSNKSDFACANAFNNSIIPEDTFFPS | |
| | | PESSCDVKLVEKSFETDTNLNFQNLSVIGFRILLLKVAGFNLLM | |
| | | TLRLWSS | |
| | TCRb | ISGHATL | 52 |
| | CDR1 | | |
| | TCRb | QFQNNGVV | 53 |
| | CDR2 | | |
| | TCRb | CASSIRHRTNTEAFF | 54 |
| | CDR3 | | |
| | TCRb | EAGVAQSPRYKIIEKRQSVAFWCNPISGHATLYWYQQILGQGPK | 55 |
| | variable | LLIQFQNNGVVDDSQLPKDRFSAERLKGVDSTLKIQPAKLEDSA | |
| | chain | VYLCASSIRHRTNTEAFFGQGTRLTVV | |
| | TCRb full | EAGVAQSPRYKIIEKRQSVAFWCNPISGHATLYWYQQILGQGPK | 56 |
| | ORF | LLIQFQNNGVVDDSQLPKDRFSAERLKGVDSTLKIQPAKLEDSA | |
| | | VYLCASSIRHRTNTEAFFGQGTRLTVVEDLNKVFPPEVAVFEPS | |
| | | EAEISHTQKATLVCLATGFFPDHVELSWWVNGKEVHSGVSTDPQ | |
| | | PLKEQPALNDSRYCLSSRLRVSATFWQNPRNHFRCQVQFYGLSE | |
| | | NDEWTQDRAKPVTQIVSAEAWGRADCGFTSVSYQQGVLSATILY | |
| | | EILLGKATLYAVLVSALVLMAMVKRKDF | |
| | TCRb full | MGTRLLCWAALCLLGAELTEAGVAQSPRYKIIEKRQSVAFWCNP | 57 |
| | ORF | ISGHATLYWYQQILGQGPKLLIQFQNNGVVDDSQLPKDRFSAER | |
| | (with leader) | LKGVDSTLKIQPAKLEDSAVYLCASSIRHRTNTEAFFGQGTRLT | |
| | | VVEDLNKVFPPEVAVFEPSEAEISHTQKATLVCLATGFFPDHVE | |
| | | LSWWVNGKEVHSGVSTDPQPLKEQPALNDSRYCLSSRLRVSATF | |
| | | WQNPRNHFRCQVQFYGLSENDEWTQDRAKPVTQIVSAEAWGRAD | |
| | | CGFTSVSYQQGVLSATILYEILLGKATLYAVLVSALVLMAMVKR | |
| | | KDF | |
| | TCRb full | EAGVAQSPRYKIIEKRQSVAFWCNPISGHATLYWYQQILGQGPK | 71 |
| | ORF | LLIQFQNNGVVDDSQLPKDRFSAERLKGVDSTLKIQPAKLEDSA | |
| | with P2A | VYLCASSIRHRTNTEAFFGQGTRLTVVEDLNKVFPPEVAVFEPS | |
| | overhang | EAEISHTQKATLVCLATGFFPDHVELSWWVNGKEVHSGVSTDPQ | |
| | | PLKEQPALNDSRYCLSSRLRVSATFWQNPRNHFRCQVQFYGLSE | |
| | | NDEWTQDRAKPVTQIVSAEAWGRADCGFTSVSYQQGVLSATILY | |
| | | EILLGKATLYAVLVSALVLMAMVKRKDFGSGATNFSLLKQAGDV | |
| | | EENPG | |
| | TCRb full | MGTRLLCWAALCLLGAELTEAGVAQSPRYKIIEKRQSVAFWCNP | 72 |
| | ORF as | ISGHATLYWYQQILGQGPKLLIQFQNNGVVDDSQLPKDRFSAER | |
| | expressed | LKGVDSTLKIQPAKLEDSAVYLCASSIRHRTNTEAFFGQGTRLT | |
| | (with leader | VVEDLNKVFPPEVAVFEPSEAEISHTQKATLVCLATGFFPDHVE | |
| | and P2A | LSWWVNGKEVHSGVSTDPQPLKEQPALNDSRYCLSSRLRVSATF | |
| | overhang) | WQNPRNHFRCQVQFYGLSENDEWTQDRAKPVTQIVSAEAWGRAD | |
| | | CGFTSVSYQQGVLSATILYEILLGKATLYAVLVSALVLMAMVKR | |
| | | KDFGSGATNFSLLKQAGDVEENPG | |
| TCR-E | TCRa | KTSINNL | 1 |
| | CDR1 | | |
| | TCRa | LIRSNEREK | 2 |
| | CDR2 | | |
| | TCRa | CATDTTSGTYKYIF | 3 |
| | CDR3 | | |
| | TCRa | SQQGEEDPQALSIQEGENATMNCSYKTSINNLQWYRQNSGRGLV | 4 |
| | variable | HLILIRSNEREKHSGRLRVTLDTSKKSSSLLITASRAADTASYF | |
| | chain | CATDTTSGTYKYIFGTGTRLKVLA | |
| | TCRa full | SQQGEEDPQALSIQEGENATMNCSYKTSINNLQWYRQNSGRGLV | 73 |
| | ORF | HLILIRSNEREKHSGRLRVTLDTSKKSSSLLITASRAADTASYF | |
| | | CATDTTSGTYKYIFGTGTRLKVLANIQNPDPAVYQLRDSKSSDK | |
| | | SVCLFTDFDSQTNVSQSKDSDVYITDKCVLDMRSMDFKSNSAVA | |
| | | WSNKSDFACANAFNNSIIPEDTFFPSPESSCDVKLVEKSFETDT | |
| | | NLNFQNLSVIGFRILLLKVAGFNLLMTLRLWSS | |
| | TCRa full | METLLGVSLVILWLQLARVNSQQGEEDPQALSIQEGENATMNCS | 74 |
| | ORF | YKTSINNLQWYRQNSGRGLVHLILIRSNEREKHSGRLRVTLDTS | |
| | (with leader) | KKSSSLLITASRAADTASYFCATDTTSGTYKYIFGTGTRLKVLA | |
| | | NIQNPDPAVYQLRDSKSSDKSVCLFTDFDSQTNVSQSKDSDVYI | |
| | | TDKCVLDMRSMDFKSNSAVAWSNKSDFACANAFNNSIIPEDTFF | |
| | | PSPESSCDVKLVEKSFETDTNLNFQNLSVIGFRILLLKVAGENL | |
| | | LMTLRLWSS | |
| | TCRb | LDFQATTM | 7 |
| | CDR1 | | |
| | TCRb | TSNEGSKAT | 8 |
| | CDR2 | | |
| | TCRb | CSARDLTSGANNEQFF | 9 |
| | CDR3 | | |
| | TCRb | GAVVSQHPSWVICKSGTSVKIECRSLDFQATTMFWYRQFPKQSL | 10 |
| | variable | MLMATSNEGSKATYEQGVEKDKFLINHASLTLSTLTVTSAHPED | |
| | chain | SSFYICSARDLTSGANNEQFFGPGTRLTVL | |
| | TCRb full | GAVVSQHPSWVICKSGTSVKIECRSLDFQATTMFWYRQFPKQSL | 75 |
| | ORF | MLMATSNEGSKATYEQGVEKDKFLINHASLTLSTLTVTSAHPED | |
| | | SSFYICSARDLTSGANNEQFFGPGTRLTVLEDLKNVFPPEVAVF | |
| | | EPSEAEISHTQKATLVCLATGFYPDHVELSWWVNGKEVHSGVCT | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRG | |
| | TCRb full | GAVVSQHPSWVICKSGTSVKIECRSLDFQATTMFWYRQFPKQSL | 76 |
| | ORF with | MLMATSNEGSKATYEQGVEKDKFLINHASLTLSTLTVTSAHPED | |
| | P2A | SSFYICSARDLTSGANNEQFFGPGTRLTVLEDLKNVFPPEVAVF | |
| | overhang | EPSEAEISHTQKATLVCLATGFYPDHVELSWWVNGKEVHSGVCT | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRGGSGATNFSLLK | |
| | | QAGDVEENPG | |
| | TCRb full | MLLLLLLLGPGSGLGAVVSQHPSWVICKSGTSVKIECRSLDFQA | 77 |
| | ORF | TTMFWYRQFPKQSLMLMATSNEGSKATYEQGVEKDKFLINHASL | |
| | (with leader) | TLSTLTVTSAHPEDSSFYICSARDLTSGANNEQFFGPGTRLTVL | |
| | | EDLKNVFPPEVAVFEPSEAEISHTQKATLVCLATGFYPDHVELS | |
| | | WWVNGKEVHSGVCTDPQPLKEQPALNDSRYCLSSRLRVSATFWQ | |
| | | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRG | |
| | TCRb full | MLLLLLLLGPGSGLGAVVSQHPSWVICKSGTSVKIECRSLDFQA | 78 |
| | ORF as | TTMFWYRQFPKQSLMLMATSNEGSKATYEQGVEKDKFLINHASL | |
| | expressed | TLSTLTVTSAHPEDSSFYICSARDLTSGANNEQFFGPGTRLTVL | |
| | (with leader | EDLKNVFPPEVAVFEPSEAEISHTQKATLVCLATGFYPDHVELS | |
| | and P2A | WWVNGKEVHSGVCTDPQPLKEQPALNDSRYCLSSRLRVSATFWQ | |
| | overhang) | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRGGSGATNFSLLKQAGDVEENPG | |
| | Beta-P2A- | MLLLLLLLGPGSGLGAVVSQHPSWVICKSGTSVKIECRSLDFQA | 79 |
| | alpha | TTMFWYRQFPKQSLMLMATSNEGSKATYEQGVEKDKFLINHASL | |
| | (with | TLSTLTVTSAHPEDSSFYICSARDLTSGANNEQFFGPGTRLTVL | |
| | disulfide) | EDLKNVFPPEVAVFEPSEAEISHTQKATLVCLATGFYPDHVELS | |
| | | WWVNGKEVHSGVCTDPQPLKEQPALNDSRYCLSSRLRVSATFWQ | |
| | | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRGGSGATNFSLLKQAGDVEENPGPMETLLGVSLVILWLQLARV | |
| | | NSQQGEEDPQALSIQEGENATMNCSYKTSINNLQWYRQNSGRGL | |
| | | VHLILIRSNEREKHSGRLRVTLDTSKKSSSLLITASRAADTASY | |
| | | FCATDTTSGTYKYIFGTGTRLKVLANIQNPDPAVYQLRDSKSSD | |
| | | KSVCLFTDFDSQTNVSQSKDSDVYITDKCVLDMRSMDFKSNSAV | |
| | | AWSNKSDFACANAFNNSIIPEDTFFPSPESSCDVKLVEKSFETD | |
| | | TNLNFQNLSVIGFRILLLKVAGFNLLMTLRLWSS | |
| | Beta-P2A- | ATGCTCCTGC TCCTTCTGCT GCTGGGACCC GGTTCCGGCC | 87 |
| | alpha | TCGGCGCTGT GGTGTCACAG CACCCTTCAT GGGTGATCTG | |
| | (with | CAAGAGTGGG ACTAGCGTGA AGATCGAGTG TCGGTCCCTG | |
| | disulfide | GACTTCCAAG CCACCACCAT GTTCTGGTAC CGGCAGTTCC | |
| | bond) | CGAAGCAGTC GTTGATGCTG ATGGCCACTT CCAACGAGGG | |
| | | CAGCAAGGCT ACCTACGAAC AGGGCGTGGA AAAGGACAAG | |
| | | TTTCTGATTA ACCACGCATC CCTGACCCTG TCGACTCTCA | |
| | | CCGTGACCTC GGCCCACCCC GAGGATAGCT CCTTCTACAT | |
| | | CTGCTCCGCG CGCGATCTGA CTTCCGGAGC CAACAACGAA | |
| | | CAGTTTTTCG GCCCTGGTAC TAGACTCACT GTGCTGGAAG | |
| | | ATCTGAAGAA CGTGTTCCCG CCCGAAGTGG CCGTGTTCGA | |
| | | GCCCTCCGAA GCCGAGATCT CCCATACTCA AAAAGCCACC | |
| | | CTTGTCTGCC TGGCTACCGG ATTCTACCCC GATCACGTGG | |
| | | AGCTCTCTTG GTGGGTGAAC GGAAAGGAAG TGCATTCCGG | |
| | | AGTCTGCACC GACCCTCAGC CGCTGAAAGA ACAGCCAGCC | |
| | | CTGAACGACT CCCGCTACTG CCTGAGCTCA CGGCTGAGAG | |
| | | TGTCCGCCAC CTTCTGGCAA AATCCTAGGA ACCACTTCCG | |
| | | GTGCCAAGTG CAGTTCTATG GACTGAGCGA GAACGACGAA | |
| | | TGGACCCAGG ACAGAGCGAA GCCGGTCACA CAGATCGTGT | |
| | | CAGCGGAGGC CTGGGGGCGC GCCGACTGTG GATTCACGTC | |
| | | GGAAAGCTAC CAGCAGGGCG TGCTGTCGGC AACCATTTTG | |
| | | TACGAGATTT TGCTCGGGAA GGCCACCCTG TACGCGGTGC | |
| | | TCGTCAGCGC CCTCGTCCTT ATGGCAATGG TCAAGCGCAA | |
| | | GGACTCCCGG GGTGGCAGCG GCGCCACCAA CTTCTCCCTG | |
| | | CTGAAGCAGG CCGGCGACGT GGAAGAAAAC CCTGGCCCCA | |
| | | TGGAAACCCT GCTGGGAGTG TCACTTGTGA TCTTGTGGCT | |
| | | GCAACTGGCG CGCGTCAACA GCCAACAGGG CGAAGAGGAT | |
| | | CCACAGGCCC TGAGCATCCA GGAAGGGGAG AACGCGACTA | |
| | | TGAACTGCAG CTACAAGACT AGCATCAACA ATCTGCAGTG | |
| | | GTACCGCCAG AACTCCGGCC GAGGGCTTGT GCACCTGATT | |
| | | CTGATTCGGA GCAATGAGCG GGAGAAGCAT TCCGGACGGC | |
| | | TCAGGGTCAC CCTGGACACC TCCAAAAAGT CGAGCTCACT | |
| | | CCTGATTACG GCCTCAAGAG CCGCTGACAC TGCTTCCTAT | |
| | | TTCTGCGCCA CTGACACTAC ATCAGGAACC TACAAGTACA | |
| | | TCTTCGGAAC CGGAACCCGC TTGAAAGTCC TGGCCAACAT | |
| | | CCAGAACCCA GACCCGGCAG TGTACCAGCT GCGGGACTCC | |
| | | AAGTCCTCGG ATAAGAGCGT GTGCCTATTC ACCGACTTCG | |
| | | ACTCCCAAAC TAACGTGTCC CAGTCCAAAG ATAGCGACGT | |
| | | GTACATCACC GACAAGTGCG TTCTGGACAT GCGCTCCATG | |
| | | GACTTCAAGT CGAACTCGGC CGTGGCCTGG TCCAACAAGT | |
| | | CCGATTTCGC GTGCGCCAAT GCGTTCAACA ACTCCATCAT | |
| | | TCCTGAGGAT ACCTTCTTCC CGTCCCCGGA GTCGTCGTGT | |
| | | GATGTGAAGC TCGTGGAGAA GTCCTTCGAA ACCGACACCA | |
| | | ACCTGAACTT TCAAAACCTT TCGGTGATCG GTTTTAGGAT | |
| | | CCTGCTGCTC AAAGTCGCCG GGTTTAATCT GCTCATGACC | |
| | | CTGCGGCTCT GGTCCTCTTA A | |
| TCR-F | TCRa | KTSINNL | 18 |
| | CDR1 | | |
| | TCRa | LIRSNEREK | 19 |
| | CDR2 | | |
| | TCRa | CATDATSGTYKYIF | 20 |
| | CDR3 | | |
| | TCRa | SQQGEEDPQALSIQEGENATMNCSYKTSINNLQWYRQNSGRGLV | 21 |
| | variable | HLILIRSNEREKHSGRLRVTLDTSKKSSSLLITASRAADTASYF | |
| | chain | CATDATSGTYKYIFGTGTRLKVLA | |
| | TCRa full | SQQGEEDPQALSIQEGENATMNCSYKTSINNLQWYRQNSGRGLV | 80 |
| | ORF | HLILIRSNEREKHSGRLRVTLDTSKKSSSLLITASRAADTASYF | |
| | | CATDATSGTYKYIFGTGTRLKVLANIQNPDPAVYQLRDSKSSDK | |
| | | SVCLFTDFDSQTNVSQSKDSDVYITDKCVLDMRSMDFKSNSAVA | |
| | | WSNKSDFACANAFNNSIIPEDTFFPSPESSCDVKLVEKSFETDT | |
| | | NLNFQNLSVIGFRILLLKVAGFNLLMTLRLWSS | |
| | TCRa full | METLLGVSLVILWLQLARVNSQQGEEDPQALSIQEGENATMNCS | 81 |
| | ORF | YKTSINNLQWYRQNSGRGLVHLILIRSNEREKHSGRLRVTLDTS | |
| | (with leader) | KKSSSLLITASRAADTASYFCATDATSGTYKYIFGTGTRLKVLA | |
| | | NIQNPDPAVYQLRDSKSSDKSVCLFTDFDSQTNVSQSKDSDVYI | |
| | | TDKCVLDMRSMDFKSNSAVAWSNKSDFACANAFNNSIIPEDTFF | |
| | | PSPESSCDVKLVEKSFETDTNLNFQNLSVIGFRILLLKVAGFNL | |
| | | LMTLRLWSS | |
| | TCRb | LDFQATTM | 24 |
| | CDR1 | | |
| | TCRb | TSNEGSKAT | 25 |
| | CDR2 | | |
| | TCRb | CSARDLTSGSLNEQFF | 26 |
| | CDR3 | | |
| | TCRb | GAVVSQHPSWVICKSGTSVKIECRSLDFQATTMFWYRQFPKQSL | 27 |
| | variable | MLMATSNEGSKATYEQGVEKDKFLINHASLTLSTLTVTSAHPED | |
| | chain | SSFYICSARDLTSGSLNEQFFGPGTRLTVL | |
| | TCRb full | GAVVSQHPSWVICKSGTSVKIECRSLDFQATTMFWYRQFPKQSL | 82 |
| | ORF | MLMATSNEGSKATYEQGVEKDKFLINHASLTLSTLTVTSAHPED | |
| | | SSFYICSARDLTSGSLNEQFFGPGTRLTVLEDLKNVFPPEVAVF | |
| | | EPSEAEISHTQKATLVCLATGFYPDHVELSWWVNGKEVHSGVCT | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRG | |
| | TCRb full | GAVVSQHPSWVICKSGTSVKIECRSLDFQATTMFWYRQFPKQSL | 83 |
| | ORF with | MLMATSNEGSKATYEQGVEKDKFLINHASLTLSTLTVTSAHPED | |
| | P2A | SSFYICSARDLTSGSLNEQFFGPGTRLTVLEDLKNVFPPEVAVF | |
| | overhang | EPSEAEISHTQKATLVCLATGFYPDHVELSWWVNGKEVHSGVCT | |
| | | DPQPLKEQPALNDSRYCLSSRLRVSATFWQNPRNHFRCQVQFYG | |
| | | LSENDEWTQDRAKPVTQIVSAEAWGRADCGFTSESYQQGVLSAT | |
| | | ILYEILLGKATLYAVLVSALVLMAMVKRKDSRGGSGATNFSLLK | |
| | | QAGDVEENPG | |
| | TCRb full | MLLLLLLLGPGSGLGAVVSQHPSWVICKSGTSVKIECRSLDFQA | 84 |
| | ORF | TTMFWYRQFPKQSLMLMATSNEGSKATYEQGVEKDKFLINHASL | |
| | (with leader) | TLSTLTVTSAHPEDSSFYICSARDLTSGSLNEQFFGPGTRLTVL | |
| | | EDLKNVFPPEVAVFEPSEAEISHTQKATLVCLATGFYPDHVELS | |
| | | WWVNGKEVHSGVCTDPQPLKEQPALNDSRYCLSSRLRVSATFWQ | |
| | | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRG | |
| | TCRb full | MLLLLLLLGPGSGLGAVVSQHPSWVICKSGTSVKIECRSLDFQA | 85 |
| | ORF as | TTMFWYRQFPKQSLMLMATSNEGSKATYEQGVEKDKFLINHASL | |
| | expressed | TLSTLTVTSAHPEDSSFYICSARDLTSGSLNEQFFGPGTRLTVL | |
| | (with leader | EDLKNVFPPEVAVFEPSEAEISHTQKATLVCLATGFYPDHVELS | |
| | and P2A | WWVNGKEVHSGVCTDPQPLKEQPALNDSRYCLSSRLRVSATFWQ | |
| | overhang) | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRGGSGATNFSLLKQAGDVEENPG | |
| | Beta-P2A- | MLLLLLLLGPGSGLGAVVSQHPSWVICKSGTSVKIECRSLDFQA | 86 |
| | alpha | TTMFWYRQFPKQSLMLMATSNEGSKATYEQGVEKDKFLINHASL | |
| | (with | TLSTLTVTSAHPEDSSFYICSARDLTSGSLNEQFFGPGTRLTVL | |
| | disulfide) | EDLKNVFPPEVAVFEPSEAEISHTQKATLVCLATGFYPDHVELS | |
| | | WWVNGKEVHSGVCTDPQPLKEQPALNDSRYCLSSRLRVSATFWQ | |
| | | NPRNHFRCQVQFYGLSENDEWTQDRAKPVTQIVSAEAWGRADCG | |
| | | FTSESYQQGVLSATILYEILLGKATLYAVLVSALVLMAMVKRKD | |
| | | SRGGSGATNFSLLKQAGDVEENPGPMETLLGVSLVILWLQLARV | |
| | | NSQQGEEDPQALSIQEGENATMNCSYKTSINNLQWYRQNSGRGL | |
| | | VHLILIRSNEREKHSGRLRVTLDTSKKSSSLLITASRAADTASY | |
| | | FCATDATSGTYKYIFGTGTRLKVLANIQNPDPAVYQLRDSKSSD | |
| | | KSVCLFTDFDSQTNVSQSKDSDVYITDKCVLDMRSMDFKSNSAV | |
| | | AWSNKSDFACANAFNNSIIPEDTFFPSPESSCDVKLVEKSFETD | |
| | | TNLNFQNLSVIGFRILLLKVAGFNLLMTLRLWSS | |
| | Beta-P2A- | ATGCTGCTGC TTCTGCTGCT TCTGGGGCCA GGTTCCGGGC | 88 |
| | alpha | TTGGTGCTGT CGTCTCTCAA CATCCGAGCT GGGTTATCTG | |
| | (with | TAAGAGTGGA ACCTCTGTGA AGATCGAGTG CCGTTCCCTG | |
| | disulfide) | GACTTTCAGG CCACAACTAT GTTTTGGTAT CGTCAGTTCC | |
| | | CGAAACAGAG TCTCATGCTG ATGGCAACTT CCAATGAGGG | |
| | | CTCCAAGGCC ACATACGAGC AAGGCGTCGA GAAGGACAAG | |
| | | TTTCTCATCA ACCATGCAAG CCTGACCTTG TCCACTCTGA | |
| | | CAGTGACCAG TGCCCATCCT GAAGACAGCA GCTTCTACAT | |
| | | CTGCAGTGCT AGAGACCTCA CTTCCGGCTC CCTGAATGAG | |
| | | CAGTTCTTCG GGCCAGGGAC ACGGCTCACC GTGCTAGAGG | |
| | | ACCTGAAAAA CGTGTTCCCA CCCGAGGTCG CTGTGTTTGA | |
| | | GCCATCAGAA GCAGAGATCT CCCACACCCA AAAGGCCACA | |
| | | CTGGTGTGCC TGGCCACAGG CTTCTACCCC GACCACGTGG | |
| | | AGCTGAGCTG GTGGGTGAAT GGGAAGGAGG TGCACAGTGG | |
| | | GGTCTGCACA GACCCGCAGC CCCTCAAGGA GCAGCCCGCC | |
| | | CTCAATGACT CCAGATACTG CCTGAGCAGC CGCCTGAGGG | |
| | | TCTCGGCCAC CTTCTGGCAG AACCCCCGCA ACCACTTCCG | |
| | | CTGTCAAGTC CAGTTCTACG GGCTCTCGGA GAATGACGAG | |
| | | TGGACCCAGG ATAGGGCCAA ACCTGTCACC CAGATCGTCA | |
| | | GCGCCGAGGC CTGGGGTAGA GCAGACTGTG GCTTCACCTC | |
| | | CGAGTCTTAC CAGCAAGGGG TCCTGTCTGC CACCATCCTC | |
| | | TATGAGATCT TGCTAGGGAA GGCCACCTTG TATGCCGTGC | |
| | | TGGTCAGTGC CCTCGTGCTG ATGGCCATGG TGAAGAGAAA | |
| | | GGATTCCAGA GGCGGCAGCG GCGCCACCAA CTTCTCCCTG | |
| | | CTGAAGCAGG CCGGCGACGT GGAAGAAAAC CCTGGCCCCA | |
| | | TGGAAACTCT CCTGGGAGTG TCTTTGGTGA TTCTATGGCT | |
| | | TCAACTGGCT AGGGTGAACA GTCAACAGGG AGAAGAGGAT | |
| | | CCTCAGGCCT TGAGCATCCA GGAGGGTGAA AATGCCACCA | |
| | | TGAACTGCAG TTACAAAACT AGTATAAACA ATTTACAGTG | |
| | | GTATAGACAA AATTCAGGTA GAGGCCTTGT CCACCTAATT | |
| | | TTAATACGTT CAAATGAAAG AGAGAAACAC AGTGGAAGAT | |
| | | TAAGAGTCAC GCTTGACACT TCCAAGAAAA GCAGTTCCTT | |
| | | GTTGATCACG GCTTCCCGGG CAGCAGACAC TGCTTCTTAC | |
| | | TTCTGTGCTA CGGACGCTAC CTCAGGAACC TACAAATACA | |
| | | TCTTTGGAAC AGGCACCAGG CTGAAGGTTT TAGCAAATAT | |
| | | CCAGAACCCT GACCCTGCCG TGTACCAGCT GAGAGACTCT | |
| | | AAATCCAGTG ACAAGTCTGT CTGCCTATTC ACCGATTTTG | |
| | | ATTCTCAAAC AAATGTGTCA CAAAGTAAGG ATTCTGATGT | |
| | | GTATATCACA GACAAATGTG TGCTAGACAT GAGGTCTATG | |
| | | GACTTCAAGA GCAACAGTGC TGTGGCCTGG AGCAACAAAT | |
| | | CTGACTTTGC ATGTGCAAAC GCCTTCAACA ACAGCATTAT | |
| | | TCCAGAAGAC ACCTTCTTCC CCAGCCCAGA AAGTTCCTGT | |
| | | GATGTCAAGC TGGTCGAGAA AAGCTTTGAA ACAGATACGA | |
| | | ACCTAAACTT TCAAAACCTG TCAGTGATTG GGTTCCGAAT | |
| | | CCTCCTCCTG AAAGTGGCCG GGTTTAATCT GCTCATGACG | |
| | | CTGCGGCTGT GGTCCAGCTG A | |
| | TRAC | NIQNPDPAVYQLRDSKSSDKSVCLFTDFDSQTNVSQSKDSDVYI | 58 |
| | (alpha | TDKTVLDMRSMDFKSNSAVAWSNKSDFACANAFNNSIIPEDTFF | |
| | constant | PSPESSCDVKLVEKSFETDTNLNFQNLSVIGFRILLLKVAGFNL | |
| | chain) | LMTLRLWSS | |
| | TRBC1 | EDLNKVFPPEVAVFEPSEAEISHTQKATLVCLATGFFPDHVELS | 59 |
| | (beta | WWVNGKEVHSGVSTDPQPLKEQPALNDSRYCLSSRLRVSATFWQ | |
| | constant | NPRNHFRCQVQFYGLSENDEWTQDRAKPVTQIVSAEAWGRADCG | |
| | chain) | FTSVSYQQGVLSATILYEILLGKATLYAVLVSALVLMAMVKRKD | |
| | | F | |
| | TRBC2 | EDLKNVFPPEVAVFEPSEAEISHTQKATLVCLATGFYPDHVELS | 60 |
| | (beta | WWVNGKEVHSGVSTDPQPLKEQPALNDSRYCLSSRLRVSATFWQ | |
| | constant | NPRNHFRCQVQFYGLSENDEWTQDRAKPVTQIVSAEAWGRADCG | |
| | chain) | FTSESYQQGVLSATILYEILLGKATLYAVLVSALVLMAMVKRKD | |
| | | SRG | |
| | MBP 83-99 | ENPVVHFFKNIVTPRTP | 61 |

In some embodiments, an exogenous TCR comprises two or more polypeptides. For example, in some embodiments, an exogenous TCR comprises a first polypeptide comprising an alpha chain and a second polypeptide comprising a beta chain.

In some embodiments, T cell receptor comprises (a) an alpha chain comprising an amino acid sequence having at least 80% identity to the amino acid sequence of SEQ ID NO: 113; and/or (b) a beta chain comprising an amino acid sequence having at least 80% identity to the amino acid sequence of SEQ ID NO: 119, wherein the T cell receptor binds specifically to a myelin basic protein (MBP) peptide complexed with a major histocompatibility complex (MHC).

In some embodiments, T cell receptor comprises (a) an alpha chain comprising an amino acid sequence having at least 85% identity to the amino acid sequence of SEQ ID NO: 113; and/or (b) a beta chain comprising an amino acid sequence having at least 85% identity to the amino acid sequence of SEQ ID NO: 119, wherein the T cell receptor binds specifically to a myelin basic protein (MBP) peptide complexed with a major histocompatibility complex (MHC).

In some embodiments, T cell receptor comprises (a) an alpha chain comprising an amino acid sequence having at least 90% identity to the amino acid sequence of SEQ ID NO: 113; and/or (b) a beta chain comprising an amino acid sequence having at least 90% identity to the amino acid sequence of SEQ ID NO: 119, wherein the T cell receptor binds specifically to a myelin basic protein (MBP) peptide complexed with a major histocompatibility complex (MHC).

In some embodiments, T cell receptor comprises (a) an alpha chain comprising an amino acid sequence having at least 95% identity to the amino acid sequence of SEQ ID NO: 113; and/or (b) a beta chain comprising an amino acid sequence having at least 95% identity to the amino acid sequence of SEQ ID NO: 119, wherein the T cell receptor binds specifically to a myelin basic protein (MBP) peptide complexed with a major histocompatibility complex (MHC).

In some embodiments, T cell receptor comprises (a) an alpha chain comprising an amino acid sequence having at least 96% identity to the amino acid sequence of SEQ ID NO: 113; and/or (b) a beta chain comprising an amino acid sequence having at least 96% identity to the amino acid sequence of SEQ ID NO: 119, wherein the T cell receptor binds specifically to a myelin basic protein (MBP) peptide complexed with a major histocompatibility complex (MHC).

In some embodiments, T cell receptor comprises (a) an alpha chain comprising an amino acid sequence having at least 97% identity to the amino acid sequence of SEQ ID NO: 113; and/or (b) a beta chain comprising an amino acid sequence having at least 97% identity to the amino acid sequence of SEQ ID NO: 119, wherein the T cell receptor binds specifically to a myelin basic protein (MBP) peptide complexed with a major histocompatibility complex (MHC).

In some embodiments, T cell receptor comprises (a) an alpha chain comprising an amino acid sequence having at least 98% identity to the amino acid sequence of SEQ ID NO: 113; and/or (b) a beta chain comprising an amino acid sequence having at least 98% identity to the amino acid sequence of SEQ ID NO: 119, wherein the T cell receptor binds specifically to a myelin basic protein (MBP) peptide complexed with a major histocompatibility complex (MHC).

In some embodiments, T cell receptor comprises (a) an alpha chain comprising an amino acid sequence having at least 99% identity to the amino acid sequence of SEQ ID NO: 113; and/or (b) a beta chain comprising an amino acid sequence having at least 99% identity to the amino acid sequence of SEQ ID NO: 119, wherein the T cell receptor binds specifically to a myelin basic protein (MBP) peptide complexed with a major histocompatibility complex (MHC).

In some embodiments, T cell receptor comprises (a) an alpha chain comprising an amino acid sequence having at least 100% identity to the amino acid sequence of SEQ ID NO: 113; and/or (b) a beta chain comprising an amino acid sequence having at least 100% identity to the amino acid sequence of SEQ ID NO: 119, wherein the T cell receptor binds specifically to a myelin basic protein (MBP) peptide complexed with a major histocompatibility complex (MHC).

In some embodiments, the amino acid sequence of the alpha chain has at least 85% identity to the amino acid sequence of SEQ ID NO: 113. In some embodiments, the amino acid sequence of the alpha chain has at least 90% identity to the amino acid sequence of SEQ ID NO: 113. In some embodiments, the amino acid sequence of the alpha chain has at least 95% identity to the amino acid sequence of SEQ ID NO: 113. In some embodiments, the amino acid sequence of the alpha chain has at least 96% identity to the amino acid sequence of SEQ ID NO: 113. In some embodiments, the amino acid sequence of the alpha chain has at least 97% identity to the amino acid sequence of SEQ ID NO: 113. In some embodiments, the amino acid sequence of the alpha chain has at least 98% identity to the amino acid sequence of SEQ ID NO: 113. In some embodiments, the amino acid sequence of the alpha chain has at least 99% identity to the amino acid sequence of SEQ ID NO: 113. In some embodiments, the amino acid sequence of the alpha chain has 100% identity to the amino acid sequence of SEQ ID NO: 113.

In some embodiments, the amino acid sequence of the alpha chain has at least 85% identity to the amino acid sequence of SEQ ID NO: 114. In some embodiments, the amino acid sequence of the alpha chain has at least 90% identity to the amino acid sequence of SEQ ID NO: 114. In some embodiments, the amino acid sequence of the alpha chain has at least 95% identity to the amino acid sequence of SEQ ID NO: 114. In some embodiments, the amino acid sequence of the alpha chain has at least 96% identity to the amino acid sequence of SEQ ID NO: 114. In some embodiments, the amino acid sequence of the alpha chain has at least 97% identity to the amino acid sequence of SEQ ID NO: 114. In some embodiments, the amino acid sequence of the alpha chain has at least 98% identity to the amino acid sequence of SEQ ID NO: 114. In some embodiments, the amino acid sequence of the alpha chain has at least 99% identity to the amino acid sequence of SEQ ID NO: 114. In some embodiments, the amino acid sequence of the alpha chain has 100% identity to the amino acid sequence of SEQ ID NO: 114.

In some embodiments, the amino acid sequence of the beta chain has at least 85% identity to the amino acid sequence of SEQ ID NO: 119. In some embodiments, the amino acid sequence of the beta chain has at least 90% identity to the amino acid sequence of SEQ ID NO: 119. In some embodiments, the amino acid sequence of the beta chain has at least 95% identity to the amino acid sequence of SEQ ID NO: 119. In some embodiments, the amino acid sequence of the beta chain has at least 96% identity to the amino acid sequence of SEQ ID NO: 119. In some embodiments, the amino acid sequence of the beta chain has at least 97% identity to the amino acid sequence of SEQ ID NO: 119. In some embodiments, the amino acid sequence of the beta chain has at least 98% identity to the amino acid sequence of SEQ ID NO: 119. In some embodiments, the amino acid sequence of the beta chain has at least 99% identity to the amino acid sequence of SEQ ID NO: 119. In some embodiments, the amino acid sequence of the beta chain has 100% identity to the amino acid sequence of SEQ ID NO: 119.

In some embodiments, the amino acid sequence of the beta chain has at least 85% identity to the amino acid sequence of SEQ ID NO: 120. In some embodiments, the amino acid sequence of the beta chain has at least 90% identity to the amino acid sequence of SEQ ID NO: 120. In some embodiments, the amino acid sequence of the beta chain has at least 95% identity to the amino acid sequence of SEQ ID NO: 120. In some embodiments, the amino acid sequence of the beta chain has at least 96% identity to the amino acid sequence of SEQ ID NO: 120. In some embodiments, the amino acid sequence of the beta chain has at least 97% identity to the amino acid sequence of SEQ ID NO: 120. In some embodiments, the amino acid sequence of the beta chain has at least 98% identity to the amino acid sequence of SEQ ID NO: 120. In some embodiments, the amino acid sequence of the beta chain has at least 99% identity to the amino acid sequence of SEQ ID NO: 120. In some embodiments, the amino acid sequence of the beta chain has 100% identity to the amino acid sequence of SEQ ID NO: 120.

In some embodiments, the amino acid sequence of the beta chain has at least 85% identity to the amino acid sequence of SEQ ID NO: 121. In some embodiments, the amino acid sequence of the beta chain has at least 90% identity to the amino acid sequence of SEQ ID NO: 121. In some embodiments, the amino acid sequence of the beta chain has at least 95% identity to the amino acid sequence of SEQ ID NO: 121. In some embodiments, the amino acid sequence of the beta chain has at least 96% identity to the amino acid sequence of SEQ ID NO: 121. In some embodiments, the amino acid sequence of the beta chain has at least 97% identity to the amino acid sequence of SEQ ID NO: 121. In some embodiments, the amino acid sequence of the beta chain has at least 98% identity to the amino acid sequence of SEQ ID NO: 121. In some embodiments, the amino acid sequence of the beta chain has at least 99% identity to the amino acid sequence of SEQ ID NO: 121. In some embodiments, the amino acid sequence of the beta chain has 100% identity to the amino acid sequence of SEQ ID NO: 121.

In some embodiments, the amino acid sequence of the beta chain has at least 85% identity to the amino acid sequence of SEQ ID NO: 122. In some embodiments, the amino acid sequence of the beta chain has at least 90% identity to the amino acid sequence of SEQ ID NO: 122. In some embodiments, the amino acid sequence of the beta chain has at least 95% identity to the amino acid sequence of SEQ ID NO: 122. In some embodiments, the amino acid sequence of the beta chain has at least 96% identity to the amino acid sequence of SEQ ID NO: 122. In some embodiments, the amino acid sequence of the beta chain has at least 97% identity to the amino acid sequence of SEQ ID NO: 122. In some embodiments, the amino acid sequence of the beta chain has at least 98% identity to the amino acid sequence of SEQ ID NO: 122. In some embodiments, the amino acid sequence of the beta chain has at least 99% identity to the amino acid sequence of SEQ ID NO: 122. In some embodiments, the amino acid sequence of the beta chain has 100% identity to the amino acid sequence of SEQ ID NO: 122.

In some embodiments, a T cell receptor comprises an amino acid sequence having at least 80% identity to the amino acid sequence of SEQ ID NO: 108. In some embodiments, a T cell receptor comprises an amino acid sequence having at least 85% identity to the amino acid sequence of SEQ ID NO: 108. In some embodiments, a T cell receptor comprises an amino acid sequence having at least 90% identity to the amino acid sequence of SEQ ID NO: 108. In some embodiments, a T cell receptor comprises an amino acid sequence having at least 95% identity to the amino acid sequence of SEQ ID NO: 108. In some embodiments, a T cell receptor comprises an amino acid sequence having at least 96% identity to the amino acid sequence of SEQ ID NO: 108. In some embodiments, a T cell receptor comprises an amino acid sequence having at least 97% identity to the amino acid sequence of SEQ ID NO: 108. In some embodiments, a T cell receptor comprises an amino acid sequence having at least 98% identity to the amino acid sequence of SEQ ID NO: 108. In some embodiments, a T cell receptor comprises an amino acid sequence having at least 99% identity to the amino acid sequence of SEQ ID NO: 108. In some embodiments, a T cell receptor comprises an amino acid sequence having at least 100% identity to the amino acid sequence of SEQ ID NO: 108.

In some embodiments, the TCR comprises one or more amino acid sequences as described in Table B. A TCR may comprise the amino acid sequence of any alpha chain CDR1, CDR2, or CDR3 as provided in Table B. In some embodiments, the alpha CDR1 of the TCR comprises the amino acid sequence of SEQ ID NO: 109. In some embodiments, the alpha CDR2 of the TCR comprises the amino acid sequence of SEQ ID NO: 110. In some embodiments, the alpha CDR3 of the TCR comprises the amino acid sequence of SEQ ID NO: 111. A TCR may comprise the amino acid sequence of any beta chain CDR1, CDR2, or CDR3 as provided in Table B. In some embodiments, the beta CDR1 of the TCR comprises the amino acid sequence of SEQ ID NO: 115. In some embodiments, the beta CDR2 of the TCR comprises the amino acid sequence of SEQ ID NO: 116. In some embodiments, the beta CDR3 of the TCR comprises the amino acid sequence of SEQ ID NO: 117.

In some embodiments, the alpha chain variable region of the TCR comprises an amino acid sequence having at least 80%, at least 85%, at least 90%, at least 95%, at least 96%, at least 97%, at least 98%, at least 99%, or 100% identity to the amino acid sequence of SEQ ID NO: 112. In some embodiments, the beta chain variable region of the TCR comprises an amino acid sequence having at least 80%, at least 85%, at least 90%, at least 95%, at least 96%, at least 97%, at least 98%, at least 99%, or 100% identity to the amino acid sequence of SEQ ID NO: 118.

In some embodiments, the alpha chain constant region of the TCR is a murine constant region. In some embodiments, the beta chain constant region of the TCR is a murine constant region.

In some embodiments, the alpha chain constant region of the TCR comprises an amino acid sequence having at least 80%, at least 85%, at least 90%, at least 95%, at least 96%, at least 97%, at least 98%, at least 99%, or 100% identity to the amino acid sequence of SEQ ID NO: 123. In some embodiments, the beta chain constant region of the TCR comprises an amino acid sequence having at least 80%, at least 85%, at least 90%, at least 95%, at least 96%, at least 97%, at least 98%, at least 99%, or 100% identity to the amino acid sequence of SEQ ID NO: 124.

In some embodiments, an engineered polypeptide comprises an amino acid sequence having at least 80% identity to the amino acid sequence of SEQ ID NO: 108. In some embodiments, an engineered polypeptide comprises an amino acid sequence having at least 85% identity to the amino acid sequence of SEQ ID NO: 108. In some embodiments, an engineered polypeptide comprises an amino acid sequence having at least 90% identity to the amino acid sequence of SEQ ID NO: 108. In some embodiments, an engineered polypeptide comprises an amino acid sequence having at least 95% identity to the amino acid sequence of SEQ ID NO: 108. In some embodiments, an engineered polypeptide comprises an amino acid sequence having at least 96% identity to the amino acid sequence of SEQ ID NO: 108. In some embodiments, an engineered polypeptide comprises an amino acid sequence having at least 97% identity to the amino acid sequence of SEQ ID NO: 108. In some embodiments, an engineered polypeptide comprises an amino acid sequence having at least 98% identity to the amino acid sequence of SEQ ID NO: 108. In some embodiments, an engineered polypeptide comprises an amino acid sequence having at least 99% identity to the amino acid sequence of SEQ ID NO: 2. In some embodiments, an engineered polypeptide comprises an amino acid sequence having 100% identity to the amino acid sequence of SEQ ID NO: 108.

In some embodiments, the present disclosure provides engineered polypeptides comprising the TCR sequences described herein.

In some embodiments, the present disclosure provides an engineered polypeptide comprising a human TCRα chain and a human TCRβ chain, wherein the TCRα chain is at least 95% identical to SEQ ID NO: 73 and the TCRβ chain is at least 95% identical to SEQ ID NO: 75; or the TCRα chain is at least 95% identical to SEQ ID NO: 80 and the TCRβ chain is at least 95% identical to SEQ ID NO: 82. In some embodiments, the TCRα chain comprises or consists of SEQ ID NO: 73 and the TCRβ chain comprises or consists of SEQ ID NO: 75; or the TCRα chain comprises or consists of SEQ ID NO: 80 and the TCRβ chain comprises or consists of SEQ ID NO: 82.

In some embodiments, the engineered polypeptides provided herein comprise from N-terminus to C-terminus: the TCRβ chain, a self-cleaving peptide sequence, and the TCRα chain. In some embodiments, the self-cleaving peptide is a P2A sequence (SEQ ID NO: 104).

In some embodiments, the engineered polypeptide comprises an amino acid sequence that is at least 95%, 96%, 97%, 98%, or 99% identical to SEQ ID NO: 79. In some embodiments, the engineered polypeptide comprises an amino acid sequence that is at least 100% identical to SEQ ID NO: 79. In some embodiments, the engineered polypeptide comprises SEQ ID NO: 79. In some embodiments, the engineered polypeptide consists of SEQ ID NO: 79.

In some embodiments, the engineered polypeptide comprises an amino acid sequence that is at least 95%, 96%, 97%, 98%, or 99% identical to SEQ ID NO: 86. In some embodiments, the engineered polypeptide comprises an amino acid sequence that is at least 100% identical to SEQ ID NO: 86. In some embodiments, the engineered polypeptide comprises SEQ ID NO: 86. In some embodiments, the engineered polypeptide consists of SEQ ID NO: 86.

In some embodiments, an engineered polypeptide comprises an amino acid sequence having at least 80% identity to the amino acid sequence of SEQ ID NO: 113. In some embodiments, an engineered polypeptide comprises an amino acid sequence having at least 85% identity to the amino acid sequence of SEQ ID NO: 113. In some embodiments, an engineered polypeptide comprises an amino acid sequence having at least 90% identity to the amino acid sequence of SEQ ID NO: 113. In some embodiments, an engineered polypeptide comprises an amino acid sequence having at least 95% identity to the amino acid sequence of SEQ ID NO: 113. In some embodiments, an engineered polypeptide comprises an amino acid sequence having at least 96% identity to the amino acid sequence of SEQ ID NO: 113. In some embodiments, an engineered polypeptide comprises an amino acid sequence having at least 97% identity to the amino acid sequence of SEQ ID NO: 113. In some embodiments, an engineered polypeptide comprises an amino acid sequence having at least 98% identity to the amino acid sequence of SEQ ID NO: 113. In some embodiments, an engineered polypeptide comprises an amino acid sequence having at least 99% identity to the amino acid sequence of SEQ ID NO: 113. In some embodiments, an engineered polypeptide comprises an amino acid sequence having at least 100% identity to the amino acid sequence of SEQ ID NO: 113.

In some embodiments, the amino acid sequence of the polypeptide further comprises an amino terminal leader sequence, optionally METLLGVSLV ILWLQLARVN (SEQ ID NO: 100).

In some embodiments, the amino acid sequence of the polypeptide comprises the amino acid sequence of SEQ ID NO: 114.

In some embodiments, an engineered polypeptide encoding a polypeptide comprising an amino acid sequence having at least 80% identity to the amino acid sequence of SEQ ID NO: 119. In some embodiments, an engineered polypeptide comprises an amino acid sequence having at least 85% identity to the amino acid sequence of SEQ ID NO: 119. In some embodiments, an engineered polypeptide comprises an amino acid sequence having at least 90% identity to the amino acid sequence of SEQ ID NO: 119. In some embodiments, an engineered polypeptide comprises an amino acid sequence having at least 95% identity to the amino acid sequence of SEQ ID NO: 119. In some embodiments, an engineered polypeptide comprises an amino acid sequence having at least 96% identity to the amino acid sequence of SEQ ID NO: 119. In some embodiments, an engineered polypeptide comprises an amino acid sequence having at least 97% identity to the amino acid sequence of SEQ ID NO: 119. In some embodiments, an engineered polypeptide comprises an amino acid sequence having at least 98% identity to the amino acid sequence of SEQ ID NO: 119. In some embodiments, an engineered polypeptide comprises an amino acid sequence having at least 99% identity to the amino acid sequence of SEQ ID NO: 119. In some embodiments, an engineered polypeptide comprises an amino acid sequence having at least 100% identity to the amino acid sequence of SEQ ID NO: 119.

In some embodiments, the amino acid sequence of the polypeptide further comprises an amino terminal leader sequence, optionally METLLGVSLV ILWLQLARVN (SEQ ID NO: 100). In some embodiments, the amino acid sequence of the polypeptide comprises the amino acid sequence of SEQ ID NO: 121.

In some embodiments, the amino acid sequence of the polypeptide further comprises a carboxy terminal P2A overhang sequence, optionally ATNFSLLKQAGDVEENPG (SEQ ID NO: 104).

In some embodiments, the amino acid sequence of the polypeptide comprises the amino acid sequence of SEQ ID NO: 120.

In some embodiments, the amino acid sequence of the polypeptide comprises the amino acid sequence of SEQ ID NO: 122.

TABLE B
Additional Exemplary TCR Sequences

| | | | SEQ |
|---|---|---|---|
| TCR | Description | Sequence | ID NO: |
| TCR-A | Beta-P2A- | ATGCTGCTGC TTCTGCTGCT TCTGGGGCCA GGTTCCGGGC | 107 |
| | alpha | TTGGTGCTGT CGTCTCTCAACATCCGAGCT GGGTTATCTG | |
| | | TAAGAGTGGA ACCTCTGTGA AGATCGAGTG | |
| | | CCGTTCCCTGGACTTTCAGG CCACAACTAT GTTTTGGTAT | |
| | | CGTCAGTTCC CGAAACAGAG TCTCATGCTGATGGCAACTT | |
| | | CCAATGAGGG CTCCAAGGCC ACATACGAGC AAGGCGTCGA | |
| | | GAAGGACAAGTTTCTCATCA ACCATGCAAG CCTGACCTTG | |
| | | TCCACTCTGA CAGTGACCAG TGCCCATCCTGAAGACAGCA | |
| | | GCTTCTACAT CTGCAGTGCT AGAGACCTCA CTTCCGGCGC | |
| | | CAATAATGAGCAGTTCTTCG GGCCAGGGAC ACGGCTCACC | |
| | | GTGCTAGAGG ATCTGAGAAA TGTGACTCCACCCAAGGTCT | |
| | | CCTTGTTTGA GCCATCAAAA GCAGAGATTG CAAACAAACA | |
| | | AAAGGCTACCCTCGTGTGCT TGGCCAGGGG CTTCTTCCCT | |
| | | GACCACGTGG AGCTGAGCTG GTGGGTGAATGGCAAGGAGG | |
| | | TCCACAGTGG GGTCAGCACG GACCCTCAGG CCTACAAGGA | |
| | | GAGCAATTATAGCTACTGCC TGAGCAGCCG CCTGAGGGTC | |
| | | TCTGCTACCT TCTGGCACAA TCCTCGAAACCACTTCCGCT | |
| | | GCCAAGTGCA GTTCCATGGG CTTTCAGAGG AGGACAAGTG | |
| | | GCCAGAGGGCTCACCCAAAC CTGTCACACA GAACATCAGT | |
| | | GCAGAGGCCT GGGGCCGAGC AGACTGTGGAATCACTTCAG | |
| | | CATCCTATCA TCAGGGGGTT CTGTCTGCAA CCATCCTCTA | |
| | | TGAGATCCTACTGGGGAAGG CCACCCTATA TGCTGTGCTG | |
| | | GTCAGTGGCC TGGTGCTGAT GGCCATGGTCAAGAAAAAAA | |
| | | ATTCCGGCAG CGGCGCCACC AACTTCTCCC TGCTGAAGCA | |
| | | GGCCGGCGACGTGGAAGAAA ACCCTGGCCC CATGGAAACT | |
| | | CTCCTGGGAG TGTCTTTGGT GATTCTATGGCTTCAACTGG | |
| | | CTAGGGTGAA CAGTCAACAG GGAGAAGAGG ATCCTCAGGC | |
| | | CTTGAGCATCCAGGAGGGTG AAAATGCCAC CATGAACTGC | |
| | | AGTTACAAAA CTAGTATAAA CAATTTACAGTGGTATAGAC | |
| | | AAAATTCAGG TAGAGGCCTT GTCCACCTAA TTTTAATACG | |
| | | TTCAAATGAAAGAGAGAAAC ACAGTGGAAG ATTAAGAGTC | |
| | | ACGCTTGACA CTTCCAAGAA AAGCAGTTCCTTGTTGATCA | |
| | | CGGCTTCCCG GGCAGCAGAC ACTGCTTCTT ACTTCTGTGC | |
| | | TACGGACACTACCTCAGGAA CCTACAAATA CATCTTTGGA | |
| | | ACAGGCACCA GGCTGAAGGT TTTAGCAAACATCCAGAACC | |
| | | CAGAACCTGC TGTGTACCAG TTAAAAGATC CTCGGTCTCA | |
| | | GGACAGCACCCTCTGCCTGT TCACCGACTT TGACTCCCAA | |
| | | ATCAATGTGC CGAAAACCAT GGAATCTGGAACGTTCATCA | |
| | | CTGACAAAAC TGTGCTGGAC ATGAAAGCTA TGGATTCCAA | |
| | | GAGCAATGGGGCCATTGCCT GGAGCAACCA GACAAGCTTC | |
| | | ACCTGCCAAG ATATCTTCAA AGAGACCAACGCCACCTACC | |
| | | CCAGTTCAGA CGTTCCCTGT GATGCCACGT TGACTGAGAA | |
| | | AAGCTTTGAAACAGATATGA ACCTAAACTT TCAAAACCTG | |
| | | TCAGTTATGG GACTCCGAAT CCTCCTGCTGAAAGTAGCCG | |
| | | GATTTAACCT GCTCATGACG CTGAGGCTGT GGTCCAGT | |
| | Beta-P2A- | MLLLLLLLGPGSGLGAVVSQHPSWVICKSGTSVKIECRSLDFQATTMF | 108 |
| | alpha | WYRQFPKQSLMLMATSNEGSKATYEQGVEKDKFLINHASLTLSTLTVT | |
| | | SAHPEDSSFYICSARDLTSGANNEQFFGPGTRLTVLEDLRNVTPPKVS | |
| | | LFEPSKAEIANKQKATLVCLARGFFPDHVELSWWVNGKEVHSGVSTDP | |
| | | QAYKESNYSYCLSSRLRVSATFWHNPRNHFRCQVQFHGLSEEDKWPEG | |
| | | SPKPVTQNISAEAWGRADCGITSASYHQGVLSATILYEILLGKATLYA | |
| | | VLVSGLVLMAMVKKKNSGSGATNFSLLKQAGDVEENPGPMETLLGVSL | |
| | | VILWLQLARVNSQQGEEDPQALSIQEGENATMNCSYKTSINNLQWYRQ | |
| | | NSGRGLVHLILIRSNEREKHSGRLRVTLDTSKKSSSLLITASRAADTA | |
| | | SYFCATDTTSGTYKYIFGTGTRLKVLANIQNPEPAVYQLKDPRSQDST | |
| | | LCLFTDFDSQINVPKTMESGTFITDKTVLDMKAMDSKSNGAIAWSNQT | |
| | | SFTCQDIFKETNATYPSSDVPCDATLTEKSFETDMNLNFQNLSVMGLR | |
| | | ILLLKVAGFNLLMTLRLWSS | |
| | TCRa CDR1 | KTSINNL | 109 |
| | TCRa CDR2 | LIRSNEREK | 110 |
| | TCRa CDR3 | CATDTTSGTY KYIF | 111 |
| | TCRa | SQQGEEDPQA LSIQEGENAT MNCSYKTSIN NLQWYRQNSG | 112 |
| | variable | RGLVHLILIR SNEREKHSGR LRVTLDTSKK SSSLLITASR | |
| | chain | AADTASYFCA TDTTSGTYKY IFGTGTRLKV LA | |
| | TCRa full | SQQGEEDPQALSIQEGENATMNCSYKTSINNLQWYRQNSGRGLVHLIL | 113 |
| | ORF | IRSNEREKHSGRLRVTLDTSKKSSSLLITASRAADTASYFCATDTTSG | |
| | | TYKYIFGTGTRLKVLANIQNPEPAVYQLKDPRSQDSTLCLFTDFDSQI | |
| | | NVPKTMESGTFITDKTVLDMKAMDSKSNGAIAWSNQTSFTCQDIFKET | |
| | | NATYPSSDVPCDATLTEKSFETDMNLNFQNLSVMGLRILLLKVAGFNL | |
| | | LMTLRLWSS | |
| | TCRa full | METLLGVSLVILWLQLARVNSQQGEEDPQALSIQEGENATMNCSYKTS | 114 |
| | ORF | INNLQWYRQNSGRGLVHLILIRSNEREKHSGRLRVTLDTSKKSSSLLI | |
| | (with leader) | TASRAADTASYFCATDTTSGTYKYIFGTGTRLKVLANIQNPEPAVYQL | |
| | | KDPRSQDSTLCLFTDFDSQINVPKTMESGTFITDKTVLDMKAMDSKSN | |
| | | GAIAWSNQTSFTCQDIFKETNATYPSSDVPCDATLTEKSFETDMNLNF | |
| | | QNLSVMGLRILLLKVAGFNLLMTLRLWSS | |
| | TCRb CDR1 | LDFQATTM | 115 |
| | TCRb CDR2 | TSNEGSKAT | 116 |
| | TCRb CDR3 | CSARDLTSGA NNEQFF | 117 |
| | TCRb | GAVVSQHPSW VICKSGTSVK IECRSLDFQA TTMFWYRQFP | 118 |
| | variable | KQSLMLMATS NEGSKATYEQ GVEKDKFLIN HASLTLSTLT | |
| | chain | VTSAHPEDSS FYICSARDLT SGANNEQFFG PGTRLTVL | |
| | TCRb full | GAVVSQHPSWVICKSGTSVKIECRSLDFQATTMFWYRQFPKQSLMLMA | 119 |
| | ORF | TSNEGSKATYEQGVEKDKFLINHASLTLSTLTVTSAHPEDSSFYICSA | |
| | | RDLTSGANNEQFFGPGTRLTVLEDLRNVTPPKVSLFEPSKAEIANKQK | |
| | | ATLVCLARGFFPDHVELSWWVNGKEVHSGVSTDPQAYKESNYSYCLSS | |
| | | RLRVSATFWHNPRNHFRCQVQFHGLSEEDKWPEGSPKPVTQNISAEAW | |
| | | GRADCGITSASYHQGVLSATILYEILLGKATLYAVLVSGLVLMAMVKK | |
| | | KNS | |
| | TCRb full | GAVVSQHPSWVICKSGTSVKIECRSLDFQATTMFWYRQFPKQSLMLMA | 120 |
| | ORF with | TSNEGSKATYEQGVEKDKFLINHASLTLSTLTVTSAHPEDSSFYICSA | |
| | P2A | RDLTSGANNEQFFGPGTRLTVLEDLRNVTPPKVSLFEPSKAEIANKQK | |
| | overhang | ATLVCLARGFFPDHVELSWWVNGKEVHSGVSTDPQAYKESNYSYCLSS | |
| | | RLRVSATFWHNPRNHFRCQVQFHGLSEEDKWPEGSPKPVTQNISAEAW | |
| | | GRADCGITSASYHQGVLSATILYEILLGKATLYAVLVSGLVLMAMVKK | |
| | | KNSGSG ATNFSLLKQA GDVEENPG | |
| | TCRb full | MLLLLLLLGPGSGLGAVVSQHPSWVICKSGTSVKIECRSLDFQATTMF | 121 |
| | ORF | WYRQFPKQSLMLMATSNEGSKATYEQGVEKDKFLINHASLTLSTLTVT | |
| | (with leader) | SAHPEDSSFYICSARDLTSGANNEQFFGPGTRLTVLEDLRNVTPPKVS | |
| | | LFEPSKAEIANKQKATLVCLARGFFPDHVELSWWVNGKEVHSGVSTDP | |
| | | QAYKESNYSYCLSSRLRVSATFWHNPRNHFRCQVQFHGLSEEDKWPEG | |
| | | SPKPVTQNISAEAWGRADCGITSASYHQGVLSATILYEILLGKATLYA | |
| | | VLVSGLVLMAMVKKKNS | |
| | TCRb full | MLLLLLLLGPGSGLGAVVSQHPSWVICKSGTSVKIECRSLDFQATTMF | 122 |
| | ORF as | WYRQFPKQSLMLMATSNEGSKATYEQGVEKDKFLINHASLTLSTLTVT | |
| | expressed | SAHPEDSSFYICSARDLTSGANNEQFFGPGTRLTVLEDLRNVTPPKVS | |
| | (with leader | LFEPSKAEIANKQKATLVCLARGFFPDHVELSWWVNGKEVHSGVSTDP | |
| | and P2A | QAYKESNYSYCLSSRLRVSATFWHNPRNHFRCQVQFHGLSEEDKWPEG | |
| | overhang) | SPKPVTQNISAEAWGRADCGITSASYHQGVLSATILYEILLGKATLYA | |
| | | VLVSGLVLMAMVKKKNS GSG ATNFSLLKQA GDVEENPG | |
| | TCRa | NIQNPEPAVYQLKDPRSQDSTLCLFTDFDSQINVPKTMESGTFITDKT | 123 |
| | Constant | VLDMKAMDSKSNGAIAWSNQTSFTCQDIFKETNATYPSSDVPCDATLT | |
| | Domain | EKSFETDMNLNFQNLSVMGLRILLLKVAGFNLLMTLRLWSS | |
| | TCRb | EDLRNVTPPKVSLFEPSKAEIANKQKATLVCLARGFFPDHVELSWWVN | 124 |
| | Constant | GKEVHSGVSTDPQAYKESNYSYCLSSRLRVSATFWHNPRNHFRCQVQF | |
| | Domain | HGLSEEDKWPEGSPKPVTQNISAEAWGRADCGITSASYHQGVLSATIL | |
| | | YEILLGKATLYAVLVSGLVLMAMVKKKNS | |

### Nucleic Acids Encoding a TCR

In some embodiments, the disclosure provides nucleic acids encoding a TCR (e.g., an exogenous TCR). Nucleic acids may be or may include deoxyribonucleic acid (DNA), ribonucleic acid (RNA) (e.g., messenger RNA), threose nucleic acid (TNA), glycol nucleic acid (GNA), peptide nucleic acid (PNA), locked nucleic acid (LNA), ethylene nucleic acid (ENA), cyclohexenyl nucleic acid (CeNA) and/or chimeras.

The nucleic acids used herein are generally engineered nucleic acids. An engineered nucleic acid is a polynucleotide (e.g., at least two nucleotides covalently linked together, and in some instances, containing phosphodiester bonds, referred to as a phosphodiester backbone) that does not occur in nature. Engineered nucleic acids include recombinant nucleic acids and synthetic nucleic acids. A recombinant nucleic acid is a molecule that is constructed by joining nucleic acids (e.g., isolated nucleic acids, synthetic nucleic acids or a combination thereof) from two different organisms (e.g., human and mouse). A synthetic nucleic acid is a molecule that is amplified or chemically, or by other means, synthesized. A synthetic nucleic acid includes those that are chemically modified, or otherwise modified, but can base pair with (bind to) naturally occurring nucleic acid molecules. Recombinant and synthetic nucleic acids also include those molecules that result from the replication of either of the foregoing.

Engineered nucleic acids of the present disclosure may be produced using standard molecular biology methods (see, e.g., Green and Sambrook, Molecular Cloning, A Laboratory Manual, 2012, Cold Spring Harbor Press). In some embodiments, nucleic acids are produced using GIBSON ASSEMBLY® Cloning (see, e.g., Gibson, D. G. et al. Nature Methods, 343-345, 2009; and Gibson, D. G. et al. Nature Methods, 901-903, 2010, each of which is incorporated by reference herein). GIBSON ASSEMBLY® typically uses three enzymatic activities in a single-tube reaction: 5′ exonuclease, the 3′ extension activity of a DNA polymerase and DNA ligase activity. The 5′ exonuclease activity chews back the 5′ end sequences and exposes the complementary sequence for annealing. The polymerase activity then fills in the gaps on the annealed domains. A DNA ligase then seals the nick and covalently links the DNA fragments together. The overlapping sequence of adjoining fragments is much longer than those used in Golden Gate Assembly, and therefore results in a higher percentage of correct assemblies. The MegaGate molecular cloning method may also be used. MegaGate is a toxin-less Gateway technology that eliminates the ccdb toxin used in Gateway recombinase cloning and instead utilizes meganuclease-mediated digestion to eliminate background vectors during cloning (see, e.g., Kramme C. et al. STAR Protoc. 2021 Oct. 22; 2 (4): 100907, incorporated herein by reference). Other methods of producing engineered polynucleotides may be used in accordance with the present disclosure.

In some embodiments, the present disclosure provides an expression cassette comprising an open reading frame comprising a nucleic acid encoding the exogenous TCR operably linked to a promoter. A promoter is a nucleotide sequence to which RNA polymerase binds to initial transcription (e.g., ATG). Promoters are typically located directly upstream from (at the 5′ end of) a transcription initiation site. In some embodiments, a promoter is a heterologous promoter. A heterologous promoter is not naturally associated with the open reading frame to which is it operably linked. In some embodiments, a promoter is an inducible promoter. An inducible promoter may be regulated in vivo by a chemical agent, temperature, or light, for example.

An open reading frame is a continuous stretch of codons that begins with a start codon (e.g., ATG), ends with a stop codon (e.g., TAA, TAG, or TGA), and encodes a polypeptide, for example, a protein. An open reading frame is operably linked to a promoter if that promoter regulates transcription of the open reading frame.

In some embodiments, the present disclosure provides a vector comprising the nucleic acid encoding the exogenous TCR or an expression vector comprising the same. In some embodiments, the vector is a plasmid. In some embodiments, the vector is a viral vector. For example, the vector may be a lentiviral vector, an adenovirus vector, an adeno-associated viral (AAV) vector, a herpes viral vector, a retroviral vector, or a baculoviral vector. A viral vector provides efficient delivery of the exogenous TCR into regulatory T cells of the disclosure. Exemplary viral vectors may be derived from lentivirus, retrovirus (e.g., Retroviridae family viral vector), adenovirus (e.g., Ad5, Ad26, Ad34, Ad35, and Ad48), parvovirus (e.g., adeno-associated viruses), coronavirus, negative strand RNA viruses such as orthomyxovirus (e.g., influenza virus), rhabdovirus (e.g., rabies and vesicular stomatitis virus), paramyxovirus (e.g., measles and Sendai), positive strand RNA viruses, such as picornavirus and alphavirus, and double stranded DNA viruses including adenovirus, herpesvirus (e.g., Herpes Simplex virus types 1 and 2, Epstein-Barr virus, cytomegalovirus, replication deficient herpes virus), and poxvirus (e.g., vaccinia, modified vaccinia Ankara (MVA), fowlpox and canarypox). Other viruses include Norwalk virus, togavirus, flavivirus, reoviruses, papovavirus, hepadnavirus, human papilloma virus, human foamy virus, and hepatitis virus, for example. Examples of retroviruses include: avian leukosis-sarcoma, avian C-type viruses, mammalian C-type, B-type viruses, D-type viruses, oncoretroviruses, HTLV-BLV group, alpharetrovirus, gammaretrovirus, spumavirus, murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus and lentiviruses.

In some embodiments, the vector is a lentiviral vector. In some embodiments, the lentiviral vectors of the present disclosure comprise a lentiviral gag, pol and rev genes and two long terminal repeats (LTRs) which flank the expression cassette comprising the nucleic acid encoding the exogenous TCR. For safety, the vector will not include any other active lentiviral genes, such as vpr, vif, vpu, nef, tat. In some embodiments, these genes have been removed or otherwise inactivated.

In some embodiments, the lentiviral vector is a self-inactivating vector. Self-inactivating vectors are vectors where the production of full-length vector RNA in transduced cells in greatly reduced or abolished altogether. This feature greatly minimizes the risk that replication-competent recombinants (RCRs) will emerge. Furthermore, it reduces the risk that that cellular coding sequences located adjacent to the vector integration site will be aberrantly expressed. Furthermore, an SIN design reduces the possibility of interference between the LTR and the promoter that is driving the expression of the transgene.

Self-inactivation is preferably achieved through the introduction of a deletion in the U3 region of the 3′ LTR of the vector DNA, i.e., the DNA used to produce the vector RNA. Thus, during reverse transcription, this deletion is transferred to the 5′ LTR of the proviral DNA. However, the elements of the LTR that are involved with polyadenylation of the viral RNA are not be modified. Together this diminishes or abolishes the production of full-length vector RNA in transduced cells.

In some embodiments, a nucleic acid encoding an exogenous TCR is an RNA (e.g., a messenger RNA (mRNA)). In some embodiments, an mRNA comprises a 5′ cap, a 5′ untranslated region (UTR), an open reading frame (ORF), a 3′ UTR, and/or a poly(A) tail.

In some embodiments, a nucleic acid is codon optimized. Codon optimization methods are known in the art. Codon optimization, in some embodiments, may be used to match codon frequencies in target and host organisms to ensure proper folding; bias GC content to increase RNA (e.g., mRNA) stability or reduce secondary structures; minimize tandem repeat codons or base runs that may impair gene construction or expression; customize transcriptional and translational control regions; insert or remove protein trafficking sequences; remove/add post-translation modification sites in encoded protein (e.g., glycosylation sites); add, remove or shuffle protein domains; insert or delete restriction sites; modify ribosome binding sites and RNA (e.g., mRNA) degradation sites; adjust translational rates to allow the various domains of the protein to fold properly; or reduce or eliminate problem secondary structures within the polynucleotide

In some embodiments, a nucleic acid encoding a TCR comprises a promoter operably linked to a coding sequence encoding the exogenous human TCR. A promoter may be a viral promoter or a natural TCR promoter. In some embodiments, a promoter is a constitutively active promoter or an inducible promoter. In some embodiments, a promoter is the eukaryotic translation elongation factor 1 alpha (EF-1 alpha) promoter and the MND promoter (myeloproliferative sarcoma virus enhancer, negative control region deleted, dl587rev primer-binding site substituted) (see, e.g., Gill, D R. et al. Gene Ther. 2001; 8:1539-46 and Astrakhan, A. et al. Blood 2012; 119:4395-4407).

A vector may also include a termination codon and/or expression enhancer elements. Any suitable vectors, promoters, enhancers and termination codons known in the art may be used. In some embodiments, an enhancer element is an optimized post-transcriptional regulatory element (oPRE), a woodchuck hepatitis virus post-transcriptional regulatory element (WPRE). A WPRE may be a wild-type WPRE or a WPRE mutant sequence (e.g., WPRE-mut6). In some embodiments, a WPRE is as described in Zanta-Boussif, M. A. et al., Gene Therapy volume 16, pages 605-619 (2009).

In some embodiments, an engineered polynucleotide comprises an open reading frame comprising a nucleotide sequence having at least 80% identity to the nucleotide sequence of SEQ ID NO: 87. In some embodiments, an engineered polynucleotide comprises an open reading frame comprising a nucleotide sequence having at least 85% identity to the nucleotide sequence of SEQ ID NO: 87. In some embodiments, an engineered polynucleotide comprises an open reading frame comprising a nucleotide sequence having at least 90% identity to the nucleotide sequence of SEQ ID NO: 87. In some embodiments, an engineered polynucleotide comprises an open reading frame comprising a nucleotide sequence having at least 95% identity to the nucleotide sequence of SEQ ID NO: 87. In some embodiments, an engineered polynucleotide comprises an open reading frame comprising a nucleotide sequence having at least 96% identity to the nucleotide sequence of SEQ ID NO: 87. In some embodiments, an engineered polynucleotide comprises an open reading frame comprising a nucleotide sequence having at least 97% identity to the nucleotide sequence of SEQ ID NO: 87. In some embodiments, an engineered polynucleotide comprises an open reading frame comprising a nucleotide sequence having at least 98% identity to the nucleotide sequence of SEQ ID NO: 87. In some embodiments, an engineered polynucleotide comprises an open reading frame comprising a nucleotide sequence having at least 99% identity to the nucleotide sequence of SEQ ID NO: 87. In some embodiments, an engineered polynucleotide comprises an open reading frame comprising a nucleotide sequence having 100% identity to the nucleotide sequence of SEQ ID NO: 87.

In some embodiments, an engineered polynucleotide encodes a polypeptide comprising an amino acid sequence having at least 80% identity to the amino acid sequence of SEQ ID NO: 79. In some embodiments, an engineered polynucleotide encodes a polypeptide comprising an amino acid sequence having at least 85% identity to the amino acid sequence of SEQ ID NO: 79. In some embodiments, an engineered polynucleotide encodes a polypeptide comprising an amino acid sequence having at least 90% identity to the amino acid sequence of SEQ ID NO: 79. In some embodiments, an engineered polynucleotide encodes a polypeptide comprising an amino acid sequence having at least 95% identity to the amino acid sequence of SEQ ID NO: 79. In some embodiments, an engineered polynucleotide encodes a polypeptide comprising an amino acid sequence having at least 96% identity to the amino acid sequence of SEQ ID NO: 79. In some embodiments, an engineered polynucleotide encodes a polypeptide comprising an amino acid sequence having at least 97% identity to the amino acid sequence of SEQ ID NO: 79. In some embodiments, an engineered polynucleotide encodes a polypeptide comprising an amino acid sequence having at least 98% identity to the amino acid sequence of SEQ ID NO: 79. In some embodiments, an engineered polynucleotide encodes a polypeptide comprising an amino acid sequence having at least 99% identity to the amino acid sequence of SEQ ID NO: 79. In some embodiments, an engineered polynucleotide encodes a polypeptide comprising an amino acid sequence having 100% identity to the amino acid sequence of SEQ ID NO: 79.

In some embodiments, the amino acid sequence of the polypeptide encoded by the polynucleotide further comprises a carboxy terminal self-cleaving peptide sequence. The self-cleaving peptide sequence may be selected from P2A, E2A, F2A, and T2A. In some embodiments, the self-cleaving peptide sequence is the sequence of ATNFSLLKQAGDVEENPG (SEQ ID NO: 104).

### Isolated Cell Populations

In some embodiments, the present disclosure provides isolated populations of cells comprising stable CD4⁺ T regulatory cells derived from a subject having multiple sclerosis, as well as compositions thereof. In some embodiments, the present disclosure provides isolated populations of cells comprising stable CD4⁺ T regulatory cells derived from a subject having multiple sclerosis, an engineered to express an exogenous human TCR, as well as compositions thereof. In some embodiments, at least 80% of the cells are stable CD4⁺ T regs comprising a hypomethylated T cell specific demethylated region (TSDR) at the FOXP3 locus.

The methylation status of the TSDR at the FOXP3 locus can be evaluated by means known in the art. For example, in some embodiments, methylation status of the TSDR at the FOXP3 locus is evaluated by bisulfite treatment and digital droplet PCR (ddPCR) using methylation-specific primers and probes. In some embodiments, methylation status of the TSDR at the FOXP3 locus is evaluated by single-cell sequencing methods.

An isolated cell population is a cell population that is removed from a human body, or removed from a sample obtained from a human body. Thus, it is considered “isolated” from the human body. A population of cells may be isolated (e.g., obtained from) a subject, or from a biological sample obtained from the subject, for example, using any known cell collection method, such as apheresis. An isolated cell population of the disclosure may be subjected to the methods described herein to produce an isolated cell population a higher number of regulatory T cells (e.g., stable regulatory T cells) relative to a population of cells obtained directly from a subject, or from a biological sample obtained from the subject, by apheresis, for example.

In some embodiments, the isolated cell population comprises stable CD4⁺ T regulatory cells, wherein at least 80% of the cells are stable CD4⁺ T regs comprising a hypomethylated T cell specific demethylated region (TSDR) at the FOXP3 locus. In some embodiments, at least 81%, 82%, 83%, 84%, 85%, 86%, 87%, 88%, 89%, 90%, 91%, 92%, 93%, 94%, 95%, 96%, 97%, 98%, or 99% of the cells are stable CD4⁺ T regs comprising a hypomethylated TSDR at the FOXP3 locus. In some embodiments, the isolated cell population comprises stable CD4+ T regulatory cells, wherein 80%, 81%, 82%, 83%, 84%, 85%, 86%, 87%, 88%, 89%, 90%, 91%, 92%, 93%, 94%, 95%, 96%, 97%, 98%, or 99% of the cells are stable CD4+T regs comprising a hypomethylated TSDR at the FOXP3 locus. In some embodiments, between 80% and 90%, between 85% and 95%, between 80% and 85%, between 85% and 90%, between 90% and 95% of the cells are stable CD4⁺ T regs comprising a hypomethylated TSDR at the FOXP3 locus. In some embodiments, at least 50%, at least 60%, at least 70%, at least 75%, at least 80%, at least 85%, at least 90%, at least 95%, at least 96%, at least 97%, at least 98%, or at least 99% of the cells of an isolated cell population are stable regulatory T cells comprising a hypomethylated TSDR at an endogenous FOXP3 locus.

In some embodiments, the isolated population comprises CD25^{+/high}CD4⁺CD127^{-/low} regulatory T cells. In some embodiments, the isolated cell population comprises CD25^{+/high}CD4⁺CD127^{-/low}FOXP3⁺ regulatory T cells. In some embodiments, the isolated cell population comprises CD25^{+/high}CD4⁺CD127^{-/low}FOXP3+CD45RA⁺ regulatory T cells.

In some embodiments, the isolated cell population comprises at least 50%, at least 60%, at least 70%, at least 75%, at least 80%, at least 85%, at least 90%, at least 95%, at least 96%, at least 97%, at least 98%, or at least 99% CD25+/highCD4+CD127-/lo regulatory T cells. In some embodiments, the isolated cell population comprises at least 50%, at least 60%, at least 70%, at least 75%, at least 80%, at least 85%, at least 90%, at least 95%, at least 96%, at least 97%, at least 98%, or at least 99% CD25^{+/high}CD4⁺CD127^{-/low}FOXP3⁺ regulatory T cells.

In some embodiments, at least 10%, at least 25%, at least 50%, at least 75%, at least 80%, at least 85%, at least 90%, at least 95%, at least 96%, at least 97%, at least 98%, or at least 99% of the regulatory T cells in the isolated population are CD45RA⁺. In some embodiments, the isolated cell population comprises at least 10%, at least 25%, at least 50%, at least 75%, at least 80%, at least 85%, at least 90%, at least 95%, at least 96%, at least 97%, at least 98%, or at least 99% CD25^{+/high}CD4⁺CD127^{-/low}CD45RA⁺ regulatory T cells.

As described herein, the present disclosure provides isolated cell populations comprising stable T regulatory cells comprising a hypomethylated TSDR at the FOXP3 locus. While the isolated populations described herein are, in some embodiments, engineered to express an exogenous TCR, the FOXP3 locus remains unmodified. Therefore, the cells of the isolated populations described herein do not comprise an engineered FOXP3 locus. An “engineered FOXP3 locus” refers to any engineered modification (e.g., a modification by the hand of man) intended to alter expression of FOXP3. Such engineered modifications include, but are not limited to, introduction of a FOXP3 transgene, introduction of a modified promoter, and/or use of an exogenous agent (e.g., gene editing system, small molecule, or peptide) intended to activate expression of FOXP3.

In some embodiments, the isolated cell population comprises at least 1×10², at least 1×10³, at least 1×10⁴, at least 1×10⁵, at least 1×10⁶, at least 1×10⁷, at least 1×10⁸, at least 1×10⁹, or at least 1×10¹⁰ stable regulatory T cells. In some embodiments, the isolated cell population comprises 1×10² to 1×10¹⁰, 1×10³ to 1×10¹⁰, 1×10⁴ to 1×10¹⁰, 1×10⁵ to 1×10¹⁰, 1×10⁶ to 1×10¹⁰, 1×10⁷ to 1×10¹⁰, 1×10⁸ to 1×10¹⁰, 1×10⁵ to 1×10⁹, 1×10⁶ to 1×10⁸, 1×10⁷ to 1×10¹⁰, or 1×10⁴ to 1×10⁶ stable regulatory T cells. In some embodiments, the isolated cell population comprises 1×10⁶ to 1×10¹⁰ stable regulatory T cells. In some embodiments, the isolated cell population comprises 1×10⁷, 2×10⁷, 3×10⁷, 4×10⁷, 5×10⁷, 6×10⁷, 7×10⁷, 8×10⁷, or 9×10⁷ stable regulatory T cells. In some embodiments, the isolated cell population comprises 1×10⁷ to 1×10¹⁰, 2×10⁷ to 1×10¹⁰, 3×10⁷ to 1×10¹⁰, 4×10⁷ to 1×10¹⁰, 5×10⁷ to 1×10¹⁰, 6×10⁷ to 1×10¹⁰, 7×10⁷ to 1×10¹⁰, 8×10⁷ to 1×10¹⁰, or 9×10⁷ to 1×10¹⁰ stable regulatory T cells.

In some embodiments, the isolated cell population comprises stable T regulatory cells comprising a hypomethylated TSDR at the FOXP3 locus, wherein the stable T regulatory cells retain markers of stability (i.e., maintain the hypomethylated TSDR at the FOXP3 locus and/or maintain the protein expression profile of CD25^{+/high}CD4⁺CD127^{-/low}FOXP3⁺) in the presence of proinflammatory conditions. Proinflammatory conditions refer to cells and factors known to drive inflammatory immune responses and can include pro-inflammatory cytokines (e.g., IL-17, IL-22, IL-21, IFNγ, IL-12, TNFα, IL-1β, IL-6, IL-1, GM-CSF, and others known in the art), immune effector cells (e.g., conventional CD4+ T cells, CD8+ effector T cells, granulocytes, etc.), and other proinflammatory mediators (e.g., prostaglandins, thrombin, histamine, and matrix proteases). The proinflammatory conditions may be in vitro or in vivo. In some embodiments, the stable regulatory T cells within an isolated cell population maintain a hypomethylated TSDR at an endogenous FOXP3 locus in the presence of pro-inflammatory conditions (for at least 3, 4, 5, 6, 7, 8, 9, 10, 11, 12, 13, 14, 15, 20, 21, 22, 23, 24, or 25 days.

In some embodiments, the isolated cell population comprises stable T regulatory cells comprising a hypomethylated TSDR at the FOXP3 locus, wherein the stable T regulatory cells retain markers of stability (i.e., maintain the hypomethylated TSDR at the FOXP3 locus and/or maintain the protein expression profile of CD25^{+/high}CD4⁺CD127^{-/low}FOXP3⁺) over time. For example, in some embodiments, the stable T regulatory cells maintain the hypomethylated TSDR at the FOXP3 locus ex vivo for at least 1, 2, 3, 4, 5, 6, 7, 8, 9, 10, 11, 12, 13, 14, 15, 16, 17, 18, 19, or 20 days after isolation from the biological sample. In some embodiments, the stable T regulatory cells maintain the hypomethylated TSDR at the FOXP3 locus ex vivo for at least 1, 2, 3, 4, 5, 6, 7, 8, 9, 10, 11, 12, 13, 14, 15, 16, 17, 18, 19, or 20 days after activation and/or expansion. In some embodiments, the stable T regulatory cells maintain the hypomethylated TSDR at the FOXP3 locus ex vivo for at least 1, 2, 3, 4, 5, 6, 7, 8, 9, 10, 11, 12, 13, 14, 15, 16, 17, 18, 19, or 20 days after transduction with an exogenous TCR. In some embodiments, the stable T regulatory cells maintain the hypomethylated TSDR at the FOXP3 locus ex vivo for at least 1, 2, 3, 4, 5, 6, 7, 8, 9, 10, 11, 12, 13, 14, 15, 16, 17, 18, 19, or 20 days after a cryopreservation freeze-thaw cycle.

In some embodiments, the stable T regulatory cells maintain the protein expression profile of CD25^{+/high}CD4⁺CD127^{-/low}FOXP3⁺ ex vivo for at least 1, 2, 3, 4, 5, 6, 7, 8, 9, 10, 11, 12, 13, 14, 15, 16, 17, 18, 19, or 20 days after isolation from the biological sample. In some embodiments, the stable T regulatory cells maintain the protein expression profile of CD25^{+/high}CD4⁺CD127^{-/low}FOXP3⁺ ex vivo for at least 1, 2, 3, 4, 5, 6, 7, 8, 9, 10, 11, 12, 13, 14, 15, 16, 17, 18, 19, or 20 days after activation and/or expansion. In some embodiments, the stable T regulatory cells maintain the protein expression profile of CD25^{+/high}CD4⁺CD127^{-/low}FOXP3⁺ ex vivo for at least 1, 2, 3, 4, 5, 6, 7, 8, 9, 10, 11, 12, 13, 14, 15, 16, 17, 18, 19, or 20 days after transduction with an exogenous TCR. In some embodiments, the stable T regulatory cells maintain the protein expression profile of CD25^{+/high}CD4⁺CD127^{-/low}FOXP3⁺ ex vivo for at least 1, 2, 3, 4, 5, 6, 7, 8, 9, 10, 11, 12, 13, 14, 15, 16, 17, 18, 19, or 20 days after a cryopreservation freeze-thaw cycle.

In some embodiments, at least 50% of the cells of an isolated cell population comprises a hypomethylated TSDR at a FOXP3 locus for at least 5 days after isolation. In some embodiments, at least 50% of the cells of an isolated cell population comprises a hypomethylated TSDR at a FOXP3 locus for at least 10 days after isolation. In some embodiments, at least 50% of the cells of an isolated cell population comprises a hypomethylated TSDR at a FOXP3 locus for at least 15 days after isolation. In some embodiments, at least 60% of the cells of an isolated cell population comprises a hypomethylated TSDR at a FOXP3 locus for at least 5 days after isolation. In some embodiments, at least 60% of the cells of an isolated cell population comprises a hypomethylated TSDR at a FOXP3 locus for at least 10 days after isolation. In some embodiments, at least 60% of the cells of an isolated cell population comprises a hypomethylated TSDR at a FOXP3 locus for at least 15 days after isolation. In some embodiments, at least 70% of the cells of an isolated cell population comprises a hypomethylated TSDR at a FOXP3 locus for at least 5 days after isolation. In some embodiments, at least 70% of the cells of an isolated cell population comprises a hypomethylated TSDR at a FOXP3 locus for at least 10 days after isolation. In some embodiments, at least 70% of the cells of an isolated cell population comprises a hypomethylated TSDR at a FOXP3 locus for at least 15 days after isolation. In some embodiments, at least 80% of the cells of an isolated cell population comprises a hypomethylated TSDR at a FOXP3 locus for at least 5 days after isolation. In some embodiments, at least 80% of the cells of an isolated cell population comprises a hypomethylated TSDR at a FOXP3 locus for at least 10 days after isolation. In some embodiments, at least 80% of the cells of an isolated cell population comprises a hypomethylated TSDR at a FOXP3 locus for at least 15 days after isolation. In some embodiments, at least 90% of the cells of an isolated cell population comprises a hypomethylated TSDR at a FOXP3 locus for at least 5 days after isolation. In some embodiments, at least 90% of the cells of an isolated cell population comprises a hypomethylated TSDR at a FOXP3 locus for at least 10 days after isolation. In some embodiments, at least 90% of the cells of an isolated cell population comprises a hypomethylated TSDR at a FOXP3 locus for at least 15 days after isolation.

In some embodiments, hypomethylation of a TSDR at the FOXP3 locus is assessed at multiple (e.g., 2 or more) timepoints during the isolation and production process. In some embodiments, the percentage of stable T regulatory cells comprising a hypomethylated TSDR at the FOXP3 locus does not decrease by more than 20% between these 2 or more timepoints.

For example, in some embodiments, hypomethylation of a TSDR at the FOXP3 locus is assessed within 1-3 days after isolation from a biological sample and again within 5, 6, 7, 8, 9, 10, or 11 days after isolation from a biological sample. In such embodiments, the percentage of stable T regulatory cells comprising a hypomethylated TSDR at the FOXP3 locus does not decrease by more than 20%, more than 19%, more than 18%, more than 17%, more than 16%, more than 15%, more than 14%, more than 13%, more than 12%, more than 11%, more than 10%, more than 9%, more than 8%, more than 7%, more than 6%, more than 5%, more than 4%, more than 3%, more than 2%, or more than 1% within 5, 6, 7, 8, 9, 10, or 11 days after isolation from a biological sample. In such embodiments, the percentage of stable T regulatory cells comprising a hypomethylated TSDR at the FOXP3 locus does not decrease by more than 10% within 5, 6, 7, 8, 9, 10, or 11 days after isolation from a biological sample. In such embodiments, the percentage of stable T regulatory cells comprising a hypomethylated TSDR at the FOXP3 locus does not decrease by more than 5% within 5, 6, 7, 8, 9, 10, or 11 days after isolation from a biological sample.

In some embodiments, hypomethylation of a TSDR at the FOXP3 locus is assessed within 1-3 days after isolation from a biological sample and again within 5, 6, 7, 8, 9, 10, or 11 days after activation and/or expansion. In some embodiments, the percentage of stable T regulatory cells comprising a hypomethylated TSDR at the FOXP3 locus does not decrease by more than 20% within 5, 6, 7, 8, 9, 10, or 11 days after activation and/or expansion. In some embodiments, hypomethylation of a TSDR at the FOXP3 locus is assessed within 1-3 days after isolation from a biological sample and again within 5, 6, 7, 8, 9, 10, or 11 days after activation and/or expansion. In such embodiments, the percentage of stable T regulatory cells comprising a hypomethylated TSDR at the FOXP3 locus does not decrease by more than 20%, more than 19%, more than 18%, more than 17%, more than 16%, more than 15%, more than 14%, more than 13%, more than 12%, more than 11%, more than 10%, more than 9%, more than 8%, more than 7%, more than 6%, more than 5%, more than 4%, more than 3%, more than 2%, or more than 1% within 5, 6, 7, 8, 9, 10, or 11 days after activation and/or expansion. In such embodiments, the percentage of stable T regulatory cells comprising a hypomethylated TSDR at the FOXP3 locus does not decrease by more than 10% within 5, 6, 7, 8, 9, 10, or 11 days after activation and/or expansion. In such embodiments, the percentage of stable T regulatory cells comprising a hypomethylated TSDR at the FOXP3 locus does not decrease by more than 5% within 5, 6, 7, 8, 9, 10, or 11 days after activation and/or expansion.

In some embodiments, hypomethylation of a TSDR at the FOXP3 locus is assessed within 1-3 days after isolation from a biological sample and again within 5, 6, 7, 8, 9, 10, or 11 days after transduction with an exogenous human TCR. In some embodiments, the percentage of stable T regulatory cells comprising a hypomethylated TSDR at the FOXP3 locus does not decrease by more than 20% within 5, 6, 7, 8, 9, 10, or 11 days after transduction with an exogenous human TCR. In some embodiments, hypomethylation of a TSDR at the FOXP3 locus is assessed within 1-3 days after isolation from a biological sample and again within 5, 6, 7, 8, 9, 10, or 11 days after transduction with an exogenous human TCR. In such embodiments, the percentage of stable T regulatory cells comprising a hypomethylated TSDR at the FOXP3 locus does not decrease by more than 20%, more than 19%, more than 18%, more than 17%, more than 16%, more than 15%, more than 14%, more than 13%, more than 12%, more than 11%, more than 10%, more than 9%, more than 8%, more than 7%, more than 6%, more than 5%, more than 4%, more than 3%, more than 2%, or more than 1% within 5, 6, 7, 8, 9, 10, or 11 days after transduction with an exogenous human TCR. In such embodiments, the percentage of stable T regulatory cells comprising a hypomethylated TSDR at the FOXP3 locus does not decrease by more than 10% within 5, 6, 7, 8, 9, 10, or 11 days after transduction with an exogenous human TCR. In such embodiments, the percentage of stable T regulatory cells comprising a hypomethylated TSDR at the FOXP3 locus does not decrease by more than 5% within 5, 6, 7, 8, 9, 10, or 11 days after transduction with an exogenous human TCR.

In some embodiments, hypomethylation of a TSDR at the FOXP3 locus is assessed within 1-3 days after isolation from a biological sample and again within 5, 6, 7, 8, 9, 10, or 11 days after a cryopreservation freeze-thaw cycle. In some embodiments, the percentage of stable T regulatory cells comprising a hypomethylated TSDR at the FOXP3 locus does not decrease by more than 20% within 5, 6, 7, 8, 9, 10, or 11 days after a cryopreservation freeze-thaw cycle. In some embodiments, hypomethylation of a TSDR at the FOXP3 locus is assessed within 1-3 days after isolation from a biological sample and again within 5, 6, 7, 8, 9, 10, or 11 days after a cryopreservation freeze-thaw cycle. In such embodiments, the percentage of stable T regulatory cells comprising a hypomethylated TSDR at the FOXP3 locus does not decrease by more than 20%, more than 19%, more than 18%, more than 17%, more than 16%, more than 15%, more than 14%, more than 13%, more than 12%, more than 11%, more than 10%, more than 9%, more than 8%, more than 7%, more than 6%, more than 5%, more than 4%, more than 3%, more than 2%, or more than 1% within 5, 6, 7, 8, 9, 10, or 11 days after a cryopreservation freeze-thaw cycle. In such embodiments, the percentage of stable T regulatory cells comprising a hypomethylated TSDR at the FOXP3 locus does not decrease by more than 10% within 5, 6, 7, 8, 9, 10, or 11 days after a cryopreservation freeze-thaw cycle. In such embodiments, the percentage of stable T regulatory cells comprising a hypomethylated TSDR at the FOXP3 locus does not decrease by more than 5% within 5, 6, 7, 8, 9, 10, or 11 days after a cryopreservation freeze-thaw cycle.

In some embodiments, hypomethylation of a TSDR at the FOXP3 locus is assessed within 1-3 days after transduction with an exogenous human TCR and again within 5, 6, 7, 8, 9, 10, or 11 days after a cryopreservation freeze-thaw cycle. In some embodiments, the percentage of stable T regulatory cells comprising a hypomethylated TSDR at the FOXP3 locus does not decrease by more than 20% within 5, 6, 7, 8, 9, 10, or 11 days after a cryopreservation freeze-thaw cycle. In some embodiments, hypomethylation of a TSDR at the FOXP3 locus is assessed within 1-3 days after isolation from a biological sample and again within 5, 6, 7, 8, 9, 10, or 11 days after a cryopreservation freeze-thaw cycle. In such embodiments, the percentage of stable T regulatory cells comprising a hypomethylated TSDR at the FOXP3 locus does not decrease by more than 20%, more than 19%, more than 18%, more than 17%, more than 16%, more than 15%, more than 14%, more than 13%, more than 12%, more than 11%, more than 10%, more than 9%, more than 8%, more than 7%, more than 6%, more than 5%, more than 4%, more than 3%, more than 2%, or more than 1% within 5, 6, 7, 8, 9, 10, or 11 days after a cryopreservation freeze-thaw cycle. In such embodiments, the percentage of stable T regulatory cells comprising a hypomethylated TSDR at the FOXP3 locus does not decrease by more than 10% within 5, 6, 7, 8, 9, 10, or 11 days after a cryopreservation freeze-thaw cycle. In such embodiments, the percentage of stable T regulatory cells comprising a hypomethylated TSDR at the FOXP3 locus does not decrease by more than 5% within 5, 6, 7, 8, 9, 10, or 11 days after a cryopreservation freeze-thaw cycle.

In some embodiments, the percentage of cells of the isolated cell population comprising the stable regulatory T cells does not decrease by more than 10, 9, 8, 7, 6, 5, 4, 3, 2, or 1 percentage point(s) during at least 5, 6, 7, 8, 9, 10, or 11 days of expansion following transduction of the stable regulatory T cells with the exogenous human TCR. In some embodiments, the percentage of cells of the isolated cell population comprising the stable regulatory T cells increases by 1, 2, 3, 4, or 5 percentage point(s) during at least 5, 6, 7, 8, 9, 10, or 11 days of expansion following transduction of the stable regulatory T cells with the exogenous human TCR. In some embodiments, the percentage of cells of the isolated cell population comprising the stable regulatory T cells does not decrease by more than 10 percentage points during at least 5 days of expansion following transduction of the stable regulatory T cells with the exogenous human TCR. In some embodiments, the percentage of cells of the isolated cell population comprising the stable regulatory T cells does not decrease by more than 10 percentage points during at least 10 days of expansion following transduction of the stable regulatory T cells with the exogenous human TCR. In some embodiments, the percentage of cells of the isolated cell population comprising the stable regulatory T cells does not decrease by more than 10 percentage points during at least 15 days of expansion following transduction of the stable regulatory T cells with the exogenous human TCR. In some embodiments, the percentage of cells of the isolated cell population comprising the stable regulatory T cells does not decrease by more than 5 percentage points during at least 5 days of expansion following transduction of the stable regulatory T cells with the exogenous human TCR. In some embodiments, the percentage of cells of the isolated cell population comprising the stable regulatory T cells does not decrease by more than 5 percentage points during at least 10 days of expansion following transduction of the stable regulatory T cells with the exogenous human TCR. In some embodiments, the percentage of cells of the isolated cell population comprising the stable regulatory T cells does not decrease by more than 5 percentage points during at least 15 days of expansion following transduction of the stable regulatory T cells with the exogenous human TCR. In some embodiments, the percentage of cells of the isolated cell population comprising the stable regulatory T cells does not decrease by more than 1 percentage point during at least 5 days of expansion following transduction of the stable regulatory T cells with the exogenous human TCR. In some embodiments, the percentage of cells of the isolated cell population comprising the stable regulatory T cells does not decrease by more than 1 percentage point during at least 10 days of expansion following transduction of the stable regulatory T cells with the exogenous human TCR. In some embodiments, the percentage of cells of the isolated cell population comprising the stable regulatory T cells does not decrease by more than 1 percentage point during at least 15 days of expansion following transduction of the stable regulatory T cells with the exogenous human TCR.

The percentage of stable regulatory T cells relative to total cells in an isolated cell population comprising regulatory T cells may be assessed about 1, about 6, about 12, about 24, about 36, about 48, about 72, about 96, or about 120 hours after transduction of cells with an exogenous human TCR. The percentage of stable regulatory T cells relative to total cells in an isolated cell population comprising regulatory T cells may be assessed 4, 5, 6, 7, 8, 9, 10, 11, 12, 13, or 14 days after transduction of cells with an exogenous human TCR. The percentage of stable regulatory T cells relative to total cells in an isolated cell population comprising regulatory T cells may be assessed about 1-21, 1-7, 4-14, 4-7, 7-10, 7-14, 10-21, or 14-21 days after transduction of cells with an exogenous human TCR.

In some embodiments, at least 5%, at least 10%, at least 15%, at least 20%, at least 30%, at least 40%, at least 50%, at least 60%, at least 70%, at least 80%, at least 90%, or at least 95% of the regulatory T cells of an isolated cell population express an exogenous TCR (e.g., following transduction of the isolated cell population with an exogenous TCR). In some embodiments, 10%-60% or 20%-50% of the regulatory T cells of an isolated cell population express an exogenous TCR. In some embodiments, at least 5%, at least 10%, at least 15%, at least 20%, at least 30%, at least 40%, at least 50%, at least 60%, at least 70%, at least 80%, at least 90%, or at least 95% of the stable regulatory T cells of an isolated cell population express an exogenous TCR (e.g., following transduction of the isolated cell population with an exogenous TCR). In some embodiments, 10%-60% or 20%-50% of the stable regulatory T cells of an isolated cell population express an exogenous TCR.

In some embodiments, the TSDR at the endogenous FOXP3 locus of stable regulatory T cells of an isolated cell population of regulatory T cells remains hypomethylated until administration of the isolated cell population to a subject. In some embodiments, the TSDR at the endogenous FOXP3 locus of stable regulatory T cells of an isolated cell population of regulatory T cells remains hypomethylated following a cryopreservation freeze-thaw cycle.

In some embodiments, regulatory T cells (e.g., stable regulatory T cells) of an isolated cell population exhibit one or more cellular functions that are associated with regulatory T cells when activated by binding the pMHC. Non-limiting examples of such cellular functions include cytokine secretion activity, expression of certain activation markers, and suppression activity. Cytokine secretion activity simply refers to the secretion of certain anti-inflammatory cytokines, such as IL-10, TGFβ, and IL-35. Activation markers include, but are not limited to CD69, 4-1BB, CD25, CD71, or CTLA-4. A regulatory T cell expresses one or more of these markers when it comes into contact with a target peptide complexed with MHC, for example.

Suppression activity refers to the suppression of activation, proliferation and cytokine production of non-regulatory T cells (e.g., CD8+ T cells and CD4+ conventional T cells) in part to suppress the immune system from becoming overactive. Regulatory T cells exhibit suppression activity when contacted with a target peptide complexed with MHC that binds to an exogenous TCR expressed by the regulatory T cells. In some embodiments, the target peptide is presented by a cell expressing the MHC. For example, in some embodiments, regulatory T cells suppress the activation, proliferation and cytokine production of conventional T cells having specificity towards a shared target peptide complexed with MHC, e.g., a shared target peptide presented by an Antigen Presenting Cell (APC). In some embodiments, regulatory T cells suppress proliferation and growth of non-regulatory T cells by at least 25%, at least 40%, at least 50%, at least 60%, at least 70%, or at least 80%, relative to a control (e.g., non-regulatory T cells in the absence of regulatory T cells).

In some embodiments, regulatory T cells suppress the production of IFN-gamma from conventional T cells by at least 25%, at least 40%, at least 50%, at least 60%, at least 70%, or at least 80%, relative to a control (e.g., conventional T cells in the absence of regulatory T cells). In some embodiments, regulatory T cells suppress the production of IFN-gamma from conventional T cells by 50%-99%, 75%-99%, or 80%-100% relative to a control (e.g., when present in a population comprising a ratio of 1:1 to 1:8 regulatory T cells compared to conventional T cells). In some embodiments, regulatory T cells suppress the production of CD71 from conventional T cells by at least 20%, at least 30%, at least 40%, at least 50%, at least 60%, or at least 70%, relative to a control (e.g., conventional T cells in the absence of regulatory T cells). In some embodiments, regulatory T cells suppress the production of CD71 of conventional T cells by 20%-90% or 30%-80% relative to a control (e.g., when present in a population comprising a ratio of 1:1 to 1:8 regulatory T cells compared to conventional T cells). In some embodiments, regulatory T cells suppress the production of CD25 from conventional T cells by at least 20%, at least 30%, at least 40%, at least 50%, at least 60%, or at least 70%, relative to a control (e.g., conventional T cells in the absence of regulatory T cells). In some embodiments, regulatory T cells suppress the production of CD25 of conventional T cells by 40%-70% relative to a control (e.g., when present in a population comprising a ratio of 1:1 to 1:8 regulatory T cells compared to conventional T cells).

In some embodiments, regulatory T cells exhibit cytokine secretion activity (e.g., secretion of IL-10) when contacted with a target peptide complexed with MHC, e.g., a target peptide presented by an APC, that binds to an exogenous TCR expressed by the regulatory T cells. In some embodiments, regulatory T cells exhibit expression of activation markers when contacted with a target peptide complexed with MHC that binds to an exogenous TCR expressed by the regulatory T cells. For example, in some embodiments, regulatory T cells exhibit expression of CD69, 4-1BB, CD25, CD71, and/or CTLA-4 when contacted with a target peptide complexed with MHC that binds to an exogenous TCR expressed by the regulatory T cells. In some embodiments, regulatory T cells exhibit suppression activity when contacted with a target peptide complexed with MHC that binds to an exogenous TCR expressed by the regulatory T cells. For example, in some embodiments, regulatory T cells suppress the activation of conventional T cells having specificity towards a shared target peptide complexed with MHC.

The cellular functions of stable regulatory T cells may be assessed about 1, about 6, about 12, about 24, about 36, about 48, about 72, about 96, or about 120 hours after transduction of cells with an exogenous human TCR. The cellular functions of stable regulatory T cells may be assessed 4, 5, 6, 7, 8, 9, 10, 11, 12, 13, or 14 days after transduction of cells with an exogenous human TCR. The cellular functions of stable regulatory T cells may be assessed about 1-21, 1-7, 4-14, 4-7, 7-10, 7-14, 10-21, or 14-21 days after transduction of cells with an exogenous human TCR.

In some embodiments, transduced regulatory T cells (e.g., transduced regulatory T cells of an isolated population) retain their cellular functionality (e.g., ability to be activated) following a cryopreservation freeze-thaw cycle. In some embodiments, transduced regulatory T cells can be activated and/or expanded following a cryopreservation freeze-thaw cycle. In some embodiments, regulatory T cells can exhibit cytokine secretion activity, expression of certain activation markers, and/or suppression activity following a cryopreservation freeze-thaw cycle.

In some embodiments, at least 5%, at least 10%, at least 25%, at least 50%, at least 75%, at least 80%, at least 85%, at least 90%, at least 95%, at least 96%, at least 97%, at least 98%, or at least 99% of a population of cells are CD25+/highCD4+CD127-/lo prior to activation and/or transduction with an exogenous human T cell receptor.

An isolated cell population comprising stable regulatory T cells may comprise a minority amount of non-regulatory T cells (e.g., conventional T cells). Non-regulatory T cells may be NK T cells, B cells, CD8+ T cells, neutrophils, eosinophils, CD14+ cells, or conventional (CD4+) T cells that are derived from peripheral blood and lymph nodes. In some embodiments, less than 25%, less than 20%, less than 15%, less than 10%, less than 5%, less than 2%, less than 1%, less than 0.5%, less than 0.1%, or less than 0.01% of the cells of an isolated cell population comprising stable regulatory T cells are non-regulatory T cells. In some embodiments, about 0.01% to about 0.1%, about 0.1% to about 0.5%, about 0.5% to about 1%, about 0.5% to about 10%, about 2% to about 5%, or about 5% to about 10% of the cells of an isolated cell population comprising regulatory T cells are non-regulatory T cells. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are non-regulatory T cells that comprise an exogenous human TCR. A conventional T cell commonly produces IL-2 and other interleukin factors. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are conventional T cells. In some embodiments, about 0.01% to about 0.1%, about 0.1% to about 0.5%, about 0.5% to about 1%, about 0.5% to about 10%, about 2% to about 5%, or about 5% to about 10% of the cells of an isolated cell population comprising regulatory T cells are conventional T cells. In some embodiments, less than 25%, less than 20%, less than 15%, less than 10%, less than 5%, less than 2%, less than 1%, less than 0.5%, less than 0.1%, or less than 0.01% of the cells of an isolated cell population comprising regulatory T cells are conventional T cells that comprise an exogenous human TCR. In some embodiments, an isolated cell population comprising regulatory T cells comprises an undetectable amount of conventional T cells. In some embodiments, an isolated cell population comprising regulatory T cells comprises an undetectable amount of conventional CD4+ T cells. In some embodiments, an isolated cell population comprising regulatory T cells comprises an undetectable amount of B cells. In some embodiments, an isolated cell population comprising regulatory T cells comprises an undetectable amount of NK T cells. In some embodiments, an isolated cell population comprising regulatory T cells comprises an undetectable amount of CD14+ cells. In some embodiments, an isolated cell population comprising regulatory T cells comprises an undetectable number of eosinophils. In some embodiments, an isolated cell population comprising regulatory T cells comprises an undetectable number of neutrophils. In some embodiments, an isolated population of cells comprising regulatory T cells comprises an undetectable amount of CD8+ T cells.

The percentage of conventional T cells or other non-regulatory T cells relative to total cells (or relative to stable regulatory T cells) may be assessed about 1, about 6, about 12, about 24, about 36, about 48, about 72, about 96, or about 120 hours after transduction of cells with an exogenous human TCR. The percentage of conventional T cells or other non-regulatory T cells relative to total cells (or relative to stable regulatory T cells) may be assessed 4, 5, 6, 7, 8, 9, 10, 11, 12, 13, or 14 days after transduction of cells with an exogenous human TCR. The percentage of conventional T cells or other non-regulatory T cells relative to total cells (or relative to stable regulatory T cells) may be assessed about 1-21, 1-7, 4-14, 4-7, 7-10, 7-14, 10-21, or 14-21 days after transduction of cells with an exogenous human TCR. The percentage of conventional T cells or other non-regulatory T cells relative to total cells (or relative to stable regulatory T cells) may be assessed 1, 2, 3, 4, 5, 6, 7, 8, 9, 10, 11, 12, 13, or 14 days after a cryopreservation freeze-thaw cycle.

In some embodiments, the ratio of regulatory T cells to conventional T cells in an isolate population of cells comprising regulatory T cells is at least 5:1, at least 10:1, at least 15:1, at least 20:1, at least 25:1, at least 30:1, at least 35:1, at least 40:1, at least 45:1, at least 50:1, at least 60:1, at least 70:1, at least 80:1, at least 90:1, or at least 100:1.

### Methods of Producing Stable Regulatory T Cells

In some embodiments, the present disclosure provides methods of producing isolated populations of cells comprising stable CD4+ T regulatory cells derived from a subject having multiple sclerosis. Conventional methods of producing antigen-specific regulatory T cells have been unable to produce clinical scale and clinical grade manufacturing of cell populations comprising a high concentration of stable antigen-specific regulatory T cells. Although polyclonal T cells have successfully been produced and administered in clinical settings, the use of stable, thymic regulatory T cells engineered with an exogenous TCR poses increased safety risks that require increased cellular purity and regulatory T cell stability. Without wishing to be bound by theory, stable thymic regulatory T cells represent a small percentage of leukocytes, and if the isolated regulatory T cell population is not highly pure, other types of leukocytes will be engineered with the exogenous TCR. In sufficient numbers, these engineered non-regulatory T cells could have a deleterious effect by causing, rather than suppressing, an immune response at the site of autoimmune disease. Through integration and modification of several biomarker selection processes, including an unexpectedly effective double selection process using CD25, and in some instances CD45RA, the methods designed herein provide the field with the tools to produce populations of antigen-specific regulatory T cells at high yield and with a high relative concentration of stable TCR-transduced regulatory T cells.

In some embodiments, the method of the present disclosure involves removal of conventional T cells and selection for cells having a CD25+/highCD4+CD127−/lo phenotype to produce cell populations comprising stable regulatory T cells. In some embodiments, the method of the present disclosure involves removal of conventional T cells, selection for cells having a CD25+/highCD4+CD127−/low phenotype to produce cell populations comprising stable regulatory T cells, and engineering of the stable regulatory T cells to comprise an exogenous human TCR that binds specifically to a target peptide complexed with an MHC. In some embodiments, a method of producing a cell population of stable regulatory T cells comprises isolating a biological sample comprising regulatory T cells from a human subject having multiple sclerosis.

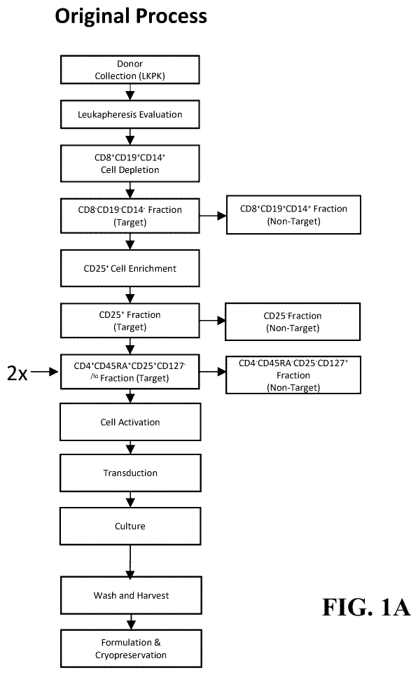

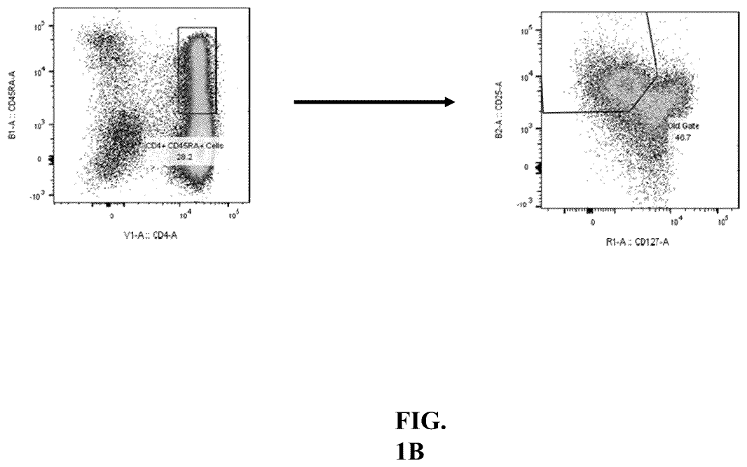

An example method of the disclosure is provided in FIGS. 1A-1B. In the embodiment provided in FIG. 1A, a biological sample (e.g., blood sample) is first isolated from a human subject having multiple sclerosis using apheresis. The cells of the biological sample are labeled with anti-CD8 (targeting non-CD4 conventional T cells), anti-CD19 (targeting B cells), and anti-CD14 (targeting monocytes) antibodies; and the labeled cells are removed from the biological sample to produce a depleted biological sample. In some embodiments, the volume of the depleted biological sample is then reduced (e.g., by removal of water/liquid) to assist with downstream processing of the sample. The sample is then labeled with an anti-CD25 antibody (e.g., a CD25-PE-Biotin antibody) and a secondary molecule (e.g., an anti-biotin microbead) to produce a CD25-enriched cell population. In some embodiments, a CD25-PE-Biotin antibody is used. The CD25-PE-Biotin antibody comprises an anti-CD25 antibody attached to a tandem conjugate of phycoerythrin (PE) and Biotin. In some embodiments, this enrichment step may be repeated more than once (e.g., two or more times). These CD25-enriched cultures are then further processed by sorting cells using a TCR gating strategy that utilizes fluorescence-activated cell sorting (FACS) or Tyto sorting (Miltenyi) as described in FIGS. 1A and 1B. Cells are first labeled with anti-CD45RA, anti-CD4, and anti-CD127 antibodies. Cells are then selected in multiple sorting steps. In each step, in some embodiments, labeled CD25+/highCD4+CD127−/lo cells are first identified by a gating strategy comprising first identifying singlets, then identifying living cells, then identifying CD4⁺ cells, then identifying CD25^{+/high}CD127^{-/low} cells from the CD4+ cells, and then identifying CD25^{high}CD45RA⁻ and CD25^{+/high}CD45RA⁺ cells. The identified population of cells (e.g., cells comprising the protein expression profile of CD4⁺CD25^{high}CD127^{-/low}CD45RA⁻ and cells comprising the expression profile of CD4⁺CD25+CD127^{-/low}CD45RA⁺) is the collected. In some embodiments, the sorting procedure is performed twice to optimize purity of the cells.

In some embodiments, the present disclosure provides a method of producing an isolated population comprising stable CD4⁺ T regulatory cells, wherein the method comprises (a) removing CD8⁺ cells and CD19⁺ cells from a biological sample obtained from a subject having multiple sclerosis to produce a depleted biological sample; (b) enriching the depleted biological sample for CD25⁺ cells to produce an enriched population; and (c) isolating CD4⁺CD25⁺CD127^{-/low} cells from the enriched population. In some embodiments, the present disclosure provides a method of producing an isolated population comprising stable CD4⁺ T regulatory cells, wherein the method comprises (a) removing CD8⁺ cells and CD19⁺ cells from a biological sample obtained from a subject having multiple sclerosis to produce a depleted biological sample; (b) enriching the depleted biological sample for CD25⁺ cells to produce an enriched population; (c) isolating CD4⁺CD25+CD127^{-/low} cells from the enriched population; and (d) engineering the population of cells of (c) to express an exogenous human T cell receptor (TCR) that binds specifically to a target peptide complexed with an MHC. In some embodiments, a step (a) further comprises removing CD14⁺ cells from the biological sample. In some embodiments, a step (a) further comprises removing CD56⁺ cells from the biological sample.

In some embodiments, the method further comprises quantifying the methylation status of a T cell specific demethylated region (TSDR) at the FOXP3 locus in the population of cells, wherein at least 80% of the cells are stable CD4⁺ T regs comprising a hypomethylated TSDR at the FOXP3 locus. In some embodiments, the methylation status of the TSDR is quantified after the isolation step of (c). In some embodiments, the methylation status of the TSDR is quantified after the isolation step of (d).

In some embodiments, the step of isolating CD4⁺CD25⁺CD127^{-/low} cells from the enriched population comprises (i) identifying a first subpopulation of CD4⁺ cells from the enriched population of step (b); (ii) identifying a second subpopulation of CD25^{+/high}CD127^{-/low} cells from the first subpopulation; and (iii) selecting CD25highCD45RA− and CD25^{+/high}CD45RA⁺ cells from the second subpopulation for isolation and excluding CD25⁺CD45RA⁻, thereby isolating the population of stable CD4⁺ T regs.

In some embodiments, the methods provided herein further comprise isolating a biological sample comprising regulatory T cells from a human subject having multiple sclerosis. In some embodiments, the sample isolation is performed using an apheresis technique (e.g., a leukapheresis technique to isolate white blood cells). In some embodiments, isolating a biological sample comprising regulatory T cells from a human subject having multiple sclerosis is performed by removing blood from the subject and separating the blood into plasma and cells. In some embodiments, an apheresis technique involves the removal of whole blood from a subject and separation of the whole blood to remove desired cell types (e.g., T cells). In some embodiments, the separation step of apheresis is performed using continuous flow centrifugation or intermittent flow centrifugation.

Cells expressing a specific biomarker (e.g., CD8⁺ cells, CD19⁺ cells, CD14⁺ cells, and/or CD56⁺ cells) may be removed from a sample (e.g., a biological sample) using FACS, an antibody pulldown assay technique, or any other method of removing cells expressing a specific biomarker that is known to a person of ordinary skill in the art. In some embodiments, cells expressing a specific biomarker may be removed from a sample by labeling those cells with a microbead (e.g., anti-CD8, anti-CD14, anti-CD19 and/or anti-CD56 microbeads) and then subjected the sample to FACS (to remove the cells expressing the specific biomarker).

A depleted biological sample is a biological sample (e.g., isolated from a human subject having multiple sclerosis) that has been processed to remove CD8⁺ cells, CD19⁺ cells, CD14⁺ cells and/or CD56⁺ cells. In some embodiments, removing CD8⁺ cells, CD19⁺ cells, CD14⁺ cells and/or CD56⁺ cells from a biological sample generates a depleted biological sample having less than 20%, less than 15%, less than 10%, less than 5%, less than 4%, or less than 3% of its total cells comprising CD8⁺ cells, CD19⁺ cells, CD14⁺ cells, and/or CD56⁺ cells. In some embodiments, removing CD8⁺ cells, CD19⁺ cells, CD14⁺ cells and/or CD56⁺ cells from a biological sample generates a depleted biological sample having less than 0.5% of its total cells comprising CD8⁺ cells.

A cell selection technique may be used to select for cells having a specific phenotype (e.g., cells expressing a specific biomarker such as CD4, CD25, or CD45RA; or cells that have no to low expression of a specific biomarker such as CD127). A cell selection technique may be a FACS technique, a Tyto device, an antibody pulldown assay technique, magnetic cell separation technique, or any other method of selecting cells expressing a specific biomarker that is known to a person of ordinary skill in the art. In some embodiments, a cell selection technique comprises labeling cells expressing a specific biomarker (e.g., CD25) with a biotinylated antibody that targets that biomarker and then performing a pulldown assay (e.g., with anti-biotin microbeads or a streptavidin complex). In some embodiments, a cell selection technique comprises labeling cells expressing a specific biomarker (e.g., CD25, CD4, and/or CD45RA) with antibodies that target that biomarker. Cells labeled with an antibody that targets a specific biomarker can then be selected from a sample (e.g., a CD25-enriched population) using FACS.

The CD25 enrichment step is then followed by a multi-step sorting process to select for CD25^{+/high}CD4⁺CD127^{-/low} regulatory T cells. In some embodiments, the sorting steps are performed on a Tyto device (Miltenyi). One of skill in the art will appreciate that software for visualizing sorted cell populations enable display of the data in various ways including dot plots and histograms. When a population is being identified based on the presence or absence of single protein, identification can be made using a dot plot or histogram. When it is necessary to identify a population based on the presence or absence of two proteins (e.g., CD25 and CD45RA), identification should be made using a dot plot.

In some embodiments, prior to the first sorting step, the CD25-enriched cell population is separated into multiple populations of cells and the first sorting step is performed simultaneously and/or sequentially in the multiple sub-populations. This provides for expedited processing of the large number of cells that need to be processed in order to isolate sufficient numbers of the small proportion of cells that are CD25^{+/high}CD4⁺CD127^{-/low}. In some embodiments, the CD25-enriched cell population is separated into 2, 3, 4, 5, or 6 or more sub-populations that are each sorted in the first sorting step. In some embodiments, after the first sorting step, the sorted sub-populations are combined prior to the second sorting step.

In some embodiments, the present disclosure provides a method of producing an isolated population comprising stable CD4⁺ T regulatory cells, wherein the method comprises (a) removing CD8⁺ cells and CD19⁺ cells from a biological sample obtained from a subject having multiple sclerosis to produce a depleted biological sample; (b) enriching the depleted biological sample for CD25⁺ cells to produce an enriched population; and (c) isolating CD4⁺CD25+CD127^{-/low} cells from the enriched population, wherein the isolating comprises (i) identifying a first subpopulation of CD4⁺ cells from the enriched population of step (b); (ii) identifying a second subpopulation of CD25^{+/high}CD127^{-/low} cells from the first subpopulation; and (iii) selecting CD25^{high}CD45RA; and CD25^{+/high}CD45RA⁺ cells from the second subpopulation for isolation and excluding CD25+CD45RA⁻, thereby isolating the population of stable CD4+ T regs

In some embodiments, selecting CD25^{+/high}CD4⁺CD127^{-/low} cells from a CD25-enriched population comprises identifying a first subpopulation CD4⁺ cells from the CD25-enriched population. A second subpopulation of CD25^{+/high}CD127^{-/low} cells is then identified from the first subpopulation. Finally, populations of CD25^{high}CD45RA⁻ and CD25^{+/high}CD45RA⁺ cells are selected from the second subpopulation for isolation.

In some embodiments, the first subpopulation comprises at least 60%, at least 70%, at least 71%, at least 72% at least 73% at least 74%, at least 75%, at least 76%, at least 77%, at least 78%, at least 79%, at least 80%, at least 81%, at least 82%, at least 83%, at least 84%, or at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% CD4+ cells.

In some embodiments, the second subpopulation comprises at least 60%, at least 70%, at least 71%, at least 72% at least 73% at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% CD25^{+/high}CD4⁺CD127^{-/low} regulatory T cells. In some embodiments, the second subpopulation comprises at least 60%, at least 70%, at least 71%, at least 72% at least 73% at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% CD25^{+/high}CD4⁺CD127^{-/low}FoxP3⁺ regulatory T cells.

In some embodiments, the population selected for isolation comprises CD25^{high}CD4⁺CD127^{-/low}CD45RA⁻ (CD25^{high}CD45RA⁻) and CD25^{+/high}CD4⁺CD127^{-/low}CD45RA⁺ (CD25^{+/high}CD45RA⁺) regulatory T cells. In some embodiments, the population selected for isolation comprises at least 30%, at least 35%, at least 40%, at least 50%, at least 55%, at least 60%, at least 65%, at least 70%, at least 75%, at least 80%, at least 85%, at least 90%, or at least 95% CD25^{high}CD45RA⁺ cells. In some embodiments, the population selected for isolation comprises at least 30%, at least 35%, at least 40%, at least 50%, at least 55%, at least 60%, at least 65%, at least 70%, at least 75%, at least 80%, at least 85%, at least 90%, or at least 95% CD25^{+/high}CD45RA⁺ cells. It is understood that percentages of CD25^{high}CD45RA⁻ and CD25^{+/high}CD45RA⁺ cells in the final population add together to equal approximately 80%, 90%, or 100% of the total cell population.

In some embodiments, the population selected for isolation comprises less than 10% CD25⁺CD4⁺CD127^{-/low}CD45RA⁻ cells. In some embodiments, the population selected for isolation comprises less than 9%, less than 8%, less than 7%, less than 6%, less than 5%, less than 4%, less than 3%, less than 2%, or less than 1% CD25⁺CD4⁺CD127^{-/low}CD45RA; cells.

In some embodiments, the methods described herein result in an isolated population of cells, wherein at least 80% of the cells are stable CD4⁺ T regs comprising a hypomethylated T cell specific demethylated region (TSDR) at the FOXP3 locus. In some embodiments, at least 81%, 82%, 83%, 84%, 85%, 86%, 87%, 88%, 89%, 90%, 91%, 92%, 93%, 94%, 95%, 96%, 97%, 98%, or 99% of the cells are stable CD4⁺ T regs comprising a hypomethylated TSDR at the FOXP3 locus. In some embodiments, the isolated cell population comprises stable CD4⁺ T regulatory cells, wherein 80%, 81%, 82%, 83%, 84%, 85%, 86%, 87%, 88%, 89%, 90%, 91%, 92%, 93%, 94%, 95%, 96%, 97%, 98%, or 99% of the cells are stable CD4+T regs comprising a hypomethylated TSDR at the FOXP3 locus. In some embodiments, between 80% and 90%, between 85% and 95%, between 80% and 85%, between 85% and 90%, between 90% and 95% of the cells are stable CD4⁺ T regs comprising a hypomethylated TSDR at the FOXP3 locus.

In some embodiments, the isolated population of cells produced by the methods described herein is selected for therapeutic use if at least 80% of the cells are stable CD4⁺ T regs comprising a hypomethylated T cell specific demethylated region (TSDR) at the FOXP3 locus. In some embodiments, the isolated population of cells is selected for therapeutic use if at least 81%, 82%, 83%, 84%, 85%, 86%, 87%, 88%, 89%, 90%, 91%, 92%, 93%, 94%, 95%, 96%, 97%, 98%, or 99% of the cells are stable CD4⁺ T regs comprising a hypomethylated TSDR at the FOXP3 locus. In some embodiments, the isolated population of cells is selected for therapeutic use if 80%, 81%, 82%, 83%, 84%, 85%, 86%, 87%, 88%, 89%, 90%, 91%, 92%, 93%, 94%, 95%, 96%, 97%, 98%, or 99% of the cells are stable CD4⁺ T regs comprising a hypomethylated TSDR at the FOXP3 locus. In some embodiments, the isolated population of cells is selected for therapeutic use if between 80% and 90%, between 85% and 95%, between 80% and 85%, between 85% and 90%, between 90% and 95% of the cells are stable CD4⁺ T regs comprising a hypomethylated TSDR at the FOXP3 locus.

In some embodiments, the methods provided herein further comprise introducing an exogenous TCR that binds specifically to a target peptide complexed with an MHC to the population of cells comprising stable CD4⁺ T regulator cells. In some embodiments, introducing the exogenous TCR comprises transfecting the population of cells with a nucleic acid encoding the exogenous TCR. In some embodiments, introducing the exogenous TCR comprises transducing the population of cells with a nucleic acid encoding the exogenous TCR. The nucleic acid encoding the exogenous TCR may be an RNA (e.g., mRNA) molecule or a DNA molecule. In some embodiments, the nucleic acid is a vector or plasmid. In some embodiments, the nucleic acid is delivered to the population of cells using a lentiviral vector, an adenovirus vector, an adeno-associated viral (AAV) vector, a herpes viral vector, a retroviral vector, or a baculoviral vector.

In some embodiments, a method or process of producing a cell population of regulatory T cells further comprises activating and/or expanding regulatory T cells of the population (e.g., following the deletion, enrichment, and engineering steps described herein). In some embodiments, the regulatory T cells are activated prior to transfection or transduction of the regulatory T cells with the exogenous TCR. In some embodiments, the regulatory T cells are activated with anti-CD28 and/or anti-CD8 antibodies. In some embodiments, activating regulatory T cells comprises contacting the regulatory T cells with the antigen that specifically binds to the exogenous TCR belonging to the isolated cell population. In some embodiments, the antibodies (e.g., the anti-CD3, anti-CD28, and/or TCR-specific antibodies) are conjugated or complexed with a bead, e.g., a magnetic bead or a polymeric bead. In some embodiments, the antibodies are covalently attached to a polymeric matrix.

In some embodiments, the methods provided herein further comprise a second activation and/or expansion step. In some embodiments, the second activation and/or expansion step is performed 5, 6, or 7 days after the initial activation and/or expansion step.

In some embodiments, activating and/or expanding the regulatory T cells comprises culturing the isolated cell population in a cell media. In some embodiments, the media comprises IL-2. In some embodiments, the media comprises about 100, 200, 300, 400, 500, 600, 700, 800, 900, 1,000, 1200, 1400, 1600, 1800, 2000, 3000, or 4000 IU/ml IL-2. In some embodiments, the media comprises 500-1500 or 800-1200 IU/ml IL-2. In some embodiments, the media comprises about 1,000 IU/ml IL-2. In some embodiments, the media comprises 1,000 IU/ml IL-2. In some embodiments, the media comprises further comprises TNFα. In some embodiments, the media comprises about 500, 750, 1000, 1500, 1750, 2000, 2250, 2500, or about 3000 IU/ml TNFα. In some embodiments, the media comprises 2000-3000 or 2250-2750 IU/ml TNFα. In some embodiments, the media comprises about 2500 IU/ml TNFα. In some embodiments, the media comprises 2500 IU/ml TNFα.

In some embodiments, activating and expanding the regulatory T cells comprises culturing cells of the population for at least 2, 3, 4, 5, 6, 7, 8, 9, or 10 days. In some embodiments, activating and expanding the regulatory T cells comprises culturing cells of the population for 2-14, 2-10, 2-5, 5-10, or 5-14 days. In some embodiments, activating and expanding the regulatory T cells comprises culturing cells of the population for no more than 15, 14, 13, or 12 days. In some embodiments, following activation and/or expansion, isolated the isolated cell population comprises at least 1×10², at least 1×10³, at least 1×10⁴, at least 1×10⁵, at least 1×10⁶, at least 1×10⁷, at least 1×10⁸, at least 1×10⁹, or at least 1×10¹⁰ stable CD25^{+/high}CD4⁺CD127^{-/low} regulatory T cells comprising a hypomethylated TSDR at an endogenous FOXP3 locus.

In some embodiments, a method or process of producing a cell population of regulatory T cells may further comprise cryopreserving the cell population. In some embodiments, a method or process of producing a cell population of regulatory T cells may further comprise thawing the cryopreserved cell population. A cryopreservation freeze-thaw cycle refers to the process of cryopreserving a cell population, and thawing the cryopreserved cell population at a later time.

### Pharmaceutical Compositions

In some embodiments, the disclosure provides pharmaceutical compositions comprising the isolated cell populations of regulatory T cells (e.g., stable regulatory T cells) described herein. In some embodiments, a pharmaceutical composition comprises a cell population of regulatory T cells (e.g., stable regulatory T cells) described herein and a pharmaceutically acceptable excipient.

As used herein, a pharmaceutically acceptable excipient may also be referred to as a pharmaceutically acceptable carrier, pharmaceutically acceptable diluent, or pharmaceutically acceptable adjuvant. Formulation of pharmaceutically-acceptable excipients and carrier solutions is well-known to those of skill in the art, as is the development of suitable dosing and treatment regimens for using the particular compositions described herein in a variety of treatment regimens.

The pharmaceutical compositions typically should be sterile and stable under the conditions of manufacture and storage. Sterile injectable formulations may be prepared using a non-toxic parenterally acceptable diluent or solvent. A pharmaceutical composition for use in accordance with the present invention may include pharmaceutically acceptable dispersing agents, wetting agents, suspending agents, isotonic agents, coatings, antibacterial and antifungal agents, carriers, excipients, salts, or stabilizers which are non-toxic to the subjects at the dosages and concentrations employed. In some embodiments, the pharmaceutical composition may comprise an organic solvent, such as but not limited to, methyl acetate, dimethyl sulfoxide (DMSO), N,N-dimethylformamide (DMF), dimethoxyethane (DME), and dimethylacetamide, including mixtures or combinations thereof.

A pharmaceutical composition may comprise an effective amount of stable regulatory T cells that is sufficient to elicit a desired biological response. For example, in some embodiments, an effective amount of stable regulatory T cells as described herein may refer to number of cells that is sufficient to improve a symptom associated with multiple sclerosis (e.g., progressive Multiple Sclerosis). As will be appreciated by the skilled artisan, the effective amount of a solution or preparation provided herein may vary depending on various factors as, for example, on the desired biological response, e.g., on the specific disease being treated, the specific symptom to be alleviated, on the cell or tissue being targeted, and on the subject's age, gender, and general health status.

In some embodiments, an effective amount of stable regulatory T cells (e.g., regulatory T cells comprising a hypomethylated TSDR at an endogenous FOXP3 locus) comprises at least 1×10², at least 1×10³, at least 1×10⁴, at least 1×10⁵, at least 1×10⁶, at least 1×10⁷, at least 1×10⁸, at least 1×10⁹, or at least 1×10¹⁰ stable regulatory T cells. In some embodiments, an effective amount of stable regulatory T cells (e.g., regulatory T cells comprising a hypomethylated TSDR at an endogenous FOXP3 locus) is 1×10² to 1×10¹⁰, 1×10³ to 1×10¹⁰, 1×10⁴ to 1×10¹⁰, 1×10⁵ to 1×10¹⁰, 1×10⁶ to 1×10¹⁰, 1×10⁷ to 1×10¹⁰, 1×10⁸ to 1×10¹⁰, 1×10⁹ to 1×10¹⁰ stable regulatory T cells. In some embodiments, an effective amount of stable regulatory T cells comprises 1×10⁷, 2×10⁷, 3×10⁷, 4×10⁷, 5×10⁷, 6×10⁷, 7×10⁷, 8×10⁷, or 9×10⁷ stable regulatory T cells. In some embodiments, an effective amount of stable regulatory T cells comprises 1×10⁷ to 1×10¹⁰, 2×10⁷ to 1×10¹⁰, 3×10⁷ to 1×10¹⁰, 4×10⁷ to 1×10¹⁰, 5×10⁷ to 1×10¹⁰, 6×10⁷ to 1×10¹⁰, 7×10⁷ to 1×10¹⁰, 8×10⁷ to 1×10¹⁰, or 9×10⁷ to 1×10¹⁰ stable regulatory T cells.

In some embodiments, the isolated cell populations (e.g., intended to be used in a pharmaceutical composition) are cryopreserved (e.g., subjected to one or more cryopreservation freeze-thaw cycles). That is, the isolated cell populations produced herein may be combined with a cryoprotecting agent, which lowers the melting temperature by forming chemical bonds with water and increasing the total concentration of solutes in the system. Non-limiting examples of cryoprotecting agents include glycerol, dimethyl sulfoxide (DMSO), ethanediol, and propanediol. While traditional methods that often include the use of serum and DMSO may be used, the disclosure also contemplates the use of freezing medium manufactured under cGMP conditions and formulated serum-free and of non-animal origin (e.g., using <10% DMSO in the freeze cocktail). Other cryopreservation techniques are also provided herein, including more advanced techniques of cooling, for example, vitrifying cells without the use of cryoprotecting agents. See, e.g., Shinshu University. “A new way to ‘freeze’ cells promises to transform the common cell-freezing practice.” ScienceDaily.com April 2019. This process of ultrarapid cooling, utilizes inkjet cell printing to cool at a rate of 10,000 degrees Celsius/second, causing near-vitrification of the cells.

### Methods of Treatment

In some embodiments, the present disclosure provides methods of treating multiple sclerosis in a subject in need thereof comprising administering an isolated population of cells comprising stable CD4⁺ T regulatory cells or a composition thereof. In some embodiments, the disclosure provides methods of administration of isolated cell populations comprising regulatory T cells as described herein (and related pharmaceutical compositions) to a subject (e.g., a subject having multiple sclerosis).

Multiple Sclerosis usually onsets with relapses and remissions in symptoms in Relapsing Remitting Multiple Sclerosis (RRMS). Approximately 80 to 85 percent of MS patients are initially diagnosed with this form of the disease. There are over 15 disease-modifying therapies for RRMS. In progressive MS, there is a slow, steady progression of the disease. Progressive MS can be relapsing (sometimes termed secondary progressive MS) or non-relapsing (sometimes termed primary progressive MS). Progressive Multiple Sclerosis is characterized by increasing clinical disability independent of acute relapsing pathology. There are currently no therapies available for Progressive Multiple Sclerosis. Over 250,000 patients are currently affected by progressive Multiple Sclerosis. Moreover, although currently approved disease modifying therapies are effective at reducing relapse occurrence in RRMS, even RRMS subjects experience progression independent of relapse (PIRA), which has not been successfully treated (Ransohoff 2023).

In progressive MS, compartmentalized inflammation, mainly localized in meningeal lymphoid aggregates, drives ongoing inflammatory demyelination during progressive Multiple Sclerosis behind an intact blood-brain barrier (Lassman, Frontiers in Immunology, 2019). Inflammatory T cells in a stable, long-lasting aggregate release inflammatory mediators that activate macrophages. It has been shown that this high density of meningeal lymphocytes and inflammation is associated with an increased level of subpial demyelination at the cortex and increased white matter lesions (Ahmed et al., JCI Insight. 2022; 7 (5)). Macrophages at the lesion edge continuously degrade myelin. PIRA in RRMS subjects has the same mechanistic basis as progression in progressive forms of MS (Ransohoff 2023).

Without wishing to be bound by theory, it is contemplated herein that targeting stable engineered regulatory T cells to the meninges by engineering them to bind MBP, which is presented by cells in the meninges of subjects affected with Multiple Sclerosis, will reduce, stop the progression of, or reverse the progression of Multiple Sclerosis, and progressive Multiple Sclerosis, in particular, by reducing the inflammatory response in the meninges.

In some embodiments, the disclosure provides methods of administering a cell population comprising regulatory T cells or a pharmaceutical composition as described herein to a subject in an effective amount to alleviate one or more symptoms of multiple sclerosis. Non-limiting examples of symptoms of Multiple Sclerosis include fatigue, vision problems, numbness in muscle tissues, muscle spasms, muscle stiffness, muscle weakness, mobility problems, pain (e.g., muscle pain), depression, anxiety, sexual problems, bladder problems, bowel problems, speech problems, and difficulty with swallowing.

In some embodiments, the disclosure provides methods of treating multiple sclerosis in a subject comprising administering a cell population comprising regulatory T cells or a pharmaceutical composition as described herein to the subject. In some embodiments, at least a portion of the population of cells are autologous cells (i.e., obtained from the same subject to which they are subsequently administered). In some embodiments, a population of cells is isolated from a subject, subjected to a method of producing a cell population (e.g., to increase the relative concentration of regulatory T cells within the population) as described herein, engineered to express an exogenous TCR, and then administered to the same subject in order to treat a disease.

In some embodiments, treating (or treatment of) a disease refers to a clinical intervention aimed to reverse, alleviate, delay the onset of, or inhibit the progress of multiple sclerosis, or one or more symptoms thereof. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., because of knowledge of genetic factors). Treatment may also be continued after symptoms have resolved, for example, to prevent or delay their recurrence.

A subject refers to an individual organism, for example, an individual human. In some embodiments, the subject is a human subject, such as a male subject or a female subject. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a research animal. In some embodiments, the subject is genetically engineered, e.g., a genetically engineered non-human subject. The subject may be male or female.

Conventional and pharmaceutically acceptable routes of administration of the include, but are not limited to, intravenous, subcutaneous, intravenous, intrathecal administration direct delivery to the selected organ (e.g., intraportal delivery to the liver), oral, inhalation (including intranasal and intratracheal delivery), intraocular, intramuscular, intradermal, intratumoral, and other parental routes of administration. Routes of administration may be combined, if desired. In some embodiments, the isolated populations or compositions thereof described herein are administered intravenously.

A cell population comprising regulatory T cells or a pharmaceutical composition may be administered as a bolus administration. In some embodiments, administration of a cell population comprising regulatory T cells or a pharmaceutical composition comprises one or more infusion of the isolated cell population or pharmaceutical composition to the subject (e.g., cells are infused through a central line, similar to a blood transfusion).

### FURTHER NUMBERED EMBODIMENTS

Further numbered embodiments of the disclosure are provided as follows:

Embodiment 1. An isolated cell population comprising regulatory T cells, wherein: regulatory T cells of the isolated cell population comprise stable regulatory T cells that (a) comprise an exogenous human T cell receptor (TCR) that binds specifically to a myelin basic protein (MBP) peptide complexed with a major histocompatibility complex (MHC) and (b) comprise a hypomethylated regulatory T cell-specific demethylation region (TSDR) at an endogenous FOXP3 locus, at least 80% of the cells of the isolated cell population are CD25+/highCD4+CD127−/lo regulatory T cells, and less than 10% of the cells of the isolated cell population express FOXP3 protein from an engineered FOXP3 locus.

Embodiment 2. The isolated population of embodiment 1, wherein the MBP peptide is an MBP 83-99 peptide comprising the amino acid sequence of SEQ ID NO: 61.

Embodiment 3. The isolated population of embodiment 1 or 2, wherein the MHC comprises HLA-DRB1*15:01.

Embodiment 4. The isolated population of embodiment 3, wherein the MHC further comprises HLA-DRA*.

Embodiment 5. The isolated population of any one of the preceding embodiments, wherein the exogenous TCR comprises a TCR alpha chain comprising (a) a CDR1-alpha sequence comprising the amino acid sequence of SEQ ID NO: 1, (b) a CDR2-alpha sequence comprising the amino acid sequence of SEQ ID NO: 2, and/or (c) a CDR3-alpha sequence comprising the amino acid sequence of SEQ ID NO: 3.

Embodiment 6. The isolated population of embodiment 5, wherein the exogenous TCR comprises a TCR alpha chain variable region comprising an amino acid sequence having at least 90%, 95%, or 100% identity to the amino acid sequence of SEQ ID NO: 4.

Embodiment 7. The isolated population of embodiment 5, wherein the exogenous TCR comprises a TCR alpha chain comprising an amino acid sequence having at least 90%, 95%, or 100% identity to the amino acid sequence of SEQ ID NO: 5 or 73.

Embodiment 8. The isolated population of any one of the preceding embodiments, wherein the exogenous TCR comprises a TCR beta chain comprising a CDR1-beta sequence comprising the amino acid sequence of SEQ ID NO: 7, a CDR2-beta sequence comprising the amino acid sequence of SEQ ID NO: 8, and/or a CDR3-beta sequence comprising the amino acid sequence of SEQ ID NO: 9.

Embodiment 9. The isolated population of embodiment 8, wherein the exogenous TCR comprises a TCR beta chain variable region comprising an amino acid sequence having at least 90%, 95%, or 100% identity to the amino acid sequence of SEQ ID NO: 10.

Embodiment 10. The isolated population of embodiment 8, wherein the exogenous TCR comprises a TCR beta chain comprising an amino acid sequence having at least 90%, 95%, or 100% identity to the amino acid sequence of SEQ ID NO: 11 or 75.

Embodiment 11. The isolated population of any one of the preceding embodiments, wherein the exogenous TCR comprises a TCR alpha chain comprising (a) a CDR1-alpha sequence comprising the amino acid sequence of SEQ ID NO: 18, (b) a CDR2-alpha sequence comprising the amino acid sequence of SEQ ID NO: 19, and/or (c) a CDR3-alpha sequence comprising the amino acid sequence of SEQ ID NO: 20.

Embodiment 12. The isolated population of embodiment 11, wherein the exogenous TCR comprises a TCR alpha chain variable region comprising an amino acid sequence having at least 90%, 95%, or 100% identity to the amino acid sequence of SEQ ID NO: 21.

Embodiment 13. The isolated population of embodiment 11, wherein the exogenous TCR comprises a TCR alpha chain comprising an amino acid sequence having at least 90%, 95%, or 100% identity to the amino acid sequence of SEQ ID NO: 22 or 80.

Embodiment 14. The isolated population of any one of the preceding embodiments, wherein the exogenous TCR comprises a TCR beta chain comprising a CDR1-beta sequence comprising the amino acid sequence of SEQ ID NO: 24, a CDR2-beta sequence comprising the amino acid sequence of SEQ ID NO: 25, and/or a CDR3-beta sequence comprising the amino acid sequence of SEQ ID NO: 26.

Embodiment 15. The isolated population of embodiment 14, wherein the exogenous TCR comprises a TCR beta chain variable region comprising an amino acid sequence having at least 90%, 95%, or 100% identity to the amino acid sequence of SEQ ID NO: 27.

Embodiment 16. The isolated population of embodiment 14, wherein the exogenous TCR comprises a TCR beta chain comprising an amino acid sequence having at least 90%, 95%, or 100% identity to the amino acid sequence of SEQ ID NO: 28 or 82.

Embodiment 17. The isolated population of any one of the preceding embodiments, wherein the exogenous TCR comprises a TCR alpha chain comprising (a) a CDR1-alpha sequence comprising the amino acid sequence of SEQ ID NO: 32, (b) a CDR2-alpha sequence comprising the amino acid sequence of SEQ ID NO: 33, and/or (c) a CDR3-alpha sequence comprising the amino acid sequence of SEQ ID NO: 34.

Embodiment 18. The isolated population of embodiment 17, wherein the exogenous TCR comprises a TCR alpha chain variable region comprising an amino acid sequence having at least 90%, 95%, or 100% identity to the amino acid sequence of SEQ ID NO: 35.

Embodiment 19. The isolated population of embodiment 17, wherein the exogenous TCR comprises a TCR alpha chain comprising an amino acid sequence having at least 90%, 95%, or 100% identity to the amino acid sequence of SEQ ID NO: 36.

Embodiment 20. The isolated population of any one of the preceding embodiments, wherein the exogenous TCR comprises a TCR beta chain comprising a CDR1-beta sequence comprising the amino acid sequence of SEQ ID NO: 38, a CDR2-beta sequence comprising the amino acid sequence of SEQ ID NO: 39, and/or a CDR3-beta sequence comprising the amino acid sequence of SEQ ID NO: 40.

Embodiment 21. The isolated population of embodiment 20, wherein the exogenous TCR comprises a TCR beta chain variable region comprising an amino acid sequence having at least 90%, 95%, or 100% identity to the amino acid sequence of SEQ ID NO: 41.

Embodiment 22. The isolated population of embodiment 20, wherein the exogenous TCR comprises a TCR beta chain comprising an amino acid sequence having at least 90%, 95%, or 100% identity to the amino acid sequence of SEQ ID NO: 42.

Embodiment 23. The isolated population of any one of the preceding embodiments, wherein the exogenous TCR is encoded as a single polypeptide, optionally wherein the polypeptide comprises an N-terminal beta domain and a C-terminal alpha domain.

Embodiment 24. The isolated population of embodiment 23, wherein the single polypeptide comprises a TCR alpha chain and a TCR beta chain, and wherein the polypeptide comprises a self-cleaving peptide sequence positioned between the TCR alpha chain and the TCR beta chain.

Embodiment 25. The isolated population of embodiment 24, wherein the self-cleaving peptide sequence is a 2A peptide sequence, optionally wherein the 2A peptide sequence is a P2A, E2A, F2A, or T2A peptide sequence.

Embodiment 26. The isolated population of any one of the preceding embodiments, wherein the exogenous TCR comprises a TCR alpha chain comprising a constant region comprising an amino acid sequence having at least 90%, 95%, or 100% identity to the amino acid sequence of SEQ ID NO: 58.

Embodiment 27. The isolated population of any one of the preceding embodiments, wherein the exogenous TCR comprises a TCR beta chain comprising a constant region comprising an amino acid sequence having at least 90%, 95%, or 100% identity to the amino acid sequence of SEQ ID NO: 59 or 60.

Embodiment 28. The isolated population of any one of the preceding embodiments, wherein the exogenous human TCR comprises one or more amino acid substitutions to cysteine residues in the TCR alpha chain constant region and the TCR beta chain constant region, and wherein the cysteine residues are capable of forming one or more disulfide bridges.

Embodiment 29. The isolated population of any one of the preceding embodiments, wherein the TCR alpha chain constant region comprises a T48C amino acid substitution relative to a TCR alpha chain constant region comprising the amino acid sequence of SEQ ID NO: 58, and wherein the TCR beta chain constant region comprises a S57C amino acid substitution relative to a TCR beta chain constant region comprising the amino acid sequence of SEQ ID NO: 60.

Embodiment 30. The isolated cell population of any one of the preceding embodiments, wherein at least 70% of the cells of the isolated cell population are stable regulatory T cells comprising a hypomethylated TSDR at an endogenous FOXP3 locus.

Embodiment 31. The isolated population of embodiment 30, wherein at least 75%, at least 80%, at least 85%, at least 90%, at least 95%, at least 96%, at least 97%, at least 98%, or at least 99% of the cells of the isolated cell population are stable regulatory T cells comprising a hypomethylated TSDR at an endogenous FOXP3 locus.

Embodiment 32. The isolated cell population of any one of the preceding embodiments, wherein at least 85%, at least 90%, or at least 95% of the cells of the isolated cell population are CD25+/highCD4+CD127−/lo regulatory T cells.

Embodiment 33. The isolated cell population of any one of the preceding embodiments, wherein at least 70% of the cells of the isolated cell population are CD25^{+/high}CD4⁺CD127^{-/low}FOXP3⁺ regulatory T cells.

Embodiment 34. The isolated cell population of embodiment 33, wherein at least 75%, at least 80%, at least 85%, at least 90%, at least 95%, at least 96%, at least 97%, at least 98%, or at least 99% of the cells of the isolated cell population are CD25^{+/high}CD4⁺CD127^{-/low}FOXP3⁺ regulatory T cells.

Embodiment 35. The isolated cell population of any one of the preceding embodiments, wherein the percentage of cells of the isolated cell population comprising the stable regulatory T cells does not decrease by more than 10, 9, 8, 7, 6, 5, 4, 3, 2, or 1 percentage point(s) during at least 5, 6, 7, 8, 9, 10, or 11 days of expansion following transduction of the stable regulatory T cells with the exogenous human TCR.

Embodiment 36. The isolated cell population of embodiment 35, wherein the percentage of cells of the isolated cell population comprising the stable regulatory T cells does not decrease by more than 10 percentage points during at least 5 days of expansion following transduction of the stable regulatory T cells with the exogenous human TCR, or wherein the percentage of cells of the isolated cell population comprising the stable regulatory T cells does not decrease by more than 10 percentage points during at least 10 days of expansion following transduction of the stable regulatory T cells with the exogenous human TCR.

Embodiment 37. The isolated cell population of embodiment 35, wherein the percentage of cells of the isolated cell population comprising the stable regulatory T cells does not decrease by more than 5 percentage points during at least 5 days of expansion following transduction of the stable regulatory T cells with the exogenous human TCR, or wherein the percentage of cells of the isolated cell population comprising the stable regulatory T cells does not decrease by more than 5 percentage points during at least 10 days of expansion following transduction of the stable regulatory T cells with the exogenous human TCR.

Embodiment 38. The isolated cell population of embodiment 35, wherein the percentage of cells of the isolated cell population comprising the stable regulatory T cells does not decrease by more than 1 percentage point during at least 5 days of expansion following transduction of the stable regulatory T cells with the exogenous human TCR, or wherein the percentage of cells of the isolated cell population comprising the stable regulatory T cells does not decrease by more than 1 percentage point during at least 10 days of expansion following transduction of the stable regulatory T cells with the exogenous human TCR.

Embodiment 39. The isolated cell population of any one of the preceding embodiments, wherein the percentage of cells of the isolated cell population comprising the stable regulatory T cells does not decrease by more than 10%, 9%, 8%, 7%, 6%, 5%, 4%, 3%, 2%, or 1% during at least 5, 6, 7, 8, 9, 10, or 11 days of expansion following transduction of the stable regulatory T cells with the exogenous human TCR.

Embodiment 40. The isolated cell population of embodiment 39, wherein the percentage of cells of the isolated cell population comprising the stable regulatory T cells does not decrease by more than 10% during at least 5 days of expansion following transduction of the stable regulatory T cells with the exogenous human TCR, or wherein the percentage of cells of the isolated cell population comprising the stable regulatory T cells does not decrease by more than 10% during at least 10 days of expansion following transduction of the stable regulatory T cells with the exogenous human TCR.

Embodiment 41. The isolated cell population of embodiment 39, wherein the percentage of cells of the isolated cell population comprising the stable regulatory T cells does not decrease by more than 5% during at least 5 days of expansion following transduction of the stable regulatory T cells with the exogenous human TCR, or wherein the percentage of cells of the isolated cell population comprising the stable regulatory T cells does not decrease by more than 5% during at least 10 days of expansion following transduction of the stable regulatory T cells with the exogenous human TCR.

Embodiment 42. The isolated cell population of embodiment 39, wherein the percentage of cells of the isolated cell population comprising the stable regulatory T cells does not decrease by more than 1% during at least 5 days of expansion following transduction of the stable regulatory T cells with the exogenous human TCR, or wherein the percentage of cells of the isolated cell population comprising the stable regulatory T cells does not decrease by more than 1% during at least 10 days of expansion following transduction of the stable regulatory T cells with the exogenous human TCR.

Embodiment 43. The isolated cell population of any one of the preceding embodiments comprising at least 1×10⁷ of the stable regulatory T cells.

Embodiment 44. The isolated cell population of embodiment 43 comprising 1×10⁷ to 1×10⁹ of the stable regulatory T cells.

Embodiment 45. The isolated cell population of any one of the preceding embodiments, wherein less than 5%, less than 2%, or less than 1% of the cells of the isolated cell population express FOXP3 protein from an engineered FOXP3 locus.

Embodiment 46. The isolated cell population of any one of the preceding embodiments, wherein less than 10% of the cells of the isolated cell population express FOXP3 protein from (a) an endogenous open reading frame operably linked to an engineered FOXP3 promoter, or (b) an exogenous FOXP3 transgene.

Embodiment 47. The isolated cell population of any one of the preceding embodiments, wherein at least 90%, at least 95%, or 100% of the cells of the isolated cell population comprise an unmodified endogenous FOXP3 gene locus.

Embodiment 48. The isolated cell population of any one of the preceding embodiments, wherein less than 25%, less than 20%, less than 15%, less than 10%, less than 5%, less than 2%, or less than 1% of the cells of the isolated cell population comprise an engineered FOXP3 gene or an engineered FOXP3 protein.

Embodiment 49. The isolated cell population of any one of the preceding embodiments, wherein at least 80%, at least 85%, at least 90%, at least 95%, or 100% of the cells of the isolated cell population do not comprise an exogenous FOXP3 gene or an exogenous FOXP3 protein.

Embodiment 50. The isolated cell population of any one of the preceding embodiments, wherein at least 90%, at least 95%, or 100% of the cells of the isolated cell population do not exhibit ectopic FOXP3 expression.

Embodiment 51. The isolated cell population of any one of the preceding embodiments, wherein at least 90%, at least 95%, or 100% of the cells of the isolated cell population express physiological levels of functional FOXP3 protein.

Embodiment 52. The isolated cell population of any one of the preceding embodiments, wherein less than 25%, less than 20%, less than 15%, less than 10%, less than 5%, less than 2%, or less than 1% of the cells of the isolated cell population overexpress functional FOXP3 protein.

Embodiment 53. The isolated cell population of any one of the preceding embodiments, wherein less than 10%, less than 5%, less than 2%, less than 1%, less than 0.5%, less than 0.1%, or less than 0.01% of the cells of the isolated cell population are conventional T cells.

Embodiment 54. The isolated cell population of any one of the preceding embodiments, wherein about 0.01% to about 0.1%, about 0.1% to about 0.5%, about 0.5% to about 1%, about 1% to about 2%, about 2% to about 5%, or about 5% to about 10% of the cells of the isolated cell population are conventional T cells.

Embodiment 55. The isolated cell population of embodiment 53 or 54, wherein conventional T cells of the isolated population comprise the exogenous human TCR.

Embodiment 56. The isolated cell population of embodiment 55, wherein about 10%, about 15%, about 20%, about 30%, about 40%, about 50%, or about 60% of the conventional T cells comprise the exogenous TCR.

Embodiment 57. The isolated cell population of any one of the preceding embodiments, wherein the ratio of regulatory T cells to conventional T cells in the isolated cell population is at least 10:1, at least 20:1, at least 30:1, at least 40:1, at least 50:1, at least 60:1, at least 70:1, at least 80:1, at least 90:1, or at least 100:1.

Embodiment 58. The isolated cell population of any one of the preceding embodiments, wherein less than 1%, less than 0.5%, less than 0.1%, or less than 0.01% of the cells of the isolated cell population are CD8+ T cells.

Embodiment 59. The isolated cell population of embodiment 58, wherein the CD8+ T cells are undetectable, optionally undetectable by fluorescence activated cell sorting (FACS).

Embodiment 60. The isolated cell population of any one of the preceding embodiments, wherein at least 10% of the regulatory T cells of the isolated cell population express the exogenous human TCR.

Embodiment 61. The isolated cell population of embodiment 60, wherein at least 15%, at least 20%, at least 30%, at least 40%, at least 50%, at least 60%, at least 70%, at least 80%, or at least 90% of the regulatory T cells of the isolated cell population express the exogenous human TCR.

Embodiment 62. The isolated cell population of any one of the preceding embodiments, wherein the percentage of CD25^{+/high}CD4⁺CD127^{-/low} regulatory T cells is assessed 1 hour to 120 days post transduction.

Embodiment 63. The isolated cell population of any one of embodiments 1-62, wherein the percentage of CD25^{+/high}CD4⁺CD127^{-/low} regulatory T cells is assessed at least 1, 12, 24, 48, 72, 96, or 120 hours post transduction.

Embodiment 64. The isolated cell population of any one of embodiments 1-62, wherein the percentage of CD25^{+/high}CD4⁺CD127^{-/low} regulatory T cells is assessed 7 to 14 days post transduction.

Embodiment 65. The isolated cell population of any one of embodiments 1-62, wherein the percentage of CD25^{+/high}CD4⁺CD127^{-/low} regulatory T cells is assessed at least 10, 15, 30, 60, or 120 days post-transduction.

Embodiment 66. The isolated cell population of any one of the preceding embodiments, wherein the TSDR at the endogenous FOXP3 locus remains hypomethylated following a cryopreservation freeze-thaw cycle at least until the cells of the isolated cell population are administered to a subject.

Embodiment 67. The isolated cell population of any one of the preceding embodiments, wherein the stable regulatory T cells of the isolated cell population exhibit one or more functions selected from: (a) cytokine secretion activity; (b) expression of activation markers associated with regulatory T cells; and (c) suppression activity.

Embodiment 68. The isolated cell population of embodiment 67, wherein the one or more functions is assessed 1 hour to 14 days post transduction.

Embodiment 69. The isolated cell population of embodiment 67, wherein the one or more functions is assessed at least 12, 24, 48, 72, 96, or 120 hours post transduction.

Embodiment 70. The isolated cell population of embodiment 67, wherein the one or more functions is assessed at least 7, 8, 9, 10, 11, 12, 13, or 14 days post transduction.

Embodiment 71. The isolated cell population of any one of embodiment 67-70, wherein the one or more functions is assessed post-cryopreservation of the isolated cell population, optionally at least 24 hours post-cryopreservation.

Embodiment 72. The isolated cell population of any one of the preceding embodiments, wherein at least 10% of the stable regulatory T cells are CD45RA+.

Embodiment 73. The isolated cell population of embodiment 72, wherein at least 25%, at least 50%, at least 75%, at least 80%, at least 85%, at least 90%, at least 95%, at least 96%, at least 97%, at least 98%, or at least 99% of the stable regulatory T cells are CD45RA⁺.

Embodiment 74. The isolated cell population of any one of the preceding embodiments, wherein at least 10% of the cells of the isolated cell population is CD25^{+/high}CD4⁺CD127^{-/low}CD45RA⁺ prior to activation and transduction with the exogenous human TCR.

Embodiment 75. The isolated cell population of any one of the preceding embodiments, wherein at least 25%, at least 50%, at least 75%, at least 80%, at least 85%, at least 90%, at least 95%, at least 96%, at least 97%, at least 98%, or at least 99% of the cells of the isolated cell population is CD25^{+/high}CD4⁺CD127^{-/low}CD45RA⁺ prior to activation and transduction with the exogenous human TCR.

Embodiment 76. The isolated population of any one of the preceding embodiments, wherein the CD25^{+/high}CD4⁺CD127^{-/low} regulatory T cells have a human leukocyte antigen (HLA)-DR15 haplotype.

Embodiment 77. The isolated population of embodiment 76, wherein the CD25^{+/high}CD4⁺CD127^{-/low} regulatory T cells comprise an HLA-DRB1*15:01 allele.

Embodiment 78. The isolated population of embodiment 77, wherein the CD25^{+/high}CD4⁺CD127^{-/low} regulatory T cells further comprise an HLA-DRB5*01:01 allele.

Embodiment 79. A method of producing the isolated cell population of any one of the preceding embodiments, the method comprising: (a) isolating a biological sample comprising regulatory T cells from a human subject having Multiple Sclerosis, optionally progressive Multiple Sclerosis; (b) removing CD8+ cells, CD19+ cells, and optionally CD14⁺ cells from the biological sample to produce a depleted biological sample; (c) selecting CD25^{+/high} cells from the depleted biological sample to produce a CD25-enriched cell population; (d) selecting CD25^{+/high}CD4⁺CD127^{-/low}CD45RA⁺ cells from the CD25-enriched cell population to produce one or more positive fractions; (e) selecting CD25^{+/high}CD4⁺CD127^{-/low}CD45RA⁺ cells from the one or more positive fractions to produce a cell population comprising stable regulatory T cells; and (f) engineering the cell population comprising stable regulatory T cells such that the stable regulatory T cells comprise the exogenous human TCR, thereby producing the isolated population of cells.

Embodiment 80. The method of embodiment 79, wherein less than 5%, less than 4%, or less than 3% of the cells of the depleted sample comprise CD8+ cells, CD19+ cells, and/or CD14+ cells.

Embodiment 81. The method of any one of embodiments 79-80, wherein less than 0.5% of the cells of the depleted sample comprise CD8+ cells.

Embodiment 82. The method of any one of embodiments 79-81, wherein the selecting of (d) comprises identifying CD4⁺CD45RA⁺ cells and then identifying CD25^{+/high}CD127^{-/low} cells from the identified CD4⁺CD45RA⁺ cells to identify a first population of CD25^{+/high}CD4⁺CD127^{-/low}CD45RA⁺ cells, then selecting the first population of CD25^{+/high}CD4⁺CD127^{-/low}CD45RA⁺ cells from the first subpopulation to produce the one or more positive fractions.

Embodiment 83. The method of any one of embodiments 79-82, wherein the selecting of (e) comprises identifying CD4⁺CD45RA⁺ cells and then identifying CD25^{+/high} CD127^{-/low} cells from the identified CD4⁺CD45RA⁺ cells to identify a second population of CD25^{+/high}CD4⁺CD127^{-/low}CD45RA⁺ cells, then selecting the second population of CD25^{+/high}CD4⁺CD127^{-/low}CD45RA⁺ cells from the second subpopulation to produce the cell population comprising stable regulatory T cells.

Embodiment 84. The method of any one of embodiments 79-83, wherein the subject has a human leukocyte antigen (HLA)-DR15 haplotype.

Embodiment 85. The method of embodiment 84, wherein the regulatory T cells comprise an HLA-DRB1*15:01 allele.

Embodiment 86. The method of any one of embodiments 79-85, wherein the subject is a male subject.

Embodiment 87. The method of any one of embodiments 79-85, wherein the subject is a female subject.

Embodiment 88. The method of any one of embodiments 79-87, wherein engineering the isolated cell population comprises transducing the cell population with a nucleic acid encoding the exogenous human TCR.

Embodiment 89. The method of any one of embodiments 79-88, further comprising activating and expanding cells of the isolated cell population to produce a cell population comprising at least 1×10⁷ stable CD25^{+/high}CD4⁺CD127^{-/low} regulatory T cells comprising a hypomethylated TSDR at an endogenous FOXP3 locus.

Embodiment 90. The method of embodiment 89, wherein the activating and expanding comprises culturing cells of the isolated cell population for at least 5, 6, 7, 8, 9, or 10 days.

Embodiment 91. The method of embodiment 89, wherein the activating and expanding comprises culturing cells of the isolated cell population for no more than 15, 14, 13, or 12 days.

Embodiment 92. The method of any one of embodiments 88-91, wherein the nucleic acid is a vector.

Embodiment 93. The method of embodiment 92, wherein the vector is a viral vector.

Embodiment 94. The method of embodiment 93, wherein the viral vector is a lentiviral vector.

Embodiment 95. The method of any one of embodiments 88-94, wherein the nucleic acid comprises a promoter operably linked to a coding sequence encoding the exogenous human TCR, optionally wherein the promoter is an EF-1 alpha promoter or an MND promoter.

Embodiment 96. The method of any one of embodiments 88-95, wherein the nucleic acid further comprises an enhancer element, optionally an optimized post-transcriptional regulatory element (oPRE) or a woodchuck hepatitis virus post-transcriptional regulatory element (WPRE), further optionally WPRE-mut6.

Embodiment 97. The method of any one of embodiments 88-96, wherein the coding sequence is codon-optimized.

Embodiment 98. The vector of any one of embodiments 88-97.

Embodiment 99. A pharmaceutical composition comprising the isolated cell population of any one of embodiments 1-78 and a pharmaceutically acceptable excipient.

Embodiment 100. A composition comprising the isolated cell population of any one of embodiments 1-78 and a cryopreservative.

Embodiment 101. A method comprising administering to a subject the pharmaceutical composition of embodiment 99, wherein the subject has Multiple Sclerosis.

Embodiment 102. The method of embodiment 101, wherein the subject has progressive Multiple Sclerosis.

Embodiment 103. The method of embodiment 102, wherein the subject has primary progressive Multiple Sclerosis (PPMS) or non-relapsing progressive Multiple Sclerosis.

Embodiment 104. The method of any one of embodiments 101-103, wherein the pharmaceutical composition is administered in an effective amount to alleviate one or more symptom of Multiple Sclerosis.

Embodiment 105. The method of any one of embodiments 101-104, wherein the isolated population is obtained from the subject.

Embodiment 106. The method of any one of embodiments 101-105, wherein the subject has an HLA-DR15 haplotype.

Embodiment 107. The method of embodiment 106, wherein the subject has an HLA-DRB1*15:01 allele.

Embodiment 108. The method of embodiment 106 or 107, wherein the subject has an HLA-DRB5*01:01 allele.

Embodiment 109. A method comprising administering to a subject the isolated population of any one of embodiments 1-78.

Embodiment 110. The method of embodiment 109, wherein the subject has Multiple Sclerosis.

Embodiment 111. The method of embodiment 110, wherein the subject has progressive Multiple Sclerosis.

Embodiment 112. The method of embodiment 111, wherein the subject has primary progressive Multiple Sclerosis (PPMS) or non-relapsing progressive Multiple Sclerosis.

Embodiment 113. The method of any one of embodiments 101-112, wherein the isolated population is obtained from the subject.

Embodiment 114. The method of any one of embodiments 101-113, wherein the subject has an HLA-DR15 haplotype.

Embodiment 115. The method of embodiment 114, wherein the subject has an HLA-DRB1*15:01 allele.

Embodiment 116. The method of embodiment 114 or 115, wherein the subject has an HLA-DRB5*01:01 allele.

Embodiment 117. The method of any one of embodiments 101-116, wherein the administering comprises intravenous administration.

Embodiment 118. The method of any one of embodiments 101-117, wherein the administering comprises one or more infusion.

Embodiment 133. An engineered polynucleotide comprising an open reading frame comprising a nucleotide sequence having at least 80% identity to the nucleotide sequence of SEQ ID NO: 107.

Embodiment 134. The engineered polynucleotide of embodiment 133, wherein the nucleotide sequence of the open reading frame has at least 85%, at least 90%, at least 95%, at least 96%, at least 97%, at least 98%, or at least 99% identity to the nucleotide sequence of SEQ ID NO: 107.

Embodiment 135. The engineered polynucleotide of embodiment 134, where the nucleotide sequence of the open reading frame has 100% identity to the nucleotide sequence of SEQ ID NO: 107.

Embodiment 136. An engineered polynucleotide encoding a polypeptide comprising an amino acid sequence having at least 80% identity to the amino acid sequence of SEQ ID NO: 108.

Embodiment 137. The engineered polynucleotide of embodiment 136, wherein the amino acid sequence of the polypeptide has at least 85%, at least 90%, at least 95%, at least 96%, at least 97%, at least 98%, or at least 99% identity to the nucleotide sequence of SEQ ID NO: 108.

Embodiment 138. The engineered polynucleotide of embodiment 136 or 137, wherein the amino acid sequence of the polypeptide comprises a first cysteine at a position corresponding to position 189 of the amino acid sequence of SEQ ID NO: 108 and/or a second cysteine at a position corresponding to position 513 of the amino acid sequence of SEQ ID NO: 108.

Embodiment 139. The engineered polynucleotide of embodiment 138, wherein the amino acid sequence of the polypeptide has 100% identity to the amino acid sequence of SEQ ID NO: 108.

Embodiment 140. The engineered polynucleotide of any one of embodiments 133-139, wherein the open reading frame encodes a human/mouse hybrid T cell receptor (TCR), e.g., wherein the variable region is a human variable region and the constant domain is a mouse constant domain.

Embodiment 141. The engineered polynucleotide of embodiment 140, wherein a human/mouse hybrid TCR comprises an alpha chain comprising the amino acid sequence of SEQ ID NO: 109, SEQ ID NO: 110, and SEQ ID NO: 111.

Embodiment 142. The engineered polynucleotide of embodiment 141, wherein a human/mouse hybrid TCR comprises an alpha chain comprising the amino acid sequence of SEQ ID NO: 112.

Embodiment 143. The engineered polynucleotide of embodiment 141, wherein a human/mouse hybrid TCR comprises an alpha chain comprising the amino acid sequence of SEQ ID NO: 113 or SEQ ID NO: 114.

Embodiment 144. The engineered polynucleotide of embodiment 141, wherein a human/mouse hybrid TCR comprises an alpha constant domain comprising the amino acid sequence of SEQ ID NO: 123.

Embodiment 145. The engineered polynucleotide of any one of embodiments 140-144, wherein a human/mouse hybrid TCR comprises a beta chain comprising the amino acid sequence of SEQ ID NO: 115, SEQ ID NO: 116, and SEQ ID NO: 117.

Embodiment 146. The engineered polynucleotide of embodiment 145, wherein a human/mouse hybrid TCR comprises a beta chain comprising the amino acid sequence of SEQ ID NO: 118.

Embodiment 147. The engineered polynucleotide of embodiment 145, wherein a human/mouse hybrid TCR comprises a beta chain comprising the amino acid sequence of SEQ ID NO: 119, SEQ ID NO: 120, SEQ ID NO: 121, or SEQ ID NO: 122.

Embodiment 148. The engineered polynucleotide of embodiment 145, wherein a human/mouse hybrid TCR comprises a beta constant domain comprising the amino acid sequence of SEQ ID NO: 124.

Embodiment 149. The engineered polynucleotide of any one of embodiments 140-148, wherein the TCR binds specifically to a myelin basic protein (MBP) peptide complexed with a major histocompatibility complex (MHC).

Embodiment 150. An engineered polynucleotide comprising an open reading frame encoding a polypeptide comprising an amino acid sequence having at least 80% identity to the amino acid sequence of SEQ ID NO: 113.

Embodiment 151. The engineered polynucleotide of embodiment 150, wherein the amino acid sequence of the polypeptide comprises an amino acid sequence having at least 85%, at least 90%, at least 95%, at least 96%, at least 97%, at least 98%, or at least 99% identity to the amino acid sequence of SEQ ID NO: 113.

Embodiment 152. The engineered polynucleotide of embodiment 151, wherein the amino acid sequence of the polypeptide comprises the amino acid sequence of SEQ ID NO: 113.

Embodiment 153. The engineered polynucleotide of any one of embodiments 150-152, wherein the amino acid sequence of the polypeptide further comprises an amino terminal leader sequence, optionally METLLGVSLV ILWLQLARVN (SEQ ID NO: 100).

Embodiment 154. The engineered polynucleotide of embodiment 153, wherein the amino acid sequence of the polypeptide comprises the amino acid sequence of SEQ ID NO: 114.

Embodiment 155. An engineered polynucleotide comprising an open reading frame encoding a polypeptide comprising an amino acid sequence having at least 80% identity to the amino acid sequence of SEQ ID NO: 119.

Embodiment 156. The engineered polynucleotide of embodiment 155, wherein the amino acid sequence of the polypeptide comprises an amino acid sequence having at least 85%, at least 90%, at least 95%, at least 96%, at least 97%, at least 98%, or at least 99% identity to the amino acid sequence of SEQ ID NO: 119.

Embodiment 157. The engineered polynucleotide of embodiment 156, wherein the amino acid sequence of the polypeptide comprises the amino acid sequence of SEQ ID NO: 119.

Embodiment 158. The engineered polynucleotide of any one of embodiments 155-157, wherein the amino acid sequence of the polypeptide further comprises an amino terminal leader sequence, optionally METLLGVSLV ILWLQLARVN (SEQ ID NO: 100).

Embodiment 159. The engineered polynucleotide of embodiment 158, wherein the amino acid sequence of the polypeptide comprises the amino acid sequence of SEQ ID NO: 121.

Embodiment 160. The engineered polynucleotide of any one of embodiments 155-159, wherein the amino acid sequence of the polypeptide further comprises a carboxy terminal self-cleaving peptide sequence, optionally selected from P2A, E2A, F2A, and T2A, and optionally comprising the sequence of GSG ATNFSLLKQA GDVEENPG (SEQ ID NO: 104).

Embodiment 161. The engineered polynucleotide of any one of embodiments 155-157, wherein the amino acid sequence of the polypeptide comprises the amino acid sequence of SEQ ID NO: 120.

Embodiment 162. The engineered polynucleotide of any one of embodiments 155-157, wherein the amino acid sequence of the polypeptide comprises the amino acid sequence of SEQ ID NO: 122.

Embodiment 163. The engineered polynucleotide of any one of the preceding embodiments, wherein the engineered polynucleotide is a messenger ribonucleic acid (mRNA).

Embodiment 164. The engineered polynucleotide of any one of embodiments 133-162, wherein the engineered polynucleotide is a deoxyribonucleic acid (DNA).

Embodiment 165. The engineered polynucleotide of embodiment 164, further comprising a promoter operably linked to the open reading frame.

Embodiment 166. The engineered polynucleotide of embodiment 165, wherein the promoter is an EF-1 alpha promoter or an MND promoter.

Embodiment 179. An engineered polypeptide comprising an amino acid sequence having at least 80% identity to the amino acid sequence of SEQ ID NO: 108.

Embodiment 180. The engineered polypeptide of embodiment 179, wherein the amino acid sequence of the polypeptide has at least 85%, at least 90%, at least 95%, at least 96%, at least 97%, at least 98%, or at least 99% identity to the nucleotide sequence of SEQ ID NO: 108.

Embodiment 181. The engineered polypeptide of embodiment 180, wherein the amino acid sequence of the polypeptide has 100% identity to the amino acid sequence of SEQ ID NO: 108.

Embodiment 182. An engineered polypeptide comprising an amino acid sequence having at least 80% identity to the amino acid sequence of SEQ ID NO: 113.

Embodiment 183. The engineered polypeptide of embodiment 182, wherein the amino acid sequence of the polypeptide comprises an amino acid sequence having at least 85%, at least 90%, at least 95%, at least 96%, at least 97%, at least 98%, or at least 99% identity to the amino acid sequence of SEQ ID NO: 113.

Embodiment 184. The engineered polypeptide of embodiment 183, wherein the amino acid sequence of the polypeptide comprises the amino acid sequence of SEQ ID NO: 113.

Embodiment 185. The engineered polypeptide of any one of embodiments 182-184, wherein the amino acid sequence of the polypeptide further comprises an amino terminal leader sequence, optionally METLLGVSLV ILWLQLARVN (SEQ ID NO: 100).

Embodiment 186. The engineered polypeptide of embodiment 185, wherein the amino acid sequence of the polypeptide comprises the amino acid sequence of SEQ ID NO: 114.

Embodiment 187. An engineered polypeptide encoding a polypeptide comprising an amino acid sequence having at least 80% identity to the amino acid sequence of SEQ ID NO: 119, wherein the amino acid sequence of the polypeptide comprises a cysteine at a position corresponding to position 175 of the amino acid sequence of SEQ ID NO: 119.

Embodiment 188. The engineered polypeptide of embodiment 187, wherein the amino acid sequence of the polypeptide comprises an amino acid sequence having at least 85%, at least 90%, at least 95%, at least 96%, at least 97%, at least 98%, or at least 99% identity to the amino acid sequence of SEQ ID NO: 119.

Embodiment 189. The engineered polypeptide of embodiment 188, wherein the amino acid sequence of the polypeptide comprises the amino acid sequence of SEQ ID NO: 119.

Embodiment 190. The engineered polypeptide of any one of embodiments 187-189, wherein the amino acid sequence of the polypeptide further comprises an amino terminal leader sequence, optionally METLLGVSLV ILWLQLARVN (SEQ ID NO: 100).

Embodiment 191. The engineered polypeptide of embodiment 190, wherein the amino acid sequence of the polypeptide comprises the amino acid sequence of SEQ ID NO: 121.

Embodiment 192. The engineered polypeptide of any one of embodiments 187-191, wherein the amino acid sequence of the polypeptide further comprises a carboxy terminal P2A overhang sequence, optionally GSG ATNFSLLKQA GDVEENPG (SEQ ID NO: 104).

Embodiment 193. The engineered polypeptide of any one of embodiments 187-189, wherein the amino acid sequence of the polypeptide comprises the amino acid sequence of SEQ ID NO: 120.

Embodiment 194. The engineered polypeptide of any one of embodiments 187-189, wherein the amino acid sequence of the polypeptide comprises the amino acid sequence of SEQ ID NO: 122.

Embodiment 195. A T cell receptor comprising (a) the amino acid sequence of the alpha chain has at least 85%, at least 90%, at least 95%, at least 96%, at least 97%, at least 98%, or at least 99% identity to the amino acid sequence of SEQ ID NO: 113 or SEQ ID NO: 114; and/or (b) the amino acid sequence of the beta chain has at least 85%, at least 90%, at least 95%, at least 96%, at least 97%, at least 98%, or at least 99% identity to the amino acid sequence of SEQ ID NO: 119, SEQ ID NO: 120, SEQ ID NO: 121, or SEQ ID NO: 122.

Embodiment 196. The T cell receptor of embodiment 195, wherein (a) the amino acid sequence of the alpha chain has 100% identity to the amino acid sequence of SEQ ID NO: 113 or SEQ ID NO: 114; and/or (b) the amino acid sequence of the beta chain has 100% identity to the amino acid sequence of SEQ ID NO: 119, SEQ ID NO: 120, SEQ ID NO: 121, or SEQ ID NO: 122.

Embodiment 197. A T cell receptor comprising an amino acid sequence having at least 80% identity to the amino acid sequence of SEQ ID NO: 108.

Embodiment 198. The T cell receptor of embodiment 197, wherein the amino acid sequence of the T cell receptor has at least 85%, at least 90%, at least 95%, at least 96%, at least 97%, at least 98%, or at least 99% identity to the amino acid sequence of SEQ ID NO: 108.

Embodiment 199. The T cell receptor of embodiment 198, wherein the amino acid sequence of the T cell receptor has 100% identity to the amino acid sequence of SEQ ID NO: 108.

Embodiment 200. A T cell receptor encoded by the engineered polynucleotide of any one of embodiments 123-166.

Embodiment 201. A T cell receptor comprising the engineered polypeptide of any one of embodiments 173-194.

Embodiment 202. The isolated population of any one of embodiments 1-74, comprising the engineered polypeptide of any one of embodiments 173-194.

### EXAMPLES

### Example 1. Manufacturing Process for Production of Autologous Stable Regulatory T Cells Expressing an Exogenous TCR

The generation of a population of cells comprising stable thymically-derived regulatory T cells that express an exogenous TCR is shown in this Example. The overall enrichment and sorting strategy is illustrated in FIGS. 1A-1B.

### Leukapheresis, Depletion, and Enrichment

First, leukapheresis from a human subject was collected, shipped to a central processing facility, and held at 4° C. until processing at about 24 hours following collection. Incoming leukapheresis product was washed in phosphate-buffered saline containing 0.5% human serum albumin (HSA) to reduce platelet count and then labeled with a cocktail of magnetic microbeads (CD8, CD14, and CD19 microbeads; Miltenyi) using an automated cell processing system.

Using magnetic cell separation technology, cytotoxic T cells (CD8⁺), B cells (CD19⁺), and monocytes (CD14⁺) were depleted prior to enrichment for CD25⁺ cells. The volume of the depleted biological sample was reduced by centrifugation. The depleted biological sample was then labeled with CD25-PE-Biotin antibody (Miltenyi) according to the manufacturer's recommendations. The cells were then washed with phosphate-buffered saline containing 0.5% HSA and labeled with anti-biotin microbeads (Miltenyi). CD25⁺ cells were then isolated using magnetic cell separation technology to produce a CD25-enriched sample.

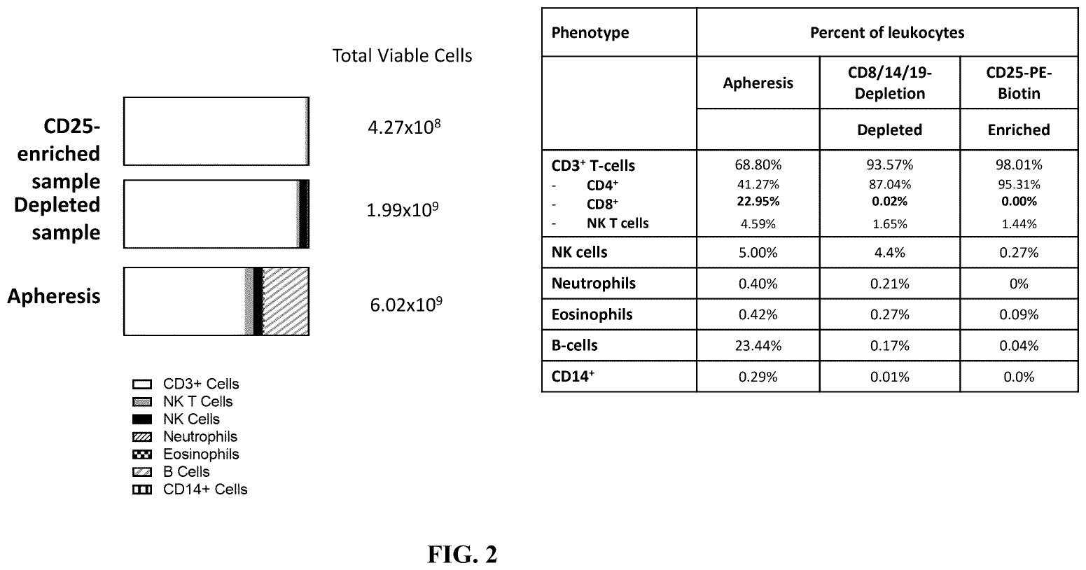

FIG. 2 shows the Viable Cells (VC) s of all cells following apheresis, removal of the CD8⁺, CD19⁺, and CD14⁺ cells (depleted sample), and selection for CD25⁺ (CD25-enriched sample) for Run 1. Approximately 98% of the total cells of the population are CD3⁺ cells following the CD25 selection step to produce the CD25-enriched sample and approximately 95% of the cells are CD4+ T cells.

Table 1 shows CD4⁺ cell and naïve Treg cell composition of the apheresis cell population and after the depletion and enrichment steps for 5 additional manufacturing runs.

TABLE 1
Composition of cell population during depletion and enrichment process

| Cells | Run 2 | Run 3 | Run 4 | Run 5 | Run 6 |
|---|---|---|---|---|---|
| Donor Sex | 33 | 59 | 29 | 37 | 26 |
| Donor Age | M | M | M | M | M |
| Donor Health Status (MS/non-MS) | MS | Healthy | Healthy | Healthy | Healthy |
| | | Donor | Donor | Donor | Donor |
| Apheresis VC | 1.1E+10 | 1.1E+10 | 8.7E+09 | 1.5E+10 | 1.1E+10 |
| Apheresis % CD4 T cells | 38% | 30% | 22% | 36% | 49% |
| Apheresis % naïve Treg | 1% | 1% | 1% | 1% | 1% |
| Post-depletion VC | 3.3E+09 | 2.6E+09 | 1.9E+09 | 4.6E+09 | 4.0E+09 |
| Post-depletion % CD4 T cells | 83% | 59% | 65% | 89% | 88% |
| Post-depletion % naïve Treg | 1% | 3% | 4% | 3% | 2% |
| Post-enrichment VC | 3.5E+09 | 2.4E+08 | 3.0E+08 | 4.6E+08 | 2.0E+08 |
| Post-enrichment % CD4 T cells | 95% | 96% | 93% | 96% | 97% |
| Post-enrichment % naïve Treg | 10% | 19% | 21% | 18% | 19% |

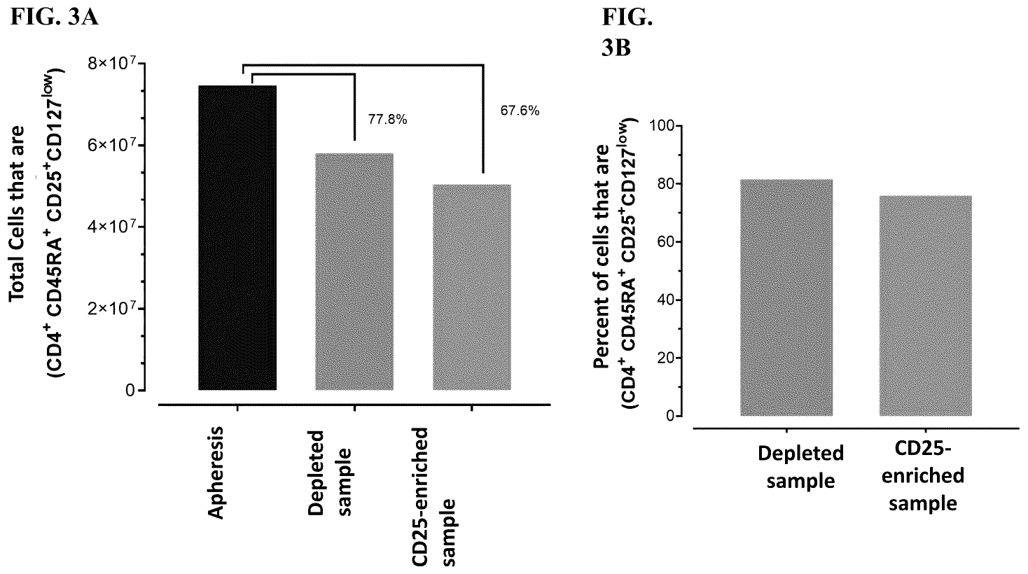

FIG. 3A shows the viable counts (VC) of naïve regulatory T cells (specifically, CD4⁺CD25⁺/highCD127−/loCD45RA+ cells) following apheresis, removal of the CD8⁺, CD19+, and CD14+ cells (depleted sample), and selection for CD25+ (CD25-enriched sample) for Run 1. FIG. 3B shows the recovery of naïve regulatory T cells (CD4+CD25+/highCD127−/loCD45RA+ cells) after each step and that approximately 80% of the CD4+CD25+/highCD127−/loCD45RA cells are recovered after the depletion step and 75% of the CD4+CD25+/highCD127−/loCD45RA+ cells are recovered after the CD25-enrichement step.

Table 2 shows VCs of naïve regulatory T cells following apheresis, depletion, and enrichment, and recovery from the previous step of naïve regulatory T cells after the depletion, and enrichment.

TABLE 2
Total viable counts (TVC) of naïve regulatory T cells
and % recovery following steps of the manufacturing process

| | Run 2 | Run 3 | Run 4 | Run 5 | Run 6 |
|---|---|---|---|---|---|
| Naïve Treg Viable Count (VC) at Apheresis | 7.6E+07 | 1.1E+08 | 1.2E+08 | 1.5E+08 | 1.4E+08 |
| Naïve Treg VC at Depletion | 4.5E+07 | 8.3E+07 | 7.2E+07 | 1.3E+08 | 6.6E+07 |
| Naïve Tregs VC following Enrichment | 3.7 + 07 | 4.6E+07 | 6.2E+07 | 8.1E+07 | 3.8E+07 |
| % recovery nTreg from apheresis following depletion | 59% | 72% | 60% | 85% | 49% |
| % nTreg Recovery from apheresis following enrichment | 49% | 40% | 52% | 53% | 28% |
| % recovery nTreg from depletion following enrichment | 84% | 60% | 81% | 60% | 63% |

### Gating Strategy

The CD25-enriched sample was then stained with fluorescently labeled anti-CD45RA, anti-CD4, and anti-CD127 antibodies and sorted by Fluorescence-Activated Cell Sorting (FACS) for the CD4⁺CD25^{+/high}CD127^{-/lo}CD45RA⁺ phenotype to produce a population of cells comprising stable regulatory T cells. FACS was performed in two rounds, with a first debulking fractional sort and a second fractional purity sort. For the debulking sort, the CD25-enriched sample was divided into multiple fractions that were sorted across multiple cell sorters. Following the debulking sort, the fractions were combined for the second round of purity sort. The purpose of fractionally sorting the cells in the debulking and/or purity sort is to expedite the sorting process by spreading the sorts over multiple machines, ensuring that the cells remain viable. The gating strategy used for the debulk and purity sorts were identical. For the gating strategy, a dot-plot based approach was used in which CD45RA⁺CD4⁺ cells were first selected, and then CD25^{+/high}CD127^{-/lo} cells were selected from the CD45RA⁺CD4⁺ cell population to optimize the purity of stable regulatory T cells in the population as determined by TSDR phenotype. As shown in FIG. 1B, this FACS gating strategy first plotted CD45RA⁺ versus CD4⁺ cells on a dot plot and selected them based on population distribution using a box gate. From the selected population of cells, CD25^{+/high} versus CD127^{-/lo} cells were plotted on a dot plot and gated using a polygon gate to select a population of CD4⁺CD25^{+/high}CD127^{-/lo} CD45RA⁺ cells for collection. This FACS strategy was developed to optimize selection of stable naïve regulatory T cells based on TSDR hypomethylation phenotype. The composition of the cell population before sorting, after debulking and after production of the population of CD4⁺CD25^{+/high}CD127^{-/lo}CD45RA⁺ cells for Run 1 is provided in Table 3.

TABLE 3
Composition of Cell Population following FACS

| | | Purity of | Total | Step recovery |
|---|---|---|---|---|
| | Total | naïve | naïve | of naïve |
| | Viable | regulatory | regulatory T | regulatory |
| | Cell Counts | T cell | cells | T cells |
| Pre-Sort | 3.6E+08 | 12% | 5.0E+07 | 76% |
| Processing | | | | |
| Post-Debulk | 2.7E+07 | 78% | 2.1E+07 | 41% |
| Sort | | | | |
| Post-Purity | 1.6E+07 | 96% | 1.6E+07 | 75% |
| Sort | | | | |

The purity and recovery of naïve Tregs during the sorting process for 5 additional manufacturing runs is shown in Table 4.

TABLE 4
Purity and recovery of naïve Tregs post-FACS sorting

| | Total nTregs | | | Post-Sort |
|---|---|---|---|---|
| | Sorted | | nTregs | nTreg Purity |
| | (Sorting input | Total nTreg | Recovery Sort | (FoxP3⁺ |
| Run | Treg) | Post-Sort | Process | Tregs) |
| Run 2 | 3.6E+07 | 1.1E+07 | 31% | 96% |
| Run 3 | 1.8E+07 | 5.5E+06 | 31% | 96% |
| Run 4 | 4.9E+07 | 1.7E+07 | 35% | 90% |
| Run 5 | 3.6E+07 | 1.1E+07 | 31% | 93% |
| Run 6 | 1.7E+07 | 5.7E+06 | 34% | 96% |

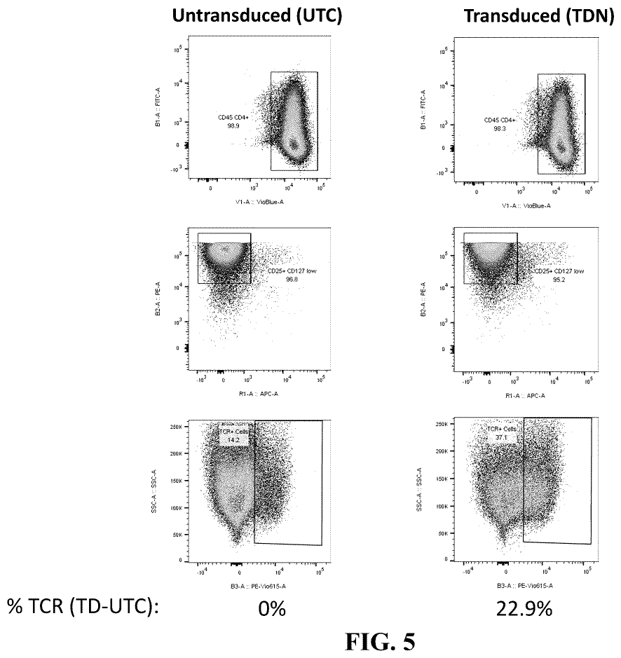

The regulatory T cells were activated after the FACS using T cell TransAct activation reagent (Miltenyi) for about 48 hours. The activated regulatory T cells were then transduced with a third generation, VSV-pseudotyped, self-inactivating lentiviral vector encoding an exogenous TCR that specifically binds to a target peptide associated with Multiple Sclerosis (e.g., progressive Multiple Sclerosis) termed ‘TCR-A’. The vector construct comprised an N-terminal TCR beta chain and a C-terminal TCR alpha chain with a linker domain comprising a GSG amino acid sequence followed by a P2A self-cleaving peptide. The TCR beta-GSG-P2A-TCR alpha fusion constructs were expressed under the EF1alpha promoter. The proportion of transduced cells, as measured by FACS analysis with an anti-Vbeta antibody, is shown in FIG. 5.

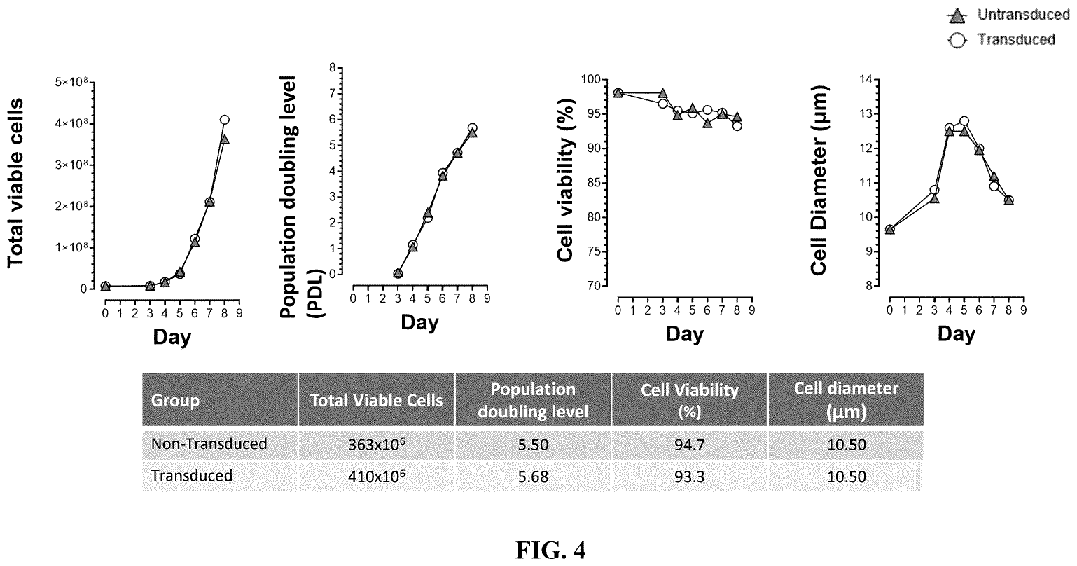

The transduced regulatory T cells were then expanded in T cell expansion media supplemented with 1,000 IU/ml IL-2 until harvest (Day 9, 10, or 11). Cell count, size, population doubling, and viability of the untransduced and transduced regulatory T cells were assessed and are shown for Run 1 (FIG. 4). Notably, the transduced regulatory T cells shared a similar growth profile with the untransduced regulatory T cells, suggesting that the exogenous TCR does not impair the growth and proliferation of the cells.

The population doubling level and % viability for 5 additional manufacturing runs is shown in Table 5. The % target dose achieved for the full process for all 6 manufacturing runs is also shown. The % target dose is calculated by determining the total final number of naïve Tregs based on the initial number of naïve Tregs prior to expansion following the sorting process and the population doubling level and assumes a transduction efficiency of 25%. The % target dose is calculated based on a target dose of 100 million transduced cells. As is shown, the manufacturing process in this example achieves a Treg product with high purity and sufficient cells to dose a patient from a single leukopak. As is used herein, PDL is population doubling level as used throughout. TDN indicates transduced cells.

TABLE 5
Properties of manufactured cells

| | Day | PDL (TDN) | % Viability | % of Target Dose |
|---|---|---|---|---|
| Run 2 | 9 | 6.8 | 94% | 370% |
| Run 3 | 9 | 7.8 | 97% | 420% |
| Run 4 | 9 | 6.4 | 94% | 450% |
| Run 5 | 9 | 6.6 | 96% | 1400% |
| Run 6 | 9 | 6.5 | N/A | 482% |

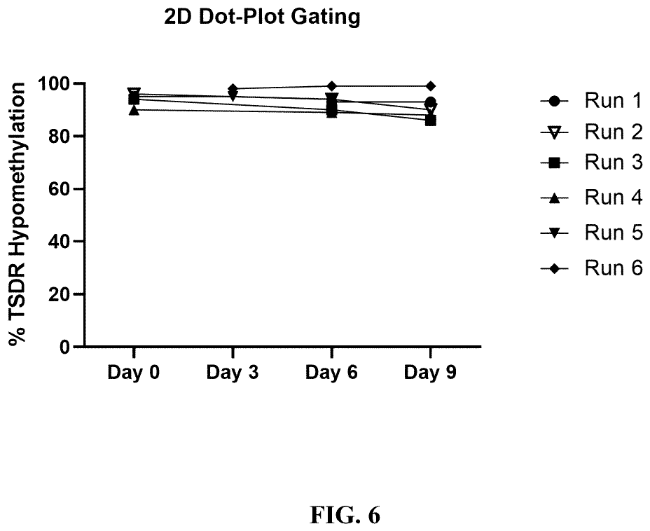

Transduced cells were collected at days 0-11 of the activation and expansion process and TSDR hypomethylation at the FOXP3 CNS2 locus was measured by a ddPCR assay. Genomic DNA (gDNA) was obtained from enriched regulatory T cells and bisulfite treated. Then a digital droplet PCR (ddPCR) assay using methylation-specific primers and probes was employed to quantify unmethylated and methylated sequences. TSDR hypomethylation status of the collected cells for all 6 transduced manufacturing runs is shown in FIG. 6. During expansion, TSDR hypomethylation levels did not decrease by more than 10% for any manufacturing run and remained above 80% for all manufacturing runs. Table 6 below shows the TSDR hypomethylation status for all 6 donor samples processed using the method described in this Example.

These data demonstrate that the regulatory T cells obtained are stable, thymically derived regulatory T cells.

TABLE 6
TSDR status of cells produced using the methods of this Example

| TSDR | Donor | | | | |
|---|---|---|---|---|---|
| Hypomethylation % | Sex | Day 0 | Day 3 | Day 6 | Day 9 |
| Run 1 | M | N/A | N/A | 93% | 93% |
| Run 2 | M | 96% | N/A | 94% | 90% |
| Run 3 | M | 94% | N/A | 90% | 86% |
| Run 4 | M | 90% | N/A | 89% | 88% |
| Run 5 | M | 95% | 95% | 94% | N/A |
| Run 6 | M | N/A | 98% | 99% | 99% |

### Activity of Tregs Following a Cryopreservation Freeze-Thaw Cycle

Regulatory T cells produced according to the methods in this example that were untransduced or transduced with the lentiviral vector encoding the TCR described herein were cryopreserved for storage. The cells were frozen and stored in a vapor-phase nitrogen storage freezer.

After a period of time in cold storage, the frozen cell population was thawed. The cell population was split into two experimental groups. The first experimental group was activated with anti-CD3 and anti-CD28 antibodies (transduced (TDN)) while the second experimental group was not (untransduced (UNT)). Both groups were expanded in media containing IL-2. Following incubation, IL-10 levels were measured after 1, 2, and 3 days.

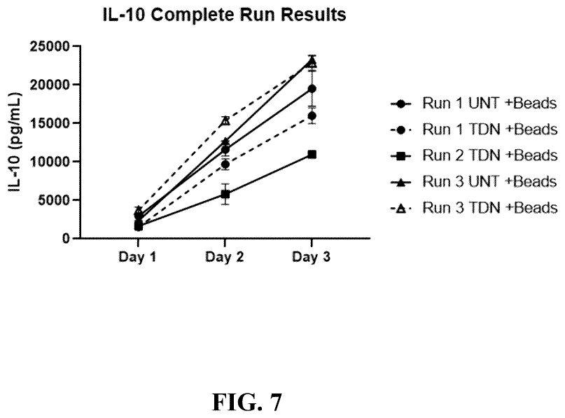

The increase in IL-10 levels for each of the runs over the three-day period for cells activated with anti-CD3 and anti-CD28 antibodies is shown in FIG. 7. The cells exhibited increasing secretion of the IL-10 cytokine over the period of three days, demonstrating that cell populations can be activated and retain their cellular functionality following a cryopreservation freeze-thaw cycle (FIG. 7). The cells that were not activated showed no increase in IL-10 levels. This data further demonstrates that the regulatory T cells retained function.

### Example 2. Gating Strategy Optimized for GMP Conditions

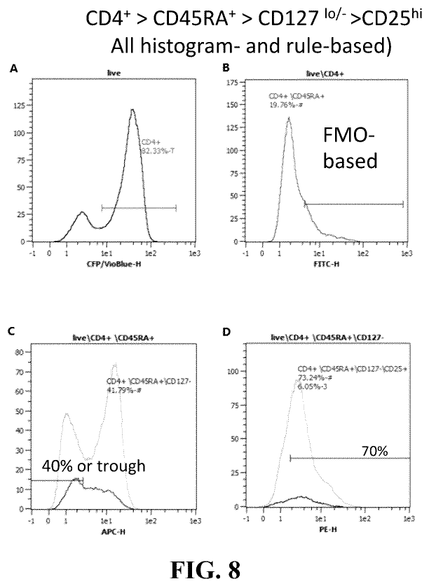

A second gating strategy was developed using a 1-D histogram-based approach rather than a 2-D dot-based approach to reduce user error in a GMP manufacturing setting. Cells were prepared as in Example 1 up to the FACS sorting step. In the FACS sorting step, the gating scheme used, shown in FIG. 8, was a one-dimensional, histogram plot-based approach in which the CD25-enriched cells were sequentially gated on CD4+, CD45RA+, CD127−/lo and CD25+/high using histograms (CD4+>CD45RA+ (histo)>CD127−/lo (histo) 40%>CD25Hi (histo) 70%). For runs with the same run number as in Example 1, both gating strategies were run in parallel on the same donor sample. Cells were transduced with TCR-A or a disulfide modified codon optimized version termed ‘TCR-E v2’

The properties of the cells prior to sorting are shown in Table 7 and the viability of the cells after each step of the manufacturing process is Table 8.

TABLE 7
Composition of cell population during depletion and enrichment process

| Cells | Run 5 | Run 6 | Run 7 | Run 8 | Run 9 |
|---|---|---|---|---|---|
| Donor Sex | 37 | 26 | 37 | 35 | 22 |
| Donor Age | M | M | M | M | F |
| Donor Health Status (MS/non-MS) | Healthy | Healthy | Healthy | Healthy | Healthy |
| | Donor | Donor | Donor | Donor | Donor |
| Apheresis VC | 1.5E+10 | 1.1E+10 | 2.3E+10 | 1.7E+10 | 1.2E+10 |
| Apheresis % CD4 T cells | 36% | 49% | 48% | 41% | 39% |
| Apheresis % naïve Treg | 1% | 1% | 1% | 1% | 1% |
| Post-depletion VC | 4.6E+09 | 4.0E+09 | 7.9E+09 | 6.0E+09 | 3.9E+09 |
| Post-depletion % CD4 T cells | 89% | 88% | 83% | 82% | 83% |
| Post-depletion % naïve Treg | 3% | 2% | 2% | 1% | 2% |
| Post-enrichment VC | 4.6E+08 | 2.0E+08 | 3.7E+08 | 3.0E+08 | 3.2E+08 |
| Post-enrichment % CD4 T cells | 96% | 97% | 97% | 97% | 95% |
| Post-enrichment % naïve Treg | 18% | 19% | 30% | 11% | 19% |

TABLE 8
VC of naïve regulatory T cells and % recovery following steps of the manufacturing process

| | Run 5 | Run 6 | Run 7 | Run 8 | Run 9 |
|---|---|---|---|---|---|
| Naïve Treg Viable Count (VC) at Apheresis | 1.5E+08 | 1.4E+08 | 3.3E+08 | 1.3E+08 | 1.3E+08 |
| Naïve Treg VC at Depletion | 1.3E+08 | 6.6E+07 | 1.7E+08 | 7.6E+07 | 9.1E+07 |
| Naïve Tregs VC following Enrichment | 8.1E+07 | 3.8E+07 | 1.1E+08 | 3.2E+07 | 6.2E+07 |
| % recovery nTreg from apheresis following depletion | 85% | 49% | 519% | 58% | 72% |
| % nTreg Recovery from apheresis following enrichment | 53% | 28% | 34% | 24% | 49% |
| % recovery nTreg from depletion following enrichment | 60% | 63% | 63% | 51% | 66% |

The purity and recovery of naïve Tregs during the sorting process for each run is shown in Table 9.

TABLE 9
Purity and recovery of naïve Tregs post-FACS sorting

| | Total | | | |
|---|---|---|---|---|
| | nTregs | | | Post-Sort |
| | Sorted | | nTregs | nTreg Purity |
| | (Sorting | Total nTreg | Recovery | FoxP3+ |
| Run | input Treg) | Post-Sort | Sort Process | Tregs) |
| Run 5 | 3.6E+07 | 1.1E+07 | 31% | 95% |
| Run 6 | 1.7E+07 | 3.7E+06 | 22% | 99% |
| Run 7 | 9.4E+07 | 2.4E+07 | 26% | 96% |
| Run 8 | 3.6E+07 | 7.3E+06 | 20% | 97% |
| Run 9 | 9.5E+06 | 3.1E+06 | 33% | 96% |

The properties of the manufactured cells are shown below in Table 10.

TABLE 10
Properties of manufactured cells

| | TCR | Day | PDL (TDN) | % Viability | % of Target Dose |
|---|---|---|---|---|---|
| Run 5 | A | 9 | 6.6 | 96% | 1400% |
| Run 6 | A | 9 | 6.5 | Not available | 482% |
| Run 7 | Ev2 | 9 | 7.8 | Not available | 2100% |
| Run 8 | Ev2 | 10 | 7.8 | Not available | 1200% |
| Run 9 | Ev2 | 10 | 7.6 | 99% | 380% |

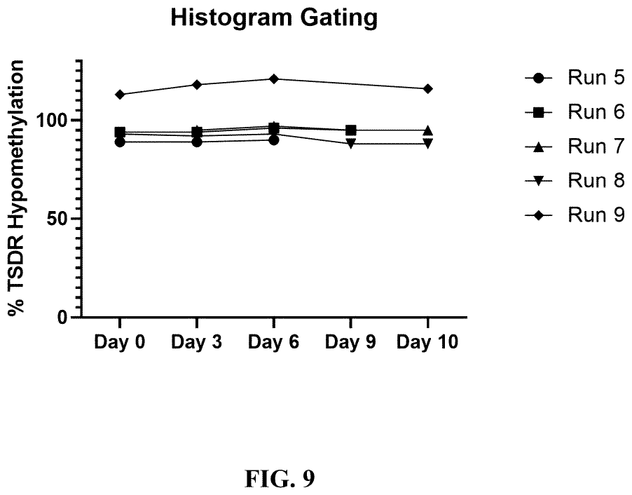

The TSDR status of the cells is shown in Table 11 below and in FIG. 9. The one run having TSDR levels above 100% is due to the donor being female. Because the TSDR assays measures the FoxP3 locus on the X chromosome, and females have one silenced (methylated) copy of the X chromosome, TSDR demethylation levels are doubled for female subjects to obtain the proportion of cells that have a hypomethylated FoxP3 locus.

TABLE 11
TSDR status of cells produced using the methods of this Example

| TSDR | | | | | | |
|---|---|---|---|---|---|---|
| Hypometh- | Donor | | | | | |
| ylation % | Sex | Day 0 | Day 3 | Day 6 | Day 9 | Day 10 |
| Run 5 | M | 89% | 89% | 90% | N/A | N/A |
| Run 6 | M | 94% | 94% | 96% | 95% | N/A |
| Run 7 | M | N/A | 95% | 97% | 95% | 95% |
| Run 8 | M | 93% | 92% | 93% | 88% | 88% |
| Run 9 | F | 113% | 118% | 121% | N/A | 116% |

### Activity of Tregs Following a Cryopreservation Freeze-Thaw Cycle

Regulatory T cells produced according to the methods in this example for runs 5-7 that were untransduced or transduced with the lentiviral vectors shown were cryopreserved for storage. The cells were frozen and stored in a vapor-phase nitrogen storage freezer.

After a period of time in cold storage, the frozen cell population was thawed. The cell population was split into two experimental groups. The first experimental group was activated with anti-CD3 and anti-CD28 antibodies while the second experimental group was not. Both groups were expanded in media containing IL-2. Following incubation, regulatory T cell activation markers and cytokine levels were measured after 1, 2, and 3 days (IL-10, CTLA-4, TGF-beta-1, and CD69).

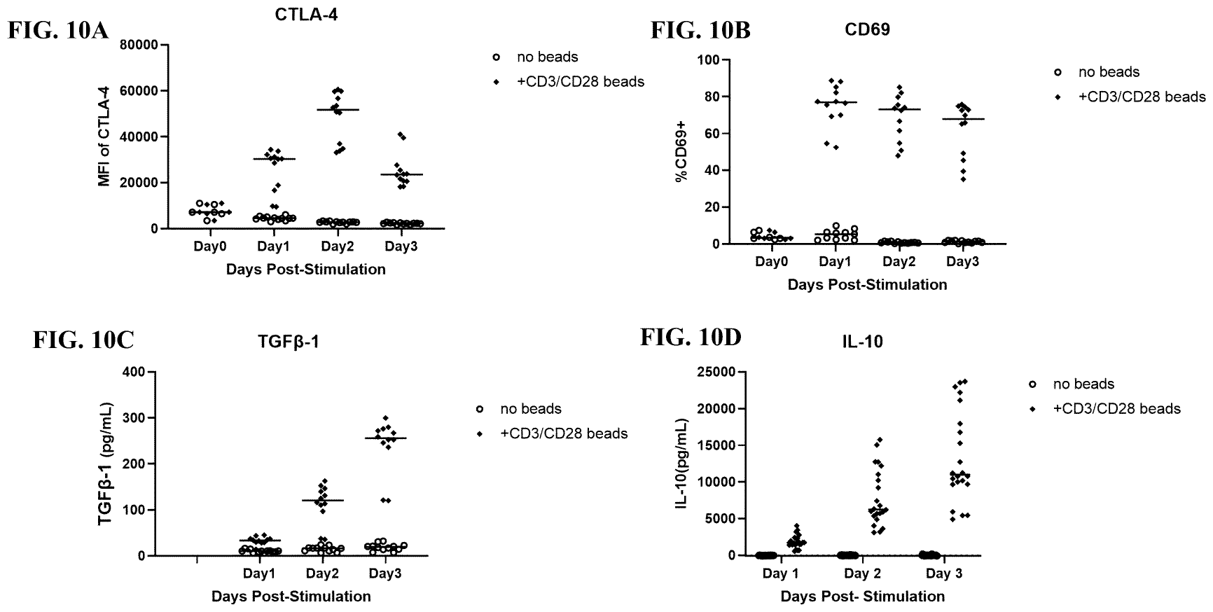

The increase in levels for each of IL-10, CTLA-4, TGF-beta-1, and CD69 for all of the runs over the three day period for cells activated with anti-CD3 and anti-CD28 beads is shown in FIG. 10A-FIG. 10B. The activated cells showed increasing levels of IL-10 (FIG. 10D), CTLA-4 (FIG. 10A), TGF-beta-1 (FIG. 10C), and CD69 (FIG. 10B) over the period, demonstrating that cell populations can be activated and retain their cellular functionality following a cryopreservation freeze-thaw cycle (FIG. 10A-FIG. 10D). The cells that were not activated showed no increase in levels. This data further demonstrates that the regulatory T cells retained function.

### Example 3: Manufacturing Strategy with Increased Stringency of Stable Treg Selection

Challenges Manufacturing Stable Tregs from Patient Populations

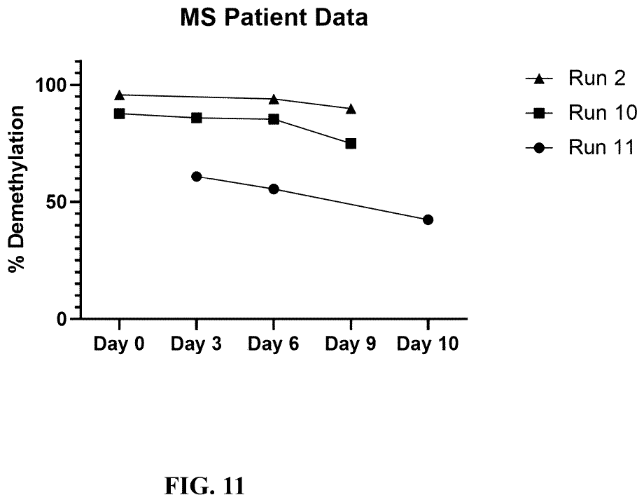

Following establishment of a manufacturing process with healthy donor samples, the manufacturing process was evaluated with samples from MS donors. For certain MS donors, a lower initial TSDR hypomethylation level and/or less stable TSDR hypomethylation levels over expansion (Day 0 to harvest) were observed compared to the results using healthy donor cells. FIG. 11 shows the change in TSDR hypomethylation over expansion for the three MS donors. For two of the three MS donors, initial TSDR was below 90% and TSDR decreased by more than 10% over the course of expansion. This is likely because of differences in Treg populations between healthy and autoimmune donors, including MS donors, making the boundary between Treg and T conventional cells less clear when sorting the cells. These results suggested that, for some donors, increasing the stringency of the gating strategy would likely improve the purity of the isolated Treg population, and consequently, the proportion of stable Tregs obtained in the process. However, an increase in stringency would likely result in a decrease in yield. Described in this example is a stricter gating approach coupled with a modified expansion protocol. The modified expansion protocol enhances the proliferation of Tregs that is needed to overcome the reduced yield due to the increased stringency of stable Treg selection.

### Evaluation of Treg Subpopulations for Inclusion in Process

The sorting strategies described in the above examples focus on the selection of naïve Tregs. Given the need for a more stringent sorting strategy for certain donors, and that this strategy would likely exclude an increased proportion of naïve Tregs, we evaluated whether the CD45RA+ selection step to select naïve Tregs was necessary in the context of a stricter sorting strategy, or whether more stringently selected antigen-experienced Tregs could be used. Naïve Tregs are CD4⁺CD25⁺CD127^{-/low}CD45RA⁺. Antigen-experienced Tregs are CD4+CD25highCD127−/loCD45−. Both of these populations are stable Tregs that will have a stable TSDR hypomethylation phenotype. To assess whether selection based on CD45RA is needed, antigen experienced (CD45RA−) and naïve (CD45RA⁺) Tregs were isolated and compared for expansion capacity and stability.

Cells were prepared as described in Example 1 prior to the FACS sorting step. Cells then underwent two rounds of FACS sorting for debulk and purity. Both the CD45RA and CD45RA⁺ population were sorted on CD4, CD25, and CD127 at the debulk sort. Cells were first identified as CD4⁺ and then a polygon gate was used to identify CD25^{+/high}CD127^{-/low} Treg set at 70% of a standard polygon gate for Treg identification and selection. The sort was the repeated and for CD45RA cells, after the identification of the CD25^{+/high}CD127^{-/low} Treg population, CD45RA cells were identified and selected. For CD45RA⁺ cells, after the identification of the CD25^{+/high}CD127^{-/low} Treg population, CD45RA⁺ cells were identified and selected. The percent naïve (CD45RA⁺) Tregs in each population is shown below in Table 12. At the conclusion of the sort, 3 groups were set up:
(a) CD45RA⁺ Tregs—Enriched CD45RA⁺ Treg Fraction
(b) CD45RA⁻ Tregs—Enriched CD45RA⁻ Treg Fraction
(c) “Spike-In” Tregs—80% Enriched CD45RA⁻ Treg Fraction+20% Enriched CD45RA⁺ Treg Fraction

TABLE 12
% naïve Treg in each condition prior to expansion

| Condition | Day 0 nTreg % |
|---|---|
| CD45RA+ | 94% |
| Spike-In | 20% |
| CD45RA− | 2% |

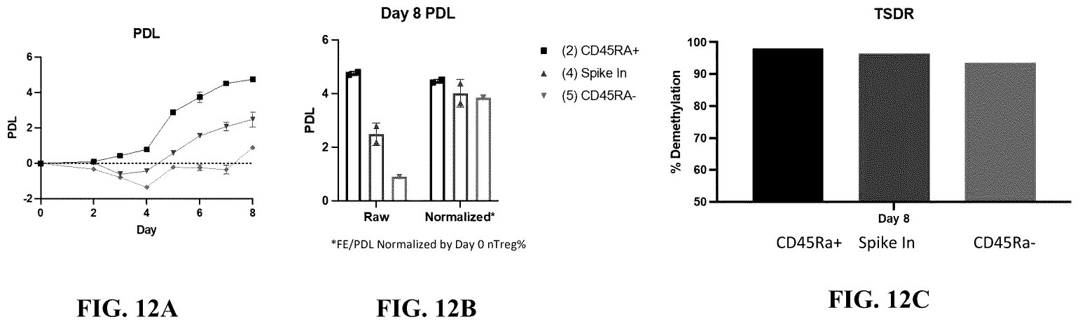

Cells were then expanded for 8 days in culture as described in Example 1. FIG. 12A-FIG. 12C show the population doubling level (PDL) and the TSDR hypomethylation following the expansion of the cells. As shown in FIG. 12A and FIG. 12C, the CD45RA⁺ population showed a dramatic increase in expansion relative to the CD45RA population along with increased TSDR hypomethylation. This suggests that CD45RA+naïve Tregs are necessary for the expansion process because of the relative expansion capacity of CD45RA+naïve Tregs compared to CD45RA Tregs. Importantly, the “spike-in” sample shows that CD45RA+naïve Tregs are still able to expand effectively in the presence of a large excess of CD45RA antigen-experienced Tregs.

### Development of a Stricter Gating Strategy for Isolation of Stable Tregs

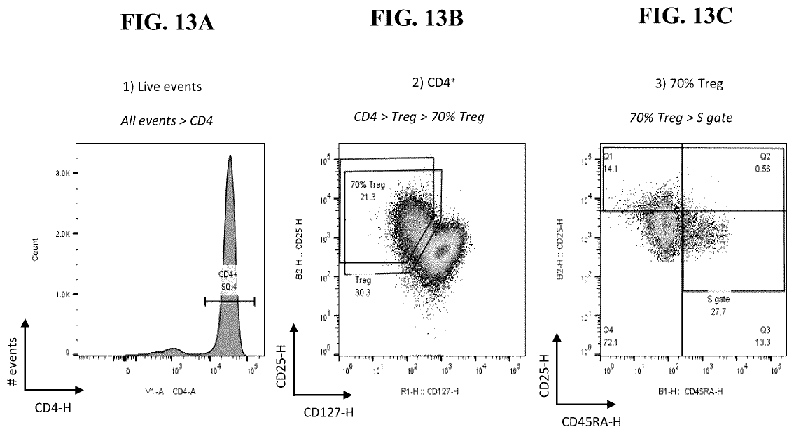

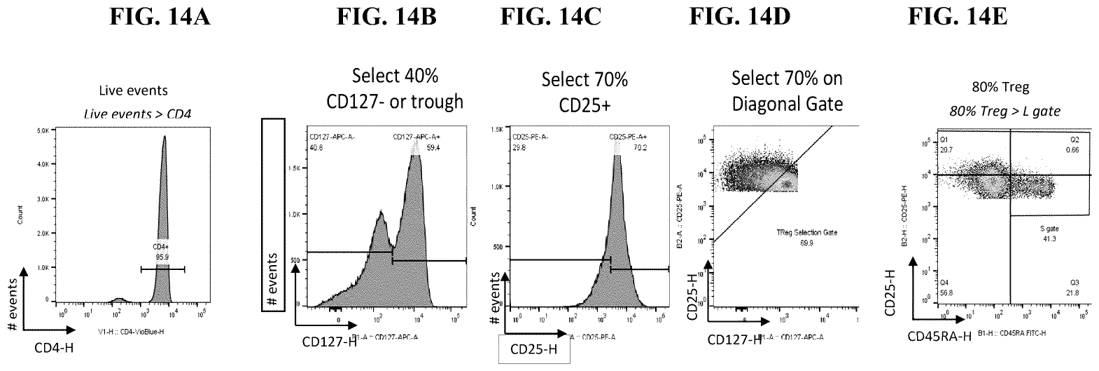

In light of the findings above, an improved gating strategy was developed for increasing the stringency of the sort while still capturing CD45RA⁺Tregs. In particular, the strategy identifies both antigen-experienced (CD4⁺CD25^{high}CD127^{-/lo}CD45RA⁻) and naïve (CD4⁺CD25⁺CD127^{-/lo}CD45RA⁺) Tregs, while specifically excluding CD4⁺CD25⁺CD127^{-lo}CD45RA⁻ non-Tregs. By separately identifying both antigen-experienced and naïve Tregs, sufficient numbers of Tregs are obtained to produce a cell therapy product from a single leukopak, and the antigen experienced Tregs have high suppressive capacity, which suppresses any non-Treg both ex vivo and in vivo. In this approach, CD4⁺ cells are identified, followed by identification of CD127^{-/lo} and CD25⁺ (either sequentially or concurrently) combined with a critical “not” or L-shaped gate as the last step of the gating scheme, to select antigen-experienced (CD4⁺CD25^{high}CD127^{-/lo} CD45RA⁻) and naïve (CD4⁺CD25⁺CD127^{-/lo}CD45RA⁺) Tregs, while specifically excluding the undesired CD4⁺CD25⁺CD127^{-/lo}CD45RA⁻ cells based on CD25 and CD45RA levels. This gating approach is shown in FIGS. 13A-FIG. 13C and FIG. 14A-FIG. 14E, with FIG. 13A-FIG. 13C showing a dot plot approach for the CD25/CD127 selection step and FIG. 14A-FIG. 14E showing a histogram approach for selecting CD127^{-/lo} followed by CD25⁺.

To describe the gating strategy in further detail, CD25 enriched cells were stained with fluorescently labeled antibodies that recognize human CD4, CD127, CD45RA cell surface markers. Cells were already labeled with CD25 antibodies in the enrichment step of the process. The cells were then sorted twice using the gating scheme described below (using the same strategy in each step) using a FACS instrument. For the dot plot-based approach, shown in FIG. 13A-FIG. 13C, in the first step of the gating process, CD4⁺ T cells were first identified from all the live cellular events (FIG. 13A). In the second step, CD25⁺CD127^{-/low} Treg cells were identified based on two-parameter flow plots, whereby spatial context of the Treg cell population could be visualized relative to another cell population (i.e., Tconv cells), CD25 and CD127 levels were visualized and a polygon gate was drawn in the upper left quadrant to capture CD25⁺CD127^{-/low} cells. That polygon gate was then shifted upwards to reduce the selected cell population to 70% of the original population, thereby increasing the stringency of selection (FIG. 13B). In the final step, the “not” or L-shaped gate was applied visualizing CD45RA and CD25 levels to select the naïve Treg (CD4⁺CD25⁺CD127^{=-/lo}CD45RA⁺) and antigen experienced Treg (CD4⁺CD25^{high}CD127^{-/low}CD45RA⁻) cell population for expansion by excluding the CD25+CD45RA cell population from the sorting gate (FIG. 13C). While both the antigen experienced and non-stable Treg populations are CD45RA⁻, they are distinguishable by CD25 levels, with the antigen-experienced Tregs being CD25high and non-stable Treg population being only CD25⁺ but not high. Because naïve Tregs are CD45RA⁺ and CD25⁺ (but not high), the threshold for determining whether CD45RA⁻ cells are CD25⁺ or CD25high is determined based on CD25 levels in the naïve Treg population, with CD25high being defined as CD25 levels being above that expressed in the naïve Treg population. The L-shaped gate is critical because it allows for the selection of two important populations of cells. CD25^{high} CD45RA⁻ antigen-experienced Tregs and CD25⁺CD45RA⁺ naïve Tregs are selected, while CD25⁺CD45RA⁻ contaminating cells are excluded.

The histogram-based approach was implemented as an operational alternative for certain manufacturing situations and yielded similar results to the dot plot strategy (FIG. 14A-FIG. 14E). Using sequential histogram flow plots, CD4⁺ T cells that had the lowest expression of CD127 (i.e., 40±2% CD127^{-/lo}) followed by the highest expression of CD25 (i.e., 70±2% CD25⁺) were selected (FIG. 14A-FIG. 14C). The cell population was refined further by selecting 70±2% of Treg cells in a CD25 versus CD127 two-parameter flow plot (FIG. 14D). Naïve Treg (CD4⁺CD25⁺CD127^{-/lo}CD45RA⁺) and antigen experienced Treg (CD4⁺CD25^{high}CD127^{-/lo}CD45RA⁻) were then identified using the same “not” or L-shaped gate as in the dot-plot based approach (FIG. 14E).

The MS donor used for Run 10 donated a second leukopak (Run 12), and that leukopak was processed using the new sorting strategy (histogram+L-shaped gate). Aside from the FACS sorting steps, all other elements of the manufacturing process were as described in Example 1. Table 13 shows the properties of the cells following enrichment and prior to sorting, demonstrating that the properties of the cells are highly similar between the two runs prior to the sorting step. In the context of Table 13 and all tables herein, N/A means that the data is not available, because it was not collected.

TABLE 13
Properties of MS donor cells prior to FACS sorting

| | | Run 12 (histogram + |
|---|---|---|
| Pre-sort | Run 10 | L-shaped gate) |
| Apheresis TVC | 6.8E+09 | 6.5E+09 |
| Apheresis % CD4 T cells | 48% | 52% |
| Apheresis % naïve Treg | 1% | 1% |
| Apheresis % Treg | N/A | 3% |
| Post-depletion TVC | 2.9E+09 | 3.0E+09 |
| Post-depletion % CD4 T cells | 77% | 79% |
| Post-depletion % naïve Treg | 1.9% | 2% |
| Post-depletion % Treg | N/A | 3% |
| Post-enrichment TVC | 1.0E+08 | 1.8E+08 |
| Post-enrichment % CD4 T cells | 96% | 95% |
| Post-enrichment % naïve Treg | 7.8% | 8% |
| Post-enrichment % Treg | N/A | 26% |

Table 14 shows properties of the cells following isolation and prior to expansion. As expected, while the purity of Tregs following sorting remains high with the strict sorting approach, the proportion of naïve Tregs is reduced relative to the original process described in Example 1 due to the inclusion of antigen-experienced Tregs in the sort strategy. Due to the increased stringency of the gating protocol, the recovery of naïve Tregs was also reduced.

TABLE 14
Properties of MS donor cells following FACS sorting

| | Treg | | | nTreg | | |
|---|---|---|---|---|---|---|
| | | Pu- | % Re- | | Pu- | % Re- |
| | Treg # | rity | covery | nTreg # | rity | covery |
| Run 10 | N/A | N/A | N/A | 2.6E+06 | 97% | 35% |
| Run 12 | 5.8E+05 | 89% | 5% | 3.9E+05 | 63% | 7.9% |
| (histogram + | | | | | | |
| L-shaped gate) | | | | | | |

The population doubling level and % viability are shown in Table 15. The % target dose achieved for the full process is also shown and was calculated as in Example 1, except that for the new sorting strategy, the target dose calculation was based on the total number of Tregs rather than the total number of naïve Tregs, as both populations are selected.

TABLE 15
Properties of manufactured cells

| | Run 10 (Example 1 | Run 12 (histogram + |
|---|---|---|
| | process) | L-shaped gate) |
| TCR | A | Ev2 |
| Day | 9 | 10 |
| PDL | 6.4 | 6.3 |
| % Viability | 86% | 90% |
| Final % FoxP3⁺ | 76% | 84% |
| TCR⁺ | 39% | N/A (assumed 25%) |
| % Target dose | 59% | 112% |

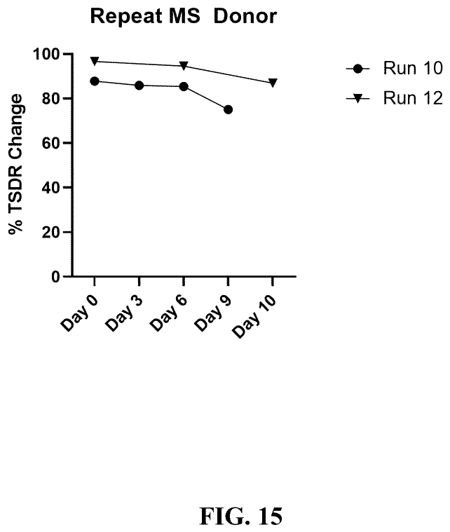

FIG. 15 and Table 16 below show the % TSDR hypomethylation following sorting and over the course of expansion. For the L-shaped gate approach, an initial TSDR hypomethylation above 95% was achieved (relative to 87.79% with the sorting approach from Example 1). Moreover, for the L-gate based approach, TSDR hypomethylation levels decreased by less than 10% over the course of expansion.

TABLE 16

| | Run 10 (Example 1 | Run 12 (histogram + |
|---|---|---|
| | process) | L-shaped gate) |
| Day 0 | 88% | 97% |
| Day 3 | 86% | N/A |
| Day 6 | 85% | 95% |
| Day 9 | 75% | N/A |
| Day 10 | N/A | 87% |

### New Expansion Process

Because the newly developed gating strategy selects a smaller number of naïve Tregs than the original process, the expansion process was modified to increase expansion of the Tregs.

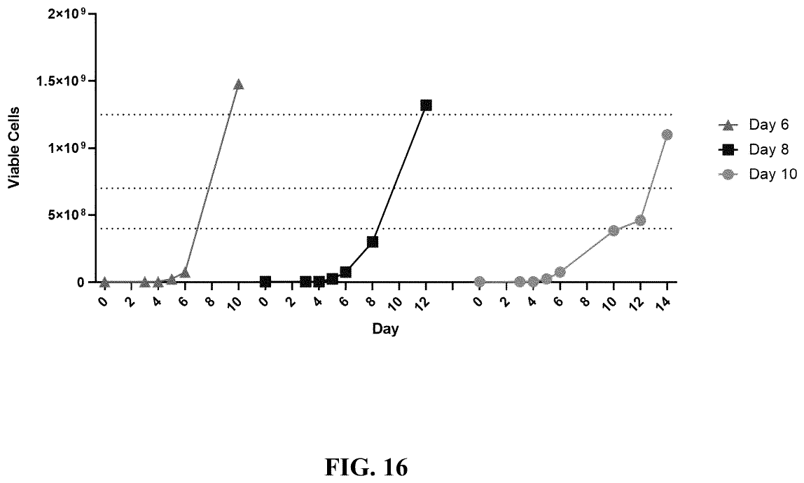

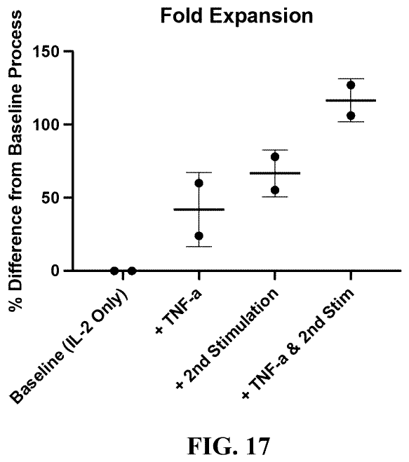

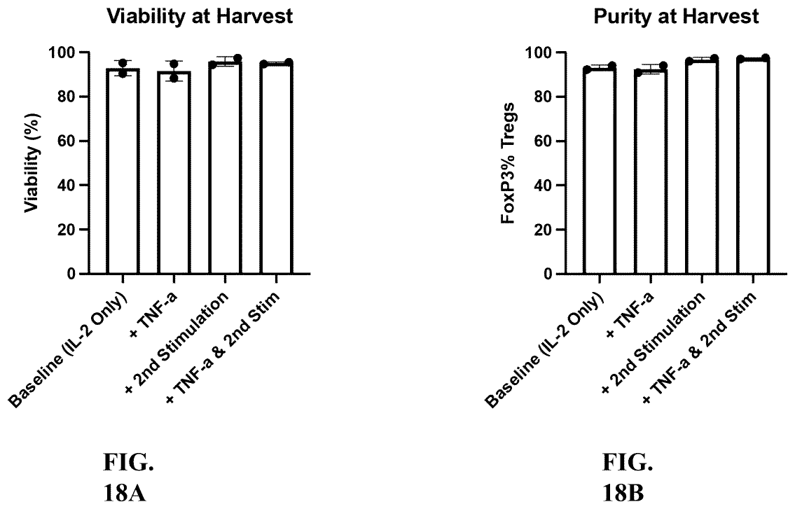

The expansion protocol was altered to add TNF-alpha to the expansion media and to add a second anti-CD28 anti-CD3 stimulation. First, the effect of restimulation was evaluated at various timepoints following transduction. For this experiment, cells were prepared according to the methods described in Example 1, except that cells were gated on CD4, CD25, and CD127 only and not CD45RA. Cells were then activated and transduced as in Example 1, with a second activation step with anti-CD3 and anti-CD28 at days 6, 8, and 10 following the first activation step. It was found that this restimulation at Day 6 gave similar expansion to restimulation at days 7 or 8 but advantageously, restimulation at day 6 of the expansion process gave the greatest increase in expansion when cells were harvested at day 10, which, critically, does not require lengthening of the expansion process (FIG. 16) Next, the effect of adding TNF-alpha to the expansion protocol was assessed. Cells prepared according to the methods described in this example (including the L-shaped gate) were expanded as described in Example 1 with or without the second stimulation at day six and with or without 2500 IU/ml TNF-alpha to assess the effect of TNF-alpha on expansion of Tregs, either alone or in combination with a second stimulation. As is shown in FIG. 17, the addition of TNF-alpha throughout the expansion process in combination with a second stimulation with anti-CD3 and anti-CD28 at day 6 of the expansion process gave the largest increase in expansion. Although, each individual change to the process also increased expansion. As is shown in FIG. 18, cells expanded in TNF-alpha in combination with a second stimulation showed the highest viability and purity at harvest relative to either process change alone or the original expansion protocol.

Tregs Generated with New Gating and Expansion Process

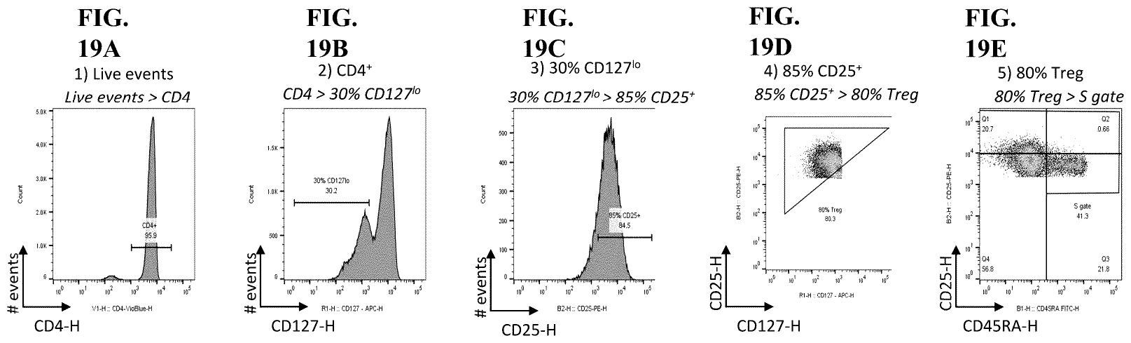

The manufacturing process described in this Example was then applied to a second repeat MS donor. The MS donor from Run 11 donated an additional leukopak. This leukopak was processed using both the dot plot and histogram based-strict sorting strategies and with the improvements to the expansion conditions described above. Minor alterations were made to the histogram-based L-shaped gate approach at the CD127/CD25 selection step (FIG. 19C). CD4⁺ T cells that had the lowest expression of CD127 (i.e., 30±2% CD127^{-/lo}) followed by the highest expression of CD25 (i.e., 85±2% CD25⁺) were selected. The cell population was further refined by selecting 85±2% of Treg cells in a CD25 versus CD127 two-parameter flow plot (FIG. 19D). The L-shaped gate itself was not altered (FIG. 19E). Prior to the sorting step, cells were processed as in Example 1 (FIG. 19A-FIG. 19E). Table 17 shows the properties of the cells following enrichment and prior to sorting, demonstrating that the properties of the cells from each run are highly similar prior to the sorting step.

TABLE 17
Properties of MS donor cells prior to FACS sorting

| | Run 11 (Example | |
|---|---|---|
| Pre-sort | 2 Process) | Run 13 |
| Apheresis TVC | 4.7E+09 | 3.6E+09 |
| Apheresis % CD4 T cells | 49% | 46% |
| Apheresis % naïve Treg | 0.9% | 1% |
| Apheresis % Treg | N/A | 5.3% |
| Post-depletion TVC | 2.2E+09 | 1.2E+09 |
| Post-depletion % CD4 T cells | 78% | 59% |
| Post-depletion % naïve Treg | 1% | 1% |
| Post-depletion % Treg | N/A | 7% |
| Post-enrichment TVC | 2.8E+08 | 1.7E+08 |
| Post-enrichment % CD4 T cells | 95% | 94% |
| Post-enrichment % naïve Treg | 8% | 7% |
| Post-enrichment % Treg | N/A | 27% |

The properties of the cells following sorting are shown in Table 18 below:

TABLE 18
Properties of MS donor cells following FACS sorting

| | Treg # | Purity | nTreg # | Purity |
|---|---|---|---|---|
| Run 11 (Example 2 process) | N/A | N/A | 6.5E+06 | 95% |
| Run 13 L-shaped Gate (Dot | 5.6E6 | 100 | 1.9E+06 | 34 |
| Plot) | | | | |
| Run 13 L-shaped Gate | 4.6E6 | 100 | 1.2E+06 | 25 |
| (Histogram) | | | | |

Table 19 below shows the properties of the manufactured cells. The % target dose was calculated as described above.

TABLE 19
Properties of manufactured cells

| | Run 11 | Run 13 L- | Run 13 L- |
|---|---|---|---|
| | (Example 2 | Shaped Gate | Shaped Gate |
| | process) | (Dot Plot) | (Histogram) |
| TCR | Ev2 | Ev2 | Ev2 |
| Day | 11 | 10 | 10 |
| PDL | 8.0 | 7.2 | 6.9 |
| % Viability | 95% | N/A | N/A |
| Final % FoxP3⁺ | 62% | 90% | 89%.3 |
| TCR⁺ | 19% | 54% | 50% |
| % Target dose | 410% | 170% | 170% |

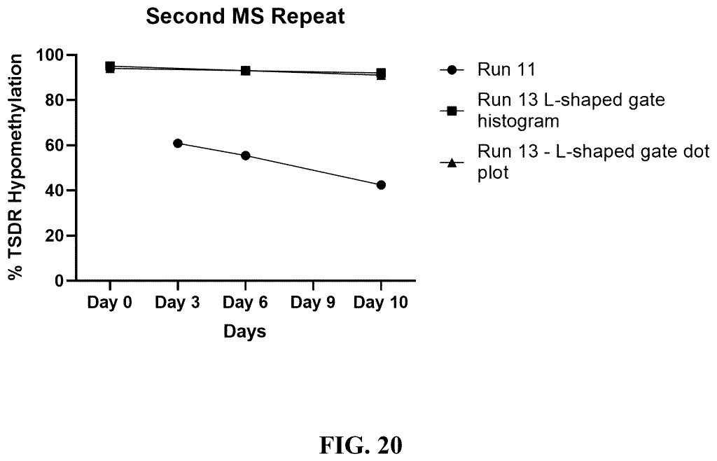

FIG. 20 and Table 20 below shows TSDR hypomethylation over expansion for this donor using the process described in Example 2 and both the dot-plot and histogram-based L-shaped gate strategies described in this example.

TABLE 20
TSDR of MS donor using original process and L-gate

| | Run 11 | Run 13 L- | Run 13 L- |
|---|---|---|---|
| | (Example 2 | shaped Gate | shaped Gate |
| | process) | (Dot Plot) | (Histogram) |
| TSDR Initial | 61% (Day 3) | 94% (Day 0) | 95% (Day 0) |
| TSDR Final | 42% (Day 10) | 91% (Day 10) | 92% (Day 10) |
| TSDR change | −19% | −3% | −3% |

The comparative data presented in this example shows that the strict gating strategy combined with the updated expansion process results in a highly stable Treg population and generates sufficient transduced Tregs to achieve the target dose.

The process was repeated with an additional MS donor and with two additional healthy donors with both L-shaped gating approaches, and similar results were achieved. Table 21 shows the properties of the cells following enrichment and prior to sorting.

TABLE 21
Composition of cell population during
depletion and enrichment process

| Cells | Run 14 | Run 15 | Run 16 |
|---|---|---|---|
| Donor Sex | 25 | 33 | 50 |
| Donor Age | M | F | F |
| Donor Health Status (MS/non-MS) | Healthy | Healthy | MS |
| | Donor | Donor | |
| Apheresis VC | 7.4E+09 | 1.4E+10 | 3.7E+09 |
| Apheresis % CD4 T cells | 50% | 39% | 34% |
| Apheresis % naïve Treg | N/A | 2% | 1% |
| Apheresis % Treg | N/A | 3% | 4% |
| Post-depletion VC | 2.6E+09 | 4.8E+09 | 1.4E+09 |
| Post-depletion % CD4 T cells | 89% | 60% | 71% |
| Post-depletion % naïve Treg | 3% | 3% | 3% |
| Post-depletion % Treg | 5% | 6% | 8% |
| Post-enrichment VC | 2.3 + 08 | 4.4E+08 | 1.7E+08 |
| Post-enrichment % CD4 T cells | 82% | 88% | 97% |
| Post-enrichment % naïve Treg | 26% | 23% | 7% |
| Post-enrichment % Treg | 41% | 45% | 29% |

The properties of the cells following sorting are shown in Table 22 below:

TABLE 22
VC of regulatory T cells and % recovery following sorting

| | Treg | | | nTreg | | |
|---|---|---|---|---|---|---|
| | | | % Re- | | | % Re- |
| | Treg # | Purity | covery | nTreg # | Purity | covery |
| Run 14 | 14E+06 | 96% | N/A | 12E+06 | 87% | N/A |
| histogram | | | | | | |
| L-shaped | | | | | | |
| Gate | | | | | | |
| Run 15 | 10E6 | 100% | N/A | 4.7E+06 | 47% | N/A |
| histogram | | | | | | |
| L-shaped | | | | | | |
| Gate | | | | | | |
| Run 15 | 12E6 | 100% | N/A | 1.1E+07 | 88% | N/A |
| dot plot | | | | | | |
| L-shaped | | | | | | |
| Gate | | | | | | |
| Run 16 | 15E6 | 99% | N/A | 1.5E+07 | 44% | N/A |
| histogram | | | | | | |
| L-shaped | | | | | | |
| Gate | | | | | | |

The properties of the manufactured cells are shown below in Table 23. Target dose was calculated as described earlier in this example.

TABLE 23
Properties of manufactured cells

| | Run 14 | Run 15 | Run 15 dot | Run 16 |
|---|---|---|---|---|
| | histogram L- | histogram L- | plot L-shaped | histogram L- |
| | shaped Gate | shaped Gate | Gate | shaped Gate |
| TCR | Ev2 | Ev2 | Ev2 | Ev2 |
| Day | 10 | 10 | 10 | 10 |
| PDL | 6.8 | 7.2 | 9.6 | 7.8 |
| % Viability | 81% | N/A | N/A | N/A |
| Final % | 92% | 93% | 93% | 95% |
| FoxP3⁺ | | | | |
| TCR⁺ | 57% | 34% | 43% | 38% |
| % Target | 390% | 370% | 2300% | 840% |
| dose | | | | |

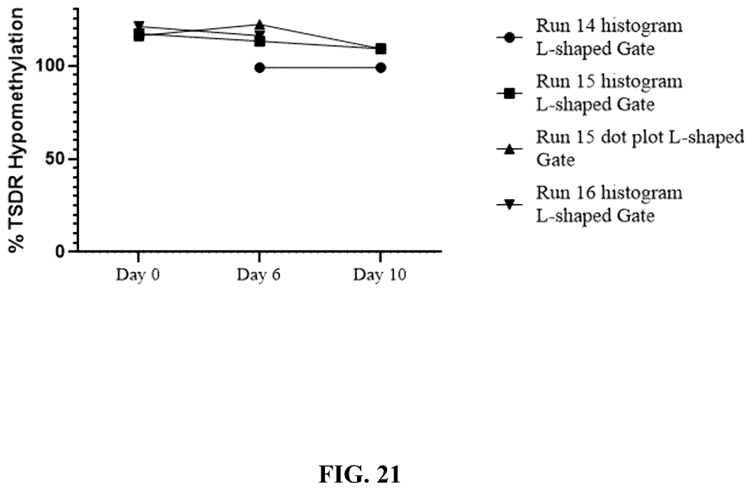

The TSDR status of the cells is shown in Table 24 below and in FIG. 21.

TABLE 24
TSDR status of cells produced using the methods of this Example

| | Donor | | | |
|---|---|---|---|---|
| TSDR Hypomethylation % | Sex | Day 0 | Day 6 | Day 10 |
| Run 14 histogram L-shaped Gate | M | N/A | 99% | 99% |
| Run 15 histogram L-shaped Gate | F | 117% | 113% | 109% |
| Run 15 dot plot L-shaped Gate | F | 116% | 122% | 109% |
| Run 16 histogram L-shaped Gate | F | 121% | 116% | N/A |

For all donors processed using the sorting and expansion strategy described in this example, a TSDR hypomethylation status above 90% post-sorting that did not decrease by more than 10% over the expansion process was achieved. This demonstrates that this process generates a highly pure and stable Treg population from a variety of donors. In addition, the process was also able to achieve sufficient numbers of transduced Tregs to achieve the target dose.

### Example 4: Assessment of TSDR Threshold Levels

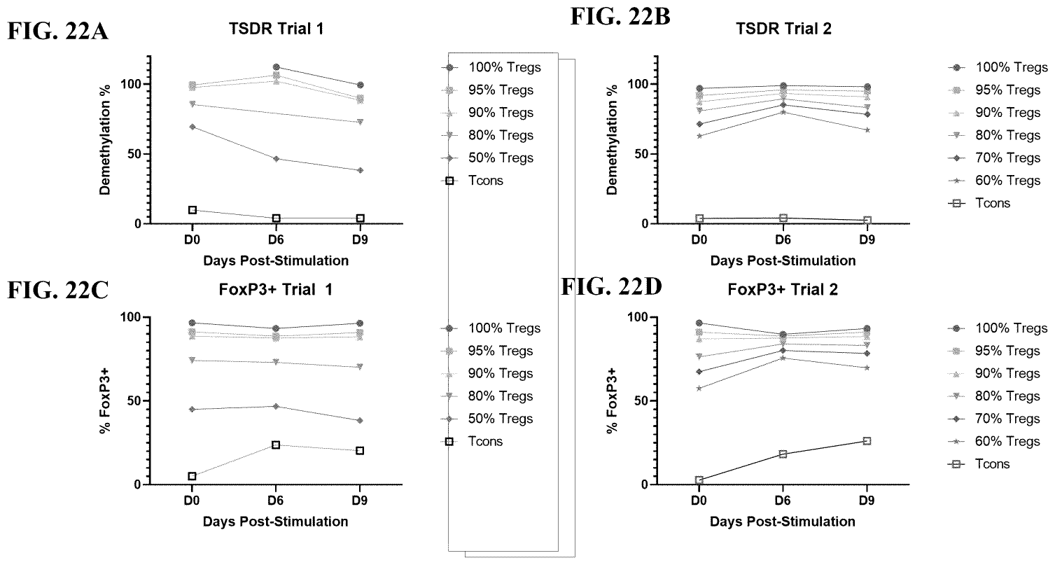

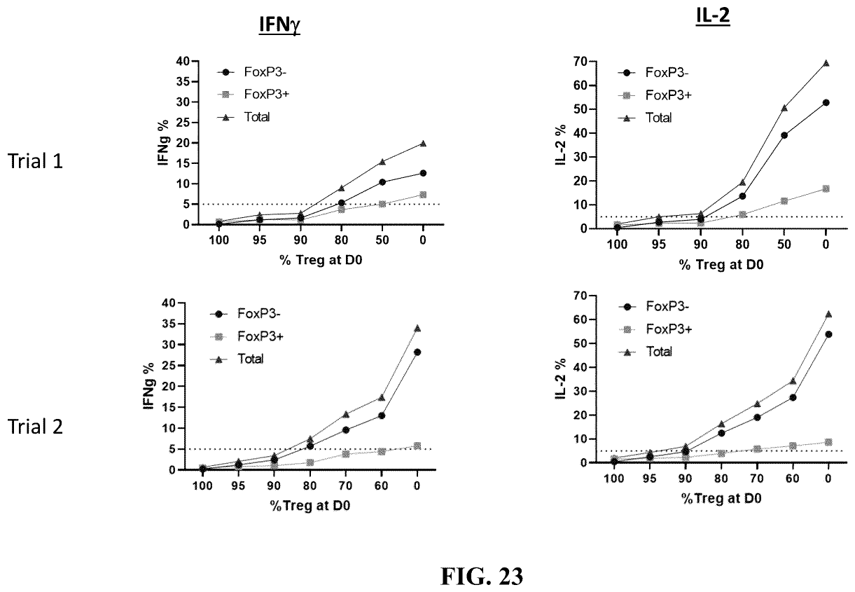

The threshold level of TSDR hypomethylation, representing the level of stable Tregs, necessary for a stable Treg cell product was investigated. We first asked what proportion of stable Treg at Day 0 to maintain a stable level of Treg cells throughout the expansion process, indicating that there was low to no outgrowth of contaminating conventional T cells or other cellular impurities. To do this we spiked conventional T cells into Tregs, each isolated from the same healthy donor, at various percentages on Day 0 and tracked the TSDR levels throughout the expansion process. TSDR hypomethylation levels and FOXP3+ levels correlated with initial Treg purity and remained stable with no more than a 10% drop in TSDR hypomethylation throughout expansion when Treg purity was greater than 60% (FIG. 22A-FIG. 22D). Next, the Treg purity of the expanded cell product was evaluated for the presence of conventional T cells and production of pro-inflammatory cytokines when activated with PMA and ionomycin. Cryopreserved cells from the harvest were thawed into fresh media and then stimulated with PMA and ionomycin for 4 hours at 37° C. and 5% CO2. Intracellular cytokine (ICC) for IFN-g and IL-2 production was measured by flow cytometry. FIG. 23 shows the proportion of FoxP3+ and FoxP3-cells expressing IL-2 and IFN-g post-activation with PMA and ionomycin. In the absence of conventional T cells, the 100% Treg product shows little to no IFN-g or IL-2 cytokines secreted, indicating that there are insignificant numbers of unstable Treg cells that expanded from the initial culture. As the initial Treg purity decreased, there was an increase in the proportion of T conventional cells secreting I IFN-g and IL-2 cytokines, with the largest increases observed when the starting Treg purity was less than 90%. This increase is likely due to the increased Tcon:Treg ratio. Upon activation, some conventional T cells upregulate FoxP3 which is why IFN-gamma and IL-2 are produced from some FoxP3+ cells. This suggests that a high TSDR level is critical to maintain a stable population of cells that are able to suppress contaminating conventional T cells.

### Example 5: Characterization of MBP83-99-Reactive TCR Constructs

Three TCRs having a high affinity for MBP83-99 peptide (amino acid sequence ENPVVHFFKNIVTPRTP) were selected for evaluation, and the sequences were assembled based on the literature and the IMGT database. TCRs targeting MBP peptide were chosen as a therapeutic for multiple sclerosis because MBP localizes to the meninges, where inflammatory processes associated with MS pathogenesis are known to occur.

A TCR having the alpha and beta chains of TCR A, described in further detail below, was cloned into lentiviral vectors in the alpha-P2A-beta and beta-P2A alpha orientation to determine the optimal configuration of the TCR components in the vector. Each of the alpha and beta chains was tagged to allow for visualization of the expression levels of each component of the TCR. The constructs were engineered with a P2A tag between the two TCR subunits, and a Myc-tag at the N-terminus of the downstream protein (TCR alpha or TCR beta), to allow detection of the downstream component. It was assumed that if the second subunit was expressed, the first encoded subunit was also expressed. The second encoded subunit was followed by an IRES and a GFP tag to confirm expression of the full ORF (EF1alpha TCR beta_P2A_cMyc_TCR alpha_IRES_EGFP or EF1apha TCR alpha_P2A_cMyc_TCR beta_IRES_EGFP).

Constructs were transduced into TCR-null Jurkat cells at an MOI of 10, 20, or 30. Cells were then incubated overnight, and fresh media was added prior to staining for flow cytometry. Cells were stained with DAPI, Anti-cMYC AX647, and CD3 BV786. Cell surface staining of CD3 indicates that the TCR assembled and expressed at the cell surface. Detection of Myc indicates the expression of the second-expressed TCR component. GFP+ cells indicate that the full construct was expressed.

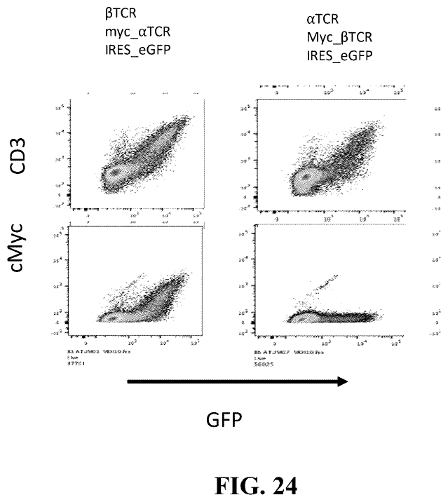

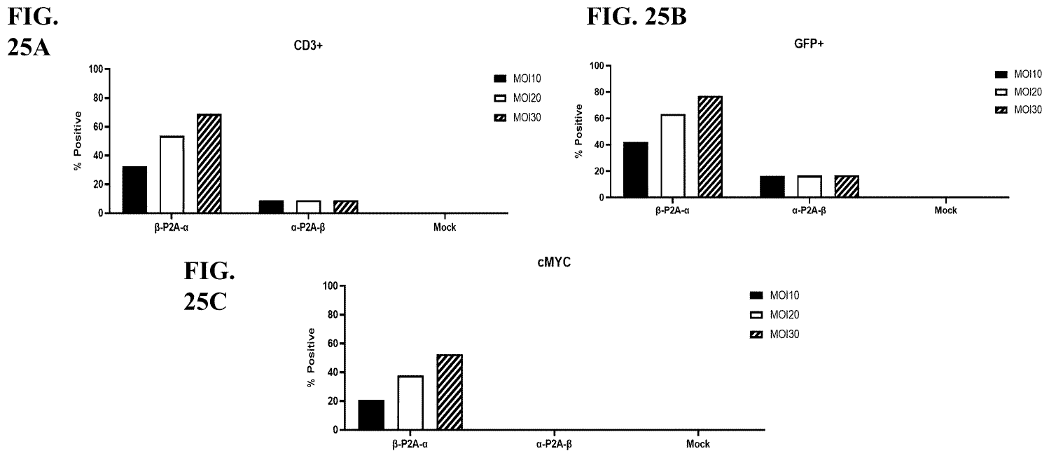

As is shown in FIG. 24, for the TCR beta_P2A_cMyc_TCR alpha_IRES_EGFP construct, flow cytometry analysis showed cell surface localization of CD3 in GFP positive cells, indicating that a TCR complex is assembled at the cell surface. cMyc-TCR beta is also detected on the cell surface, indicating expression and cell surface localization of TCR alpha and TCR beta. However, as seen in FIG. 25A-FIG. 25C, less CD3 (FIG. 25A) and cMyc (FIG. 25C) were detected at the cell surface of the TCR alpha_P2A_cMyc_TCR beta_IRES_EGFP construct relative to the construct with the opposite orientation of TCR alpha and TCR beta, which suggests poor TCR alpha expression or cell surface localization. The proportion of GFP positive cells was also decreased (FIG. 25B), suggesting that TCR expression is reduced with the TCR alpha_P2A_cMyc_TCR beta_IRES_EGFP construct relative to the TCR beta_P2A_cMyc_TCR alpha_IRES_EGFP construct. Based on this data, constructs were generated with TCR beta upstream of TCR alpha going forward.

Each TCR (TCR-A, TCR-B, and TCR-C) was then cloned into a vector comprising an N-terminal TCR beta chain and a C-terminal TCR alpha chain with a linker domain comprising a GSG amino acid sequence followed by a P2A self-cleaving peptide. The TCR beta-GSG-P2A-TCR alpha fusion constructs were expressed under the EF1alpha promoter. The constructs used in Examples 5-10 also included a GFP tag at the C-terminus of TCR alpha to allow for in vitro detection.

TCR-A comprised a TCR alpha variable domain having the amino acid sequence of SEQ ID NO: 4 (which includes a CDR1-alpha sequence comprising the amino acid sequence of SEQ ID NO: 1, a CDR2-alpha sequence comprising the amino acid sequence of SEQ ID NO: 2, and a CDR3-alpha sequence comprising the amino acid sequence of SEQ ID NO: 3) and a TCR beta variable domain having the amino acid sequence of SEQ ID NO: 10 (which includes a CDR1-beta sequence comprising the amino acid sequence of SEQ ID NO: 7, a CDR2-beta sequence comprising the amino acid sequence of SEQ ID NO: 8, and a CDR3-beta sequence comprising the amino acid sequence of SEQ ID NO: 9).

TCR-B comprised a TCR alpha variable domain having the amino acid sequence of SEQ ID NO: 21 (which includes a CDR1-alpha sequence comprising the amino acid sequence of SEQ ID NO: 18, a CDR2-alpha sequence comprising the amino acid sequence of SEQ ID NO: 19, and a CDR3-alpha sequence comprising the amino acid sequence of SEQ ID NO: 20) and a TCR beta variable domain having the amino acid sequence of SEQ ID NO: 27 (which includes a CDR1-beta sequence comprising the amino acid sequence of SEQ ID NO: 24, a CDR2-beta sequence comprising the amino acid sequence of SEQ ID NO: 25, and a CDR3-beta sequence comprising the amino acid sequence of SEQ ID NO: 26).

TCR-C comprised a TCR alpha variable domain having the amino acid sequence of SEQ ID NO: 35 (which includes a CDR1-alpha sequence comprising the amino acid sequence of SEQ ID NO: 32, a CDR2-alpha sequence comprising the amino acid sequence of SEQ ID NO: 33, and a CDR3-alpha sequence comprising the amino acid sequence of SEQ ID NO: 34) and a TCR beta variable domain having the amino acid sequence of SEQ ID NO: 41 (which includes a CDR1-beta sequence comprising the amino acid sequence of SEQ ID NO: 38, a CDR2-beta sequence comprising the amino acid sequence of SEQ ID NO: 39, and a CDR3-beta sequence comprising the amino acid sequence of SEQ ID NO: 40).

The TCRs were expressed in TCR-null Jurkat cells to assess the ability of the TCRs to be expressed at the cell surface and to activate T cells in the absence of endogenous TCRs.

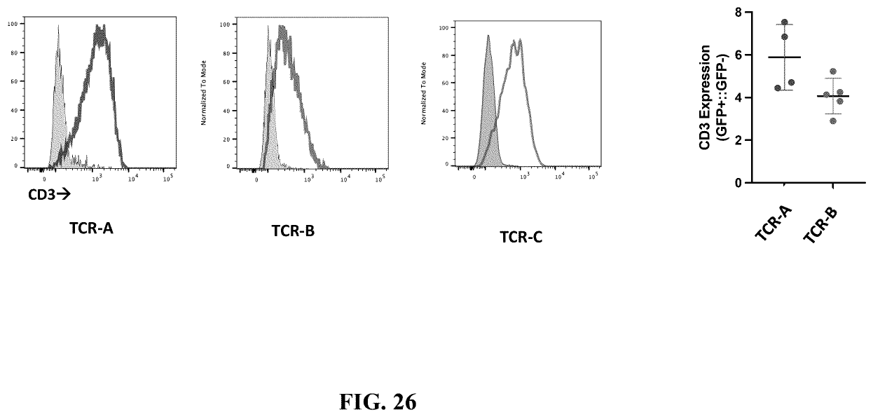

TCR-null Jurkats were plated at 1×105 per well in a 96-well plate and transduced with lentiviral vectors encoding one of the three TCRs. Cells were then expanded, assessed for TCR expression, and collected for downstream assays to assess function. Surface expression of each of the TCRs was verified by flow cytometric detection of CD3 expression and a lentiviral GFP-reporter (FIG. 26). All three of the tested TCRs demonstrated surface expression in the TCR-null Jurkats.

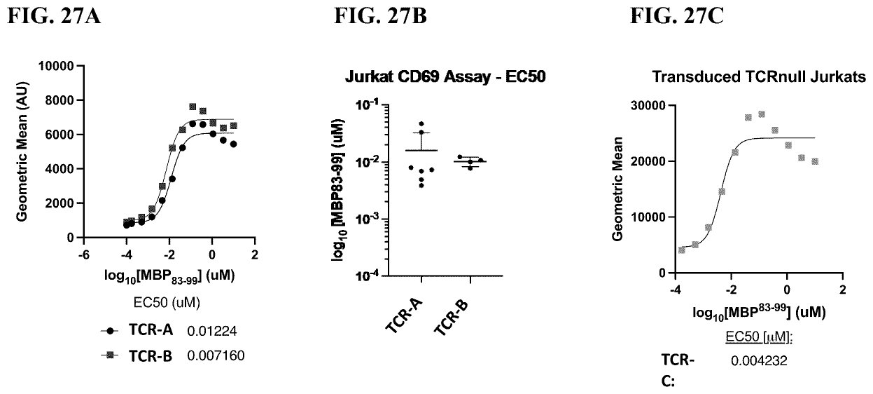

To test responsiveness of the TCRs to MBP complexed with MHC, the transduced Jurkat cells were co-cultured with BLS2b cells (B cells exclusively expressing HLA-DRA*01:01 and HLA-DRB1*15:01) and MBP83-99 peptide overnight. Peptide was titrated 4-fold at the concentrations shown. Flow cytometry was used to measure upregulation of CD69 surface expression (FIG. 27A-FIG. 27C). This data demonstrates that all three TCRs are expressed at the cell surface and are capable of activating TCR-null Jurkat cells.

TCR-A and TCR-B were independently expressed in conventional CD4 T cells to assess the ability of the TCRs to be expressed at the cell surface and to activate T cells in the presence of endogenous TCRs.

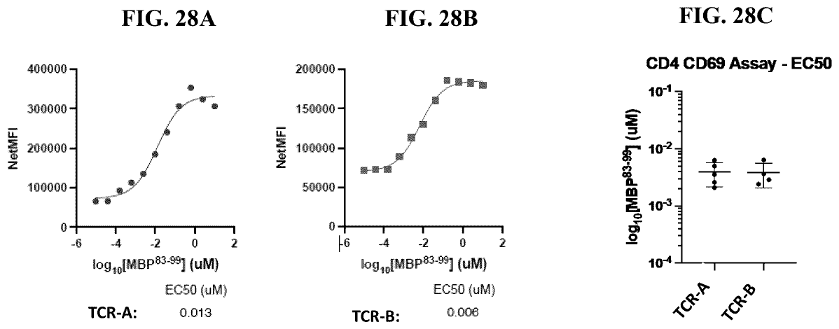

For each of the TCRs, CD4 T cells transduced with the TCRs were plated at 100,000 cells per well, across a row (12 wells) of a 96 well round bottom plate. 50,000 BLS2b cells (B cells exclusively expressing solely HLA-DRA*01:01 and HLA-DRB1*15:01) were added to each well containing transduced T cells and MBP83-99 peptide was serially titrated across the plate. The cells were cultured with peptide overnight for 20 hours in a humidified 37° C. 5% CO2 incubator. The following day, the cells were prepared for analysis by flow cytometry by staining with fluorescently labeled antibodies (anti-CD4 BV605, anti-CD69 BV421, anti-CD20 PE). Data was acquired on a BioRad ZE5 cytometer and analyzed in FlowJo v10. Transduced T cells were identified as CD20−, CD4+, GFP+ singlet events. T cell activation was determined by level of CD69 expression (FIG. 28A-FIG. 28C). This data demonstrates that both TCR-A and TCR-B are able activate conventional T cells.

### Example 6: Properties of Regulatory T Cells Expressing an Exogenous TCR

Regulatory T cells transduced with lentiviral vectors encoding the TCRs from Example 5 were tested in functional assays for their ability to be activated and have suppressive function in response to the MBP83-99 peptide when presented by HLA. Tregs were isolated from PBMC using the EasySep™ Human CD4+CD127lowCD25+ Regulatory T Cell Isolation Kit (Stemcell Technologies) and transduced and expanded as described in Example 1.

To test whether transduced regulatory T cells could be activated, the transduced regulatory T cells, or untransduced controls, were incubated at 37° C. with Mitomycin C treated peripheral blood mononuclear cells (PBMCs) isolated from HLA DRB1*15:01 donor, MBP83-99 peptide at a concentration of 0-100 UM (at 0, 0.000001, 0.00001, 0.0001, 0.001, 0.01, 0.1, 1, 10, and 100 μM) and 200 U/ml IL-2 in T cell culture medium. The ratio of PBMC to regulatory T cells was 4:1. Transduced regulatory T cells were differentiated from untransduced regulatory T cells by the GFP integrated in the lentiviral vector. Following incubation, regulatory T activation markers were measured by FACS after 1 day and cytokine levels were measured after 3 days.

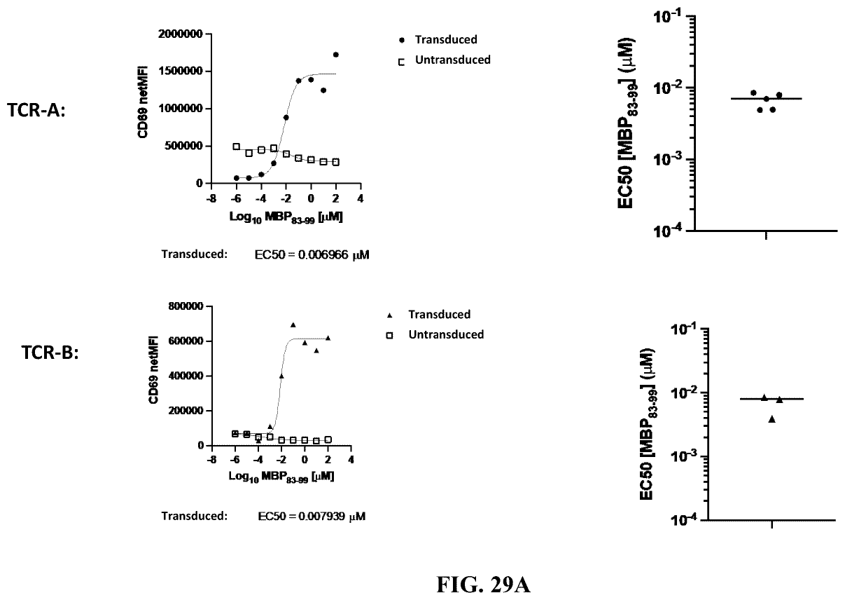

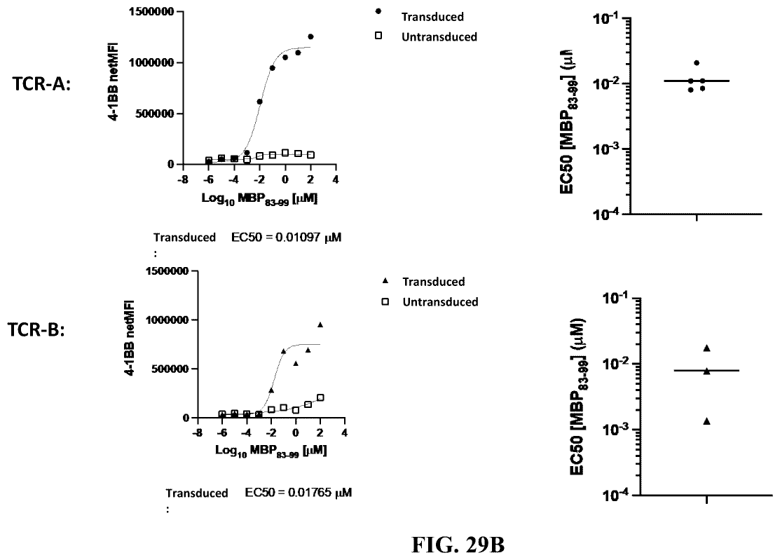

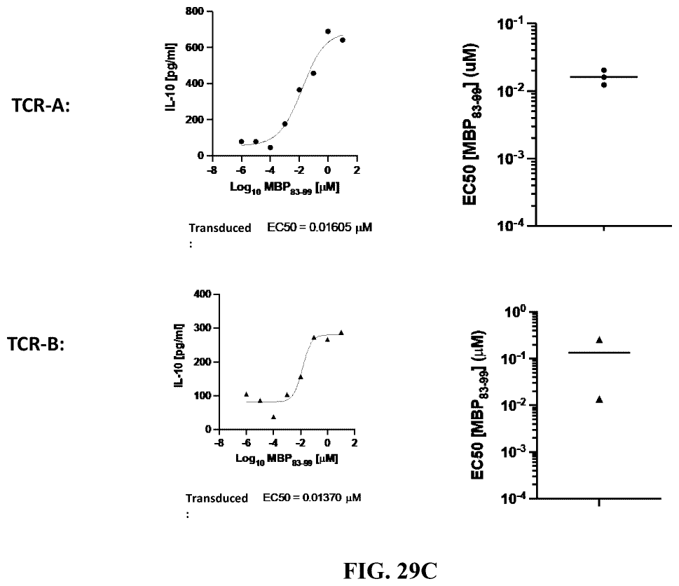

Representative graphs of CD69 expression levels on the regulatory T cell populations transduced with each of the two TCRs (TCR-A and TCR-B) and non-transduced regulatory T cells over a range of MBP83-99 peptide are provided in FIG. 29A. TCR-A provided an EC50 for CD69 of 7.0 nM; and TCR-B provided an EC50 for CD69 of 7.9 nM.

Representative graphs of 41-BB expression levels on the regulatory T cell populations transduced with each of the two TCRs (TCR-A and TCR-B) and non-transduced regulatory T cells over a range of MBP83-99 peptide are provided in FIG. 29B. TCR-A provided an EC50 for 4-1BB of 11.0 nM; and TCR-B provided an EC50 for 4-1BB of 18.0 nM.

Representative graphs of IL-10 expression levels on the regulatory T cell populations transduced with each of the two TCRs (TCR-A and TCR-B) and non-transduced regulatory T cells over a range of MBP83-99 peptide are provided in FIG. 29C. TCR-A provided an EC50 for IL-10 of 16.1 nM; and TCR-B provided an EC50 for IL-10 of 13.7 nM.

As shown, regulatory T cells expressing either TCR-A or TCR-B showed increased levels of CD69, 4-1BB, and IL-10 in response to MBP83-99 presented by PBMC relative to regulatory T cells that did not express an exogenous TCR. These data demonstrate that the transduced regulatory T cells are activated in response to MBP83-99 and exhibit desirable characteristics.

To test the ability of transduced regulatory T to suppress conventional T cell function, activation of conventional T cells expressing either TCR-A or TCR-B was stimulated with MBP83-99 peptide presented by PBMCs isolated from an HLA DRB1*15:01 donor in the presence of regulatory T cells expressing either TCR-A or TCR-B. Activation of conventional T cells was assessed by the presence of activation markers and cytokine release.

Regulatory T cells were incubated with Mitomycin C treated PBMCs isolated from an HLA DRB1*15:01 donor, MBP83-99 peptide at a concentration of 1 μM or 0.1 μM, conventional CD4 T cells expressing TCR-A or TCR-B, and 200 U/ml IL-2. The ratio of PBMC to conventional T cells was 4:1. Suppression of conventional T cells was measured at varying ratios of regulatory T cells to conventional T cells (1:1, 1:2, 1:4, and 1:8). Cells were incubated for 4 days. Following incubation, T cell activation markers were measured by FACS and cytokine levels were measured.

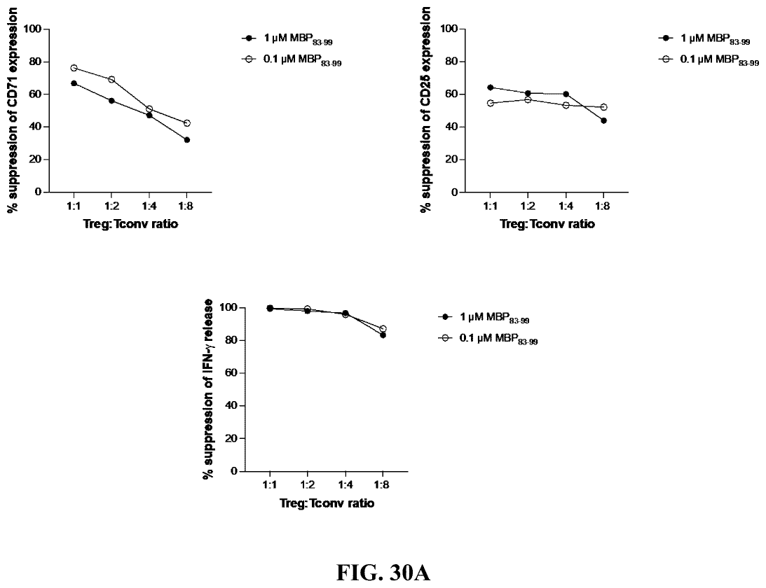

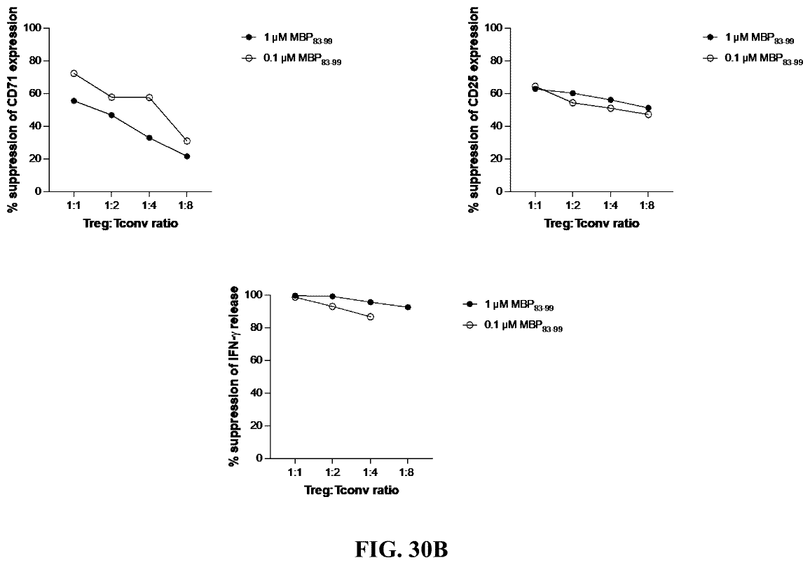

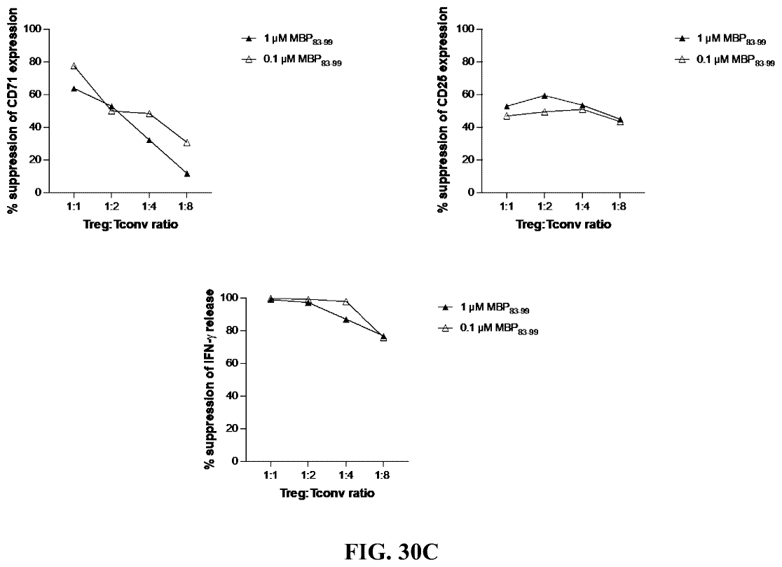

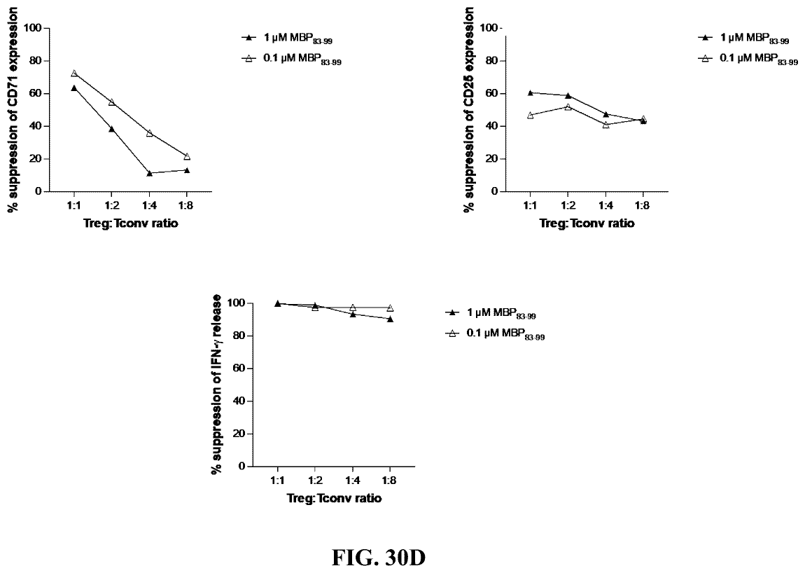

FIG. 30A shows the suppression of conventional T cells expressing TCR-A by regulatory T cells expressing TCR-A. Percent suppression of CD71, CD25, and IFN-γ at 1 μM and 0.1 M MBP83-99 peptide are provided. FIG. 30B shows the suppression of conventional T cells expressing TCR-B by regulatory T cells expressing TCR-A. Percent suppression of CD71, CD25, and IFN-γ at 1 μM and 0.1 μM MBP83-99 peptide are provided. FIG. 30C shows the suppression of conventional T cells expressing TCR-A by regulatory T cells expressing TCR-B. Percent suppression of CD71, CD25, and IFN-γ at 1 μM and 0.1 μM MBP83-99 peptide are provided. FIG. 30D shows the suppression of conventional T cells expressing TCR-B by regulatory T cells expressing TCR-B. Percent suppression of CD71, CD25, and IFN-γ at 1 μM and 0.1 μM MBP83-99 peptide are provided.

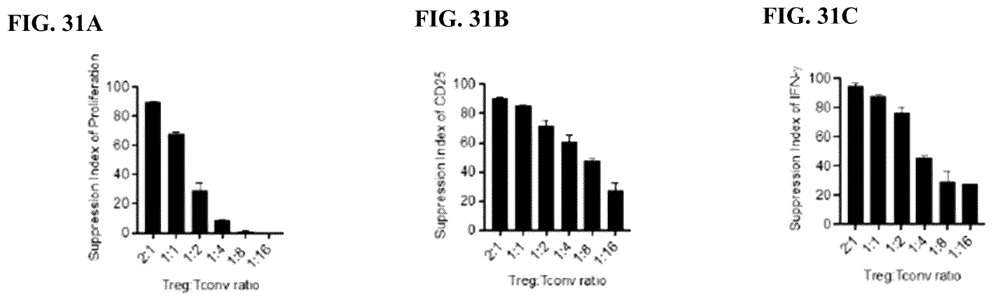

The ability of regulatory T cells expressing TCR-A to suppress polyclonal (untransduced) conventional T cells was measured using the same method. For polyclonal conventional T cells, suppression of proliferation (FIG. 31A), CD25 (FIG. 31B), and interferon gamma (FIG. 31C) at the Treg:Tcon ration shown was measured.

These data demonstrate that stable regulatory T cells expressing an exogenous TCR were able to suppress conventional T cells. Specifically, regulatory T cells expressing an exogenous TCR targeting MBP83-99 were capable of suppressing activation and cytokine release by conventional T cells. Collectively, these data suggest that the populations of cells comprising regulatory T cells are capable of suppressing the immune response through an MBP-specific mechanism and thus should be useful in treating subjects having Multiple Sclerosis (e.g., progressive Multiple Sclerosis).

### Example 7: Characterization of T Cells Expressing Engineered T Cell Receptors

Cysteine substitutions were introduced into the TCR alpha and TCR beta sequences of TCR-A and TCR-B to introduce a disulfide bond and increase pairing of the TCR at the cell surface. For each TCR, a T48C mutation was introduced relative to the TRAC constant region (SEQ ID NO. 58) and a S57C mutation was introduced relative to the TRBC2 constant region (SEQ ID NO. 60). Engineered TCR-E v1 (middle line) is an engineered version of TCR-A having the additional engineered disulfide bond (amino acid sequence of v1 corresponds to SEQ ID NO: 79); and Engineered TCR-E v2 has the amino acid sequence of SEQ ID NO: 79 and is encoded by the codon-optimized nucleic acid sequence of SEQ ID NO: 87. Engineered TCR-F v1 (middle line) is an engineered version of TCR-B having the additional engineered disulfide bond (amino acid sequence of v1 corresponds to SEQ ID NO: 86); and Engineered TCR-F v2 (top line) has the amino acid sequence of SEQ ID NO: 86 and encoded by the codon-optimized nucleic acid sequence of SEQ ID NO: 88.

TCRs were expressed in TCR-null Jurkat cells to assess the ability of engineered TCRs to be expressed at the cell surface and to activate T cells in the absence of endogenous TCRs.

TCR-null Jurkats were plated at 1×105 per well in a 96-well plate and transduced with lentiviral vectors encoding one of the TCRs. Cells were then expanded and collected for downstream assays to assess function.

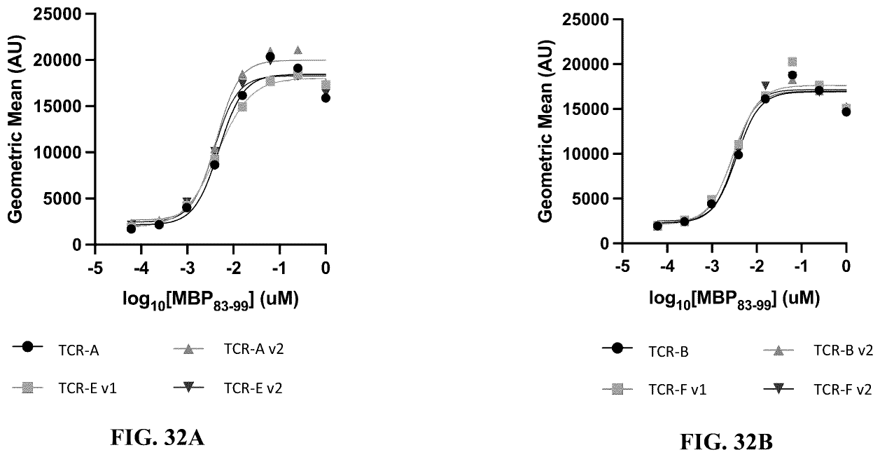

To test responsiveness of cells expressing the TCRs to MBP complexed with MHC, the transduced Jurkat cells were co-cultured with BLS2b cells (B cells exclusively expressing HLA-DRA*01:01 and HLA-DRB1*15:01) and MBP83-99 peptide overnight. The peptide was titrated 4-fold at the concentrations shown in FIGS. 32A-32B. Flow cytometry was used to measure upregulation of CD69 surface expression (FIGS. 32A-32B and Table 25). These data demonstrated that the Jurkats expressing each of the TCRs were responsive to increasing concentrations of antigen.

TABLE 25
CD69 expression in TCR-transduced Jurkat cells

| | Max Response | EC-50 (nM) |
|---|---|---|
| TCR-A | 1.00 | 0.0042 |
| TCR-A v2 | 1.03 | 0.0039 |
| TCR-E Engineered v1 | 0.95 | 0.0040 |
| TCR-E Engineered v2 | 0.97 | 0.0037 |
| TCR-B | 1.00 | 0.0030 |
| TCR-B v2 | 1.03 | 0.0026 |
| TCR-F Engineered v1 | 1.03 | 0.0030 |
| TCR-F Engineered v2 | 1.04 | 0.0029 |

The TCRs were also expressed in conventional CD4+ T cells to assess cell surface expression of the TCRs and T cell activation in the presence of endogenous TCRs.

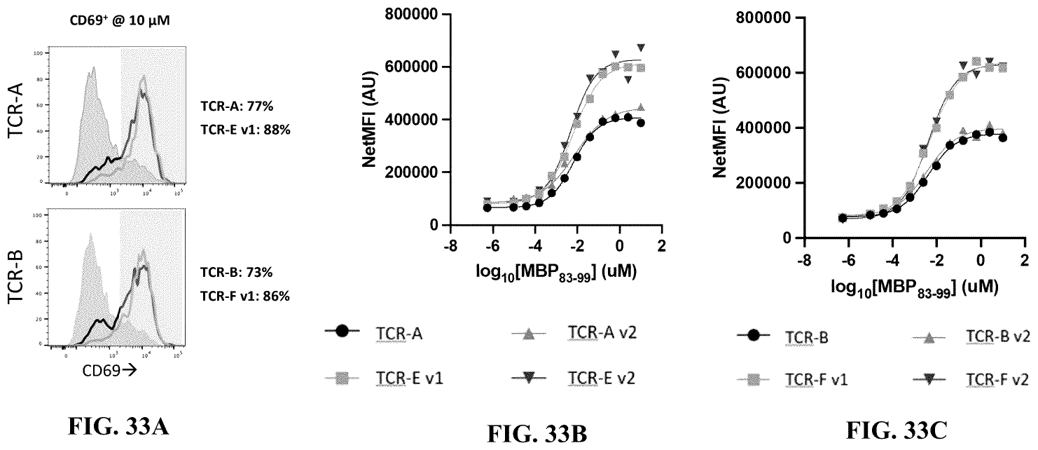

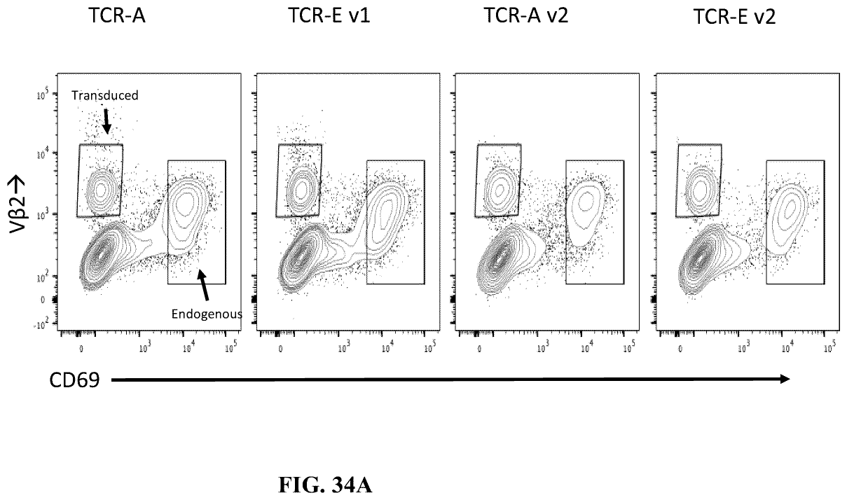

CD4+ T cells transduced with each of the TCRs were plated at 100,000 cells per well, across a row (12 wells) of a 96 well round bottom plate. 50,000 BLS2b cells (B cells exclusively expressing solely HLA-DRA*01:01 and HLA-DRB1*15:01) were added to each well containing transduced T cells and MBP83-99 peptide was serially titrated across the plate. The cells were cultured with peptide overnight for 20 hours in a humidified 37° C. 5% CO2 incubator. The following day, the cells were prepared for analysis by flow cytometry by staining with fluorescently labeled antibodies (anti-CD4 BV605, anti-CD69 BV421, anti-CD20 PE). Data was acquired on a BioRad ZE5 cytometer and analyzed in FlowJo v10. Transduced T cells were identified as CD20⁻, CD4⁺, GFP⁺ singlet events. T cell activation was determined by level of CD69 expression (FIGS. 33A-33C and Table 26).

TABLE 26
CD69 expression in TCR-transduced CD4⁺ T cells

| | Max Response | EC-50 (μM) |
|---|---|---|
| TCR-A | 1.00 | 0.0076 |
| TCR-A v2 | 1.05 | 0.0059 |
| TCR-E Engineered v1 | 1.36 | 0.0050 |
| TCR-E Engineered v2 | 1.44 | 0.0056 |
| TCR-B | 1.00 | 0.0075 |
| TCR-B v2 | 1.04 | 0.0055 |
| TCR-F Engineered v1 | 1.57 | 0.0052 |
| TCR-F Engineered v2 | 1.59 | 0.0045 |

These data demonstrated that the expression level of markers of cell activation (e.g., CD69) are higher in CD4⁺ T cells expressing the engineered TCRs (e.g., TCR-E Engineered v2 and TCR-F Engineered v2), relative to cells expressing the parental versions (TCR-A and TCR-B).

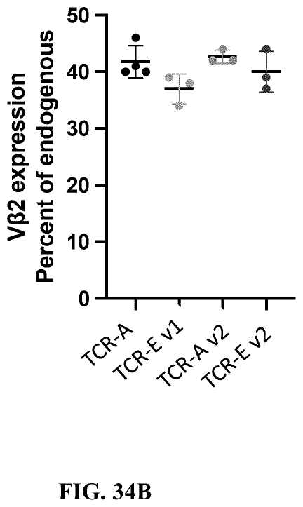

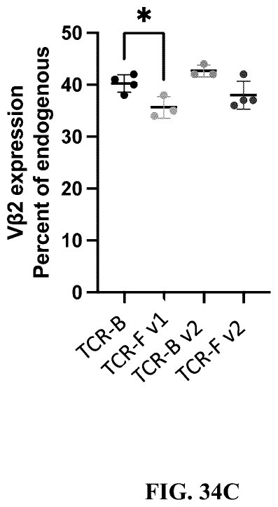

Surface expression of each of the TCRs was verified by flow cytometric detection of Vβ2 expression and CD69 expression (FIG. 34A). Expression of Vβ2 correlates with surface-level expression of the TCR (FIGS. 34B-34C). These data demonstrate that the engineered TCRs (e.g., TCR-E Engineered v2 and TCR-F Engineered v2) do not significantly impact the surface expression of these TCRs in conventional T cells, relative to the parental versions (TCR-A and TCR-B).

### Example 8. Activation of Regulatory T Cells Expressing Engineered T Cell Receptors

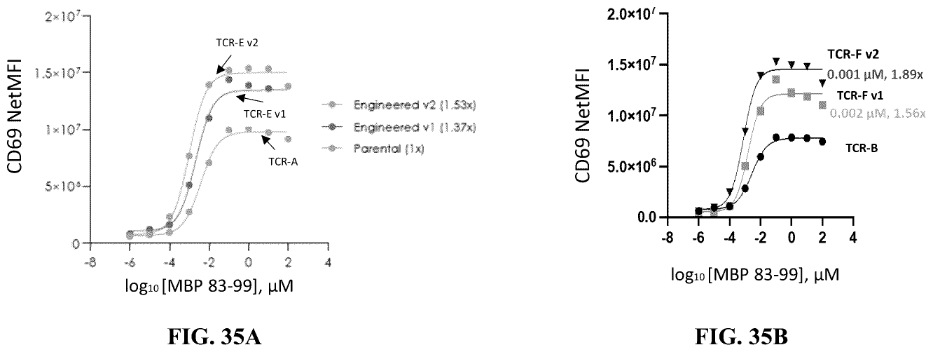

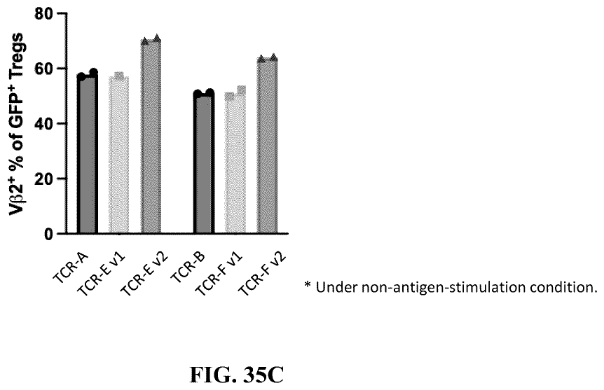

Regulatory T (Treg) cells were prepared as described in Example 6. The isolated Tregs were transduced with the TCRs described herein (TCR-A, TCR-E, TCR E-v2, TCR-B, TCR-F, and TCR-F v2). The Treg cells were then contacted with BLS2b cells, which are B cells expressing only HLA-DRA*01:01 and HLA-DRB1*15:01, and MBP83-99 peptide to measure Treg activation as demonstrated by expression of CD69, as described in Example 6. FIGS. 35A-35B show graphs depicting the activation of the Tregs in response to increasing doses of the MBP peptide.

As shown in FIG. 35A, the activation of Tregs expressing TCR-E Engineered v2 was 1.53 times greater than activation of Tregs expressing the parental version (TCR-A), while the activation of Tregs expressing TCR-E Engineered v1 was 1.37 times greater than activation of Tregs expressing the parental version (TCR-A).

As shown in FIG. 35B, the activation of Tregs expressing TCR-F Engineered v2 was 1.89 times greater than activation of Tregs expressing the parental version (TCR-B), while the activation of Tregs expressing TCR-F Engineered v1 was 1.56 times greater than activation of Tregs expressing the parental version (TCR-B).

Surface expression of each of the TCRs was verified by flow cytometric detection of Vβ2 expression. A higher percentage of cells transduced with TCR-E Engineered v2 or TCR-F Engineered v2 expressed Vβ2, relative to the parental controls and Engineered v1 versions, respectively (FIG. 35C). Expression of Vβ2 correlates with surface-level expression of the TCR.

These data demonstrate that the level of activation of Tregs transduced with the engineered TCRs of the disclosure (e.g., a TCR encoded by a nucleotide sequence having 100% identity to the nucleotide sequence of SEQ ID NO: 87 or 88) in response to target antigen MBP83-99 peptide is higher relative to the corresponding parental (non-engineered) and Engineered v1 TCRs. Furthermore, these data demonstrate that Tregs transduced with the Engineered v2 versions of TCR-E and TCR-F had higher levels of TCR expression on their cell surface.

### Example 9: Suppressive Capacity of Engineered TCR Constructs in Regulatory T Cells

Regulatory T cells were transduced with lentiviral vectors encoding one of TCR-A, TCR-B, TCR-E v1, TCR-E v2, TCR-F v1, and TCR-F v2 as in Example 6. Transduced regulatory T cells were then tested in functional assays for their affinity and response to the MBP83-99 peptide.

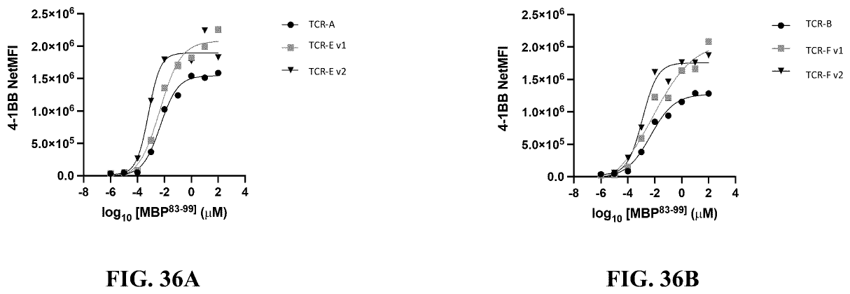

To test which TCR had the highest affinity and maximum response, a lentiviral transduced population of cells comprising regulatory T cells were incubated at 37° C. with Mitomycin C treated peripheral blood mononuclear cells (PBMCs) isolated from HLA DRB1*15:01 donor, MBP83-99 peptide at the concentrations shown in FIGS. 36A-36B, and 200 U/ml IL-2 in T cell culture medium. The ratio of PBMC to regulatory T cells was 4:1. Following incubation, regulatory T activation markers were measured by FACS after 1 day and cytokine levels were measured after 3 days.

Representative graphs of 4-1BB expression levels on the regulatory T cell populations transduced with the TCRs over a range of MBP83-99 peptide are provided in FIGS. 36A-36B and Table 27. These data demonstrate that the expression level of markers of activation (e.g., 4-1BB) of regulatory T cells expressing the engineered TCRs of the disclosure (e.g., TCR-E Engineered v1 and Engineered v2, TCR-F Engineered v1 and Engineered v2) are higher relative to regulatory T cells expressing the corresponding parental TCRs (TCR-A and TCR-B).

TABLE 27
4-1BB expression in TCR-transduced regulatory T cells

| | Max Response | EC-50 (μM) |
|---|---|---|
| TCR-A | 1.00 | 0.0050 |
| TCR-E Engineered v1 | 1.35 | 0.0046 |
| TCR-E Engineered v2 | 1.23 | 0.0065 |
| TCR-B | 0.83 | 0.0047 |
| TCR-F Engineered v1 | 1.35 | 0.0070 |
| TCR-F Engineered v2 | 1.14 | 0.0012 |

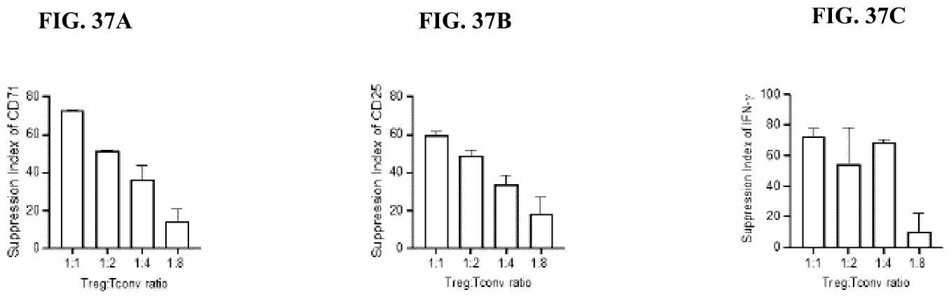

The ability of regulatory T cells transduced with TCR-E v2 to suppress conventional T cell function was assessed using the suppression assay described in Example 6. The ability to suppress conventional T cells transduced with TCR-B was measured. Suppression of CD25 (FIG. 37A), CD71 (FIG. 37B), and interferon gamma (FIG. 37C) at the Treg:Tcon ratios shown was measured. The data presented herein suggest that Treg transduced with TCR-Ev2 are able to effectively suppress TCR-B transduced conventional T cells.

### Example 10. Stability of Regulatory T Cells Under Pro-Inflammatory Conditions

The stability of Tregs prepared according to Example 6 and expressing TCR-A under inflammatory conditions was assessed. An isolated cell population comprising CD4⁺CD25⁺CD127^{low} regulatory T cells prepared according to the methods described in Example 6 and transduced with TCR-A was expanded for 8 days with human T-activator CD3/CD28 Dynabeads (Life Technologies, Cat #11132D) and human recombinant IL-2. Following expansion, the purity of this isolated cell population comprising regulatory T cells sample was characterized by immunostaining using a flow cytometry and TSDR hypomethylation using droplet digital PCR (ddPCR). The isolated cell population was cryopreserved and stored in liquid nitrogen tank.

The cryopreserved cell population was then thawed for cell culture. Cells were plated into 96 round bottom tissue-culture-treated plates at 50,000 cells per well in 200 μL X-VIVO 15 serum-free hematopoietic cell medium (Lonza, Cat #04-418Q). Sample wells were supplemented with 1000 U/mL IL-2 and varying concentrations of a mixture of pro-inflammatory cytokines (IL-1β, IL-6, IL-12, IL-17A, IFN-β, IFN-γ, TGF-β1, and TNF-α). Regulatory T cells were stimulated with 25,000 T-Activator Dynabeads per well. Every 3 or 4 days, regulatory T cells in each well were split into 2 wells, and culture supernatants were replaced with fresh X-VIVO 15 medium supplemented with fresh Dynabeads and cytokines. On Days 3, 6, 9, 13, 16, 20, and 23 of culturing and expansion, one well of split cells were used for FACS-based immunophenotyping to determine the cell count of regulatory T cells (viable CD4+Foxp3+ cells) on each of those days.

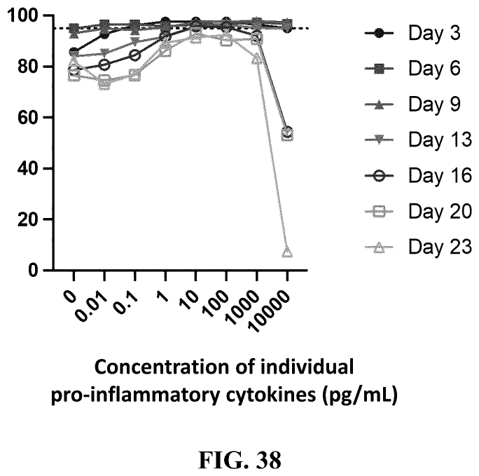

Incubation of the isolated cell population comprising regulatory T cells with the mixture of IL-2, IL-1β, IL-6, IL-12, IL-17A, IFN-β, IFN-γ, TGF-31, and TNF-α demonstrated that the regulatory T cells maintained expression of stability markers CD4 and FOXP3. As shown in FIG. 38, the percentage of regulatory T cells having CD4+/FOXP3+ expression remained relatively constant for at least 23 days in the presence of pro-inflammatory cytokines (at concentrations of up to 1,000 μg/mL of each cytokine in the mixture).

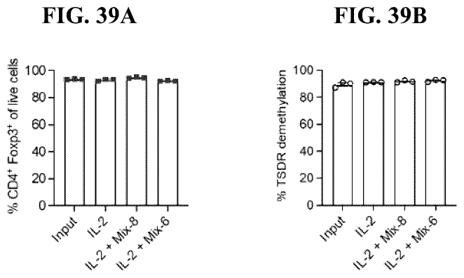

Having determined the optimal levels of cytokines for this experiment, the assay was repeated with 1000 U/mL IL-2 and 1000 μg/ml of each inflammatory cytokine listed above (Mix-8) or with IL-1β, IL-6, IL-12, IL-17A, IFN-g, and TNF-α (Mix-6.) The results are shown in FIG. 39A-FIG. 39B. The Tregs were stable for 3 days in either cytokine mixture. Similar results were observed with Tregs transduced with TCR-Ev2. These data show that regulatory T cells are stable in the presence of pro-inflammatory conditions for at least 23 days.

### Example 11: In Vivo Efficacy of MBP₈₃₋₉₉-Targeting TCR Tregs

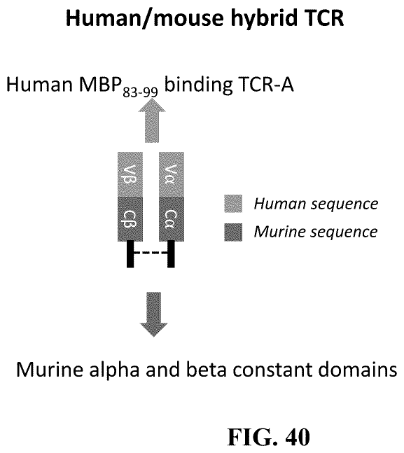

TCR-A was modified to replace the human alpha and beta constant domains with murine constant domains for suitability of expression in a mouse EAE model (‘murinized TCR-A’). A schematic of the TCR is shown in FIG. 40. The alpha and beta chains of the TCR were cloned into a vector comprising an N-terminal TCR beta chain and a C-terminal TCR alpha chain with a linker domain comprising a GSG amino acid sequence followed by a P2A self-cleaving peptide.

EAE is a mouse model of neuroinflammation commonly used in MS research. This model is suitable for the study of pharmacologic properties of the surrogate Treg drug product in the context of CNS inflammation. These experiments were conducted in an HLA-DRA*01:01/-DRB1*15:01-transgenic, murine MHC-II-deficient mice. The expression of HLA-DRA*01:01/-DRB1*15:01 ensures that chimeric TCR-A-expressing Tregs encounters their cognate antigen in the CNS. Importantly, the MBP83-99 epitope is conserved between mice and humans. Furthermore, the HLA-DRA*01:01/-DRB1*15:01 MHC-II complex in the mice used in these experiments is also chimeric, with the CD4-binding domain encoded by mouse sequences (Rich 2004), ensuring optimal interaction with the murine CD4 coreceptor expressed by murinized TCR-A Tregs.

In the active EAE model, disease is induced by immunization with MOG35-55 peptide in CFA as well as pertussis toxin, which induces a neuroinflammatory disease characterized by ascending paralysis. Importantly, MOG35-55 is also presented by HLA-DRA*01:01/-DRB1*15:01 and has been shown to cause disease in this context (Ji 2013).

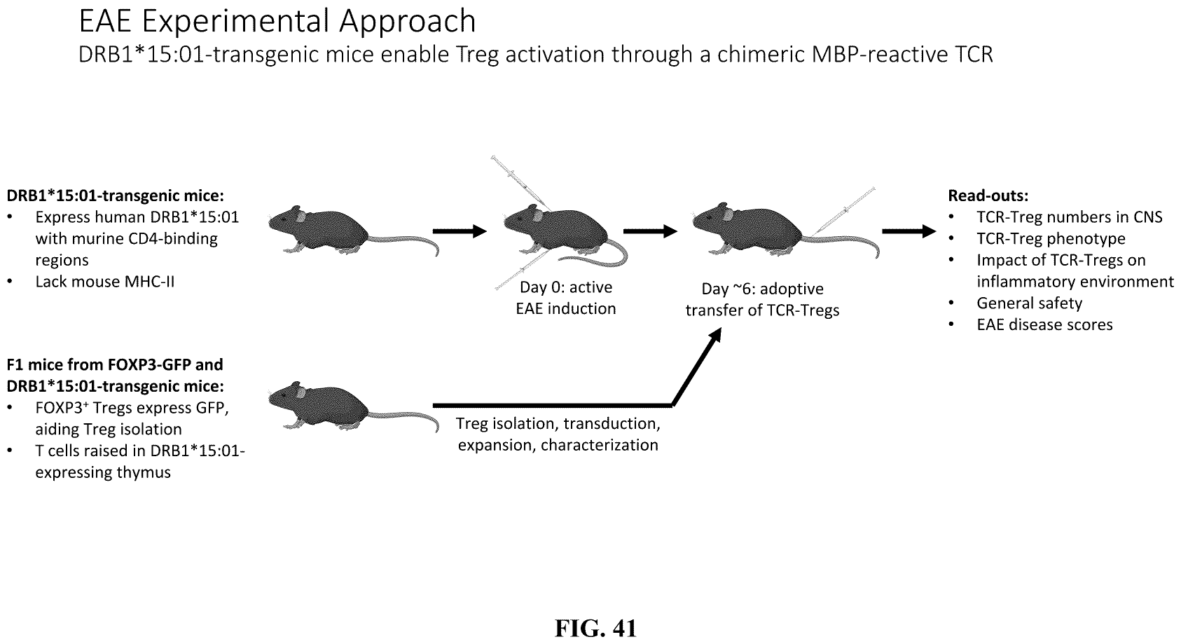

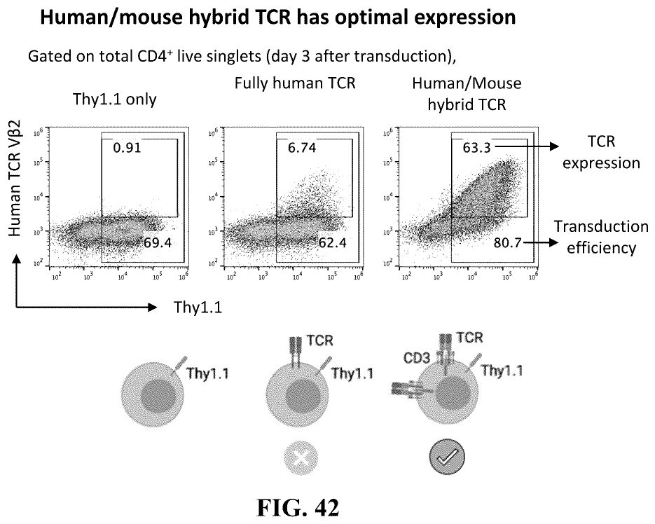

The protocol for these studies is shown in FIG. 41. mice expressing Fox-3-GFP were crossed with DRA*01:01/-DRB1*15:01-transgenic mice. GFP+ Treg were isolated from the F1 mice and transduced with the murinized TCR-A. FIG. 42 shows cell surface expression of murinized TCR-A in the murine Tregs as determined by flow cytometry. EAE was then induced in the DRA*01:01/-DRB1*15:01 transgenic mice and, on day 6, the isolated Tregs were administered to the mice by intra-orbital injection. EAE scores were measured daily, and brain, spinal cord, and spleen samples were collected at days 11, 16, and 19 post EAE induction to assess GFP+ Treg localization.

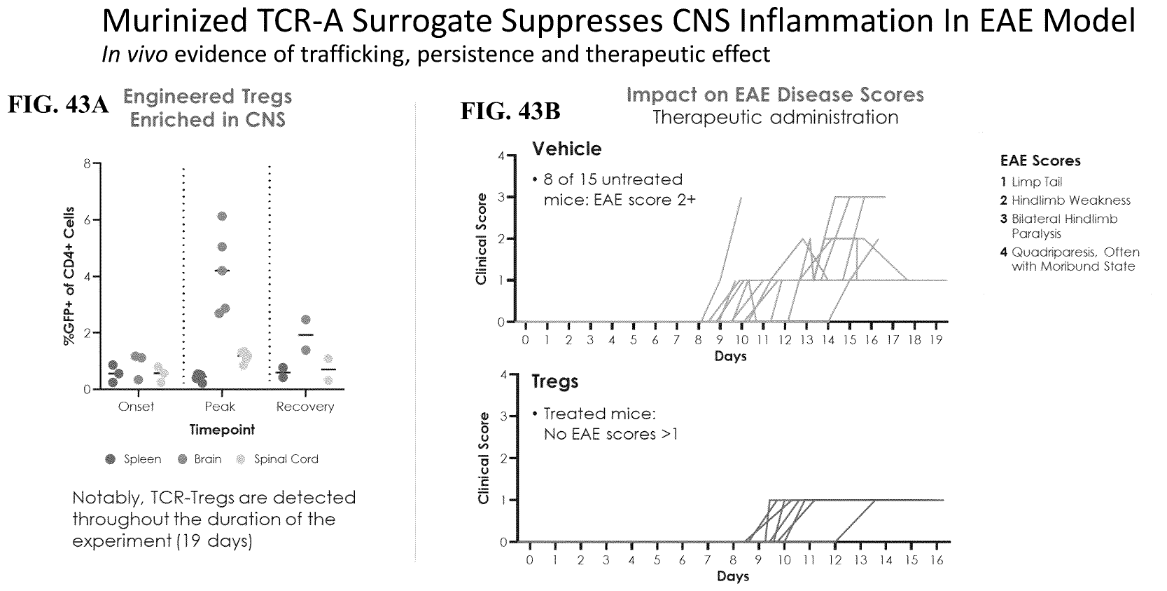

The results are shown in FIG. 43A-FIG. 43B. FIG. 43A shows GFP+ Treg localization over the course of the experiment. For each of onset, peak, and recovery, measurements for the spleen, brain, and spinal cord are shown on the left, middle, and right respectively. GFP+ TCR Treg were localized in the brain and spinal cord and detected throughout the duration of the experiment. FIG. 43B shows that 8 of 15 mice treated with vehicle showed EAE scores above 2, whereas none of the Treg-treated mice developed EAE scores above 1. Together, these results indicate that Tregs engineered with a TCR having the variable domains of TCR-A and TCR-E shows promise in treating MS in an in vivo model.

1.-153. (canceled)

154. An isolated population of cells comprising stable CD4+ T regulatory cells (T regs) derived from a subject having multiple sclerosis,
wherein at least 80% of the cells are stable CD4+ T regs comprising a hypomethylated T cell specific demethylated region (TSDR) at the FOXP3 locus.

155. The isolated population of claim 154, wherein the cells comprise an exogenous human T cell receptor (TCR) that binds specifically to myelin basic protein (MBP) peptide complexed with a major histocompatibility complex (MHC).

156. The isolated population of claim 155, wherein the MBP peptide is an MBP 83-99 peptide comprising the amino acid sequence of SEQ ID NO: 61.

157. The isolated population of claim 155, wherein the MHC comprises HLA-DRB1*15:01 and wherein the MHC further comprises HLA-DRA*01:01.

158. The isolated population of claim 155, wherein the exogenous TCR comprises:
(a) a TCRα chain variable region comprising a CDR1 comprising the amino acid sequence of SEQ ID NO: 1, a CDR2 comprising the amino acid sequence of SEQ ID NO: 2, and a CDR3 comprising the amino acid sequence of SEQ ID NO: 3; and
(b) a TCRβ chain variable region comprising a CDR1 comprising the amino acid sequence of SEQ ID NO: 7, a CDR2 comprising the amino acid sequence of SEQ ID NO: 8, and a CDR3 comprising the amino acid sequence of SEQ ID NO: 9.

159. The isolated population of claim 155, wherein the exogenous TCR is encoded as a single polypeptide comprising an amino acid sequence having at least 90%, 95%, or 100% identity to the amino acid sequence of SEQ ID NO: 79 or wherein the exogenous TCR is encoded as a single polypeptide comprising an amino acid sequence having at least 90%, 95%, or 100% identity to the amino acid sequence of SEQ ID NO: 97, optionally wherein the single polypeptide comprises the amino acid sequence of SEQ ID NO: 79 and is encoded by a nucleotide sequence having at least 90%, 95%, or 100% identity to the nucleotide sequence of SEQ ID NO: 87.

160. The isolated population of claim 155, wherein the exogenous TCR comprises:
(a) a TCRα chain variable region comprising a CDR1 comprising the amino acid sequence of SEQ ID NO: 18, a CDR2 comprising the amino acid sequence of SEQ ID NO: 19, and a CDR3 comprising the amino acid sequence of SEQ ID NO: 20; and
(b) a TCRβ chain variable region comprising a CDR1 comprising the amino acid sequence of SEQ ID NO: 24, a CDR2 comprising the amino acid sequence of SEQ ID NO: 25, and a CDR3 comprising the amino acid sequence of SEQ ID NO: 26.

161. The isolated population of claim 160, wherein the exogenous TCR is encoded as a single polypeptide comprising an amino acid sequence having at least 90%, 95%, or 100% identity to the amino acid sequence of SEQ ID NO: 31 or wherein the exogenous TCR is encoded as a single polypeptide comprising an amino acid sequence having at least 90%, 95%, or 100% identity to the amino acid sequence of SEQ ID NO: 86, optionally wherein the single polypeptide comprises the amino acid sequence of SEQ ID NO: 86 and is encoded by a nucleotide sequence having at least 90%, 95%, or 100% identity to the nucleotide sequence of SEQ ID NO: 88.

162. The isolated population of claim 154, wherein:
a) the stable CD4+ T regs do not express a FOXP3 protein from an engineered FOXP3 locus;
b) the TSDR is the CNS2 region of FOXP3;
c) the MHC is MHC Class I or MHC Class II;
d) at least 85%, at least 90%, at least 95%, at least 96%, at least 97%, at least 98%, or at least 99% of the cells are stable CD4+ T regs comprising a hypomethylated TSDR at an endogenous FOXP3 locus;
e) least 80%, at least 85%, at least 90%, or at least 95% of the cells are CD4⁺CD25⁺CD127^{-/lo}; and/or
f) at least 80%, at least 85%, at least 90%, or at least 95% of the cells are CD4⁺CD25⁺CD127^{-/lo}FOXP3⁺.

163. The isolated population of claim 154, comprising at least 4×10⁷ stable CD4+ T regs.

164. The isolated population of claim 154, wherein the stable CD4+ T regs have a human leukocyte antigen (HLA)-DR15 haplotype, wherein the stable CD4+ T regs comprise an HLA-DRB1*15:01 allele and wherein the stable CD4+ T regs further comprise an HLA-DRB5*01:01 allele.

165. A pharmaceutical composition comprising the isolated population of claim 154 and a pharmaceutically acceptable excipient.

166. A method of treating multiple sclerosis comprising administering to a subject the pharmaceutical composition of claim 165.

167. The method of claim 166, wherein the pharmaceutical composition is administered in an effective amount to alleviate one or more symptoms of multiple sclerosis.

168. The method of claim 166, wherein:
a) the administering comprises intravenous administration;
b) the administering comprises one or more infusions;
c) the cells of the isolated population are autologous relative to the subject; and/or
d) the subject has progressive Multiple Sclerosis or relapsing remitting multiple sclerosis, optionally wherein the subject has primary progressive Multiple Sclerosis (PPMS) or non-relapsing progressive Multiple Sclerosis.


SEQUENCE LISTING (124 sequences)

SEQ ID NO: 1 - protein, 7 aa, Homo sapiens
KTSINNL

SEQ ID NO: 2 - protein, 9 aa, Homo sapiens
LIRSNEREK

SEQ ID NO: 3 - protein, 14 aa, Homo sapiens
CATDTTSGTYKYIF

SEQ ID NO: 4 - protein, 112 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLA

SEQ ID NO: 5 - protein, 253 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 6 - protein, 273 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 7 - protein, 8 aa, Homo sapiens
LDFQATTM

SEQ ID NO: 8 - protein, 9 aa, Homo sapiens
TSNEGSKAT

SEQ ID NO: 9 - protein, 16 aa, Homo sapiens
CSARDLTSGANNEQFF

SEQ ID NO: 10 - protein, 118 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVL

SEQ ID NO: 11 - protein, 297 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 12 - protein, 318 aa, synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 13 - protein, 311 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 14 - protein, 118 aa, synthetic construct
GAVVSQHPSWVISKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVL

SEQ ID NO: 15 - protein, 297 aa, synthetic construct
GAVVSQHPSWVISKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 16 - protein, 318 aa, synthetic construct
GAVVSQHPSWVISKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 17 - protein, 606 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 18 - protein, 7 aa, Homo sapiens
KTSINNL

SEQ ID NO: 19 - protein, 9 aa, Homo sapiens
LIRSNEREK

SEQ ID NO: 20 - protein, 14 aa, Homo sapiens
CATDATSGTYKYIF

SEQ ID NO: 21 - protein, 112 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDATSGTYKYIFGTGTRLKVLA

SEQ ID NO: 22 - protein, 253 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDATSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 23 - protein, 273 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDATSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 24 - protein, 8 aa, Homo sapiens
LDFQATTM

SEQ ID NO: 25 - protein, 9 aa, Homo sapiens
TSNEGSKAT

SEQ ID NO: 26 - protein, 16 aa, Homo sapiens
CSARDLTSGSLNEQFF

SEQ ID NO: 27 - protein, 118 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVL

SEQ ID NO: 28 - protein, 297 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 29 - protein, 318 aa, synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 30 - protein, 311 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 31 - protein, 606 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDATSGTYKYIFGTGTRLKVLANIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 32 - protein, 5 aa, Homo sapiens
TSINN

SEQ ID NO: 33 - protein, 7 aa, Homo sapiens
IRSNERE

SEQ ID NO: 34 - protein, 13 aa, Homo sapiens
CATDSGGSYIPTF

SEQ ID NO: 35 - protein, 111 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDSGGSYIPTFGRGTSLIVHP

SEQ ID NO: 36 - protein, 252 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 37 - protein, 272 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 38 - protein, 5 aa, Homo sapiens
SQVTM

SEQ ID NO: 39 - protein, 7 aa, Homo sapiens
ANQGSEA

SEQ ID NO: 40 - protein, 15 aa, Homo sapiens
CSAWPSGQGTYGYTF

SEQ ID NO: 41 - protein, 116 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSAWPSGQGTYGYTFGSGTRLTVV

SEQ ID NO: 42 - protein, 293 aa, Homo sapiens
SAVISQKPSRDICQRGTSLTIQCQVDSQVTMMFWYRQQPGQSLTLIATANQGSEATYESGFVIDKFPISRPNLTFSTLTVSNMSPEDSSIYLCSAWPSGQGTYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 43 - protein, 314 aa, synthetic construct
SAVISQKPSRDICQRGTSLTIQCQVDSQVTMMFWYRQQPGQSLTLIATANQGSEATYESGFVIDKFPISRPNLTFSTLTVSNMSPEDSSIYLCSAWPSGQGTYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPG

SEQ ID NO: 44 - protein, 308 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSAWPSGQGTYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 45 - protein, 602 aa, synthetic construct
MLSLLLLLLGLGSVFSAVISQKPSRDICQRGTSLTIQCQVDSQVTMMFWYRQQPGQSLTLIATANQGSEATYESGFVIDKFPISRPNLTFSTLTVSNMSPEDSSIYLCSAWPSGQGTYGYTFGSGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDSGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 46 - protein, 7 aa, Homo sapiens
KTSINNL

SEQ ID NO: 47 - protein, 9 aa, Homo sapiens
LIRSNEREK

SEQ ID NO: 48 - protein, 12 aa, Homo sapiens
CATDGNGNQFYF

SEQ ID NO: 49 - protein, 110 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDGNGNQFYFGTGTSLTVIP

SEQ ID NO: 50 - protein, 251 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDGNGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 51 - protein, 271 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDGNGNQFYFGTGTSLTVIPNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 52 - protein, 7 aa, Homo sapiens
ISGHATL

SEQ ID NO: 53 - protein, 8 aa, Homo sapiens
QFQNNGVV

SEQ ID NO: 54 - protein, 15 aa, Homo sapiens
CASSIRHRTNTEAFF

SEQ ID NO: 55 - protein, 115 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSIRHRTNTEAFFGQGTRLTVV

SEQ ID NO: 56 - protein, 292 aa, Homo sapiens
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSIRHRTNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 57 - protein, 311 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSIRHRTNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 58 - protein, 141 aa, Homo sapiens
NIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 59 - protein, 177 aa, Homo sapiens
EDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 60 - protein, 179 aa, Homo sapiens
EDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 61 - protein, 17 aa, Homo sapiens
ENPVVHFFKNIVTPRTP

SEQ ID NO: 62-66 - skipped (5 entries)

SEQ ID NO: 67 - protein, 332 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 68 - protein, 311 aa, synthetic construct
MLLLLLLLGPGSGLGAVVSQHPSWVISKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 69 - protein, 332 aa, synthetic construct
MLLLLLLLGPGSGLGAVVSQHPSWVISKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 70 - protein, 332 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 71 - protein, 313 aa, synthetic construct
EAGVAQSPRYKIIEKRQSVAFWCNPISGHATLYWYQQILGQGPKLLIQFQNNGVVDDSQLPKDRFSAERLKGVDSTLKIQPAKLEDSAVYLCASSIRHRTNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPG

SEQ ID NO: 72 - protein, 332 aa, synthetic construct
MGTRLLCWAALCLLGAELTEAGVAQSPRYKIIEKRQSVAFWCNPISGHATLYWYQQILGQGPKLLIQFQNNGVVDDSQLPKDRFSAERLKGVDSTLKIQPAKLEDSAVYLCASSIRHRTNTEAFFGQGTRLTVVEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDFGSGATNFSLLKQAGDVEENPG

SEQ ID NO: 73 - protein, 253 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 74 - protein, 273 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 75 - protein, 297 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 76 - protein, 318 aa, synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 77 - protein, 311 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 78 - protein, 332 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 79 - protein, 606 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 80 - protein, 253 aa, Homo sapiens
SQQGEEDPQALSIQEGENATMNCSYKTSINNLQWYRQNSGRGLVHLILIRSNEREKHSGRLRVTLDTSKKSSSLLITASRAADTASYFCATDATSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 81 - protein, 273 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDATSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 82 - protein, 297 aa, Homo sapiens
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 83 - protein, 318 aa, synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 84 - protein, 311 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 85 - protein, 332 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPG

SEQ ID NO: 86 - protein, 606 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGSLNEQFFGPGTRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDATSGTYKYIFGTGTRLKVLANIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 87 - DNA, 1821 bp, synthetic construct
  source 1..1821: mol_type = other DNA
atgctcctgctccttctgctgctgggacccggttccggcctcggcgctgtggtgtcacagcacccttcatgggtgatctgcaagagtgggactagcgtgaagatcgagtgtcggtccctggacttccaagccaccaccatgttctggtaccggcagttcccgaagcagtcgttgatgctgatggccacttccaacgagggcagcaaggctacctacgaacagggcgtggaaaaggacaagtttctgattaaccacgcatccctgaccctgtcgactctcaccgtgacctcggcccaccccgaggatagctccttctacatctgctccgcgcgcgatctgacttccggagccaacaacgaacagtttttcggccctggtactagactcactgtgctggaagatctgaagaacgtgttcccgcccgaagtggccgtgttcgagccctccgaagccgagatctcccatactcaaaaagccacccttgtctgcctggctaccggattctaccccgatcacgtggagctctcttggtgggtgaacggaaaggaagtgcattccggagtctgcaccgaccctcagccgctgaaagaacagccagccctgaacgactcccgctactgcctgagctcacggctgagagtgtccgccaccttctggcaaaatcctaggaaccacttccggtgccaagtgcagttctatggactgagcgagaacgacgaatggacccaggacagagcgaagccggtcacacagatcgtgtcagcggaggcctgggggcgcgccgactgtggattcacgtcggaaagctaccagcagggcgtgctgtcggcaaccattttgtacgagattttgctcgggaaggccaccctgtacgcggtgctcgtcagcgccctcgtccttatggcaatggtcaagcgcaaggactcccggggtggcagcggcgccaccaacttctccctgctgaagcaggccggcgacgtggaagaaaaccctggccccatggaaaccctgctgggagtgtcacttgtgatcttgtggctgcaactggcgcgcgtcaacagccaacagggcgaagaggatccacaggccctgagcatccaggaaggggagaacgcgactatgaactgcagctacaagactagcatcaacaatctgcagtggtaccgccagaactccggccgagggcttgtgcacctgattctgattcggagcaatgagcgggagaagcattccggacggctcagggtcaccctggacacctccaaaaagtcgagctcactcctgattacggcctcaagagccgctgacactgcttcctatttctgcgccactgacactacatcaggaacctacaagtacatcttcggaaccggaacccgcttgaaagtcctggccaacatccagaacccagacccggcagtgtaccagctgcgggactccaagtcctcggataagagcgtgtgcctattcaccgacttcgactcccaaactaacgtgtcccagtccaaagatagcgacgtgtacatcaccgacaagtgcgttctggacatgcgctccatggacttcaagtcgaactcggccgtggcctggtccaacaagtccgatttcgcgtgcgccaatgcgttcaacaactccatcattcctgaggataccttcttcccgtccccggagtcgtcgtgtgatgtgaagctcgtggagaagtccttcgaaaccgacaccaacctgaactttcaaaacctttcggtgatcggttttaggatcctgctgctcaaagtcgccgggtttaatctgctcatgaccctgcggctctggtcctcttaa

SEQ ID NO: 88 - DNA, 1821 bp, synthetic construct
  source 1..1821: mol_type = other DNA
atgctgctgcttctgctgcttctggggccaggttccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagacctcacttccggctccctgaatgagcagttcttcgggccagggacacggctcaccgtgctagaggacctgaaaaacgtgttcccacccgaggtcgctgtgtttgagccatcagaagcagagatctcccacacccaaaaggccacactggtgtgcctggccacaggcttctaccccgaccacgtggagctgagctggtgggtgaatgggaaggaggtgcacagtggggtctgcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacctgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggccatggtgaagagaaaggattccagaggcggcagcggcgccaccaacttctccctgctgaagcaggccggcgacgtggaagaaaaccctggccccatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacgctacctcaggaacctacaaatacatctttggaacaggcaccaggctgaaggttttagcaaatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaatgtgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagctga

SEQ ID NO: 89-90 - skipped (2 entries)

SEQ ID NO: 91 - protein, 249 aa, synthetic construct
SQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 92 - protein, 269 aa, synthetic construct
METLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 93 - protein, 291 aa, synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 94 - protein, 312 aa, synthetic construct
GAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPG

SEQ ID NO: 95 - protein, 305 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 96 - protein, 326 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPG

SEQ ID NO: 97 - protein, 596 aa, synthetic construct
MLLLLLLLGPGSGLGAVVSQHPSWVICKSGTSVKIECRSLDFQATTMFWYRQFPKQSLMLMATSNEGSKATYEQGVEKDKFLINHASLTLSTLTVTSAHPEDSSFYICSARDLTSGANNEQFFGPGTRLTVLEDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNSGSGATNFSLLKQAGDVEENPGPMETLLGVSLVILWLQLARVNSQQGEEDPQALSIQEGENATMNCSYKTSINNLQWYRQNSGRGLVHLILIRSNEREKHSGRLRVTLDTSKKSSSLLITASRAADTASYFCATDTTSGTYKYIFGTGTRLKVLANIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 98 - protein, 136 aa, Mus musculus
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 99 - protein, 172 aa, Mus musculus
DLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 100 - protein, 20 aa, synthetic construct
METLLGVSLVILWLQLARVN

SEQ ID NO: 101 - protein, 14 aa, synthetic construct
MLLLLLLLGPGSGL

SEQ ID NO: 102 - protein, 8 aa, synthetic construct
DXEXNPGP

SEQ ID NO: 103 - protein, 18 aa, Thosea asigna virus
EGRGSLLTCGDVEENPGP

SEQ ID NO: 104 - protein, 19 aa, Teschovirus A
ATNFSLLKQAGDVEENPGP

SEQ ID NO: 105 - protein, 20 aa, Equine rhinitis A virus
QCTNYALLKLAGDVESNPGP

SEQ ID NO: 106 - protein, 22 aa, Enterovirus B
VKQTLNFDLLKLAGDVESNPGP

SEQ ID NO: 107 - DNA, 1788 bp, synthetic construct
  source 1..1788: mol_type = other DNA
atgctgctgcttctgctgcttctggggccaggttccgggcttggtgctgtcgtctctcaacatccgagctgggttatctgtaagagtggaacctctgtgaagatcgagtgccgttccctggactttcaggccacaactatgttttggtatcgtcagttcccgaaacagagtctcatgctgatggcaacttccaatgagggctccaaggccacatacgagcaaggcgtcgagaaggacaagtttctcatcaaccatgcaagcctgaccttgtccactctgacagtgaccagtgcccatcctgaagacagcagcttctacatctgcagtgctagagacctcacttccggcgccaataatgagcagttcttcgggccagggacacggctcaccgtgctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattccggcagcggcgccaccaacttctccctgctgaagcaggccggcgacgtggaagaaaaccctggccccatggaaactctcctgggagtgtctttggtgattctatggcttcaactggctagggtgaacagtcaacagggagaagaggatcctcaggccttgagcatccaggagggtgaaaatgccaccatgaactgcagttacaaaactagtataaacaatttacagtggtatagacaaaattcaggtagaggccttgtccacctaattttaatacgttcaaatgaaagagagaaacacagtggaagattaagagtcacgcttgacacttccaagaaaagcagttccttgttgatcacggcttcccgggcagcagacactgcttcttacttctgtgctacggacactacctcaggaacctacaaatacatctttggaacaggcaccaggctgaaggttttagcaaacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgactgagaaaagctttgaaacagatatgaacctaaactttcaaaacctgtcagttatgggactccgaatcctcctgctgaaagtagccggatttaacctgctcatgacgctgaggctgtggtccagt

SEQ ID NO: 108 - protein, 596 aa, synthetic construct; identical to SEQ ID NO: 97

SEQ ID NO: 109 - protein, 7 aa, Homo sapiens
KTSINNL

SEQ ID NO: 110 - protein, 9 aa, Homo sapiens
LIRSNEREK

SEQ ID NO: 111 - protein, 14 aa, Homo sapiens
CATDTTSGTYKYIF

SEQ ID NO: 112 - protein, 112 aa, Homo sapiens; identical to SEQ ID NO: 4

SEQ ID NO: 113 - protein, 249 aa, Homo sapiens; identical to SEQ ID NO: 91

SEQ ID NO: 114 - protein, 269 aa, Homo sapiens; identical to SEQ ID NO: 92

SEQ ID NO: 115 - protein, 8 aa, Homo sapiens
LDFQATTM

SEQ ID NO: 116 - protein, 9 aa, Homo sapiens
TSNEGSKAT

SEQ ID NO: 117 - protein, 16 aa, Homo sapiens
CSARDLTSGANNEQFF

SEQ ID NO: 118 - protein, 118 aa, Homo sapiens; identical to SEQ ID NO: 10

SEQ ID NO: 119 - protein, 291 aa, Homo sapiens; identical to SEQ ID NO: 93

SEQ ID NO: 120 - protein, 312 aa, synthetic construct; identical to SEQ ID NO: 94

SEQ ID NO: 121 - protein, 305 aa, synthetic construct; identical to SEQ ID NO: 95

SEQ ID NO: 122 - protein, 326 aa, synthetic construct; identical to SEQ ID NO: 96

SEQ ID NO: 123 - protein, 137 aa, Homo sapiens
NIQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 124 - protein, 173 aa, Homo sapiens
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS